(19) United States
(12) Patent Application Publication
(10) Pub. No.: US 2026/0309715 A1
(43) Pub. Date: Oct. 8, 2026

(54) NOVEL LILRB2 ANTIBODIES AND USES THEREOF

(71) Applicants: THE BOARD OF REGENTS OF THE UNIVERSITY OF TEXAS SYSTEM, Austin, TX (US); IMMUNE-ONC THERAPEUTICS, INC., Palo Alto, CA (US)

(72) Inventors: Zhiqiang AN, Houston, TX (US); Ningyan ZHANG, Houston, TX (US); Zhiqiang KU, Houston, TX (US); Chengcheng ZHANG, Southlake, TX (US); Xiaoye LIU, Dallas, TX (US); Heyu CHEN, Dallas, TX (US); JingJing XIE, Plano, TX (US); Maria Jose COSTA, Palo Alto, TX (US); An SONG, Palo Alto, TX (US); X. Charlene LIAO, Palo Alto, TX (US)

(73) Assignees: THE BOARD OF REGENTS OF THE UNIVERSITY OF TEXAS SYSTEM, Austin, TX (US); IMMUNE-ONC THERAPEUTICS, INC., Palo Alto, CA (US)

(21) Appl. No.: 19/559,692

(22) Filed: Mar. 6, 2026

Related U.S. Application Data
(63) Continuation of application No. 17/759,800, filed on Jan. 28, 2021, now Pat. No. 12,596,122.
(60) Provisional application No. 62/970,496, filed on Feb. 5, 2020.

(51) Int. Cl. G01N 33/575; A61K 39/00; A61K 39/395; A61K 45/06; A61K 47/68; A61P 35/02; C07K 16/28

(52) U.S. Cl. CPC G01N 33/5759; A61K 39/3955; A61K 45/06; A61K 47/6849; A61P 35/02; C07K 16/2803; A61K 2039/505; C07K 2317/76; C07K 2317/92

(57) ABSTRACT

Provided herein are antibodies binding to LILRB2 and the uses of the antibodies in detecting and treating cancer and autoimmune diseases.

### PRIORITY CLAIM

This application is a continuation of U.S. application Ser. No. 17/759,800, filed Jul. 29, 2022, as a national phase application under 35 U.S.C. § 371 of International Application No. PCT/US2021/015362, filed Jan. 28, 2021, which claims benefit of priority to U.S. Provisional Application Ser. No. 62/970,496, filed Feb. 5, 2020, the entire contents of which are hereby incorporated by reference.

### SEQUENCE LISTING

This application contains a Sequence Listing XML, which has been submitted electronically and is hereby incorporated by reference in its entirety. Said Sequence Listing XML, created on Mar. 6, 2026, is named UTSHP0359USC1.xml, and is 721,477 bytes in size.

### BACKGROUND

### 1. Field

The present disclosure relates generally to the fields of medicine, oncology, immunology and immuno-oncology. More particular, the disclosure relates to antibodies that bind to LILRBs and can treat cancers, including leukemia and solid tumors.

### 2. Description of Related Art

Current immune checkpoint blockade strategies have been successful in treating certain types of solid cancer. However, most cancer patients do not respond to current checkpoint blockade or they relapse after treatment. Additionally, checkpoint blockade monotherapies have not been successful against most hematologic malignancies including multiple myeloma and leukemia.

It is believed that tumor microenvironment (TME) plays a critical role in regulating immune responses to tumors. Among the complex factors and components that constitute the tumor microenvironment, myeloid derived suppressor cells (MDSC), tumor associated macrophages (TAMs) and extracellular matrix all play critical role in suppressing the immune responses to tumor.

Recently, it has been shown that inhibitory leukocyte immunoglobulin-like receptors (LILRBs) and a related immunoreceptor tyrosine-based inhibitory motif (ITIM)-containing receptor, LAIR1, have tumor-promoting functions in various hematopoietic and solid cancer cells and in the immunosuppressive tumor microenvironment. ITIM-containing receptors are expressed on a wide range of immune cells and transduce signals by recruitment of phosphatases SHP-1, SHP-2, or SHIP, leading to negative regulation of immune cell activation. Similar to CTLA-4 and PD-1, LILRBs are considered immune checkpoint factors.

LILRB2 has been identified as a key regulator of myeloid cell phenotype in vitro and in vivo as its activation by various ligands suppresses the pro-inflammatory activity of myeloid cells. Because myeloid cells with a suppressive/anti-inflammatory phenotype can down-regulate the activation, proliferation and cytotoxic activity of T cells in the solid tumor microenvironment, therapeutic blocking of LILRB2 in myeloid-rich solid tumors has the potential to reactivate or enhance anti-tumor immune responses in patients presenting with disease unresponsive/relapsed to T cell checkpoint inhibitors.

LILRBs may inhibit activities of a number of immune cell types facilitating tumour immune escape. LILRB2 belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on myeloid cells; it binds to multiple types of ligands, including HLA class I molecules, ANGPTLs, myelin inhibitors (including Nogo66, MAG, and OMgp), and β-amyloid, transducing a negative signal that inhibits stimulation of an immune response. It is thought to control inflammatory responses and cytotoxicity to help focus the immune response and limit autoreactivity. Multiple transcript variants encoding different isoforms have been found for this gene.

Conversely, by agonising LILRB family of receptors, we may be able to suppress immune response or inflammations found in autoimmune or inflammatory diseases.

### SUMMARY

Thus, in one aspect, the present disclosure provides an isolated monoclonal antibody or an antigen-binding fragment thereof that binds specifically to LILRB2. In certain embodiments, the antibody or antigen-binding fragment, when bound to LILRB2, modulates the activation of LILRB2. In certain embodiments, the antibody or antigen-binding fragment, when bound to LILRB2, activates LILRB2. In certain embodiments, the antibody or antigen-binding fragment, when bound to LILRB2, suppresses activation of LILRB2. In certain embodiments, the antibody or antigen-binding fragment, when bound to LILRB2, specifically blocks binding of MHC and other ligands to LILRB2.

In one aspect, the isolated monoclonal antibody or an antigen-binding fragment thereof comprises a heavy chain (HC) variable region (VH) and a light chain (LC) variable region (VL) comprising the clone-paired CDR sequences as set forth in Table 2; and variants thereof wherein one or more of the LC-CDRs has one, two, or three amino acid substitutions, additions, deletions, or combinations thereof. The isolated monoclonal antibody or an antigen binding fragment thereof of claim 1, wherein the isolated monoclonal antibody is a murine, a rodent, a rabbit, a chimeric, humanized, or human antibody. The isolated monoclonal antibody or an antigen-binding fragment thereof may have VH and VL chains with amino acid sequences at least 90% or 95% identical to the clone-paired sequences of Appendices II and IV, respectively. The isolated monoclonal antibody or an antigen-binding fragment thereof may have VH and VL chains encoded by nucleic acid sequences at least 80% or 90% identical to the clone-paired sequences of Appendices I and III, respectively. The isolated monoclonal antibody or an antigen-binding fragment thereof of may have VH and VL chains with amino acid sequences identical to the clone-paired sequences of Appendices II and IV, respectively. The isolated monoclonal antibody or an antigen binding fragment thereof may have VH and VL chains encoded by nucleic acid sequences identical to the clone-paired sequences of Appendices I and III, respectively.

The variants may be those where one or more of the HC-CDRs or LC-CDRs has one, two, or three amino acid substitutions, additions, deletions, or combinations thereof. In certain embodiments, each CDR is defined in accordance with Kabat definition, the Chothia definition, the combination of Kabat definition and Chothia definition, the AbM definition, or the contact definition of CDR.

In another aspect, the present disclosure provides an isolated monoclonal antibody or an antigen-binding fragment thereof, which competes for the same epitope with an antibody having clone-paired heavy and light chain CDR sequences from Table 2. In certain embodiments, the epitope bound by the antibody or antigen-binding fragment is located within the linker region between the D1 and D2 domain of human LILRB2.

In certain embodiments, the isolated monoclonal antibody described herein is a chimeric, humanized, or human antibody. In certain embodiments, isolated monoclonal antibody described herein is of the IgG1, IgG2, IgG3 or IgG4 type. In certain embodiments, the antigen-binding fragment described herein is a recombinant ScFv (single chain fragment variable) antibody, Fab fragment, F(ab′)2 fragment, or Fv fragment.

In another aspect, there is provided a pharmaceutical composition comprising an isolated monoclonal antibody or an antigen-binding fragment thereof as provided herein, and at least one pharmaceutically acceptable carrier.

In another aspect, there is provided an isolated nucleic acid that encodes the isolated monoclonal antibody or an antigen-binding fragment thereof as provided herein.

In another aspect, there is provided a vector comprising the isolated nucleic acid as provided herein.

In another aspect, there is provided a host cell comprising the vector as provided herein. The host cell may be a mammalian cell. The host cell may be a CHO cell.

In another aspect, there is provided a hybridoma encoding or producing the isolated monoclonal antibody as provided herein.

In another aspect, there is provided a process of producing an antibody. The method may comprise culturing the host cell as provided herein under conditions suitable for expressing the antibody and recovering the antibody.

In another aspect, there is provided a chimeric antigen receptor (CAR) protein comprising an antigen-binding fragment as provided herein.

In another aspect, there is provided an isolated nucleic acid that encodes a CAR protein as provided herein.

In another aspect, there is provided an engineered cell comprising the isolated nucleic acid as provided herein. In certain embodiments, the cell is a T cell, NK cell, or myeloid cell.

In another, there is provided a method of treating or ameliorating the effect of a cancer in a subject, the method comprising administering to the subject a therapeutically effective amount of the antibody or an antigen-binding fragment thereof as defined herein.

The method may reduce or eradicate the tumor burden in the subject, may and/or slow tumor growth rate, may reduce the number of tumor cells, may reduce tumor size, may reduce tumor infiltration, may reduce tumor metastasis, may eradicate the tumor in the subject. The cancer may be a solid tumor or hematologic malignancy.

In certain embodiments, the cancer is a solid tumor including adrenal cancer, bile duct carcinoma, bone cancer, brain cancer, breast cancer, cervical cancer, choriocarcinoma, colon cancer, colorectal cancer, esophageal cancer, eye cancer, gastric cancer, glioblastoma, head and neck cancer, kidney cancer, liver cancer, lung cancer, mesothelioma, melanoma, merkel cell cancer, nasopharyngeal carcinoma, neuroblastoma, oral cancer, ovarian cancer, pancreatic cancer, penile cancer, pinealoma, prostate cancer, renal cell cancer, retinoblastoma, sarcoma, skin cancer, testicular cancer, thymic carcinoma, thyroid cancer, uterine cancer, and vaginal cancer.

In some embodiments, the cancer is a metastatic, recurrent or drug-resistant cancer.

In some embodiments, said cancer is hematologic malignancies including acute lymphocytic leukemia (ALL), acute myeloid leukemia (AML), B-cell leukemia, blastic plasmacytoid dendritic cell neoplasm (BPDCN), chronic lymphoblastic leukemia (CLL), chronic myelomonocytic leukemia (CMML), chronic myelocytic leukemia (CML), pre-B acute lymphocytic leukemia (Pre-B ALL), diffuse large B-cell lymphoma (DLBCL), extranodal NK/T-cell lymphoma, hairy cell leukemia, HHV8-associated primary effusion lymphoma, plasmablastic lymphoma, primary CNS lymphoma, primary mediastinal large B-cell lymphoma, T-cell/histiocyte-rich B-cell lymphoma, heavy chain disease, Hodgkin's lymphoma, non-Hodgkin's lymphoma, Waldenstrom's macroglobulinemia, multiple myeloma (MM), myelodysplastic syndromes (MDS), myeloproliferative neoplasms, and polycythemia vera.

The antibody or an antigen-binding fragment thereof may be administered intravenously, intra-arterially, intra-tumorally, or subcutaneously.

In certain embodiments, the method may further comprise administering to the subject one or more drugs selected from the group consisting of administering to the subject one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICAR, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRP1α antibody or fusions protein, a CD70 antibody, and CLL1 antibody, a CD123 antibody, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

The isolated monoclonal antibody or an antigen binding fragment thereof may comprise an antitumor drug linked thereto. The antitumor drug may be linked to said antibody through a photolabile linker. The antitumor drug may be linked to said antibody through an enzymatically-cleaved linker. The antitumor drug may a toxin, a radioisotope, a cytokine, or an enzyme.

In another embodiment, there is provided a method of detecting a cancer cell or cancer stem cell in a sample or subject comprising (a) contacting a subject or a sample from the subject with the antibody or an antigen-binding fragment thereof as defined herein; and (b) detecting binding of said antibody to a cancer cell or cancer stem cell in said subject or sample. The sample may be a body fluid or biopsy, or blood, bone marrow, sputum, tears, saliva, mucous, serum, urine or feces. Detection may comprise immunohistochemistry, flow cytometry, immunoassays (including ELISA, RIA etc.) or Western blot. The method may further comprise performing steps (a) and (b) a second time and determining a change in detection levels as compared to the first time. The isolated monoclonal antibody or an antigen binding fragment thereof may further comprise a label, such as a peptide tag, an enzyme, a magnetic particle, a chromophore, a fluorescent molecule, a chemo-luminescent molecule, or a dye. The isolated monoclonal antibody or an antigen binding fragment thereof may be conjugated to a liposome or nanoparticle.

In still an additional aspect, there is provided a method of treating or ameliorating the effect of an autoimmune disease in a subject, the method comprising administering to the subject a therapeutically effective amount of the antibody or an antigen-binding fragment thereof as defined herein. The antibody or an antigen-binding fragment thereof may be administered intravenously, intra-arterially, intra-tumorally, or subcutaneously. The method may further comprise administering to the subject one or more drugs selected from the group consisting of a steroid or an NSAID. The autoimmune disease may be Guillain-Barre syndrome, Chronic inflammatory demyelinating polyneuropathy, ankylosing spondylitis, psoriatic arthritis, enteropathic arthritis, reactive arthritis, undifferentiated spondyloarthropathy, juvenile spondyloarthropathy, Behcet's disease, enthesitis, ulcerative colitis, Crohn's disease, irritable bowel syndrome, inflammatory bowel disease, fibromyalgia, chronic fatigue syndrome, pain conditions associated with systemic inflammatory disease, systemic lupus erythematosus, Sjogren's syndrome, rheumatoid arthritis, juvenile rheumatoid arthritis, juvenile onset diabetes mellitus (also known as Type I diabetes mellitus), Wegener's granulomatosis, polymyositis, dermatomyositis, inclusion body myositis, multiple endocrine failure, Schmidt's syndrome, autoimmune uveitis, Addison's disease, Grave's Disease, Hashimoto's thyroiditis, autoimmune thyroid disease, pernicious anemia, gastric atrophy, chronic hepatitis, lupoid hepatitis, atherosclerosis, multiple sclerosis, amyotrophic lateral sclerosis, hypoparathyroidism, Dressler's syndrome, myasthenia gravis, Eaton-Lambert syndrome, autoimmune thrombocytopenia, idiopathic thrombocytopenia purpura, hemolytic anemia, pemphigus vulgaris, pemphigus, dermatitis herpetiformis, alopecia, scleroderma, progressive systemic sclerosis, CREST syndrome (calcinosis, Raynaud's phenomenon, esophageal dysmotility, sclerodactyly, and telangtasia), adult onset diabetes mellitus (also known as Type II diabetes mellitus), mixed connective tissue disease, polyarteritis nodosa, systemic necrotizing vasculitis, glomerulonephritis, atopic dermatitis, atopic rhinitis, Goodpasture's syndrome, Chagas' disease, sarcoidosis, rheumatic fever, asthma, anti-phospholipidsyndrome, erythema multiforme, Cushing's syndrome, autoimmune chronic active hepatitis, allergic disease, allergic encephalomyelitis, transfusion reaction, leprosy, malaria, leshmaniasis, trypanosomiasis, Takayasu's arteritis, polymyalgia rheumatica, temporal arteritis, shistosomiasis, giant cell arteritis, eczema, lymphomatoid granulomatosis, Kawasaki's disease, endophthalmitis, psoriasis, erythroblastosis fetalis, eosinophilic faciitis, Shulman's syndrome, Felty's syndrome, Fuch's cyclitis, IgA nephropathy, Henoch-Schonlein purpura, graft versus host disease, transplantation rejection, tularemia, periodic fever syndromes, pyogenic arthritis, Familial Mediterranean Fever, TNF-receptor associated periodic syndrome (TRAPS), Muckle-Wells syndrome, or hyper-IgD syndrome.

Also provided is monoclonal antibody that binds to LILRB2 and (a) does not bind to LILRA or another LILRB; (b) binds to LILRB2 Domain 1 or 4; (c) activates or antagonizes LILRB2; (d) enhances monocyte inflammatory potential; (e) prevents myeloid-derived suppressor cell function; and/or (f) inhibits leukemia cell migration and/or infiltration in vivo.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D. Screening specific monoclonal antibodies for LILRB2. (FIG. 1A) Representative flow cytometric profiles showing that monoclonal antibodies bind to LILRB2 reporter cells. Binding of monoclonal antibodies was screened using flow cytometer on LILRB2 reporter cells. Bound antibodies were detected with Allophycocyanin (APC)-labeled goat anti-human IgG secondary antibody. (FIG. 1B) Quantification of binding ability of monoclonal antibodies to LILRB2 reporter cells. B2-7, B2-15, B2-16, B2-17, B2-19, B2-8, B2-24, B2-25, B2-10, B2-12 and B2-18 highly bind to LILRB2 reporter cells. (FIGS. 1C-D) Quantification of binding ability of monoclonal antibodies B2-7, B2-15, B2-16, B2-17, B2-19, B2-8, B2-24, B2-25, B2-10, B2-12 and B2-18 to LILRAs, LILRBs and LAIR1 reporter cells.

FIGS. 2A-B. Antibodies B2-8, B2-24, B2-10, B2-10 and B2-15 increase GFP signaling of LILRB2 reporter cells. (FIG. 2A) GFP signaling of LILRB2 reporter cells incubated with soluble antibodies. (FIG. 2B) GFP signaling of LILRB2 reporter cells incubated with soluble antibodies combined with K562.

FIGS. 3A-C. Antibodies B2-7, B-15, B2-16, B2-17 and B2-19 block GFP signaling of LILRB2 reporter cells activated by coated ANGPTL2. (FIG. 3A) Representative flow cytometric profiles showed that coated ANGPTL2 stimulates GFP expression in LILRB2 reporter cells. (FIG. 3B) Representative flow cytometric profiles showed that B2-7, B-15, B2-16, B2-17 and B2-19 effectively block GFP expression in LILRB2 reporter cells activated by coated ANGPTL2. (FIG. 3C) Dose-dependent inhibitory ability of B2-7, B-15, B2-16, B2-17 and B2-19 to GFP expression induced by coated ANGPTL2. Blocking potency (IC₅₀) of B2-19, B2-16, B2-7, B2-15 and B2-17 was 48.54 ng/ml, 131.4 ng/ml, 221.1 ng/ml, 341.3 ng/ml and 405.1 ng/ml respectively.

FIGS. 4A-C. Antibodies B2-7, B-15, B2-16, B2-17 and B2-19 block GFP signaling of LILRB2 reporter cells activated by coated SEMA4A. (FIG. 4A) Representative flow cytometric profiles showed that coated SEMA4A stimulates GFP expression in LILRB2 reporter cells. (FIG. 4B) Representative flow cytometric profiles showed that B2-7, B-15, B2-16, B2-17 and B2-19 effectively block GFP expression in LILRB2 reporter cells activated by coated SEMA4A. (FIG. 4C) Dose-dependent inhibition of B2-7, B-15, B2-16, B2-17 and B2-19 to GFP expression induced by coated SEMA4A. Blocking potency (IC₅₀) of B2-19, B2-16, B2-7, B2-15 and B2-17 was 167.1 ng/ml, 449 ng/ml, 701 ng/ml, 1001 ng/ml and 1034 ng/ml respectively.

FIGS. 5A-C. Antibodies B2-7, B-15, B2-16, B2-17 and B2-19 block GFP signaling of LILRB2 reporter cells activated by HLA-G overexpressed on K562 cells. (FIG. 5A) Representative flow cytometric profiles showed that HLA-G overexpressed on K562 cells stimulates GFP expression in LILRB2 reporter cells. (FIG. 5B) Representative flow cytometric profiles showed that B2-7, B-15, B2-16, B2-17 and B2-19 effectively block GFP expression in LILRB2 reporter cells activated by HLA-G overexpressed on K562 cells. (FIG. 5C) Quantification of GFP percentage shown in FIG. 5B.

FIGS. 6A-C. The effect of LILRB2 antibodies on LPS response in primary human monocytes. (FIGS. 6A-B) Representative flow cytometric profiles showed that surface CD86 and intracellular TNFα staining of cells gated on CD33+ monocytes. PBMCs were cultured for 48 hours with anti-LILRB2 antibody (10 ng/ml) followed by 6 hours of LPS stimulation (50 ng/ml) in the presence of brefeldin A. (FIG. 6C) Quantification of fold changes was defined by the ratio of mean fluorescence intensity (MFI) of CD86 and TNFα in anti-LILRB2 treated samples relative to their respective MFI in IgG treated samples shown in FIG. 6A-B. MFI values represent cells gated on CD33+ monocytes.

FIGS. 7A-J. Antagonistic LILRB2 mAbs inhibit the development of leukemia cells in C1498-LILRB2 tumor-bearing model. (FIG. 7A) Human LILRB2 expression on mouse C1498 parental cells and human LILRB2 retroviral transduced C1498 cells. (FIG. 7B) LILRB2 promotes the death of leukemia-bearing mice. Kaplan-Meier survival curve of humanized NSG mice which were i.v. injected with LILRB2-overexpressed or control (ctrl) C1498 cells (1×10⁶ cells per mouse). (FIG. 7C-D) Representative flow cytometry plots and summary of leukemia cell infiltration in bone marrow (BM), peripheral blood (PB), liver (LV) and spleen (SP) from C57BL/6 which were i.v. injected with LILRB2-overexpressed or ctrl C1498 cells (1×10⁶ cells per mouse). (FIG. 7E) Kaplan-Meier survival curve of C57BL/6 mice which were i.v. injected with LILRB2-overexpressed or ctrl C1498 cells (1×10⁶ cells per mouse). (FIG. 7F-G) Representative flow cytometry plots and summary of myeloid cell infiltration in peripheral blood (PB), from C57BL/6 which were i.v. injected with LILRB2-overexpressed or ctrl C1498 cells (1×10⁶ cells per mouse). (FIG. 7H-I) Representative flow cytometry plots and summary of leukemia cell infiltration in peripheral blood (PB) from C56BL/6 mice with the treatment of LALAPG mutated anti-LILRB2 antibodies or IgG control after leukemia cell transplant. LILRB2-overexpressed C1498 cells (1×10⁶ cells per mouse) were injected into C57BL/6 mice followed by treatment with LALAPG Fc mutated anti-LILRB2 antibodies or LALAPG Fc mutated IgG. The percentage of leukemia cell (GFP+) from peripheral blood (PB) was determined by flow cytometry 20 days after transplantation. (FIG. 7J) Summary of myeloid cell infiltration in peripheral blood (PB) from C56BL/6 mice treated at indicated time after leukemia cell transplant with LALAPG mutated anti-LILRB2 antibodies or LALAPG Fc mutated IgG control.

FIGS. 8A-F. Antagonistic LILRB2 mAbs inhibit the development of leukemia cells in MLL-AF9 leukemia model. (FIG. 8A) The expression of LILRB2 on PIRB-KO MLL-AF9 leukemia cells transduced with LILRB2 (LILRB2) or Control (Ctrl) leukemia cells. (FIG. 8B-C) Representative flow cytometry plots and summary of leukemia cell infiltration in peripheral blood (PB) from C56BL/6 mice transplanted with PirB-KO MLL-AF9 leukemia cells transduced with LILRB2 (LILRB2) or control PirB-KO MLL-AF9 (Ctrl) leukemia cells. (FIG. 8D) Kaplan-Meier survival curve of leukemia mice which were transplanted with LILRB2-overexpressed or Ctrl PirB-KO MLL-AF9 cells. (FIG. 8E-F) Representative flow cytometry plots and summary of leukemia cell infiltration in peripheral blood (PB) from C56BL/6 mice transplanted with PirB-KO LILRB2 leukemia cells and followed by the treatment of LALAPG mutated anti-LILRB2 antibodies or IgG control.

FIGS. 9A-G. Anti-LILRB2 antibodies inhibit the migration and infiltration of AML cells. (FIG. 9A) LILRB2 expression on THP-1 cells was confirmed using a commercial phycoerythrin (PE)-anti-LILRB2 antibody. (FIG. 9B) Short-term (20 h) infiltration of leukemia cells in NSG mice treated with LALAPG Fc mutated anti-LILRB2 antibodies or LALAPG Fc mutated IgG control after leukemia transplant. THP-1 cells (1×10⁷ cells per mouse) were injected into NSG mice followed by treatment with LALAPG mutated IgG control or anti-LILRB2 antibodies immediately. The numbers of leukemia cells (GFP+) from bone marrow (BM), liver (LV) and spleen (SP) were determined by flow cytometry 20 hours after transplantation and normalized to number in peripheral blood (PB). (FIG. 9C) Percentage of leukemia cells (GFP+) in indicated organs such as liver (LV), bone marrow (BM), spleen (SP) and peripheral blood (PB) at day 21 post-transplant in NSG mice treated with LALAPG mutated anti-LILRB2 antibodies or IgG control after THP-1 injection. (FIG. 9D) Body weight for each mouse shown in FIG. 9B was measured at indicated day after THP-1 cells injection. (FIG. 9E) Comparison of the size of livers from NSG mice at 28 days after THP-1 transplant and treated with IgG or anti-LILRB2 antibodies containing the LALAPG Fc mutations. (FIG. 9F) Quantification of liver weight shown in FIG. 9E normalized to respective individual mouse body weight. (FIG. 9G) Survival curve of NSG mice treated with LALAPG Fc mutated IgG control or anti-LILRB2 antibodies after THP-1 transplantation.

FIGS. 10A-E. Antagonistic LILRB2 mAbs inhibit the development of leukemia cells in patient-derived xenograft (PDX) model. (FIG. 10A) LILRB2 expression pattern on primary leukemia cells from AML M5 patients. (FIG. 10B) Analysis of correlation between LILRB2 mRNA levels and the overall survival of patients with AML-M5 (n=132, divided into two groups based on gene expression) in TCGA database (https://xena.ucsc.edu) by Kaplan-Meier long-rank test. (FIG. 10C) Human CD45+CD33⁺ leukemia cells infiltration in peripheral blood (PB), bone marrow (BM), spleen (SP) and liver (LV) of NSG mice transplanted with primary AML-M5 leukemia samples and treated with anti-LILRB2 antibodies or control IgG. (FIG. 10D) Representative bright-field microcopy image of primary AML-M5 leukemia cell cultured in the present of LALAPG mutated anti-LILRB2 antibodies or IgG control. Anti-LILRB2 treated cells showed more adherent differentiation morphology. (FIG. 10E) Intracellular expression of CD68 on primary AML-M5 leukemia cell cultured in the present of LALAPG mutated anti-LILRB2 antibodies or IgG control.

FIGS. 11A-G. Antagonistic LILRB2 mAbs can prevent the T cell suppressive function of Myeloid-Derived Suppressor Cells (MDSC) in vitro. (FIG. 11A) One representative histogram showed that antagonistic LILRB2 mAbs attenuated the suppressive function of MDSC towards CD8+ T cells. The MDSC were isolated from the PBMC of patients with solid tumor, by depleting HLD-DRbright cells and then enriching the CD14+ cells, using autoMACS. MDSC were cocultured with T cells from the same donor (E: T=1), which was pre-stained with CSFE to monitor the cell proliferation. 10 μg/mL LALAPG mutated IgG, B2-7, or B2-19 was added into the cell culture. The percentage of proliferative T cells, indicated by reduced intensity of CFSE signal, was determined by flow cytometry 5 days after treatments. (FIG. 11B) Quantification of the effects of anti-LILRB2 mAbs on the inhibitory functions of MDSC towards T cells. The percentage of proliferative T cells (left panel: CD8+ T cells, right panel CD4+ T cells), indicated by reduced intensity of CFSE signal, was determined by flow cytometry 5-7 days after treatments. (FIG. 11C) Anti-LILRB2 mAbs attenuated the inhibitory functions of MDSC towards T cells, accessed by measuring the IFN-γ secretion in the supernatants of T cell cultures. (FIG. 11D) Anti-LILRB2 mAbs decreased the expression of M2 macrophage markers, while increased the expression of M1 macrophage markers, on MDSC. The MDSC isolated from peripheral blood of patients with solid tumors were cultured with 10 μg/mL anti-LILRB2 antibodies for 7 days. The expression of CD163, CD206 and CD86 were analyzed by flow cytometry. (FIG. 11E) Anti-LILRB2 mAbs decreased the M2 markers expression, while increased M1 markers expression, on monocyte-derived macrophage from a healthy donor. The monocytes isolated from the peripheral blood of a health donor were cultured and incubated with 10 μg/mL anti-LILRB2 antibodies for 7 days. The expression of CD163, CD206 and CD86 were analyzed by flow cytometry. (FIG. 11F) Anti-LILRB2 mAbs decreased the M2 markers expression, while increased M1 markers expression, on the cell surface of tumor associated macrophage/monocyte in ascites from a patient with ovarian cancer. The CD14+ cells were isolated from the ascites by autoMACS and cultured with anti-LILRB2 mAbs with IgG4 Fc for 7 days. CD163, CD206 and CD86 were analyzed by flow cytometry. (FIG. 11G) Anti-LILRB2 mAbs increased the M1 macrophage cytokines and chemokine secreted from MDSC from 4 to 5 patients with solid tumors.

FIGS. 12A-B. Antibody sequence analysis of positive phages. (FIG. 12A) Phylogenic tree of the heavy chain variable region (VH) and light chain variable region (VL). (FIG. 12B) ELISA binding to LILRB2 of the 24 positive phages.

FIGS. 13A-B. ELISA binding EC₅₀ to LILRB2. (FIG. 13A) ELISA binding curves of LILRB2 antibodies. (FIG. 13B) Calculated EC₅₀ values of LILRB2 antibodies.

FIGS. 14A-E. Binding specificity of LILRB2 antibodies. (FIGS. 14A-B) ELISA binding of LILRB2 antibodies to antigens of other members in LILR family. (FIGS. 14C-E) Comparison of ELISA binding curves to LILRB2 and LILRA1 of antibody (FIG. 14C) B2-10, (FIG. 14D) B2-12 and (FIG. 14E) B2-18.

FIG. 15. Epitope binning of LILRB2 antibodies. The epitope binning was performed in a sandwich format on Octet RED 96 System. Each antibody was loaded on protein A biosensor as 1^{st} antibody. After blocking of biosensor with non-relevant IgG, LILRB2 antigen was then captured and the biosensors were further incubated with the rest of other antibodies (2nd antibodies). “+” indicates the 1^{st} antibody blocked the signal of the 2nd antibody.

FIGS. 16A-D. Binding domains on LILRB2 by the antibodies. (FIG. 16A) Schematic diagram shown different truncated ECD proteins with Fc fusions. (FIG. 16B) SDS-PAGE of purified fusion proteins. (FIG. 16C) ELISA binding of antibodies to different truncated proteins. (FIG. 16D) Summary of antibody binding domains.

FIGS. 17A-F. Mapping of key residues on D1 and D4. (FIGS. 17A-B) Alignment of the (FIG. 17A (SEQ ID NOS: 593-595)) D1 and the (FIG. 17B (SEQ ID NOS: 596-597)) D4 domain of LILRB2 and LILRB1. The regions that are different between LILRB2 and LILRB1 and are exposed and locate in the loop regions are boxed. (FIGS. 17C-D) the sequences of mutations on the (FIG. 17C (SEQ ID NOS: 598-605)) D1 and the (FIG. 17D (SEQ ID NOS: 606-613)) D4 domain. (FIGS. 17E-F) Binding loss of antibodies to mutants of the (FIG. 17E) D1 and the (FIG. 17F) D4 domain based on ELISA. The percent of binding with each mutant relative to the wildtype B2-ECD were plotted as stack bar graphs.

FIG. 18. Affinities of blocking antibodies. Antibodies were captured on protein A biosensors. The sensors were dipped in serially diluted LILRB2 solution for 300secs to allow association and then dipped into kinetic buffer for 600 secs to allow dissociation. The association and dissociation curves are shown in blue solid lines and the two phases are divided by dotted lines.

FIGS. 19A-B. ELISA binding measurement of antibodies to human and non-human primate (cynomolgus monkey, cyno) LILRB2 recombinantly produced in HEK293 cells. (FIG. 19A) ELISA binding to human LILRB2. (FIG. 19B) ELISA binding to cyno-LILRB2. The fusion proteins of the extracellular domain (ECD) of human LILRB2 or NHP-LILRB2 with Fc of mouse IgG2a were used to coat ELISA plates and antibodies were titrated in different concentrations as indicated in the graph.

FIGS. 20A-B. Measurements of cell surface LILRB2 binding by monoclonal antibodies by flow cytometry. (FIG. 20A) Quantification of binding ability of monoclonal antibodies to LILRB2 reporter cells. Monoclonal antibodies were screened using flow cytometer on LILRB2 reporter cells labeled with Allophycocyanin (APC) goat anti-human IgG secondary antibody. (FIG. 20B) Representative flow cytometric profiles showing that HCB2-5 and HCB2-10 monoclonal antibodies highly bind to LILRB2 reporter cells and HCB2-2 slightly bind to LILRB2 reporter cells. NC: non-labeled reporter cells. ISO: incubate the reporter cells only (ISO) with secondary Ab (anti-human Fc specific) conjugated with APC. The number indicates mean fluorescence intensity (MFI) of APC or AF647.

FIGS. 21A-B. Detection of LILRB2 antibodies binding to cyno-LILRB2 expressed on cell surface. HEK293 cells stably expressing full length cyno-LILRB2 with N-terminus FLAG tag was used to detect antibody binding by flow cytometry. An isotype human IgG1 was used as control shown as black line and solid peaks indicate the binding of the LILRB2 antibody to cyno-LILRB2 on cell surface. Mouse-anti-Flag monoclonal antibody was used for detection by flow cytometry.

FIGS. 22A-22B. Determination of engagement of GFP reporter signaling by immobilized LILRB2 antibodies. NC, negative control and IgG for isotype control.

FIGS. 23A-B. Determination of soluble antibody engagement of GFP signaling in LILRB2 reporter cells in the present or absence of K562 cells. (FIG. 23A) GFP signaling of LILRB2 reporter cells incubated with soluble antibodies. (FIG. 23B) GFP signaling of LILRB2 reporter cells incubated with soluble antibodies combined with K562.

FIGS. 24A-B. Specific binding of LILRB2 by LILRB2 monoclonal antibodies. (FIG. 24A) Representative flow cytometric profiles showing that only positive control mAbs (POS), but not LILRB2 mAbs bind to LILRAs reporter cells (RC). (FIG. 24B) Representative flow cytometric profiles showing that LILRB2 mAbs bind to LILRB2-expressing reporter cells, but not to reporter cells expressing LILRBs and LAIR1. Binding of positive control mAbs (POS) demonstrate expression level of each receptor. Binding of LILRB2 monoclonal antibodies was detected using flow cytometry on LILRAs, LILRBs or LAIR1-expressing reporter cells using a Allophycocyanin (APC)-labeled goat anti-human IgG Fc specific secondary antibody. Non: non-stained reporter cells. NEG: cells incubated only with secondary Ab. POS: cells incubated with commercial antibody conjugated with APC or AF647 for the respective LIL or LAIR-1 receptors. The number indicates mean fluorescence intensity (MFI) of APC or AF647.

FIGS. 25A-B. Blocking activity of LILRB2 antibodies assayed in GFP signaling of LILRB2 reporter cells activated by HLA-G overexpressed on K562 cells. (FIG. 25A) Representative flow cytometric profiles showed that HLA-G overexpressed on K562 cells stimulates GFP induction of LILRB2 reporter cells. (FIG. 25B) Representative flow cytometric profiles showed that HCB2-5 and HCB2-10 effectively block GFP signaling of LILRB2 reporter cells activated by HLA-G overexpressing K562 cells. IgG, isotype control.

FIGS. 26A-C. Blocking activity of LILRB2 antibodies assayed in GFP signaling of LILRB2 reporter cells activated by coated ANGPTL2. (FIG. 26A) Representative flow cytometric profiles showed that HCB2-5 and HCB2-10 effectively block GFP signaling of LILRB2 reporter cells activated by coated ANGPTL2. (FIG. 26B) Quantification of GFP percentage shown in FIG. 26A. (FIG. 26C) Dose-dependent inhibitory ability of HCB2-5 and HCB2-10 to GFP signaling induced by coated ANGPTL2.

FIGS. 27A-C. Blocking activity of HCB2-5 and HCB2-10 assayed in GFP signaling of LILRB2 reporter cells activated by coated SEMA4A. (FIG. 27A) Representative flow cytometric profiles showed that HCB2-5 and HCB2-10 effectively block GFP signaling of LILRB2 reporter cells activated by coated SEMA4A. (FIG. 27B) Quantification of GFP percentage shown in FIG. 27A. (FIG. 27C) Dose-dependent inhibitory ability of HCB2-5 and HCB2-10 to GFP signaling induced by coated SEMA4A.

FIGS. 28A-B. Antibody VH and VL phylogenic trees.

FIG. 29. Binding of LILRB2 antibodies on reporter cell line expressing ectodomain of LILR family proteins on cell surface. Bound antibodies were detected with anti-human Fc-specific secondary antibody (2^{nd} Ab) conjugated to AF647. Expression of each LILR was confirmed using commercially available antibodies directly conjugated with AF647 or APC (Ctl+Ab). All incubations were performed for 30 minutes at 4° C. Data shown is mean fluorescence intensity after sample acquisition in flow cytometer.

FIG. 30. Binding of LILRB2 antibodies on leukocytes from human whole blood harvested from healthy donors. LILRB2 antibodies were directly conjugated with AF647. One hundred microliters of whole blood were incubated with antibodies for cell surface markers and LILRB2 antibodies, following protocols available in the literature (Hensley et al., J Vis Exp 2012; 67:4302). Data shown is averaged geometric mean fluorescence intensity±standard error of the mean (s.e.m., N=2 donors) after sample acquisition in flow cytometer (BD FACS Celesta) and subtracted by background fluorescence of stained samples in which LILRB2 antibodies were omitted.

FIG. 31. Binding of LILRB2 antibodies on HEK293 stably expressing full length human LILRB2. Fifty thousand cells were incubated with a dilution series (40-0.0006 μg/mL) of LILRB2 antibodies in a final volume of 100 μL. Bound antibodies were detected with anti-human Fc-specific secondary antibody conjugated to AF647. All incubations were performed for 30 minutes at 4° C. Data shown is averaged geometric mean fluorescence intensity±standard error of the mean (s.e.m.) after sample acquisition (in duplicates) in flow cytometer (BD FACS Celesta) and subtracted by background fluorescence of samples incubated with secondary antibody only.

FIG. 32. Binding of LILRB2 antibodies on CD14⁺CD16⁻ monocytes isolated from human PBMC from healthy donors. Fifty thousand cells were incubated for 30 minutes at 4° C. with a dilution series (40-0.0006 μg/mL) of LILRB2 antibodies directly conjugated to AF647 in a final volume of 100 μL. Data shown is averaged geometric mean of fluorescence intensity from duplicate samples acquired in a flow cytometer (BD FACS Celesta) from one donor and is representative of 2 experiments with cells isolated from different donors.

FIG. 33. Inhibition of HLA-G-His (5 μg/mL) binding on HEK293 cells stably expressing full length LILRB2 in the presence of a dilution series (40-0.0098 μg/mL) of competing LILRB2 antibodies. Bound HLA-G was detected by flow cytometry using an anti-His antibody directly conjugated to APC. All incubations were performed for 30 minutes at 4° C. Data shown is averaged geometric mean of fluorescence intensity±standard error of the mean (s.e.m.) from duplicate samples acquired in a flow cytometer (BD FACS Celesta).

FIG. 34. Inhibition of SEMA4A-hFc-AF647 (5 μg/mL) binding on HEK293_LILRB2 cells in the presence of a dilution series (40-0.0098 μg/mL) of competing LILRB2 antibodies. Incubation was performed for 30 minutes at 4° C. Data shown is averaged geometric mean of fluorescence intensity±standard error of the mean (s.e.m.) from duplicate samples acquired in a flow cytometer (BD FACS Celesta).

FIG. 35. Effect of LILRB2 blocking antibodies on levels of TNF-α secreted by PBMC stimulated with 50 ng/mL LPS. Data shown is from 2 donors and it is representative from 6 donors (from total of N=6 donors). PBMC isolated from healthy donors were incubated (in duplicates) with LPS (Sigma-Aldrich) and various concentrations of antibodies for 3 days. Cytokines were measured in the culture media supernatant using a Human Cytokine Premixed Magnetic Luminex Performance Assay.

FIG. 36. Effect of LILRB2 blocking antibodies on levels of IFN-γ secreted by PBMC stimulated with 50 ng/mL LPS. Data shown is from 2 donors and it is representative from 5 donors (from total of N=6 donors). PBMC isolated from healthy donors were incubated (in duplicates) with LPS (Sigma-Aldrich) and various concentrations of antibodies for 3 days. Cytokines were measured in the culture media supernatant using a Human Cytokine Premixed Magnetic Luminex Performance Assay.

FIG. 37. Effect of LILRB2 blocking antibodies (40 μg/mL) on levels of IL-12p40 secreted by PBMC stimulated with 50 ng/mL LPS (from total of N=3 donors). PBMC isolated from healthy donors were incubated (in duplicates) with LPS (Sigma-Aldrich) and 40 μg/mL of B2-19 antibody for 3 days. IL-12p40 concentration was measured in the culture media supernatant using a human IL-12 ELISA assay (BD Biosciences)

FIG. 38. Effect of LILRB2 blocking antibodies on levels of IFN-γ secreted by PBMC stimulated with 10 ng/mL anti-CD3 activating antibody HIT3a. Data shown is from 2 donors and it is representative from 6 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 39. Effect of LILRB2 blocking antibodies on levels of TNF-α secreted by PBMC stimulated with 10 ng/mL anti-CD3 activating antibody HIT3a. Data shown is from 2 donors and it is representative from 6 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 40. Effect of LILRB2 blocking antibodies on levels of GM-CSF secreted by PBMC stimulated with 10 ng/mL anti-CD3 activating antibody HIT3a. Data shown is from 2 donors and it is representative from 6 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 41. Effect of LILRB2 blocking antibodies on levels of IL-1a secreted by PBMC stimulated with 10 ng/mL anti-CD3 activating antibody HIT3a. Data shown is from 2 donors and it is representative from 6 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 42. Effect of LILRB2 blocking antibodies on levels of IL-1β secreted by PBMC stimulated with 10 ng/mL anti-CD3 activating antibody HIT3a. Data shown is from 2 donors and it is representative from 6 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 43. Effect of LILRB2 blocking antibodies on levels of IL-6 secreted by PBMC stimulated with 10 ng/mL anti-CD3 activating antibody HIT3a. Data shown is from 2 donors and it is representative from 5 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 44. Effect of LILRB2 blocking antibodies on levels of CXCL2 secreted by PBMC stimulated with 10 ng/mL anti-CD3 activating antibody HIT3a. Data shown is from 2 donors and it is representative from 6 donors (from total of N=6 donors). PBMC isolated from healthy donors were incubated (in duplicates) with HIT3a (Biolegend) and various concentrations of antibodies for 3 days. Cytokines were measured in the culture media supernatant using a Human Cytokine Premixed Magnetic Luminex Performance Assay.

FIG. 45. Effect of 10 μg/mL B2-19 antibody on monocyte-derived macrophage cell surface markers. CD14⁺CD16⁻ monocytes isolated from human PBMC from healthy donors were differentiated into macrophages for 6 days in the presence of 100 ng/ml of human CSF-1 followed by 24 hours incubation with 100 ng/ml of human CSF-1, 20 ng/ml human IL-4 and B2-19 antibody or isotype control. Cells were detached and stained for flow cytometric analysis (FACS Celesta) using standard protocols. Data shown is fold change of geometric mean fluorescence intensity (MFI) for cells treated with B2-19 versus cells treated with isotype control.

FIG. 46. Effect of 40 μg/mL B2-19 antibody on cell surface expression of CD25 on CD8⁺ T cells. PBMC isolated from healthy donors were incubated 10 ng/ml with HIT3a (Biolegend) and 40 μg/mL of B2-19 antibody for 3 days. CD8⁺ T cells were analyzed for cell surface CD25 expression by flow cytometric analysis (FACS Celesta) using standard protocols. Data shown is percent change in geometric mean fluorescence intensity (MFI) for cells treated with B2-19 versus cells treated with isotype control.

FIG. 47. B2-19 antibody enhances the production of cytokines and chemokines in immature DC treated for 2 days with IL-10 (to induce tolerogenic DC). Each line represents the result from a different donor.

FIG. 48. B2-19 antibody enhanced the pro-inflammatory phenotype of DC treated with LPS, as evidenced by changes in expression of several cell surface markers. Each line represents the result from a different donor.

FIG. 49. B2-19 antibody displays the expected pharmacokinetics profile (CL and half-life) of a human IgG4 dosed at 5 mg/kg in C57BL/6J wild-type mice.

FIGS. 50A-B. (FIG. 50A) B2-19 antibody monotherapy reduces tumor growth rate in humanized NSG-SGM3 mice xenografted with SK-MEL-5 melanoma cell line. (FIG. 50B) B2-19 antibody monotherapy causes tumor growth inhibition in humanized NSG-SGM3 mice xenografted with SK-MEL-5 melanoma cell line.

FIG. 51. Representative flow cytometric profiles showed that coated human ANGPTL2 (hANGPLT2) and mouse Angpt12 (mAngpt12) stimulates GFP expression in LILRB2 reporter cells and B2-19 effectively block GFP expression in LILRB2 reporter cells activated by coated hANGPLT2 and mAngpt12.

FIG. 52. Representative flow cytometric profiles showed that coated human CD1d (hCD1d) and mouse CD1d (mCD1d) stimulates GFP expression in LILRB2 reporter cells and B2-19 effectively block GFP expression in LILRB2 reporter cells activated by coated hCD1d and mCD1d.

### DESCRIPTION OF ILLUSTRATIVE EMBODIMENTS

The inventors determined that LILRB2 plays critical roles in regulation of both innate and adaptive immunity. LILRB2 is expressed on several types of immune cells, such as normal monocytes, dendritic cells, granulocytes and myeloid derived suppressor cells (MDSCs). The inventors have isolated a panel of novel monoclonal antibodies recognizing LILRB2 protein, which can be used for the treatment of cancer and autoimmune diseases. Within this panel of anti-LILRB2 antibodies, there are examples of antagonists and agonists of LILRB2 signalling.

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### I. DEFINITION

It is to be understood that both the foregoing general description and the following detailed description are exemplary and explanatory only and are not restrictive of the invention as claimed. In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The term “antibody” refers to an intact immunoglobulin of any isotype, or a fragment thereof that can compete with the intact antibody for specific binding to the target antigen, and includes, for instance, chimeric, humanized, fully human, and bispecific antibodies. An “antibody” is a species of an antigen binding protein. An intact antibody will generally comprise at least two full-length heavy chains and two full-length light chains, but in some instances can include fewer chains such as antibodies naturally occurring in camelids which can comprise only heavy chains. Antibodies can be derived solely from a single source, or can be “chimeric,” that is, different portions of the antibody can be derived from two different antibodies as described further below. The antigen binding proteins, antibodies, or binding fragments can be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the term also encompasses peptibodies.

Naturally occurring antibody structural units typically comprise a tetramer. Each such tetramer typically is composed of two identical pairs of polypeptide chains, each pair having one full-length “light” (in certain embodiments, about 25 kDa) and one full-length “heavy” chain (in certain embodiments, about 50-70 kDa). The amino-terminal portion of each chain typically includes a variable region of about 100 to 110 or more amino acids that typically is responsible for antigen recognition. The carboxy-terminal portion of each chain typically defines a constant region that can be responsible for effector function. Human light chains are typically classified as kappa and lambda light chains. Heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, typically, the variable and constant regions are joined by a “J” region of about 12 or more amino acids, with the heavy chain also including a “D” region of about 10 more amino acids. See, e.g., Fundamental Immunology, Ch. 7 (Paul, W., ed., 2^{nd} ed. Raven Press, N.Y. (1989)) (incorporated by reference in its entirety for all purposes). The variable regions of each light/heavy chain pair typically form the antigen binding site.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target.

The variable regions typically exhibit the same general structure of relatively conserved framework regions (FR) joined by three hyper variable regions, also called complementarity determining regions or CDRs. The CDRs from the two chains of each pair typically are aligned by the framework regions, which can enable binding to a specific epitope. From N-terminal to C-terminal, both light and heavy chain variable regions typically comprise the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), Chothia & Lesk, J. Mol. Biol., 196:901-917 (1987) or Chothia et al., Nature, 342:878-883 (1989).

In certain embodiments, an antibody heavy chain binds to an antigen in the absence of an antibody light chain. In certain embodiments, an antibody light chain binds to an antigen in the absence of an antibody heavy chain. In certain embodiments, an antibody binding region binds to an antigen in the absence of an antibody light chain. In certain embodiments, an antibody binding region binds to an antigen in the absence of an antibody heavy chain. In certain embodiments, an individual variable region specifically binds to an antigen in the absence of other variable regions.

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28: 214-8 (2000). The Chothia definition is similar to the Kabat definition, but the Chothia definition takes into account positions of certain structural loop regions. See, e.g., Chothia et al., J. Mol. Biol., 196:901-17 (1986); Chothia et al., Nature, 342:877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

By convention, the CDR regions in the heavy chain are typically referred to as H1, H2, and H3 and are numbered sequentially in the direction from the amino terminus to the carboxy terminus. The CDR regions in the light chain are typically referred to as L1, L2, and L3 and are numbered sequentially in the direction from the amino terminus to the carboxy terminus.

The term “light chain” includes a full-length light chain and fragments thereof having sufficient variable region sequence to confer binding specificity. A full-length light chain includes a variable region domain, VL, and a constant region domain, CL. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, VH, and three constant region domains, CH1, CH2, and CH3. The VH domain is at the amino-terminus of the polypeptide, and the CH domains are at the carboxyl-terminus, with the CH3 being closest to the carboxy-terminus of the polypeptide. Heavy chains can be of any isotype, including IgG (including IgG1, IgG2, IgG3 and IgG4 subtypes), IgA (including IgA1 and IgA2 subtypes), IgM and IgE.

A bispecific or bifunctional antibody typically is an artificial hybrid antibody having two different heavy/light chain pairs and two different binding sites. Bispecific antibodies can be produced by a variety of methods including, but not limited to, fusion of hybridomas or linking of Fab′ fragments. See, e.g., Songsivilai et al., Clin. Exp. Immunol., 79:315-321 (1990); Kostelny et al., J. Immunol., 148:1547-1553 (1992).

The term “antigen” refers to a substance capable of inducing adaptive immune responses. Specifically, an antigen is a substance which serves as a target for the receptors of an adaptive immune response. Typically, an antigen is a molecule that binds to antigen-specific receptors but cannot induce an immune response in the body by itsself. Antigens are usually proteins and polysaccharides, less frequently also lipids. Suitable antigens include without limitation parts of bacteria (coats, capsules, cell walls, flagella, fimbrai, and toxins), viruses, and other microorganisms. Antigens also include tumor antigens, e.g., antigens generated by mutations in tumors. As used herein, antigens also include immunogens and haptens.

An “antigen binding protein” (“ABP”) as used herein means any protein that binds a specified target antigen. In the instant application, the specified target antigen is the LILRB protein or fragment thereof. “Antigen binding protein” includes but is not limited to antibodies and antigen-binding fragment thereof. Peptibodies are another example of antigen binding proteins.

The term “antigen-binding fragment” as used herein refers to a portion of a protein which is capable of binding specifically to an antigen. In certain embodiment, the antigen-binding fragment is derived from an antibody comprising one or more CDRs, or any other antibody fragment that binds to an antigen but does not comprise an intact native antibody structure. In certain embodiments, the antigen-binding fragment is not derived from an antibody but rather is derived from a receptor. Examples of antigen-binding fragment include, without limitation, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a single domain antibody (sdAb), a camelid antibody or a nanobody, a domain antibody, and a bivalent domain antibody. In certain embodiments, an antigen-binding fragment is capable of binding to the same antigen to which the parent antibody binds. In certain embodiments, an antigen-binding fragment may comprise one or more CDRs from a particular human antibody grafted to a framework region from one or more different human antibodies. In certain embodiments, the antigen-binding fragment is derived from a receptor and contains one or more mutations. In certain embodiments, the antigen-binding fragment does not bind to the natural ligand of the receptor from which the antigen-binding fragment is derived.

A “Fab fragment” comprises one light chain and the CH1 and variable regions of one heavy chain. The heavy chain of a Fab molecule cannot form a disulfide bond with another heavy chain molecule.

A “Fab′ fragment” comprises one light chain and a portion of one heavy chain that contains the VH domain and the CH1 domain and also the region between the CH1 and CH2 domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the CH1 and CH2 domains, such that an interchain disulfide bond is formed between the two heavy chains. A F(ab′)₂ fragment thus is composed of two Fab′ fragments that are held together by a disulfide bond between the two heavy chains.

An “Fc” region comprises two heavy chain fragments comprising the CH1 and CH2 domains of an antibody. The two heavy chain fragments are held together by two or more disulfide bonds and by hydrophobic interactions of the CH3 domains.

The “Fv region” comprises the variable regions from both the heavy and light chains but lacks the constant regions.

“Single-chain antibodies” are Fv molecules in which the heavy and light chain variable regions have been connected by a flexible linker to form a single polypeptide chain, which forms an antigen binding region. Single chain antibodies are discussed in detail in International Patent Application Publication No. WO 88/01649 and U.S. Pat. Nos. 4,946,778 and 5,260,203, the disclosures of which are incorporated by reference.

A “domain antibody” is an immunologically functional immunoglobulin fragment containing only the variable region of a heavy chain or the variable region of a light chain. In some instances, two or more VH regions are covalently joined with a peptide linker to create a bivalent domain antibody. The two VH regions of a bivalent domain antibody can target the same or different antigens.

A “bivalent antigen binding protein” or “bivalent antibody” comprises two antigen binding sites. In some instances, the two binding sites have the same antigen specificities. Bivalent antigen binding proteins and bivalent antibodies can be bispecific, see, infra. A bivalent antibody other than a “multispecific” or “multifunctional” antibody, in certain embodiments, typically is understood to have each of its binding sites identical.

A “multispecific antigen binding protein” or “multispecific antibody” is one that targets more than one antigen or epitope.

A “bispecific,” “dual-specific” or “bifunctional” antigen binding protein or antibody is a hybrid antigen binding protein or antibody, respectively, having two different antigen binding sites. Bispecific antigen binding proteins and antibodies are a species of multispecific antigen binding protein antibody and can be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553. The two binding sites of a bispecific antigen binding protein or antibody will bind to two different epitopes, which can reside on the same or different protein targets.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity are described in the following.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope. For example, the LILRB2 specific antibodies of the present invention are specific to LILRB2. In some embodiments, the antibody that binds to LILRB2 has a dissociation constant (Kd) of ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M). The dissociation constant Kd used herein refers to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the Kd value can be appropriately determined by using flow cytometry.

The term “compete” when used in the context of antigen binding proteins (e.g., antibody or antigen-binding fragment thereof) that compete for the same epitope means competition between antigen binding proteins as determined by an assay in which the antigen binding protein (e.g., antibody or antigen-binding fragment thereof) being tested prevents or inhibits (e.g., reduces) specific binding of a reference antigen binding protein (e.g., a ligand, or a reference antibody) to a common antigen (e.g., LILRB or a fragment thereof). Numerous types of competitive binding assays can be used to determine if one antigen binding protein competes with another, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using 1-125 label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol. 32:77-82). Typically, such an assay involves the use of purified antigen bound to a solid surface or cells bearing either of these, an unlabelled test antigen binding protein and a labeled reference antigen binding protein. Competitive inhibition is measured by determining the amount of label bound to the solid surface or cells in the presence of the test antigen binding protein. Usually the test antigen binding protein is present in excess. Antigen binding proteins identified by competition assay (competing antigen binding proteins) include antigen binding proteins binding to the same epitope as the reference antigen binding proteins and antigen binding proteins binding to an adjacent epitope sufficiently proximal to the epitope bound by the reference antigen binding protein for steric hindrance to occur. Additional details regarding methods for determining competitive binding are provided in the examples herein. Usually, when a competing antigen binding protein is present in excess, it will inhibit (e.g., reduce) specific binding of a reference antigen binding protein to a common antigen by at least 40-45%, 45-50%, 50-55%, 55-60%, 60-65%, 65-70%, 70-75% or 75% or more. In some instances, binding is inhibited by at least 80-85%, 85-90%, 90-95%, 95-97%, or 97% or more.

The term “epitope” as used herein refers to the specific group of atoms or amino acids on an antigen to which an antibody binds. The epitope can be either linear epitope or a conformational epitope. A linear epitope is formed by a continuous sequence of amino acids from the antigen and interacts with an antibody based on their primary structure. A conformational epitope, on the other hand, is composed of discontinuous sections of the antigen's amino acid sequence and interacts with the antibody based on the 3D structure of the antigen. In general, an epitope is approximately five or six amino acid in length. Two antibodies may bind the same epitope within an antigen if they exhibit competitive binding for the antigen.

A “cell”, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ, e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell; a cell from an endocrine system or organ, e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte); a cell from a nervous system or organ, e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph); a cell from a respiratory system or organ, e.g., a pneumocyte (a type I pneumocyte and a type II pneumocyte), a clara cell, a goblet cell, and an alveolar macrophage; a cell from circular system or organ (e.g., myocardiocyte and pericyte); a cell from digestive system or organ, e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, and a liver cell (e.g., a hepatocyte and Kupffer cell); a cell from integumentary system or organ, e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell), a muscle cell (e.g., myocyte), an adipocyte, a fibroblast, and a tendon cell; a cell from urinary system or organ (e.g., a podocyte, a juxtaglomerular cell, an intraglomerular mesangial cell, an extraglomerular mesangial cell, a kidney proximal tubule brush border cell, and a macula densa cell); and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell.

The term “chimeric antigen receptor” or “CAR” as used herein refers to an artificially constructed hybrid protein or polypeptide containing an antigen binding domain of an antibody (e.g., a single chain variable fragment (scFv)) linked to a domain or signaling, e.g., T-cell signaling or T-cell activation domains, that activates an immune cell, e.g., a T cell or a NK cell (see, e.g., Kershaw et al., supra, Eshhar et al., Proc. Natl. Acad. Sci. USA, 90 (2): 720-724 (1993), and Sadelain et al., Curr. Opin. Immunol. 21 (2): 215-223 (2009)). CARs are capable of redirecting the immune cell specificity and reactivity toward a selected target in a non-MHC-restricted manner, taking advantage of the antigen-binding properties of monoclonal antibodies. The non-MHC-restricted antigen recognition confers immune cells expressing CARs on the ability to recognize an antigen independent of antigen processing, thus bypassing a major mechanism of tumor escape. In addition, when expressed in T-cells, CARs advantageously do not dimerize with endogenous T-cell receptor (TCR) alpha and beta chains.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Examples of parameters that can be employed in determining percent identity for polypeptides or nucleotide sequences using the GAP program can be found in Needleman et al., 1970, J. Mol. Biol. 48:443-453.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 or other number of contiguous amino acids of the target polypeptide.

The term “link” as used herein refers to the association via intramolecular interaction, e.g., covalent bonds, metallic bonds, and/or ionic bonding, or inter-molecular interaction, e.g., hydrogen bond or noncovalent bonds.

Leukocyte immunoglobulin-like receptor subfamily B member 2 (LILRB2) is a protein that in humans is encoded by the LILRB2 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on myeloid cells; it binds to multiple types of ligands, including HLA class I molecules, ANGPTLs, myelin inhibitors (including Nogo66, MAG, and OMgp), and and β-amyloid, transducing a negative signal that inhibits stimulation of an immune response. It is thought to control inflammatory responses and cytotoxicity to help focus the immune response and limit autoreactivity.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The terms “polypeptide” or “protein” means a macromolecule having the amino acid sequence of a native protein, that is, a protein produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The term also includes amino acid polymers in which one or more amino acids are chemical analogs of a corresponding naturally-occurring amino acid and polymers. The terms “polypeptide” and “protein” specifically encompass LILRB antigen binding proteins, antibodies, or sequences that have deletions from, additions to, and/or substitutions of one or more amino acid of antigen-binding protein. The term “polypeptide fragment” refers to a polypeptide that has an amino-terminal deletion, a carboxyl-terminal deletion, and/or an internal deletion as compared with the full-length native protein. Such fragments can also contain modified amino acids as compared with the native protein. In certain embodiments, fragments are about five to 500 amino acids long. For example, fragments can be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains. In the case of a LILRB-binding antibody, useful fragments include but are not limited to a CDR region, a variable domain of a heavy and/or light chain, a portion of an antibody chain or just its variable region including two CDRs, and the like.

The pharmaceutically acceptable carriers useful in this invention are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “therapeutically effective amount” or “effective dosage” as used herein refers to the dosage or concentration of a drug effective to treat a disease or condition. For example, with regard to the use of the monoclonal antibodies or antigen-binding fragments thereof disclosed herein to treat cancer, a therapeutically effective amount is the dosage or concentration of the monoclonal antibody or antigen-binding fragment thereof capable of reducing the tumor volume, eradicating all or part of a tumor, inhibiting or slowing tumor growth or cancer cell infiltration into other organs, inhibiting growth or proliferation of cells mediating a cancerous condition, inhibiting or slowing tumor cell metastasis, ameliorating any symptom or marker associated with a tumor or cancerous condition, preventing or delaying the development of a tumor or cancerous condition, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### II. LILRB2 RELATED DISEASES

LILRB2 has been identified as a key regulator of myeloid cell phenotype. The activation of LILRB2 suppresses the pro-inflammatory activity of myeloid cells. While myeloid cells with a suppressive/anti-inflammatory phenotype can down-regulate the activation, proliferation and cytotoxic activity of T cells, modulation of LILRB2 has the potential in therapeutic use in conditions and disorders including cancer, autoimmune diseases, and inflammatory diseases.

While hyperproliferative diseases can be associated with any disease which causes a cell to begin to reproduce uncontrollably, the prototypical example is cancer.

Examples of cancer can be generally categorized into solid tumors and hematologic malignancies. Solid tumors include but are not limited to, adrenal cancer, bile duct carcinoma, bone cancer, brain cancer (e.g., astrocytoma, brain stem glioma, craniopharyngioma, ependymoma, hemangioblastoma, medulloblastoma, meningioma, oligodendroglioma, spinal axis tumor), breast cancer (including acoustic neuroma, basal breast carcinoma, ductal carcinoma and lobular breast carcinoma), cervical cancer, choriocarcinoma, colon cancer, colorectal cancer, esophageal cancer, eye cancer, gastric cancer, glioblastoma, head and neck cancer, kidney cancer (including Wilms tumor), liver cancer (including hepatocellular carcinoma (HCC)), lung cancer (including bronchogenic carcinoma, non-small cell lung cancer (squamous/non-squamous), bronchioloalveolar cell lung cancer, papillary adenocarcinomas), mesothelioma, melanoma, merkel cell cancer, nasopharyngeal carcinoma, neuroblastoma, oral cancer, ovarian cancer, pancreatic cancer, penile cancer, pinealoma, prostate cancer, renal cell cancer, retinoblastoma, sarcoma (including chondrosarcoma, Ewing's sarcoma, fibrosarcoma, leiomyosarcoma, liposarcoma, myxosarcoma, osteogenic sarcoma, rhabdomyosarcoma, synovial sarcoma), skin cancer (including basal cell carcinoma, sebaceous gland carcinoma, squamous cell carcinoma), testicular cancer (including seminoma), thymic carcinoma, thyroid cancer (e.g., medullary thyroid carcinoma, papillary thyroid carcinoma), uterine cancer, and vaginal cancer.

Hematologic malignancies include but are not limited to blastic plasmacytoid dendritic cell neoplasm (BPDCN), heavy chain disease, leukemias (including but not limited to acute lymphocytic leukemia (ALL), acute myeloid leukemia (AML) (including but not limited to acute promyelocytic leukemia (APL) or M3 AML, acute myelomonocytic leukemia or M4 AML, acute monocytic leukemia or M5 AML), B-cell leukemia, chronic lymphoblastic leukemia (CLL), chronic myelomonocytic leukemia (CMML), chronic myelocytic leukemia (CML), pre-B acute lymphocytic leukemia (Pre-B ALL), diffuse large B-cell lymphoma (DLBCL), extranodal NK/T-cell lymphoma, hairy cell leukemia, HHV8-associated primary effusion lymphoma, plasmablastic lymphoma, primary CNS lymphoma, primary mediastinal large B-cell lymphoma, T-cell/histiocyte-rich B-cell lymphoma), lymphomas (including but not limited to Hodgkin's lymphoma, non-Hodgkin's lymphoma, Waldenstrom's macroglobulinemia), multiple myeloma (MM), myelodysplastic syndromes (MDS), myeloproliferative neoplasms, and polycythemia vera.

Immunotherapy holds great promise to achieve long-lasting anti-tumor effects. Immune checkpoint PD-1 and CTLA-4 blockade therapies have been successful in treating some types of cancers but not others. These immunotherapies target inhibitory molecules on T cells to reactivate dysfunctional T cells within the tumor microenvironment (TME). Other populations of immune cells, including monocytic cells, are present in the TME in even larger numbers than T cells. In fact, monocyte-derived macrophages are the most abundant immune cell population in tumor tissues. While these innate cells possess the capacity to kill tumor cells and to prime or reactivate T cells, they become dysfunctional in TME and turn into MDSCs and tumor-associated macrophages (TAMs) that support tumor development and suppress immune surveillance and attack. MDSCs, including monocytic MDSCs (M-MDSCs) and polymorphonuclear MDSCs (PMN-MDSCs), represent a heterogeneous population of immature myeloid cells that fail to terminally differentiate. TAMs are a mixed macrophage population in TME. They are anti-inflammatory and correlated with a poor prognosis. Despite their phenotypic plasticity, MDSCs and TAMs are defined by their immunosuppressive function. Removing, reprogramming, or blocking trafficking of these immune-suppressive monocytic cells is becoming an attractive anti-cancer therapeutic strategy.

LILRB2 is expressed on MDSCs and TAMs in TME. Therapeutic blocking of LILRB2 in myeloid-rich solid tumors has the potential to reactivate or enhance anti-tumor immune responses in patients presenting with disease unresponsive/relapsed to T cell checkpoint inhibitors.

LILRB2 expression on myeloid cells may regulate systems involved in autoimmune and inflammatory diseases. Therapeutic activating or agonizing LILRB2 has the potential to treat autoimmune or inflammatory diseases.

Autoimmune or inflammatory diseases include, but are not limited to, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenia purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pemacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma, systemic scleroderma, progressive systemic sclerosis (PSS), systemic sclerosis (SS), Sjogren's syndrome, stiff-man syndrome, systemic lupus erythematosus (SLE), Takayasu arteritis, temporal arteritis/giant cell arteritis, inflammatory bowel disease (IBD), ulcerative colitis, Cohn's disease, intestinal mucosal inflammation, wasting disease associated with colitis, uveitis, vitiligo and Wegener's granulomatosis, Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy, ventilator induced lung injury, viral infections, autoimmune diabetes and the like. Inflammatory disorders include, for example, chronic and acute inflammatory disorders.

### III. MONOCLONAL ANTIBODIES AND PRODUCTION THEREOF

The monoclonal antibodies described herein can be prepared using standard methods, followed by screening, characterization and functional assessment. Variable regions can be sequenced and then subcloned into a human expression vector to produce the chimeric antibody genes, which are then expressed and purified. These chimeric antibodies can be tested for antigen binding, signaling blocking, and in xenograft experiments. The monoclonal antibodies described herein can also be prepared using phage display method, in which a large library of phage displayed human scFv is panned against the target protein. The human scFv selected to specifically binding to the target protein can be sequenced and then subcloned into a human expression vector to produce the desired human antibody.

### A. General Methods

It will be understood that monoclonal antibodies binding to LILRB2 will have several applications. These include the production of diagnostic kits for use in detecting and diagnosing cancer, as well as for cancer therapies. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The classical methods for generating monoclonal antibodies (MAbs) generally begin along the same lines as those for preparing polyclonal antibodies. The first step for both these methods is immunization of an appropriate host. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984).

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). Fusion procedures usually produce viable hybrids at low frequencies, about 1×10-6 to 1×10-8. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Recently, additional methods for generating mAb, such as scFv phage display, have been developed (see CM Hammers and JR Stanley, Antibody phage display: technique and applications, J Invest Dermatol (2014) 134: e17). Generally, a panel of human mAbs that bind to a target protein, e.g., human LILRB2, are generated by panning a large diversity of human scFv phage displayed antibody library.

To generate the human scFv phage displayed antibody library, RNA is extracted from the chosen cell source, e.g., peripheral blood mononuclear cells. The RNA is then reversed-transcribed into cDNA, which is used for PCR of the VH and VL chains of the encoded antibodies. Defined sets of primers specific for the different VH and VL chain region gene families allow the amplification of all transcribed rearranged variable regions within a given immunoglobulin repertoire, reflecting all antibody specificities in a particular individual.

The VH and VL PCR products that represent the antibody repertoire are ligated into a phage display vector that is engineered to express the VH and VL as an scFv fused to the pIII minor capsid protein of a filamentous bacteriophage of E. coli that was originally derived from the M13 bacteriophage. This generates a library of phages, each of which expresses on its surface a scFv and harbors the vector with the respective nucleotide sequence within.

The library is then screened for phage binding to a target antigen through its expressed surface scFv by a technique called bio-panning. In short, the target protein is coated on solid phase for incubation with phage libraries. After washing and elution, antigen enriched phages are recovered and used for next rounds of phage panning. After at least three rounds of phage panning, single bacterial colonies are picked for phage ELISA and other functional/genetic analysis.

The positive hits are sequenced for the scFv region and are converted to full human IgG heavy and light chain constructs, which are used to generate the mAb of interest using the methods disclosed supra. For example, the IgG expressing plasmids are cotransfected into Expi293 cells using transfection reagent PEI. After 7 days of expression, supernatants are harvested, and antibodies are purified by affinity chromatography using protein A resin.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

### B. Antibodies of the Present Disclosure

### 1. Antibodies to LILRB2

Antibodies or antigen-binding fragments thereof according to the present disclosure may be defined, in the first instance, by their binding specificity, which in this case is for LILRB2. Those of skill in the art, by assessing the binding specificity/affinity of a given antibody using techniques well known to those of skill in the art, can determine whether such antibodies fall within the scope of the instant claims.

In one aspect, there are provided antibodies and antigen-binding fragments specifically bind to LILRB2. In some embodiments, when bound to LILRB2, such antibodies modulate the activation of LILRB2. In certain embodiments, the antibody or antigen-binding fragment, when bound to LILRB2, activates LILRB2. In certain embodiments, the antibody or antigen-binding fragment, when bound to LILRB2, suppresses activation of LILRB2. In certain embodiments, the antibody or antigen-binding fragment, when bound to LILRB2, can specifically interfere with, block or reduce the interaction between LILRB2 and its binding partners. In certain embodiments, the antibody or antigen-binding fragment provided herein is capable of inhibiting the immunosuppressive activity of MDSCs and other solid tumor-infiltrating myeloid cells, such as tumor-associated macrophages (TAMs) and tolerogenic dendritic cells (DCs). In certain embodiments, the antibodies or antigen-binding fragments provided herein specifically or selectively bind to human LILRB2.

In some embodiments, the antibodies or antigen-binding fragments bind specifically to human LILRB2 and/or substantially inhibits binding of human LILRB2 to HLA-G, ANGPTLs, SEMA4A by at least about 20%-40%, 40-60%, 60-80%, 80-85%, or more (for example, by an assay as disclosed in the Example). In some embodiments, the antibody or antigen-binding fragment has a Kd of less (binding more tightly) than 10-6, 10-7, 10-8, 10-9, 10-10, 10-11, 10-12, 10-13 M. In some embodiments, the antibody or antigen-binding fragment has an IC₅₀ for blocking the binding of HLA-G, ANGPTLs, SEMA4A to LILRB2 of less than 10 μM, 10 μM to 1 μM, 1000 nM to 100 nM, 100 nM to 10 nM, 10 nM to 1 nM, 1000 μM to 500 μM, 500 μM to 200 μM, less than 200 μM, 200 μM to 150 μM, 200 μM to 100 μM, 100 μM to 10 μM, 10 μM to 1 pM.

In some embodiments, the antibodies or antigen-binding fragments provided herein having the clone-paired CDRs illustrated in Table 2.

In certain embodiments, the antibodies may be defined by their variable sequence, which include additional “framework” regions. The antibody is characterized by clone-paired heavy chain and light chain amino acid sequences from Appendices I and III. Furthermore, the antibodies sequences may vary from these sequences, particularly in regions outside the CDRs. For example, the amino acids may vary from those set out above by a given percentage, e.g., 80%, 85%, 90%, 91%, 92%, 93%, 94%, 95%, 96%, 97%, 98% or 99% homology, or the amino acids may vary from those set out above by permitting conservative substitutions (discussed below). Each of the foregoing apply to the amino acid sequences of Appendices I and III. In another embodiment, the antibody derivatives of the present disclosure comprise VL and VH domains having up to 0, 1, 2, 3, 4, 5, 6, 7, 8, 9, 10 or more conservative or non-conservative amino acid substitutions, while still exhibiting the desired binding and functional properties.

While the antibodies of the present disclosure were generated as IgG's, it may be useful to modify the constant regions to alter their function. The constant regions of the antibodies typically mediate the binding of the antibody to host tissues or factors, including various cells of the immune system (e.g., effector cells) and the first component (Clq) of the classical complement system. Thus, the term “antibody” includes intact immunoglobulins of types IgA, IgG, IgE, IgD, IgM (as well as subtypes thereof), wherein the light chains of the immunoglobulin may be of types kappa or lambda. Within light and heavy chains, the variable and constant regions are joined by a 35 “J” region of about 12 or more amino acids, with the heavy chain also including a “D” region of about 10 more amino acids. See generally, Fundamental Immunology Ch. 7 (Paul, W., ed., 2^{nd} ed. Raven Press, N.Y. (1989).

The present disclosure further comprises nucleic acids which hybridize to nucleic acids encoding the antibodies disclosed herein. In general, the nucleic acids hybridize under moderate or high stringency conditions to nucleic acids that encode antibodies disclosed herein and also encode antibodies that maintain the ability to specifically bind to an LILRB2. A first nucleic acid molecule is “hybridizable” to a second nucleic acid molecule when a single stranded form of the first nucleic acid molecule can anneal to the second nucleic acid molecule under the appropriate conditions of temperature and solution ionic strength (see Sambrook et al., MOLECULAR CLONING: A LABORATORY MANUAL, 3^{rd} ed., Cold Spring Harbor Press, Cold Spring Harbor, N.Y. 2001). The conditions of temperature and ionic strength determine the “stringency” of the hybridization. Typical moderate stringency hybridization conditions are 40% formamide, with 5X or 6×SSC and 0.1% SDS at 42° C. High stringency hybridization conditions are 50% formamide, 5X or 6×SSC (0.15M NaCl and 0.015M Na-citrate) at 42° C. or, optionally, at a higher temperature (e.g., 57° C., 59° C., 60° C., 62° C., 63° C., 65° C. or 68° C.). Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the higher the stringency under which the nucleic acids may hybridize. For hybrids of greater than 100 nucleotides in length, equations for calculating the melting temperature have been derived (see Sambrook et al., supra). For hybridization with shorter nucleic acids, e.g., oligonucleotides, the position of mismatches becomes more important, and the length of the oligonucleotide determines its specificity (see Sambrook et al., supra).

### 2. Exemplary Epitopes and Competing Antigen Binding Proteins

In another aspect, the present disclosure provides epitopes to which anti-LILRB2 antibodies bind. In some embodiments, epitopes that are bound by the antibodies described herein are useful. In certain embodiments, an epitope provided herein can be utilized to isolate antibodies or antigen binding proteins that bind to LILRB2. In certain embodiments, an epitope provided herein can be utilized to generate antibodies or antigen binding proteins which bind to LILRB2. In certain embodiments, an epitope or a sequence comprising an epitope provided herein can be utilized as an immunogen to generate antibodies or antigen binding proteins that bind to LILRB2. In certain embodiments, an epitope described herein or a sequence comprising an epitope described herein can be utilized to interfere with biological activity of LILRB2.

In some embodiments, antibodies or antigen-binding fragments thereof that bind to any of the epitopes are particularly useful. In some embodiments, an epitope provided herein, when bound by an antibody, modulates the biological activity of LILRB2. In some embodiments, an epitope provided herein, when bound by an antibody, activates LILRB2. In some embodiments, an epitope provided herein, when bound by an antibody, suppress the activation of LILRB2. In some embodiments, an epitope provided herein, when bound by an antibody, block the interaction between LILRB2 and its binding partners.

In some embodiments, the domain(s)/region(s) containing residues that are in contact with or are buried by an antibody can be identified by mutating specific residues in LILRB2 and determining whether the antibody can bind the mutated LILRB2 protein. By making a number of individual mutations, residues that play a direct role in binding or that are in sufficiently close proximity to the antibody such that a mutation can affect binding between the antibody and antigen can be identified. From knowledge of these amino acids, the domain(s) or region(s) of the antigen that contain residues in contact with the antigen binding protein or covered by the antibody can be elucidated. Such a domain can include the binding epitope of an antigen binding protein.

In another aspect, the present disclosure provides antigen-binding proteins that compete with one of the exemplified antibodies or antigen-binding fragment binding to the epitope described herein for specific binding to LILRB2. Such antigen binding proteins can also bind to the same epitope as one of the herein exemplified antibodies or the antigen-binding fragment, or an overlapping epitope. Antigen-binding proteins that compete with or bind to the same epitope as the exemplified antibodies are expected to show similar functional properties. The exemplified antibodies include those described above, including those with the heavy and light chain variable regions and CDRs included in Table 2, heavy and light chains as shown in Appendices I and III, and heavy and light chain coding regions as shown in Appendices II and IV.

### C. Engineering of Antibody Sequences

In various embodiments, one may choose to engineer sequences of the identified antibodies for a variety of reasons, such as improved expression, improved cross-reactivity or diminished off-target binding. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns. Recombinant full-length IgG antibodies may be generated by subcloning heavy and light chain Fv DNAs from the cloning vector into an IgG plasmid vector, transfected into 293 Freestyle cells or CHO cells, and antibodies collected a purified from the 293 or CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

### 1. Antigen Binding Modifications

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Modified antibodies may be made by any technique known to those of skill in the art, including expression through standard molecular biological techniques, or the chemical synthesis of polypeptides. Methods for recombinant expression are addressed elsewhere in this document.

### 2. Fc Region Modifications

The antibodies disclosed herein can also be engineered to include modifications within the Fc region, typically to alter one or more functional properties of the antibody, such as serum half-life, complement fixation, Fc receptor binding, and/or effector function (e.g., antigen-dependent cellular cytotoxicity). Furthermore, the antibodies disclosed herein can be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat. The antibodies disclosed herein also include antibodies with modified (or blocked) Fc regions to provide altered effector functions. See, e.g., U.S. Pat. No. 5,624,821; WO2003/086310; WO2005/120571; WO2006/0057702. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. This mutation has been reported to abolish the heterogeneity of inter-heavy chain disulfide bridges in the hinge region.

In one embodiment, the hinge region of CH1 is modified such that the number of cysteine residues in the hinge region is increased or decreased. This approach is described further in U.S. Pat. No. 5,677,425. The number of cysteine residues in the hinge region of CH1 is altered, for example, to facilitate assembly of the light and heavy chains or to increase or decrease the stability of the antibody. In another embodiment, the antibody is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022. In yet other embodiments, the Fc region is altered by replacing at least one amino acid residue with a different amino acid residue to alter the effector function(s) of the antibodies. For example, one or more amino acids selected from amino acid residues 234, 235, 236, 237, 297, 318, 320 and 322 can be replaced with a different amino acid residue such that the antibody has an altered affinity for an effector ligand but retains the antigen binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acid residues within amino acid positions 231 and 239 are altered to thereby alter the ability of the antibody to fix complement. This approach is described further in PCT Publication WO 94/29351. In yet another example, the Fc region is modified to increase or decrease the ability of the antibodies to mediate antibody dependent cellular cytotoxicity (ADCC) and/or to increase or decrease the affinity of the antibodies for an Fcγ receptor by modifying one or more amino acids at the following positions: 238, 239, 243, 248, 249, 252, 254, 255, 256, 258, 264, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072. Moreover, the binding sites on human IgG1 for FcγR1, FcγRII, FcγRIII and FcRn have been mapped and variants with improved binding have been described. Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In one embodiment, the Fc region is modified to decrease the ability of the antibodies to mediate effector function and/or to increase anti-inflammatory properties by modifying residues 243 and 264. In one embodiment, the Fc region of the antibody is modified by changing the residues at positions 243 and 264 to alanine. In one embodiment, the Fc region is modified to decrease the ability of the antibody to mediate effector function and/or to increase anti-inflammatory properties by modifying residues 243, 264, 267 and 328.

In one embodiment, the Fc region is modified to abolish the ability of the antibodies to mediate effector function by modifying residues 234, 235 and 329 to alanine or glycine (L234A-L235A-P329G).

In still another embodiment, the antibody comprises a particular glycosylation pattern. For example, an aglycosylated antibody can be made (i.e., the antibody lacks glycosylation). The glycosylation pattern of an antibody may be altered to, for example, increase the affinity or avidity of the antibody for an antigen. Such modifications can be accomplished by, for example, altering one or more of the glycosylation sites within the antibody sequence. For example, one or more amino acid substitutions can be made that result removal of one or more of the variable region framework glycosylation sites to thereby eliminate glycosylation at that site. Such aglycosylation may increase the affinity or avidity of the antibody for antigen. See, e.g., U.S. U.S. Pat. Nos. 5,714,350 and 6,350,861.

An antibody may also be made in which the glycosylation pattern includes hypofucosylated or afucosylated glycans, such as a hypofucosylated antibodies or afucosylated antibodies have reduced amounts of fucosyl residues on the glycan. The antibodies may also include glycans having an increased amount of bisecting GlcNac structures. Such altered glycosylation patterns have been demonstrated to increase the ADCC ability of antibodies. Such modifications can be accomplished by, for example, expressing the antibodies in a host cell in which the glycosylation pathway was been genetically engineered to produce glycoproteins with particular glycosylation patterns. These cells have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (α (1,6)-fucosyltransferase), such that antibodies expressed in the Ms704, Ms705, and Ms709 cell lines lack fucose on their carbohydrates. The Ms704, Ms705, and Ms709 FUT8−/−cell lines were created by the targeted disruption of the FUT8 gene in CHO/DG44 cells using two replacement vectors (see U.S. Patent Publication No. 20040110704. As another example, EP 1 176 195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1 176 195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn (297)-linked carbohydrates, also resulting in hypofucosylation of antibodies expressed in that host cell. Antibodies with a modified glycosylation profile can also be produced in chicken eggs, as described in PCT Publication WO 06/089231. Alternatively, antibodies with a modified glycosylation profile can be produced in plant cells, such as Lemna (U.S. Pat. No. 7,632,983). Methods for production of antibodies in a plant system are disclosed in the U.S. Pat. Nos. 6,998,267 and 7,388,081. PCT Publication WO 99/54342 describes cell lines engineered to express glycoprotein-modifying glycosyl transferases (e.g., β (1,4)-N-acetylglucosaminyltransferase III (GnTIII)) such that antibodies expressed in the engineered cell lines exhibit increased bisecting GlcNac structures which results in increased ADCC activity of the antibodies.

Alternatively, the fucose residues of the antibodies can be cleaved off using a fucosidase enzyme; e.g., the fucosidase a-L-fucosidase removes fucosyl residues from antibodies. Antibodies disclosed herein further include those produced in lower eukaryote host cells, in particular fungal host cells such as yeast and filamentous fungi have been genetically engineered to produce glycoproteins that have mammalian- or human-like glycosylation patterns. A particular advantage of these genetically modified host cells over currently used mammalian cell lines is the ability to control the glycosylation profile of glycoproteins that are produced in the cells such that compositions of glycoproteins can be produced wherein a particular N-glycan structure predominates (see, e.g., U.S. Pat. Nos. 7,029,872 and 7,449,308). These genetically modified host cells have been used to produce antibodies that have predominantly particular N-glycan structures.

In addition, since fungi such as yeast or filamentous fungi lack the ability to produce fucosylated glycoproteins, antibodies produced in such cells will lack fucose unless the cells are further modified to include the enzymatic pathway for producing fucosylated glycoproteins (see for example, PCT Publication WO2008112092). In particular embodiments, the antibodies disclosed herein further include those produced in lower eukaryotic host cells and which comprise fucosylated and nonfucosylated hybrid and complex N-glycans, including bisected and multiantennary species, including but not limited to N-glycans such as GlcNAc (1-4)Man3GlcNAc2; Gal(1-4)GlcNAc(1-4)Man3GlcNAc2; NANA (1-4) Gal(1-4)GlcNAc(1-4)Man3GlcNAc2. In particular embodiments, the antibody compositions provided herein may comprise antibodies having at least one hybrid N-glycan selected from the group consisting of GlcNAcMan5GlcNAc2; GalGlcNAcMan5GlcNAc2; and NANAGalGlcNAcMan5GlcNAc2. In particular aspects, the hybrid N-glycan is the predominant N-glycan species in the composition. In further aspects, the hybrid N-glycan is a particular N-glycan species that comprises about 30%, 40%, 50%, 60%, 70%, 80%, 90%, 95%, 97%, 98%, 99%, or 100% of the hybrid N-glycans in the composition.

In particular embodiments, the antibody compositions provided herein comprise antibodies having at least one complex N-glycan selected from the group consisting of GlcNAcMan3GlcNAc2; GalGlcNAcMan3GlcNAc2; NANAGalGlcNAcMan3GlcNAc2; GlcNAc2Man3GlcNAc2; GalGlcNAc2Man3GlcNAc2; Gal2GlcNAc2Man3GlcNAc2; NANAGal2GlcNAc2Man3GlcNAc2; and NANA2Gal2GlcNAc2Man3GlcNAc2. In particular aspects, the complex N-glycan is the predominant N-glycan species in the composition. In further aspects, the complex N-glycan is a particular N-glycan species that comprises about 30%, 40%, 50%, 60%, 70%, 80%, 90%, 95%, 97%, 98%, 99%, or 100% of the complex N-glycans in the composition. In particular embodiments, the N-glycan is fusosylated. In general, the fucose is in an α1,3-linkage with the GlcNAc at the reducing end of the N-glycan, an a1,6-linkage with the GlcNAc at the reducing end of the N-glycan, an a1,2-linkage with the Gal at the non-reducing end of the N-glycan, an α1,3-linkage with the GlcNac at the non-reducing end of the N-glycan, or an a1,4-linkage with a GlcNAc at the non-reducing end of the N-glycan.

Therefore, in particular aspects of the above the glycoprotein compositions, the glycoform is in an α1,3-linkage or a1,6-linkage fucose to produce a glycoform selected from the group consisting of Man5GlcNAc2(Fuc), GlcNAcMan5GlcNAc2(Fuc), Man3GlcNAc2(Fuc), GlcNAcMan3GlcNAc2(Fuc), GlcNAc2Man3GlcNAc2(Fuc), GalGlcNAc2Man3GlcNAc2(Fuc), Gal2GlcNAc2Man3GlcNAc2(Fuc), NANAGal2GlcNAc2Man3GlcNAc2(Fuc), and NANA2Gal2GlcNAc2Man3GlcNAc2(Fuc); in an α1,3-linkage or a1,4-linkage fucose to produce a glycoform selected from the group consisting of GlcNAc(Fuc)Man5GlcNAc2, GlcNAc(Fuc)Man3GlcNAc2, GlcNAc2(Fuc1-2)Man3GlcNAc2, GalGlcNAc2(Fuc1-2)Man3GlcNAc2, Gal2GlcNAc2(Fuc1-2)Man3GlcNAc2, NANAGal2GlcNAc2(Fuc1-2)Man3GlcNAc2, and NANA2Gal2GlcNAc2(Fuc1-2)Man3GlcNAc2; or in an a1,2-linkage fucose to produce a glycoform from group consisting of selected the Gal(Fuc)GlcNAc2Man3GlcNAc2, Gal2(Fuc1-2)GlcNAc2Man3GlcNAc2, NANAGal2 (Fuc1-2) GlcNAc2Man3GlcNAc2, and NANA2Gal2(Fuc1-2)GlcNAc2Man3GlcNAc2.

In further aspects, the antibodies comprise high mannose N-glycans, including but not limited to, Man8GlcNAc2, Man7GlcNAc2, Man6GlcNAc2, Man5GlcNAc2, Man4GlcNAc2, or N-glycans that consist of the Man3GlcNAc2 N-glycan structure. In further aspects of the above, the complex N-glycans further include fucosylated and non-fucosylated bisected and multiantennary species. As used herein, the terms “N-glycan” and “glycoform” are used interchangeably and refer to an N-linked oligosaccharide, for example, one that is attached by an asparagine-N-acetylglucosamine linkage to an asparagine residue of a polypeptide. N-linked glycoproteins contain an N-acetylglucosamine residue linked to the amide nitrogen of an asparagine residue in the protein.

### D. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5 ×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the VH C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stablizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338 describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

### E. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### V. TREATMENT OF CANCER

### A. Formulation and Administration

The present disclosure provides pharmaceutical compositions comprising anti-LILRB antibodies and antigens for generating the same. Such compositions comprise a prophylactically or therapeutically effective amount of an antibody or a fragment thereof, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Antibodies of the present disclosure, as described herein, can be formulated for parenteral administration, e.g., formulated for injection via the intradermal, intravenous, intraarterial, intramuscular, subcutaneous, intra-tumoral or even intraperitoneal routes. The antibodies could alternatively be administered by a topical route directly to the mucosa, for example by nasal drops, inhalation, or by nebulizer. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### B. Cell Therapies

In another aspect, the present disclosure provides immune cells which express a chimeric antigen receptor (CAR). In some embodiment, The CAR comprises an antigen-binding fragment provided herein. In an embodiment, the CAR protein includes from the N-terminus to the C-terminus: a leader peptide, an anti-LILRB2 heavy chain variable domain, a linker domain, an anti-LILRB2 light chain variable domain, a human IgG1-CH2-CH3 domain, a spacer region, a CD28 transmembrane domain, a 4-1BB intracellular co-stimulatory signaling and a CD3 ζ intracellular T cell signaling domain.

Also provided are methods for immunotherapy comprising administering an effective amount of the immune cells of the present disclosure. In one embodiment, a medical disease or disorder is treated by transfer of an immune cell population that elicits an immune response. In certain embodiments of the present disclosure, cancer or infection is treated by transfer of an immune cell population that elicits an immune response. Provided herein are methods for treating or delaying progression of cancer in an individual comprising administering to the individual an effective amount of an antigen-specific cell therapy.

The immune cells may be T cells (e.g., regulatory T cells, CD4+ T cells, CD8+ T cells, or gamma-delta T cells), NK cells, invariant NK cells, NKT cells, or macrophages. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from healthy human subjects, healthy volunteers, or healthy donors. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided that the cells obtained are subject-compatible in that they can be introduced into the subject. Allogeneic donor cells may or may not be human-leukocyte-antigen (HLA)-compatible. To be rendered subject-compatible, allogeneic cells can be treated to reduce immunogenicity.

The immune cells can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In particular embodiments, NK cells are engineered to express a TCR. The NK cells may be further engineered to express a CAR. Multiple CARs and/or TCRs, such as to different antigens, may be added to a single cell type, such as T cells or NK cells.

Suitable methods of modification are known in the art. See, for instance, Sambrook et al., supra; and Ausubel et al., CURRENT PROTOCOLS IN MOLECULAR BIOLOGY, Greene Publishing Associates and John Wiley & Sons, NY, 1994. For example, the cells may be transduced to express a T cell receptor (TCR) having antigenic specificity for a cancer antigen using transduction techniques described in Heemskerk et al. (2008) and Johnson et al. (2009).

In some embodiments, the cells comprise one or more nucleic acids introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

### C. Combination Therapies

It may also be desirable to provide combination treatments using antibodies of the present disclosure in conjunction with additional anti-cancer therapies. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the antibody and the other includes the other agent.

Alternatively, the antibody may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several 10 days (2, 3, 4, 5, 6 or 7) to several weeks (1, 2, 3, 4, 5, 6, 7 or 8) lapse between the respective administrations.

It also is conceivable that more than one administration of either the anti-LILRB2 antibody or the other therapy will be desired. Various combinations may be employed, where the antibody is “A,” and the other therapy is “B,” as exemplified below:

A/B/A B/A/B B/B/A A/A/B B/A/A A/B/B B/B/B/A
B/B/A/B A/A/B/B A/B/A/B A/B/B/A B/B/A/A
B/A/B/A B/A/A/B B/B/B/A A/A/A/B B/A/A/A A/B/A/A
A/A/B/A A/B/B/B B/A/B/B B/B/A/B

Other combinations are contemplated. To kill cells, inhibit cell growth, inhibit metastasis, inhibit angiogenesis or otherwise reverse or reduce the malignant phenotype of tumor cells, using the methods and compositions of the present invention, one may contact a target cell or site with an antibody and at least one other therapy. These therapies would be provided in a combined amount effective to kill or inhibit proliferation of cancer cells. This process may involve contacting the cells/site/subject with the agents/therapies at the same time.

Particular agents contemplated for combination therapy with antibodies of the present disclosure include chemotherapy and hematopoietic stem cell transplantation. Chemotherapy may include cytarabine (ara-C) and an anthracycline (most often daunorubicin), high-dose cytarabine alone, all-trans-retinoic acid (ATRA) in addition to induction chemotherapy, usually an anthracycline, histamine dihydrochloride (Ceplene) and interleukin 2 (Proleukin) after the completion of consolidation therapy, gemtuzumab ozogamicin (Mylotarg) for patients aged more than 60 years with relapsed AML who are not candidates for high-dose chemotherapy, clofarabine, as well as targeted therapies, such as kinase inhibitors, farnesyl transferase inhibitors, decitabine, and inhibitors of MDR1 (multidrug-resistance protein), or arsenic trioxide or relapsed acute promyelocytic leukemia (APL).

In certain embodiments, the agents for combination therapy are one or more drugs selected from the group consisting of a topoisomerase inhibitor, an anthracycline topoisomerase inhibitor, an anthracycline, a daunorubicin, a nucleoside metabolic inhibitor, a cytarabine, a hypomethylating agent, a low dose cytarabine (LDAC), a combination of daunorubicin and cytarabine, a daunorubicin and cytarabine liposome for injection, Vyxeos®, an azacytidine, Vidaza®, a decitabine, an all-trans-retinoic acid (ATRA), an arsenic, an arsenic trioxide, a histamine dihydrochloride, Ceplene®, an interleukin-2, an aldesleukin, Proleukin®, a gemtuzumab ozogamicin, Mylotarg®, an FLT-3 inhibitor, a midostaurin, Rydapt®, a clofarabine, a farnesyl transferase inhibitor, a decitabine, an IDH1 inhibitor, an ivosidenib, Tibsovo®, an IDH2 inhibitor, an enasidenib, Idhifa®, a smoothened (SMO) inhibitor, a glasdegib, an arginase inhibitor, an IDO inhibitor, an epacadostat, a BCL-2 inihbitor, a venetoclax, Venclexta®, a platinum complex derivative, oxaliplatin, a kinase inhibitor, a tyrosine kinase inhibitor, a PI3 kinase inhibitor, a BTK inhibitor, an ibrutinib, IMBRUVICAR, an acalabrutinib, CALQUENCE®, a zanubrutinib, a PD-1 antibody, a PD-L1 antibody, a CTLA-4 antibody, a LAG3 antibody, an ICOS antibody, a TIGIT antibody, a TIM3 antibody, a CD40 antibody, a 4-1BB antibody, a CD47 antibody, a SIRPla antibody or fusions protein, a CD70 antibody, and CLL1 antibody, a CD123 antibody, an antagonist of E-selectin, an antibody binding to a tumor antigen, an antibody binding to a T-cell surface marker, an antibody binding to a myeloid cell or NK cell surface marker, an alkylating agent, a nitrosourea agent, an antimetabolite, an antitumor antibiotic, an alkaloid derived from a plant, a hormone therapy medicine, a hormone antagonist, an aromatase inhibitor, and a P-glycoprotein inhibitor.

### VI. ANTIBODY CONJUGATES

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody-drug conjugates have emerged as a breakthrough approach to the development of cancer therapeutics. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

Antibody conjugates are also preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA). Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugate contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6a-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl) propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### VII. IMMUNODETECTION METHODS

In still further embodiments, the present disclosure concerns immunodetection methods for binding, purifying, removing, quantifying and otherwise generally detecting LILRB-related cancers. While such methods can be applied in a traditional sense, another use will be in quality control and monitoring of vaccine and other virus stocks, where antibodies according to the present disclosure can be used to assess the amount or integrity (i.e., long term stability) of H1 antigens in viruses. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of LILRBs also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing LILRB-related cancers and contacting the sample with a first antibody in accordance with the present disclosure, as the case may be, under conditions effective to allow the formation of immunocomplexes.

These methods include methods for detecting or purifying LILRBs or LILRB-related cancer cells from a sample. The antibody will preferably be linked to a solid support, such as in the form of a column matrix, and the sample suspected of containing the LILRB-related cancer cells will be applied to the immobilized antibody. The unwanted components will be washed from the column, leaving the LILRB-expressing cells immunocomplexed to the immobilized antibody, which is then collected by removing the organism or antigen from the column.

The immunobinding methods also include methods for detecting and quantifying the amount of LILRB-related cancer cells or related components in a sample and the detection and quantification of any immune complexes formed during the binding process. Here, one would obtain a sample suspected of containing LILRB-related cancer cells and contact the sample with an antibody that binds LILRBs or components thereof, followed by detecting and quantifying the amounts of immune complexes formed under the specific conditions. In terms of antigen detection, the biological sample analyzed may be any sample that is suspected of containing LILRB-related cancers, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to LILRBs. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### 1. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the LILRB-related cancer cells is added to the wells. After binding and washing to remove non-specifically bound immune complexes, the bound antigen may be detected. Detection may be achieved by the addition of another anti-LILRB antibody that is linked to a detectable label. This type of ELISA is a simple “sandwich ELISA.” Detection may also be achieved by the addition of a second anti-LILRB2 antibody, followed by the addition of a third antibody that has binding affinity for the second antibody, with the third antibody being linked to a detectable label.

In another exemplary ELISA, the samples suspected of containing the LILRB2-related cancer cells are immobilized onto the well surface and then contacted with the anti-LILRB2 antibodies of the disclosure. After binding and washing to remove non-specifically bound immune complexes, the bound anti-LILRB2 antibodies are detected. Where the initial anti-LILRB2 antibodies are linked to a detectable label, the immune complexes may be detected directly. Again, the immune complexes may be detected using a second antibody that has binding affinity for the first anti-LILRB2 antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

### 2. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### 3. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### 4. Immunodetection Kits

In still further embodiments, the present disclosure concerns immunodetection kits for use with the immunodetection methods described above. As the antibodies may be used to detect LILRB-related cancer cells, the antibodies may be included in the kit. The immunodetection kits will thus comprise, in suitable container means, a first antibody that binds to an LILRB, and optionally an immunodetection reagent.

In certain embodiments, the antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of LILRBs, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### 5. Flow Cytometry and FACS

The antibodies of the present disclosure may also be used in flow cytometry or FACS. Flow cytometry is a laser- or impedance-based technology employed in many detection assays, including cell counting, cell sorting, biomarker detection and protein engineering. The technology suspends cells in a stream of fluid and passing them through an electronic detection apparatus, which allows simultaneous multiparametric analysis of the physical and chemical characteristics of up to thousands of particles per second. Flow cytometry is routinely used in the diagnosis disorders, especially blood cancers, but has many other applications in basic research, clinical practice and clinical trials.

Fluorescence-activated cell sorting (FACS) is a specialized type of cytometry. It provides a method for sorting a heterogenous mixture of biological cells into two or more containers, one cell at a time, based on the specific light scattering and fluorescent characteristics of each cell. In general, the technology involves a cell suspension entrained in the center of a narrow, rapidly flowing stream of liquid. The flow is arranged so that there is a large separation between cells relative to their diameter. A vibrating mechanism causes the stream of cells to break into individual droplets. Just before the stream breaks into droplets, the flow passes through a fluorescence measuring station where the fluorescence of each cell is measured. An electrical charging ring is placed just at the point where the stream breaks into droplets. A charge is placed on the ring based immediately prior to fluorescence intensity being measured, and the opposite charge is trapped on the droplet as it breaks form the stream. The charged droplets then fall through an electrostatic deflection system that diverts droplets into containers based upon their charge.

In certain embodiments, to be used in flow cytometry or FACS, the antibodies of the present disclosure are labeled with fluorophores and then allowed to bind to the cells of interest, which are analyzed in a flow cytometer or sorted by a FACS machine.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

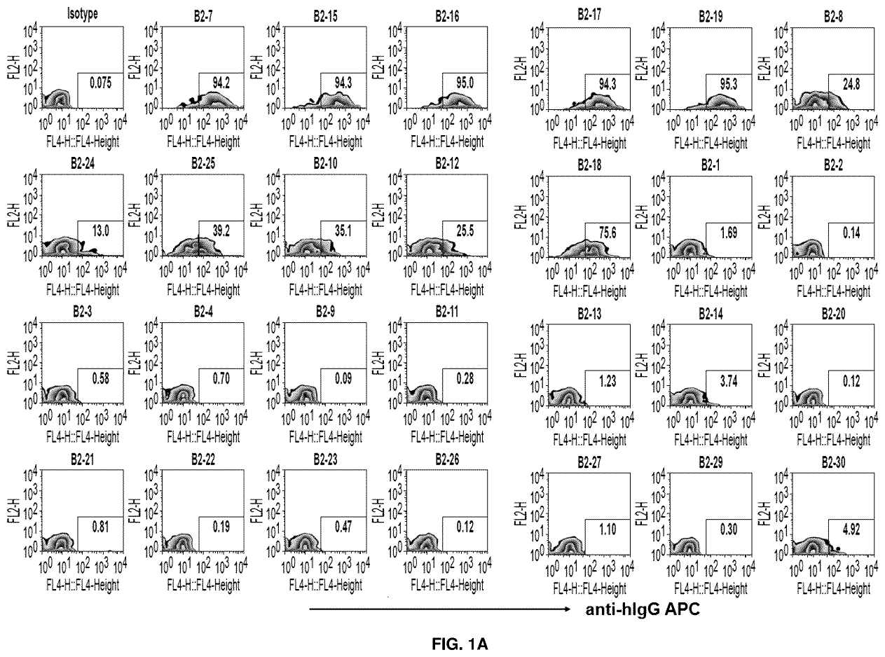

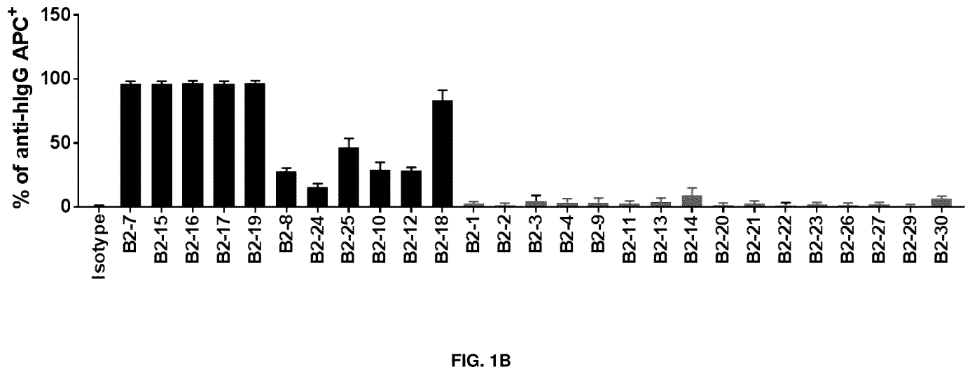

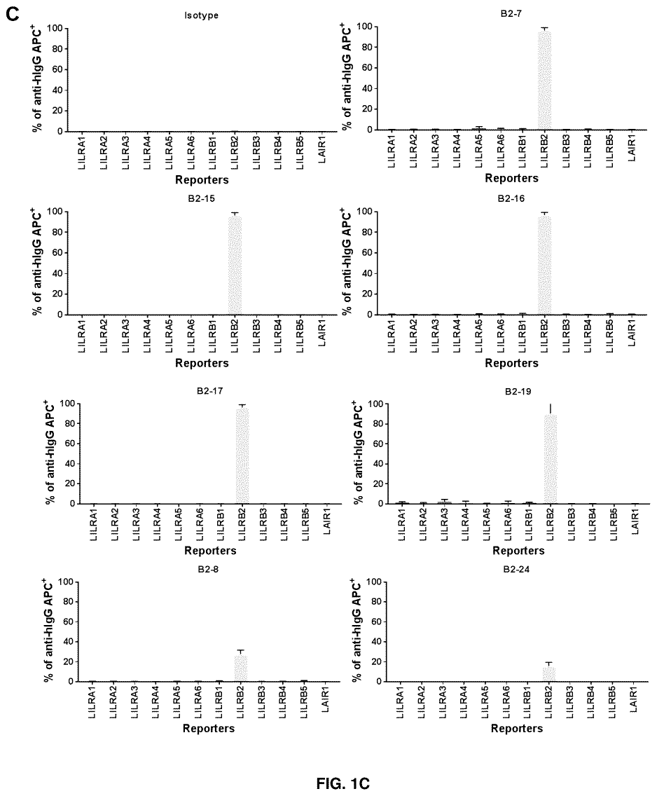

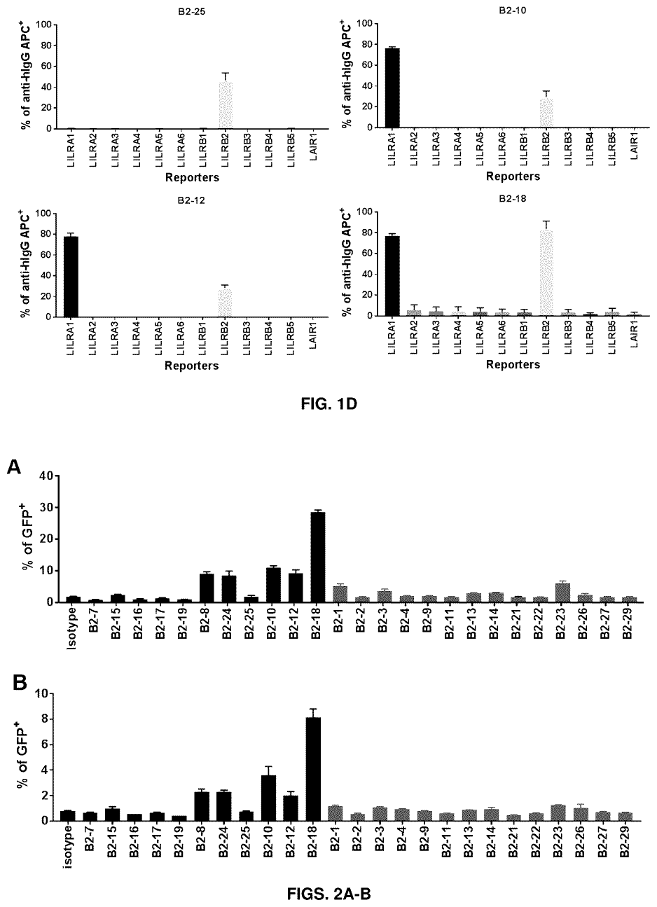

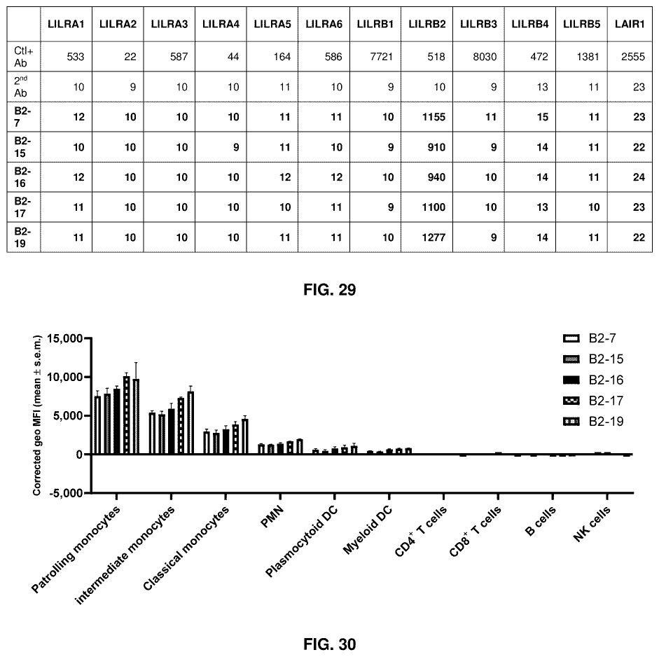

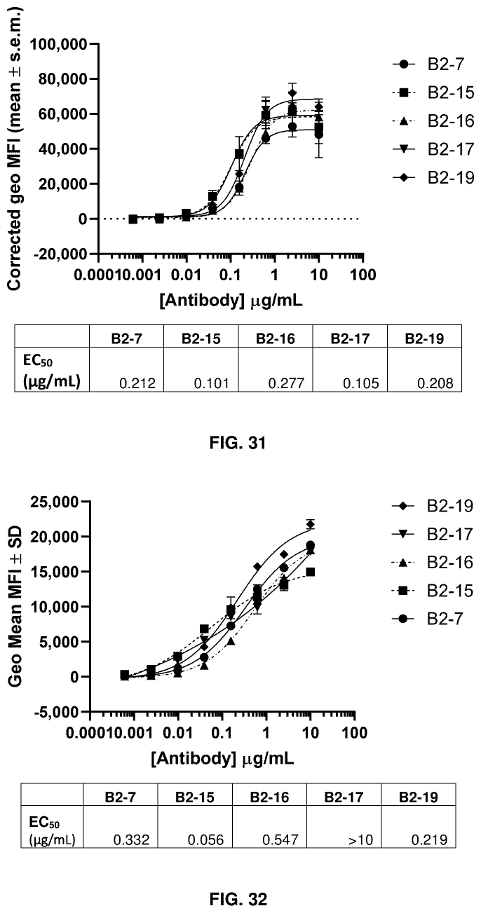

Screening specific monoclonal antibodies for LILRB2. The inventors employed a stable reporter cell system to detect the binding ability of selected monoclonal antibodies to LILRB2. In this chimeric receptor reporter system, the extracellular domain (ECD) of LILRB2 is fused with transmembrane/intracellular domains of PILRb that associates with the adaptor protein DAP12 containing an immunoreceptor tyrosine-based activation motif. 27 monoclonal antibodies were screened. The LILRB2 reporter cells were incubated with selected monoclonal antibody, then labeled with (APC) goat anti-human IgG secondary antibody. Flow cytometer analysis demonstrated that more than 95% LILRB2 reporter cells were labeled with clones B2-7, B2-15, B2-16, B2-17, B2-18, B2-19. A lower percentage of LILRB2 reporter cells were labeled with clones B2-8, B2-10, B2-12, B2-24, B2-25. Other clones did not have binding ability to LILRB2 reporter cells (FIGS. 1A-B). Because members of LILRAs and LILRBs exhibit high sequence homology, the inventors detected the potential cross-reactivity of B2-7, B2-15, B2-16, B2-17, B2-18, B2-19, B2-8, B2-10, B2-12, B2-24 and B2-25 to LILRBAs and LILRBs. The inventors generated LILRBAs and LILRBs reporters stably transduced with each receptor's extracellular domain (ECD). Flow cytometry analysis showed that B2-7, B2-15, B2-16, B2-17, B2-18, B2-19, B2-8, B2-24, B2-25 did not cross-react with LILRAs and other LILRBs except LILRB2. B2-10, B2-12 and B2-18 nonspecifically bound to LILRA1 (FIGS. 1C-D and FIG. 29). Therefore, these were eight antibodies identified as specific monoclonal antibodies for LILRB2. In addition, the inventors demonstrated that B2-7, B2-15, B2-16, B2-17 and B2-19 only bind myeloid cells in samples of human peripheral blood (FIG. 30), thereby further confirming binding on cells expressing endogenous LILRB2. The inventors also confirmed that B2-7, B2-15, B2-16, B2-17 and B2-19 bind HEK293 cells stably expressing full length LILRB2 (HEK293_LILRB2) (FIG. 31) and primary monocytes (FIG. 32) in a dose-dependent manner. The binding EC₅₀ values range from 0.101 to 0.277 μg/mL in HEK293_LILRB2 (FIG. 31) and from 0.056 to >10 μg/mL in primary monocytes (FIG. 32).

Screening potential antagonistic and agonistic antibodies for LILRB2. Typically, receptor blocking antibodies are targeted to the ligand-binding site and compete with the ligand and/or block the interaction of receptor with its ligand. LILRB2 ligands include angiopoietin-like proteins (ANGPTLs), semaphorin 4A (SEMA4A), TIM-3, CD-1 molecules, amyloid-β (Aβ) oligomer, classical human leukocyte antigen (HLA) class I molecules (HLA-A, HLA-B, HLA-C) and non-classical HLA-class I molecules (HLA-E, HLA-F, HLA-G, and HLA-H). The LILRB2 reporter system has provided a powerful tool to screen the potential antagonistic antibody for LILRB2. When the chimeric receptor is activated by ligand binding to the ECD of LILRB2, ZAP70 or Syk kinase is recruited to the immunoreceptor tyrosine-based activation motif of the adaptor DAP12 and activates NFATs to promote GFP expression driven by the NFAT-responsive promoter. An antibody that significantly decreased GFP signaling was consider potential antagonistic antibody.

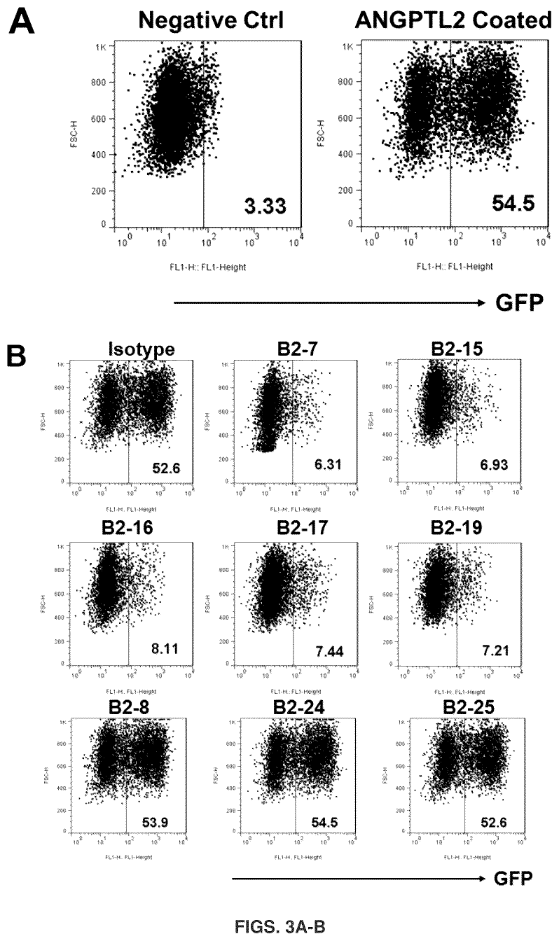

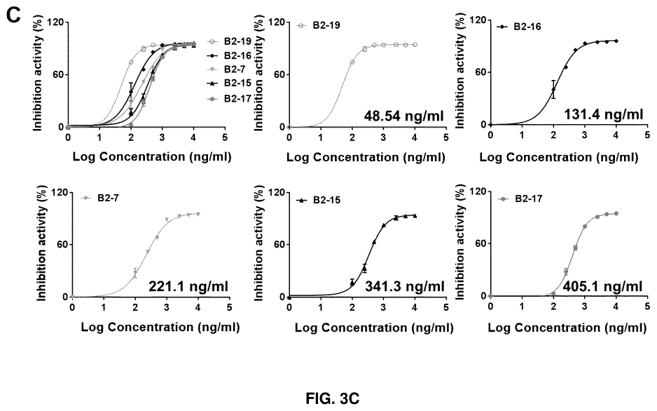

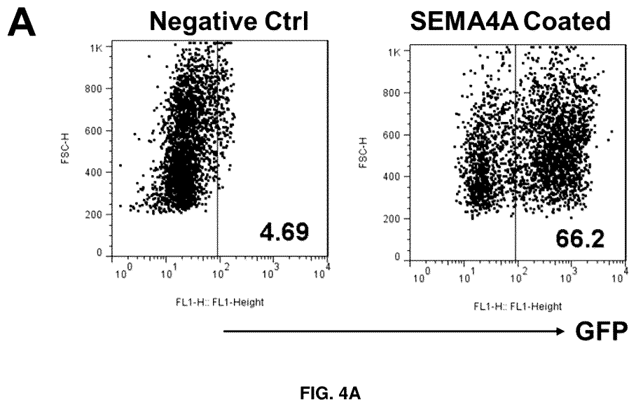

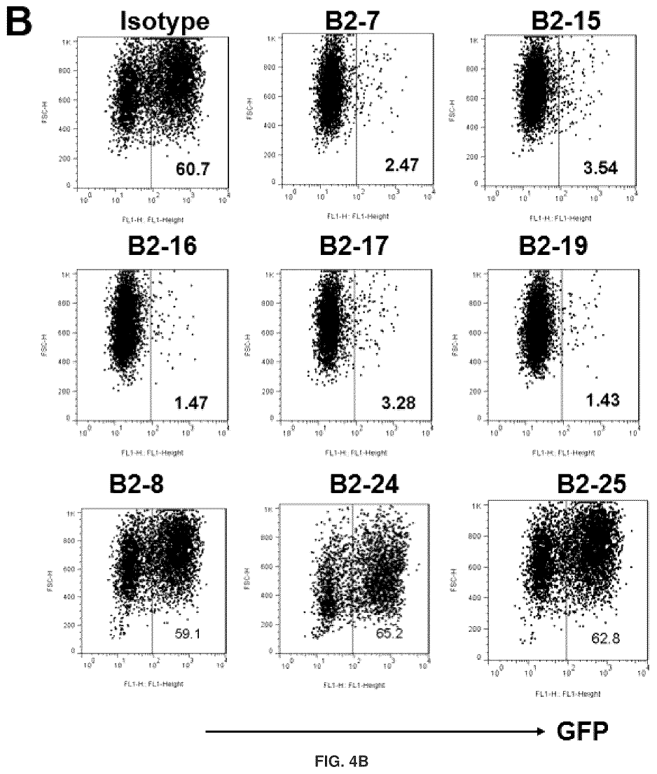

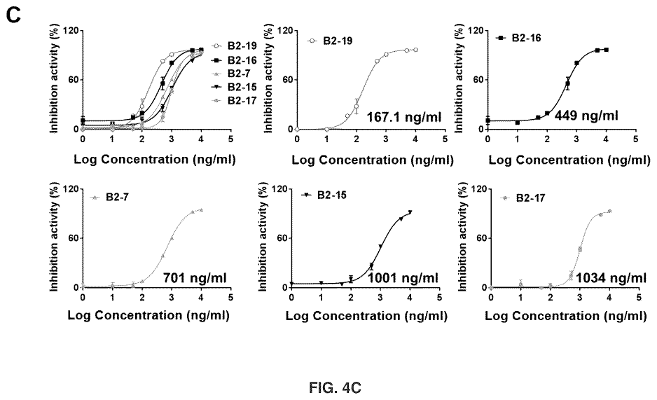

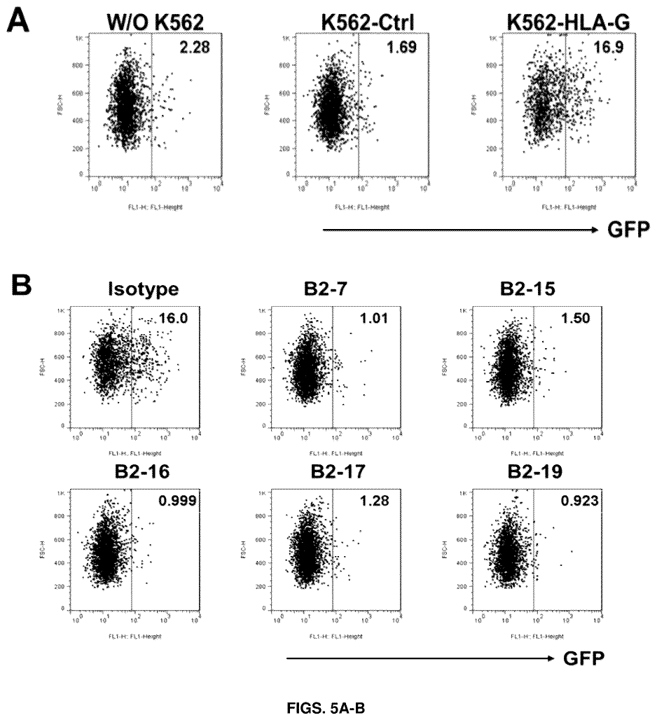

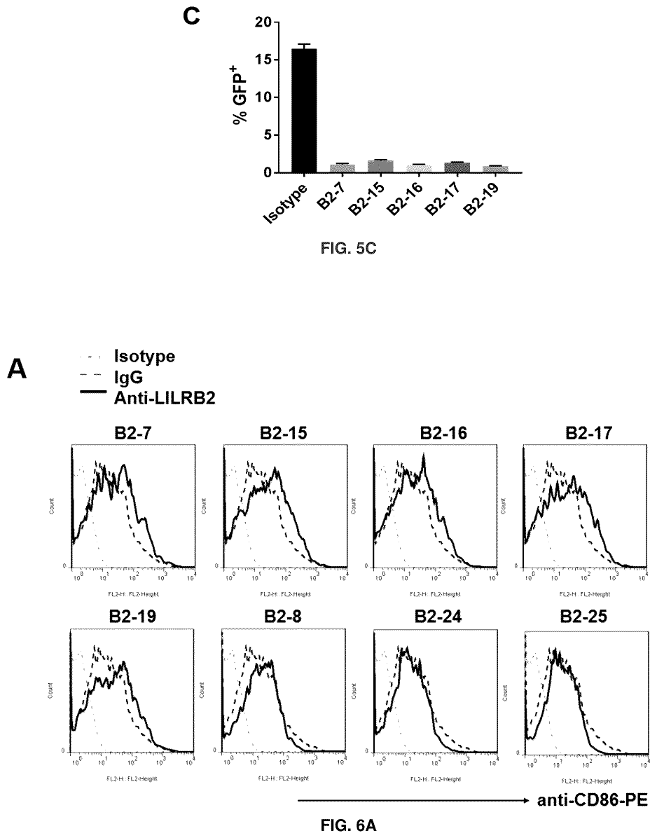

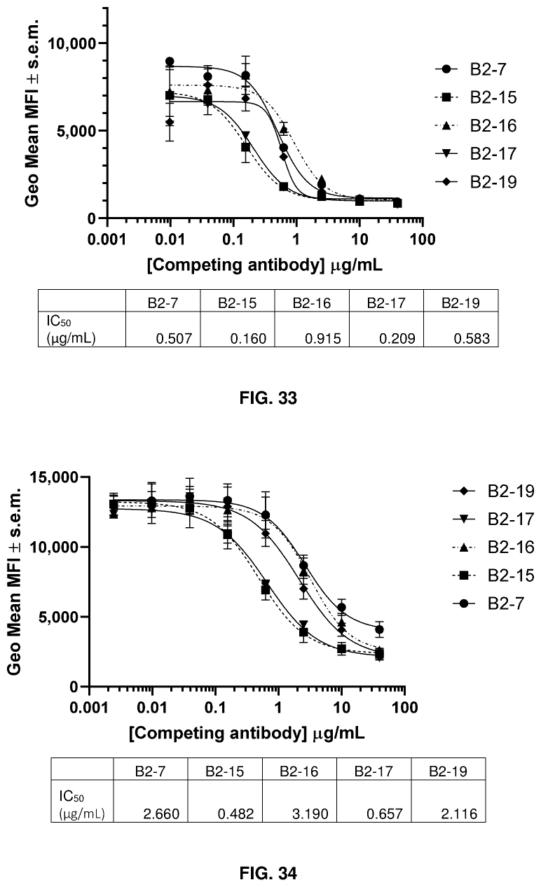

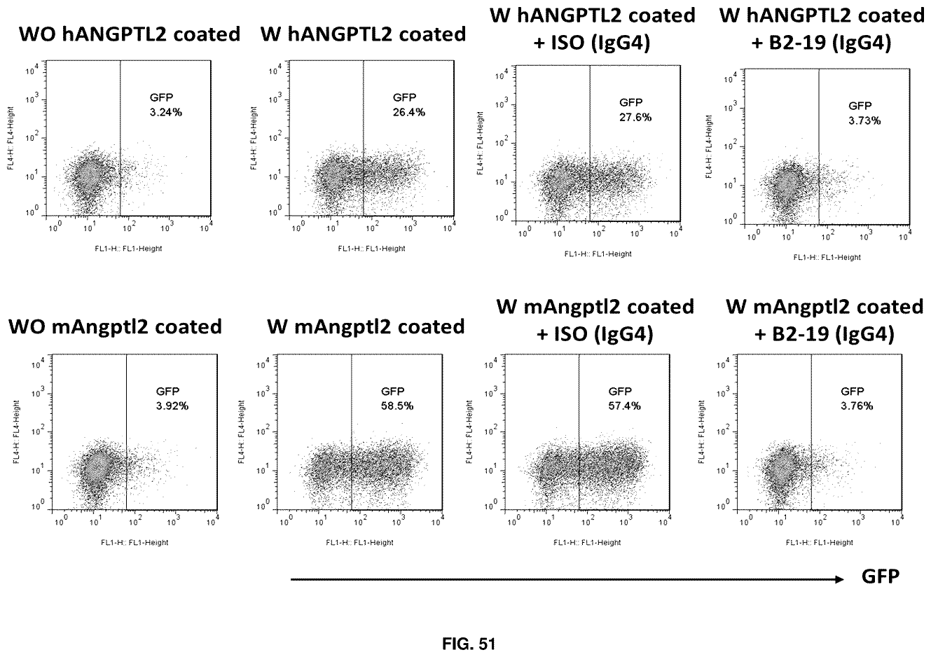

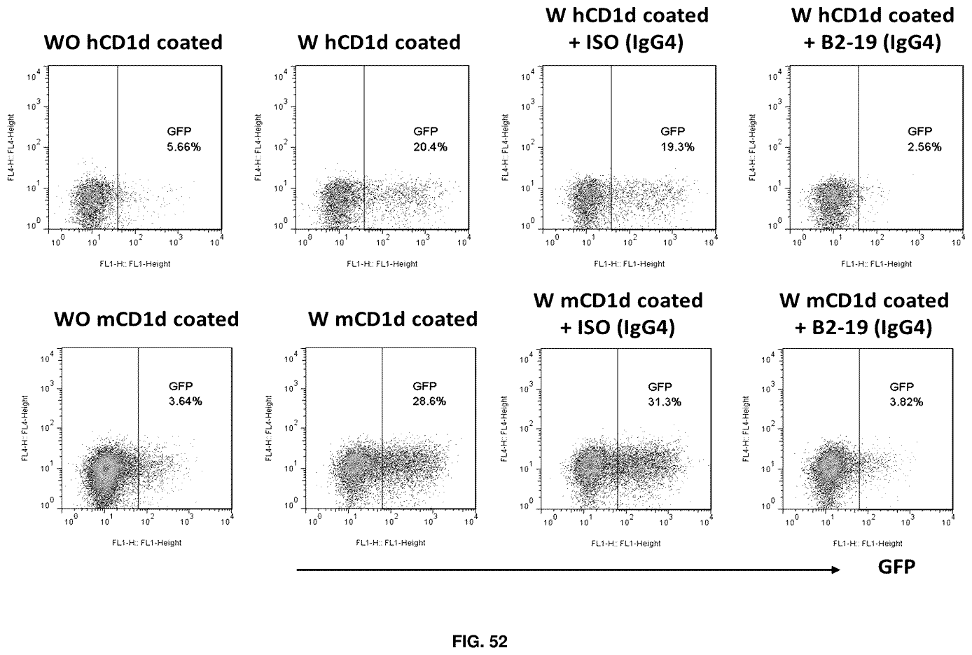

The functions of LILRB2 mAbs to block the activation of LILRB2 reporter cells stimulated by coated ANGPTL2 (FIGS. 3A-C; FIG. 51) and SEMA4A (FIGS. 4A-C) were tested. The results showed that B2-7, B2-15, B2-16, B2-17, and B2-19 could prevent the activation of LILRB2 reporter cells by the coated ligands in a dose-dependent manner. B2-19 antibody was also able to block the activation of LILRB2 reporter cells stimulated by coated CD1d (FIG. 52). The LILRB2 reporter cells were also cocultured with K562 cells overexpressed with HLA-G and then treated with LILRB2 mAbs. The results also showed that these LILRB2 mAbs blocked the activation of LILRB2 reporter cells stimulated by HLA-G on K562 cells (FIGS. 5A-C). LILRB2 antibodies were also shown to block binding of HLA-G and SEMA4A to HEK293 cells expressing full length LILRB2 in a dose-dependent manner (FIG. 33 and FIG. 34, respectively). These data proved that B2-7, B2-15, B2-16, B2-17, and B2-19 are antagonistic LILRB2 mAbs.

Contrastingly, several LILRB2 mAbs (B2-8, B2-24, B2-25, B2-10, B2-12 and B2-18) can activate the LILRB2 reporter cells when added into cell culture medium (FIG. 2A) or the cocultured with K562 (FIG. 2B), demonstrating that these LILRB2 mAbs are agonistic antibodies.

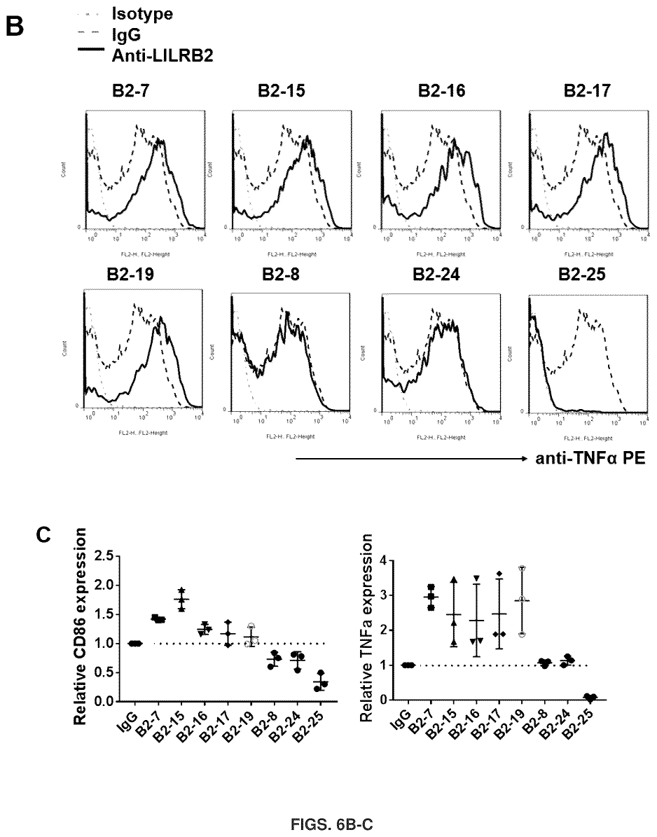

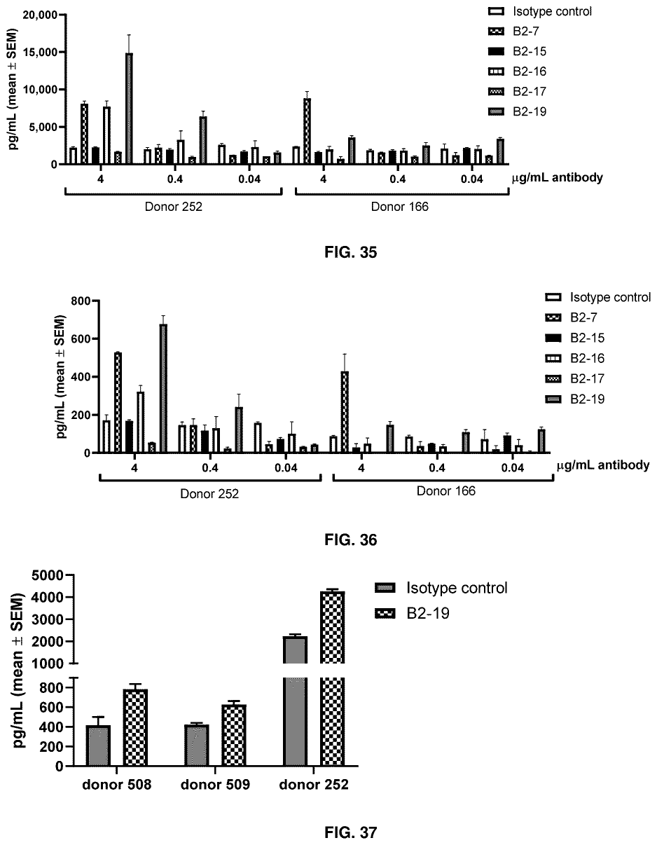

The effect of anti-LILRB2 antibodies on LPS response in primary human monocytes. LILRB2 is expressed on hematopoietic stem cells, monocytes, macrophages, dendritic cells, basophils in some individuals, decidual macrophages, mast cell progenitors, endothelial cells and osteoclasts but not on lymphoid cells. LILRB2 is classified as an immune inhibitory receptor and associated with down modulation of immune response. To determine the function of screened anti-LILRB2 antibodies, peripheral blood mononuclear cell (PBMC) based functional assays were conducted to assess whether screened antibodies could amplify or inhibit monocyte activation. B2-7, B2-15, B2-16, B2-17 and B2-19 could enhance CD86 and TNFα levels in the presence of LPS. B2-8, B2-24 and B2-25 seemed has no effect on TNFα levels, even though showed an inhibition on CD86 levels with LPS stimulation (FIGS. 6A-C). B2-7, B2-16 and B2-19 could enhance IFN-γ (FIG. 35) and TNF-α (FIG. 36) secretion from PBMC stimulated with LPS. B2-19 could also enhance secretion of IL-12p40 from PBMC stimulated with LPS (FIG. 37). The inventors identified B2-7, B2-15, B2-16, B2-17 and B2-19 LILRB2-specific antibodies that enhanced monocyte inflammatory potential in response to LPS stimulation. All these antibodies are functional antagonistic antibodies. B2-8, B2-24 and B2-25 are potential active antibodies for LILRB2.

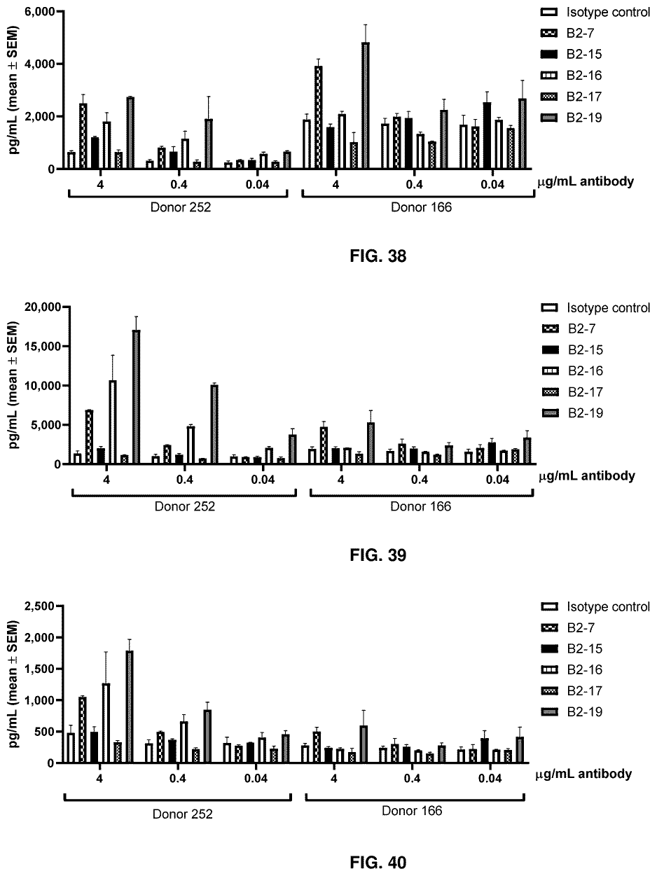

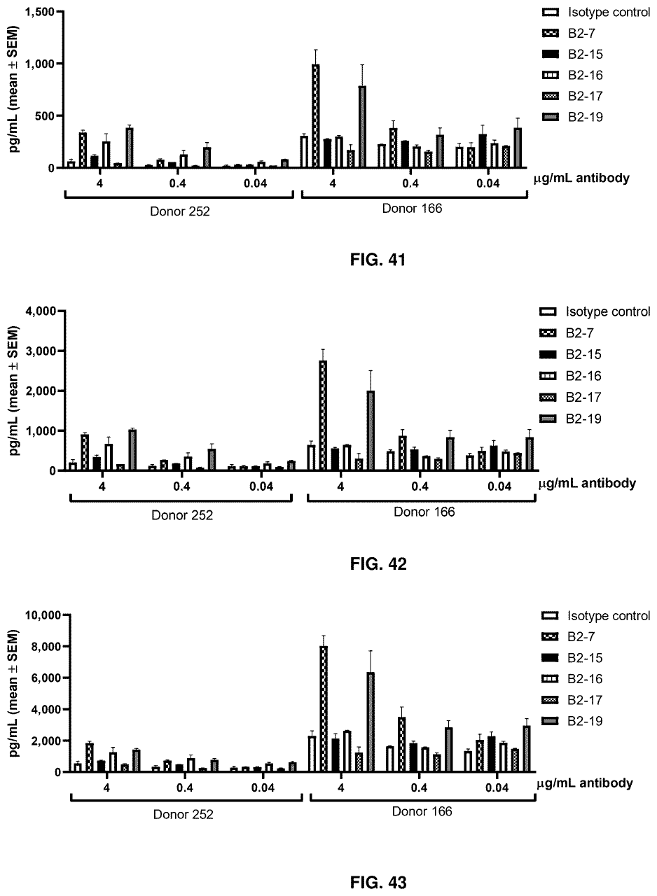

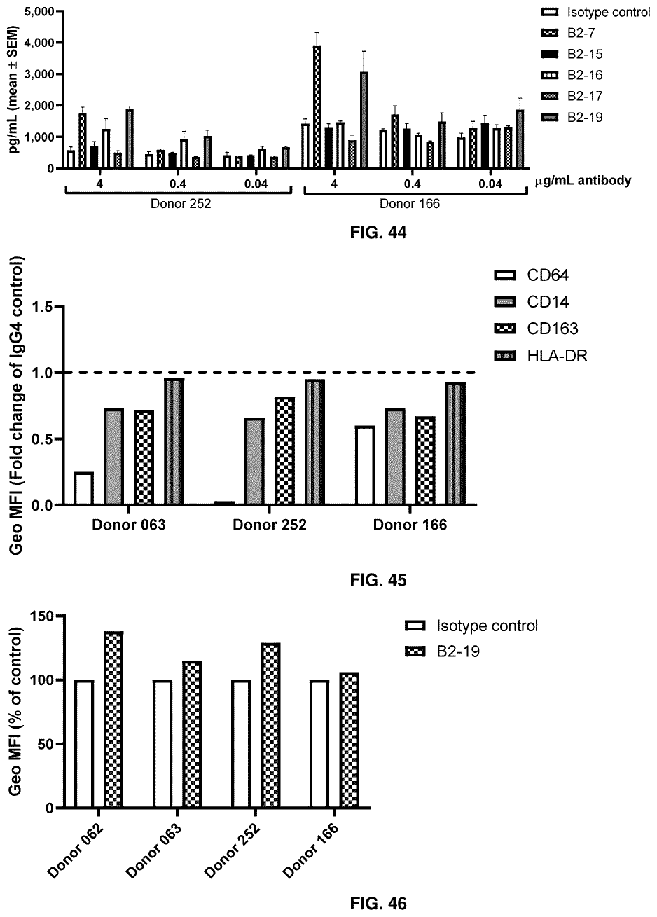

The effect of anti-LILRB2 antibodies on PBMC stimulated by anti-CD3 activating antibody. The inventors evaluated the indirect effect of LILRB2 blockade on T cell activation by an activating anti-CD3 antibody. B2-7 and B2-19 antibodies were able to enhance the secretion of IFN-γ (FIG. 38), TNF-α (FIG. 39), GM-CSF (FIG. 40), IL-1α (FIG. 41), IL-1β (FIG. 42), IL-6 (FIG. 43) and CXCL2 (FIG. 44). B2-19 was also able to enhance the cell surface expression of CD25 on CD8⁺ T cells (FIG. 46). The inventors identified B2-7 and B2-19 LILRB2-specific antibodies that enhanced T cell activation.

The effect of anti-LILRB2 antibodies on monocyte derived M2a macrophages. The inventors evaluated the effect of B2-19 on the phenotype of M2a macrophages in vitro. In M2a macrophages differentiated from monocytes of several donors, B2-19 decreased the expression of CD64, CD163 and CD14 (FIG. 45). The inventors identified B2-19 as a LILRB2-specific antibody capable of modulating the phenotype of M2a macrophages.

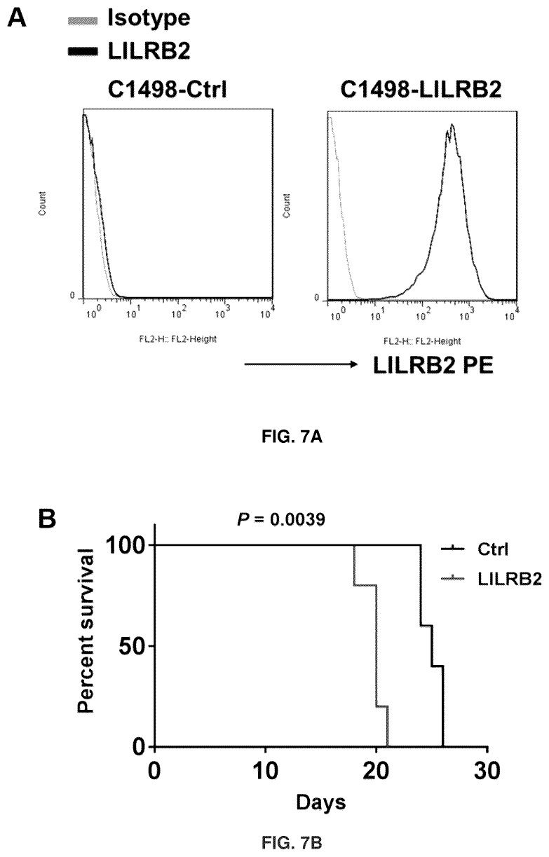

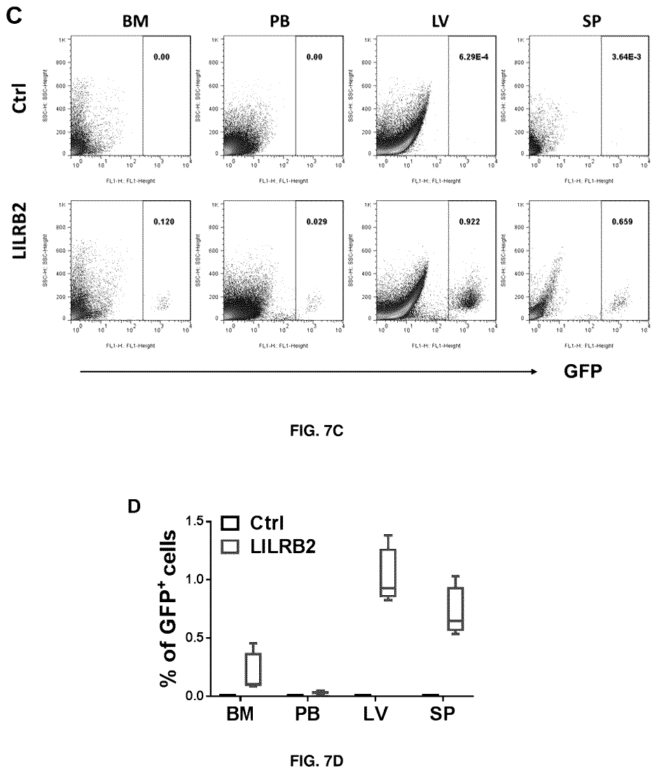

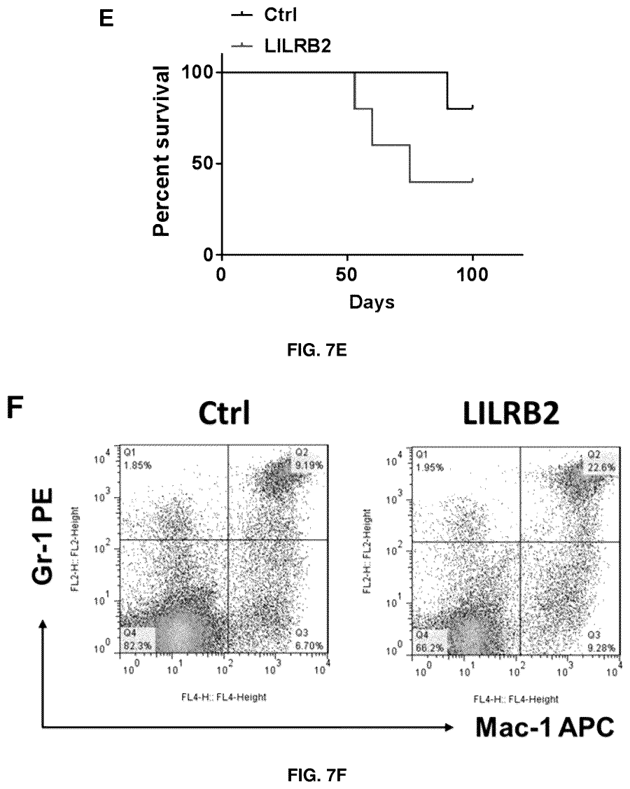

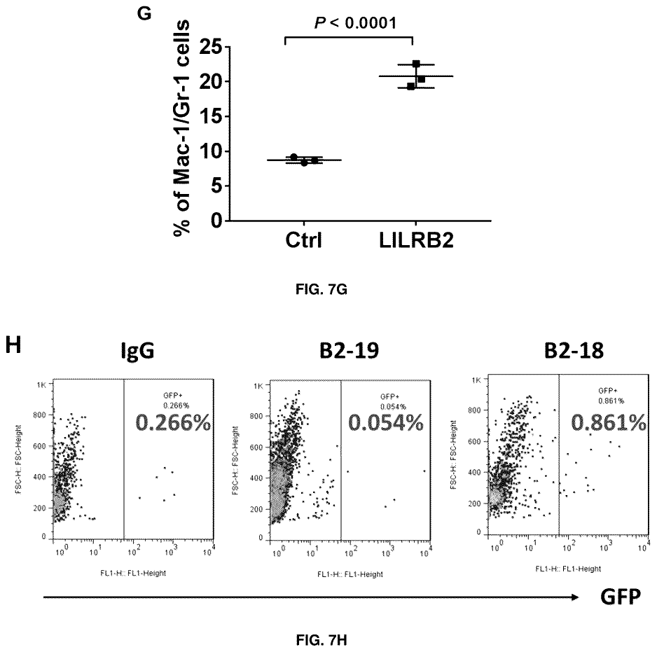

Anti-LILRB2 antibodies inhibit the development of leukemia cells in C1498-LILRB2 tumor-bearing model. The inventors evaluated whether LILRB2 blockade could inhibit tumor progression in a C1498 leukemia model. A C1498 subline overexpressing human LILRB2 (C1498-LILRB2) was generated by retroviral transduction. LILRB2 is stably expressed on C1498-LILRB2 (FIG. 7A). LILRB2 could promote leukemia development. Overexpressed LILRB2 promoted the leukemia development in NSG mice (FIG. 7B). LILRB2 overexpression have worse leukemia infiltration (FIG. 7C-D), survival curve (FIG. 7E) and myoid cell infiltration (FIG. 7G). Furthermore, LALAPG variants of B2-7 and B2-19 containing Fc mutations were generated to eliminate complement binding and fixation as well as Fc-y dependent antibody-dependent cell-mediated cytotoxicity (ADCC) in both murine IgG2a and human IgG1. Treatment with LALAPG mutant antagonistic antibody significantly suppressed myeloid leukemia growth ((FIG. 7H-J).

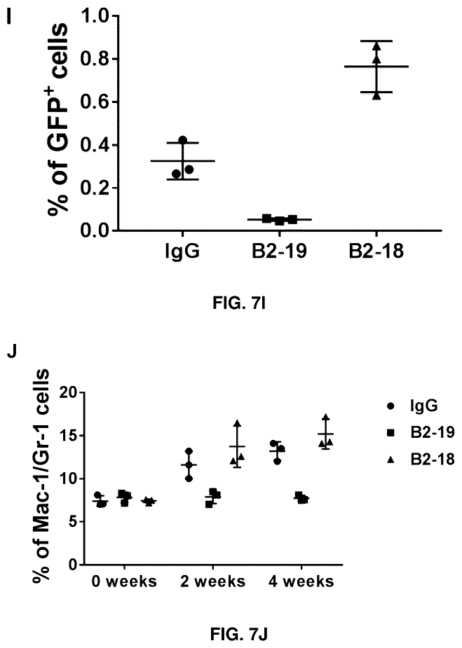

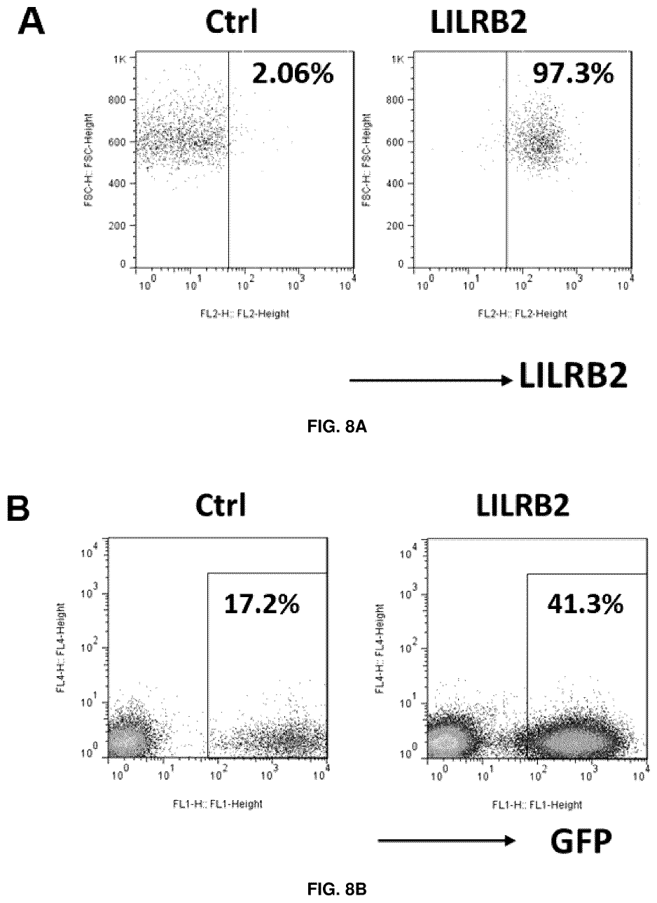

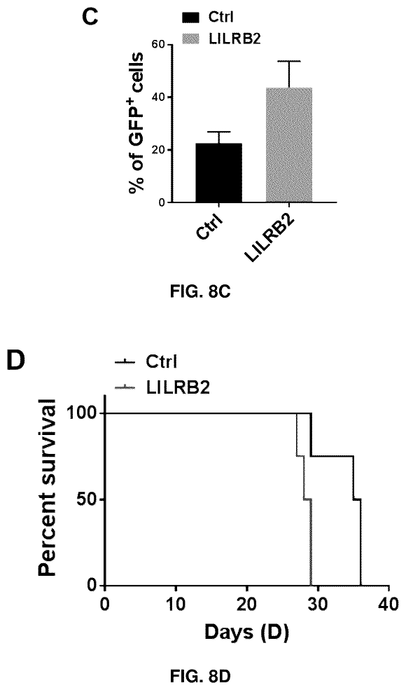

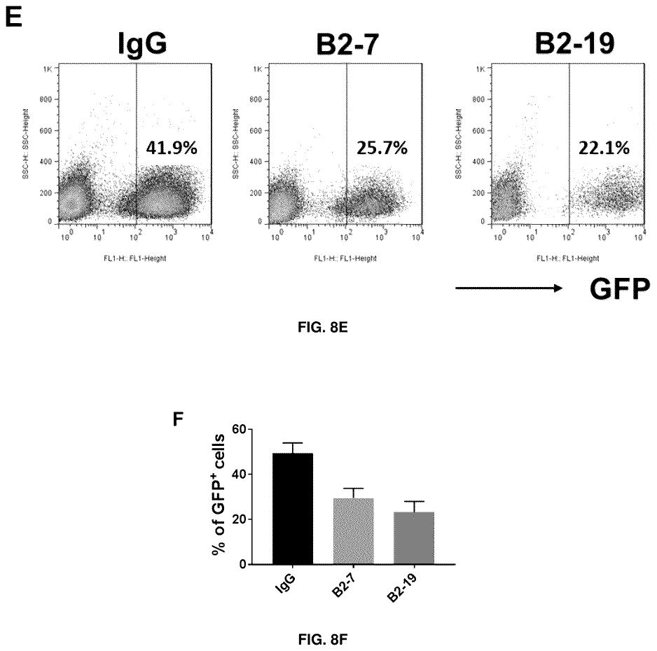

Anti-LILRB2 antibodies inhibit the development of leukemia cells in MLL-AF9 model. The inventors evaluated whether LILRB2 blockade could inhibit tumor progression in an MLL-AF9 leukemia model. A PirB-KO MLL-AF9 leukemia subline overexpressing human LILRB2 (LILRB2) was generated by retroviral transduction and mouse transplantation. Expression of LILRB2 could be detected on PirB-KO MLL-AF9 LILRB2 or control (Ctrl) leukemia cells (FIG. 8A). In this MLL-AF9 model, LILRB2 overexpression have worse leukemia infiltration (FIG. 8B-C), survival curve (FIG. 8D). Furthermore, treatment with LALAPG mutant antagonistic antibody significantly suppressed leukemia infiltration (FIG. 8E-F).

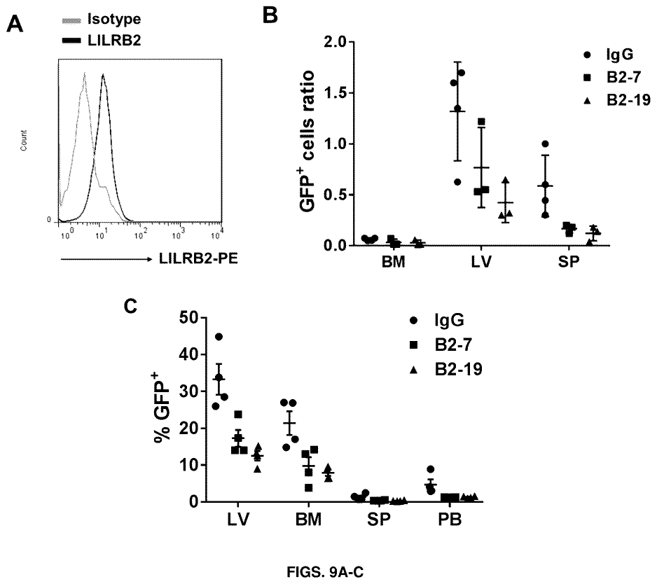

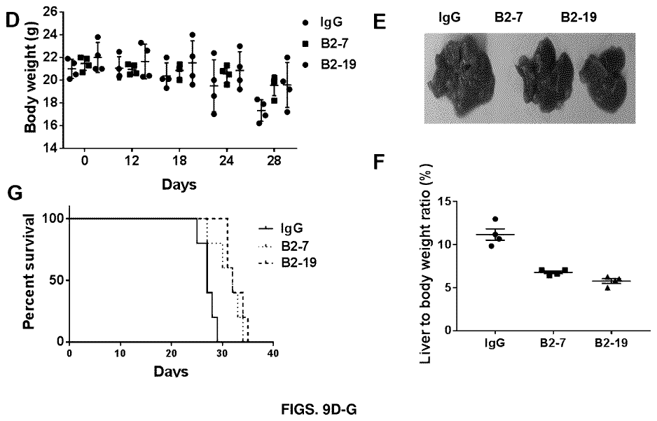

LILRB2 blockage inhibits the migration and infiltration of AML cells. LILRB2 plays various roles in cancer biology as well. Expression of LILRB2 could be detected on THP-1, a human leukemia cell line (FIG. 9A). The inventors used humanized mouse xenograft model to test LILRB2 function on leukemia. Blockage of LILRB2 reduced homing and engraftment of leukemia cells to hematopoietic organs (FIGS. 9B-C), resulting in delayed body weight loss (FIG. 9D), less severe infiltration of myeloid leukemia cells into liver (FIGS. 9E-F), prolonged survival of xenografted mice. Together, blockade of LILRB2 reduced leukemia migration and infiltration.

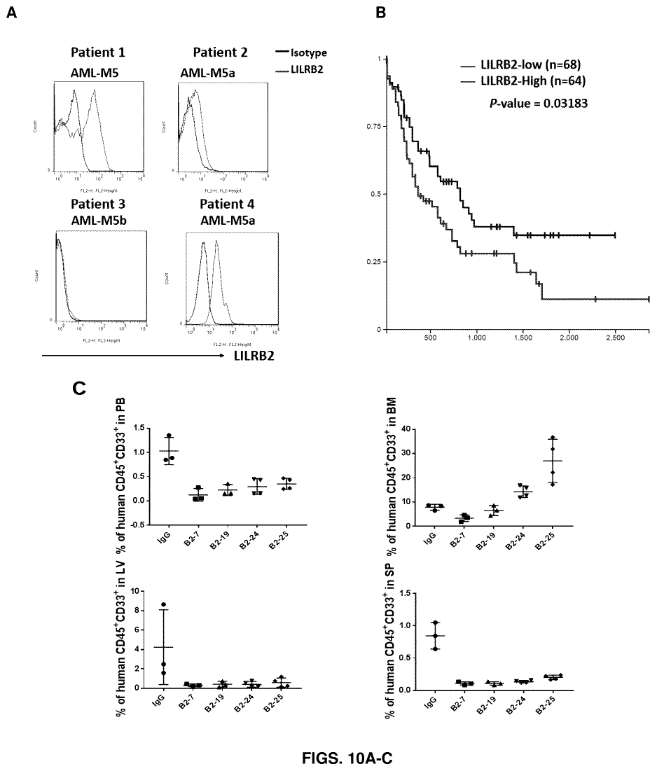

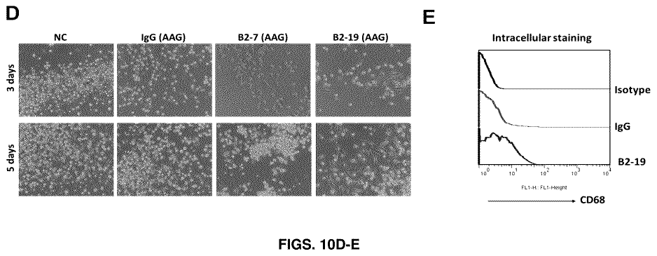

Antagonistic LILRB2 mAbs inhibit the development of leukemia cells in patient-derived xenograft (PDX) model. Flow cytometry analysis showed that of LILRB2 expressed on some cases of primary monocytic AML (M5) (FIG. 10A). In TCGA database, LILRB2 mRNA level was negatively correlated with the AML patient survival (FIG. 10B). In PDX model, LALAPG mutant antagonistic antibodies B2-7 and B2-19 significantly suppressed leukemia infiltration (FIG. 10C). In vitro culture system, LALAPG mutant antagonistic antibody promoted the differentiation of AML-M5 leukemia cells (FIG. 10D-E).

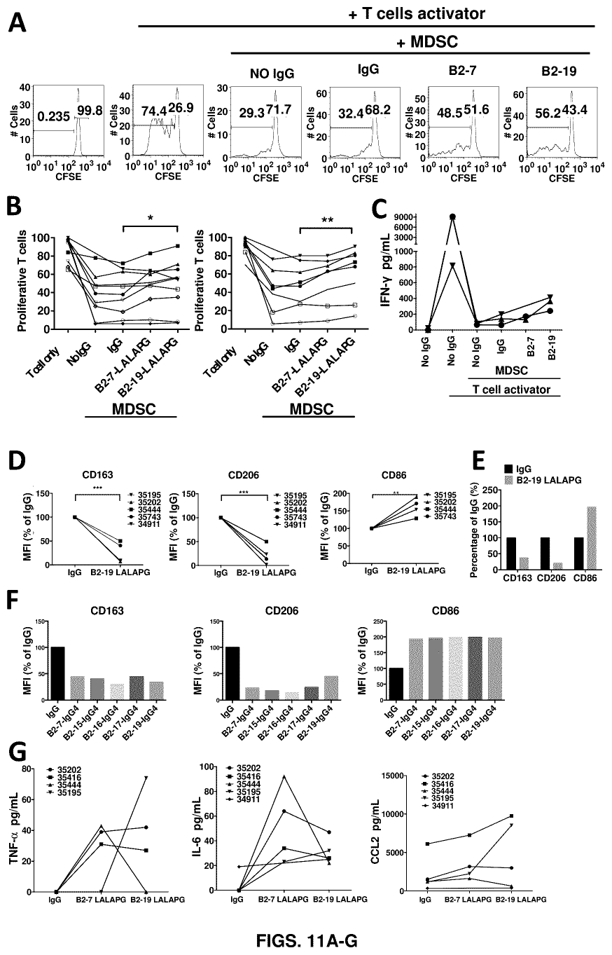

Antagonistic LILRB2 mAbs can prevent the T cell suppressive function of Myeloid-Derived Suppressor Cells (MDSC) in vitro and polarize CD14+ cells from cancer-derived ascites towards a pro-inflammatory phenotype. The function of antagonistic LILRB2 antibodies in T cell suppressive function of MDSC was tested in vitro. Paired MDSC and T cells from the same tumor patient were cocultured and treated with antagonistic LILRB2 antibodies. The treatment of B2-7 and B2-19 antibodies significantly attenuated the inhibition of T cell proliferation induced by MDSC, possibly by converting immune suppressive myeloid cells to proinflammatory myeloid cells (FIGS. 11A-E and FIG. 11G). B2-7, B2-15, B2-16, B2-17 and B2-19 were also able to decrease anti-inflammatory markers CD163 and CD206 and increase pro-inflammatory marker CD86 in primary CD14⁺ cells isolated from cancer-derived ascites (FIG. 11F).

### Example 2

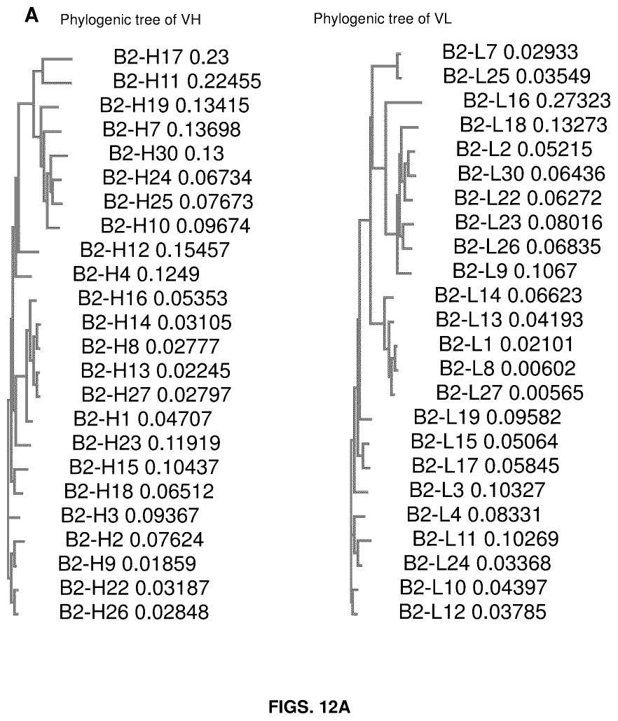

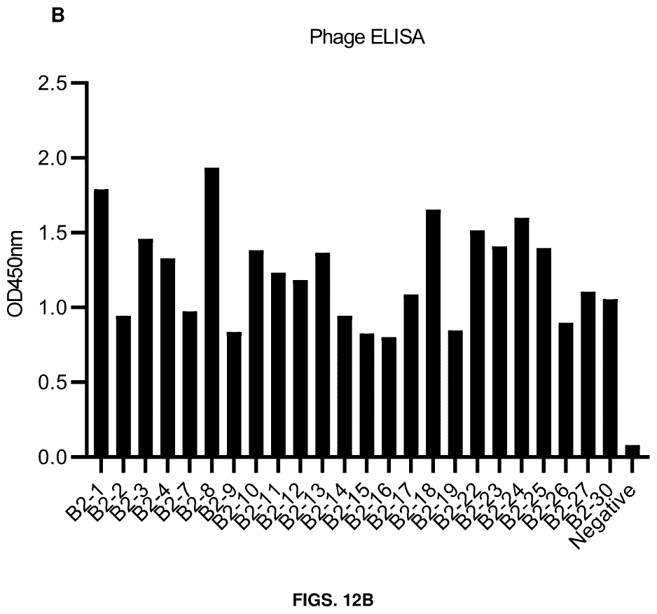

Generation and characterization of LILRB2 antibodies. LILRB2 antibodies were generated by panning phage displayed human antibody scFv library. The LILRB2-His antigen was coated on solid phase for 3 rounds of panning. In each round of phage panning, the LILRB1-His antigen was used for de-selection. After phage panning, single bacterial colonies were picked for phage ELISA testing. The positive ones in phage ELSIA were sequenced and analyzed of theirs scFv region. As shown in FIG. 12A, 24 unique antibody sequences were obtained. The phages presenting the scFv showed binding to LILRB2 antigen in phage ELISA (FIG. 12B). These 24 scFvs were then converted to full human IgG1 for further analysis.

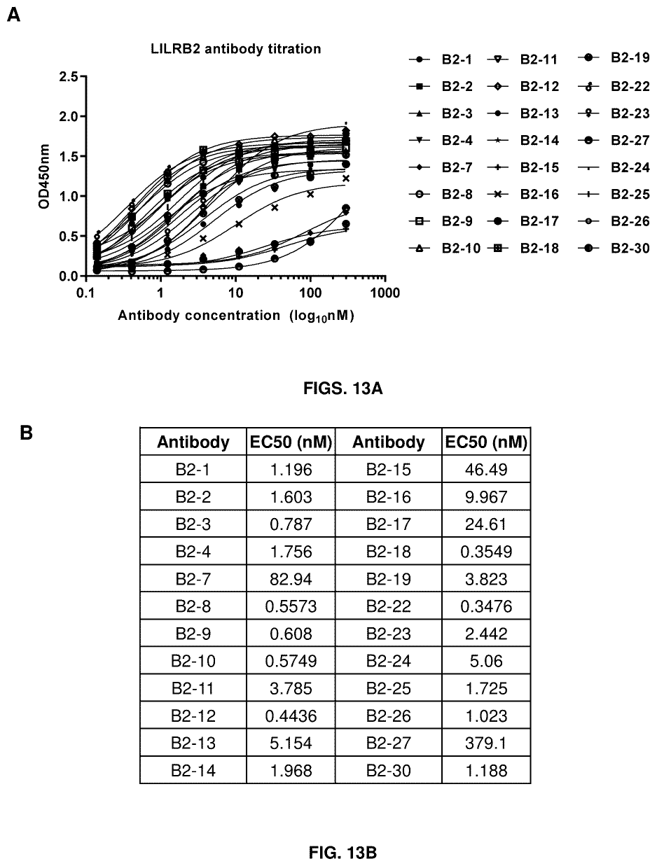

ELISA binding EC₅₀ of LILRB2 antibodies. The ELISA binding EC₅₀ of these 24 antibodies were measured by indirect ELISA. Briefly, ELISA plates were coated with LILRB2-His antigen. Serially diluted antibodies were incubated with antigen coated pates. After incubation, HRP-conjugated goat anti-human Fc secondary antibody was incubated with the plates. The TMB substrate was added for color development and measurement of absorbance at 450 nm. As shown in FIGS. 13A-B, these antibodies bind to LILRB2 in a dose-dependent manner (FIG. 13A) and their EC₅₀ values range from 0.355 to 46.49 nM (FIG. 13B).

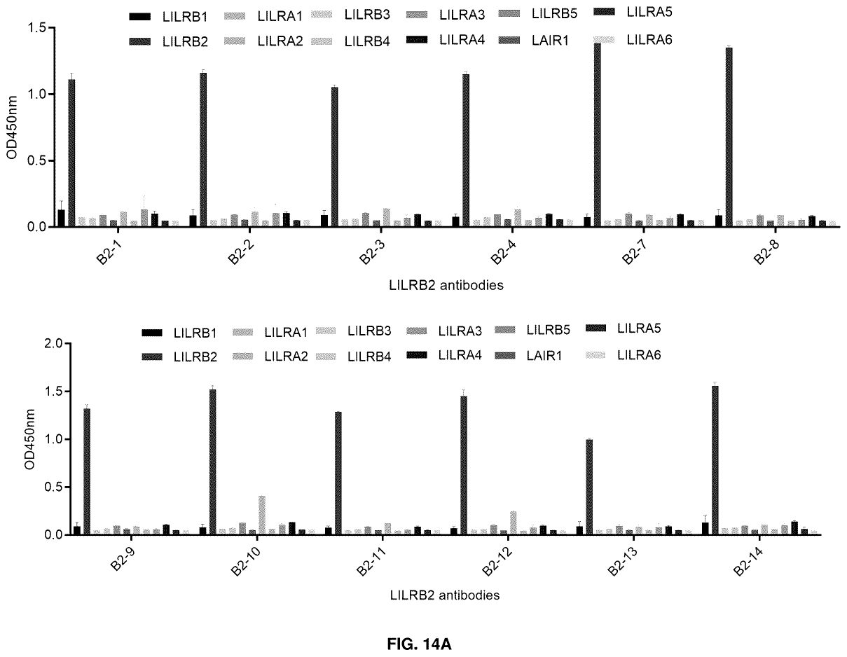

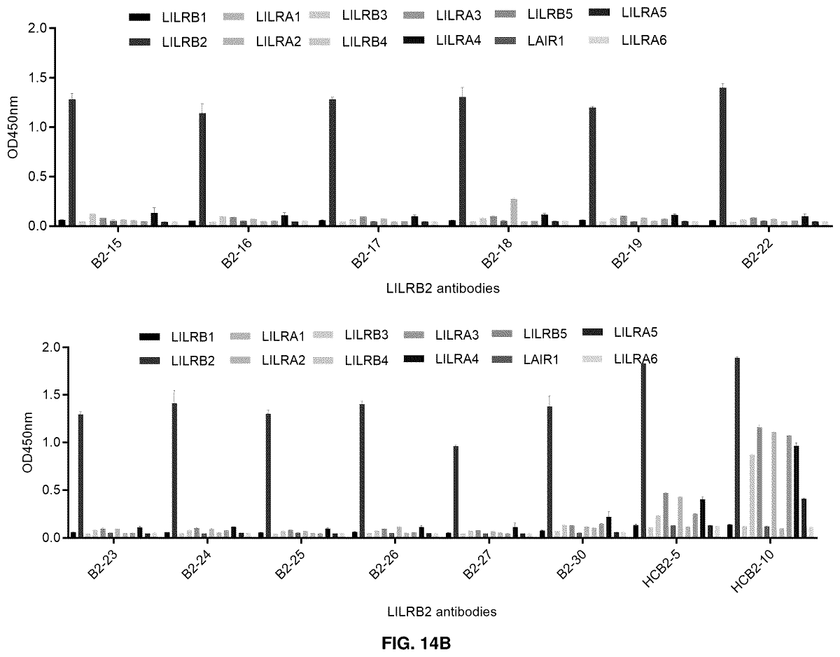

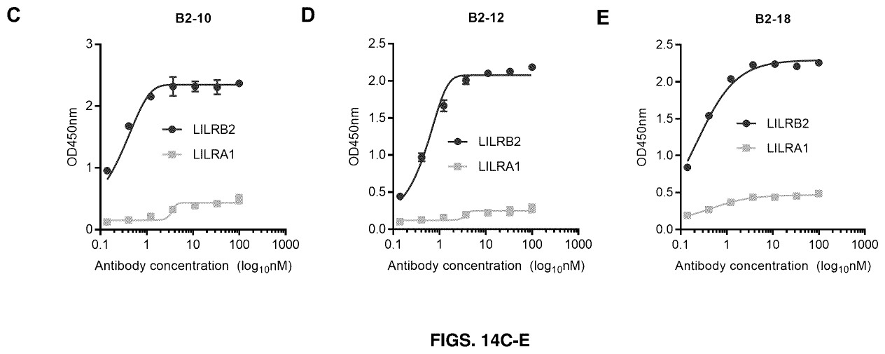

Binding specificity of LILRB2 antibodies. The binding specificity of the LILRB2 antibodies were evaluated by determining their ELISA binding activities with antigens for the other members of LILR family. As shown in FIGS. 14A-B, of the 24 antibodies, 21 showed very specific binding to LILRB2, 3 antibodies (B2-10, -12, -18) showed weak cross binding to LILRA1. A further titration of these 3 antibodies with LILRB2 and LILRA1 antigen showed that the binding abilities of these three antibodies to LILRB2 are much more potent (>100 times) than to LILRA1 (FIG. 14C).

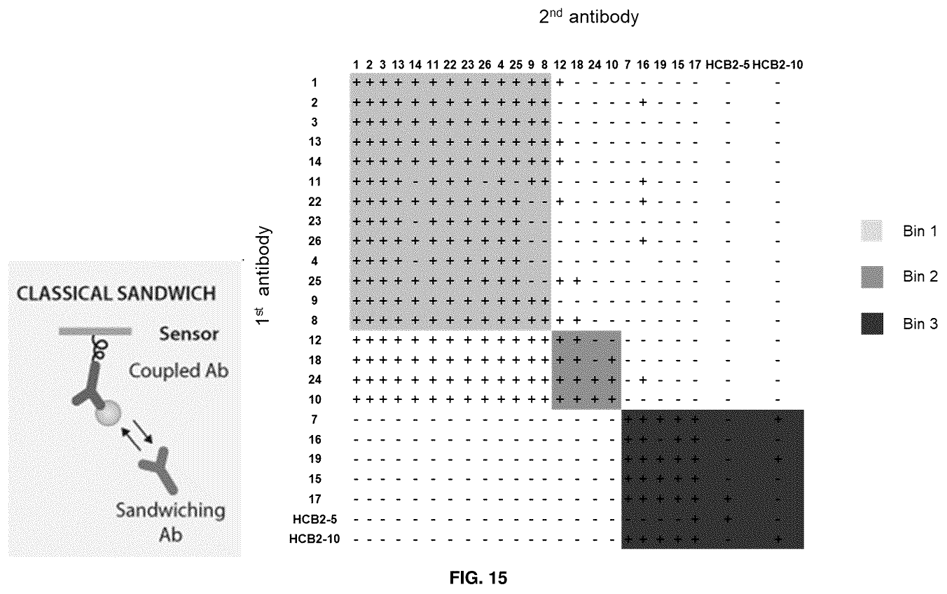

Epitope binning of LILRB2 antibodies. Epitope binning was performed for 22 antibodies on OctetRED96 system by a sandwich format. Each antibody was cross-binned with the rest of antibodies to determine whether they could compete with each other. As shown in FIG. 15, these antibodies can be divided into 3 bins. In each bin, the antibodies compete with each other for LILRB2 binding. Antibodies in bin 1 and bin 2 showed overlap for their blocking profile.

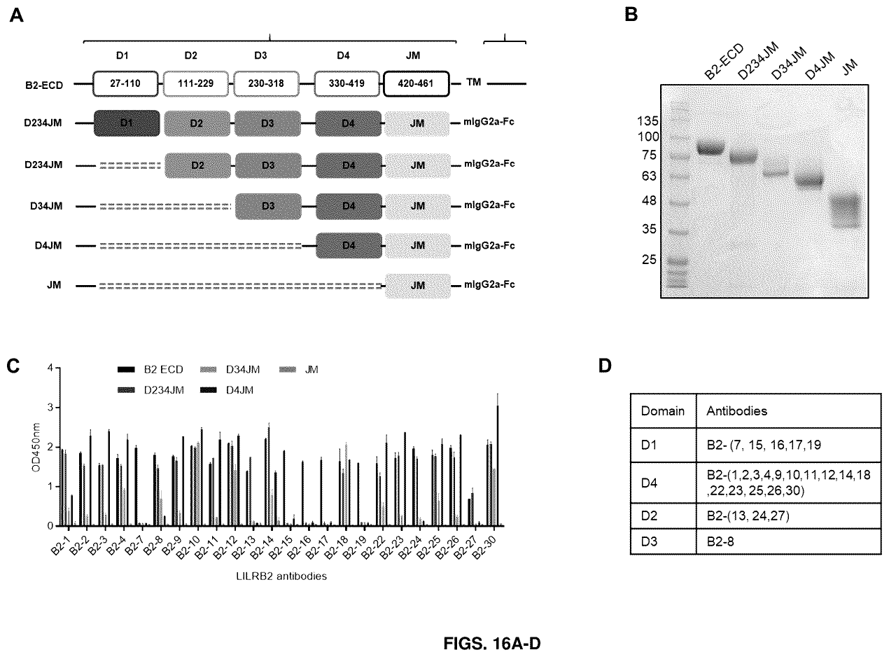

Domain mapping of LILRB2 antibodies. LILRB2 has four Ig-like domains (D1, D2, D3 and D4) and a juxta membrane domain (JM). To determine the binding domain of LILRB2 antibodies, the full extracellular domain (ECD) or the truncated ECD of LILRB2 were fused to Fc fragment of mouse IgG2a for recombinant protein expression (FIGS. 16A-B). The binding abilities of LILRB2 antibodies to these domain proteins were then determined. Deletion of D1 domain resulted in loss of binding to antibodies B2-7,-15, -16, -17 and -19, but not to other antibodies, indicating these antibodies bind to the D1 domain of LILRB2 (FIG. 16C). Similarly, the binding domains of other antibodies were determined and summarized (FIG. 16D).

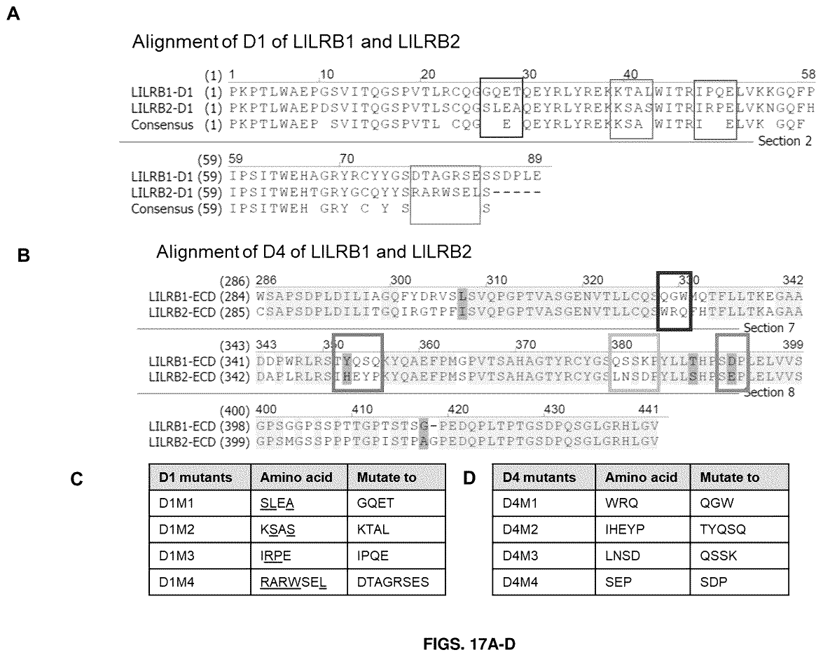

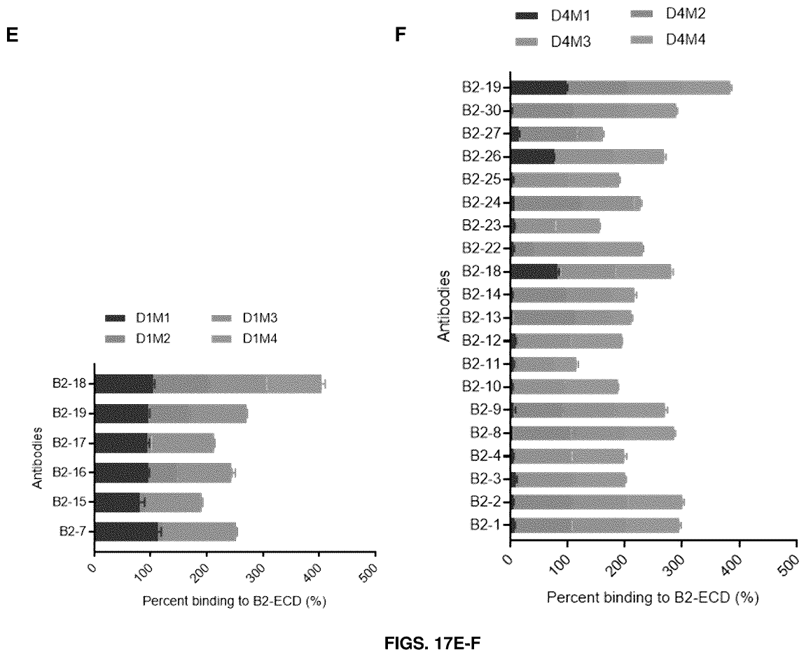

Key residues for antibody binding. To fine map the key residues for antibody binding, a series of mutant B2-ECD proteins were produced for determining antibody binding. Because most antibodies bind to the D1 or D4 domain, the inventors designed mutations only on D1 or D4 domain and the mutations were based on two criteria: 1) residues that are different between LILRB2 and LILRB1; (2) residues that are exposed and locate in the flexible loop region. As shown in FIGS. 17A-C, four regions on D1 domain of LILRB2 were mutated to the correspondent sequences on LILRB1. Similarly, four regions on D4 domain of LILRB2 were also mutated (FIGS. 17B-D). The four D1 mutants were used to test binding with the 5 antibodies (B2-7,-15,-16,-17 and -19) that bind to the D1 domain, and the antibody B2-18, which binds to the D4 region, was used as a control. As shown in FIG. 17E, D1M4 and D1M2 mutants completely abolished binding by antibodies B2-7,-15 and 17, indicating that the two regions are key to the binding activities of these antibodies; D1M4 completely, while D1M2 partially, abolished binding by antibodies B2-16 and 19, indicating that the M4 region is key to the binding activities of the two antibodies. As a control, none of these mutants abolished binding by antibody B2-18. By similar method, key regions to the binding activities of other antibodies on the D4 domain were determined (FIG. 17F).

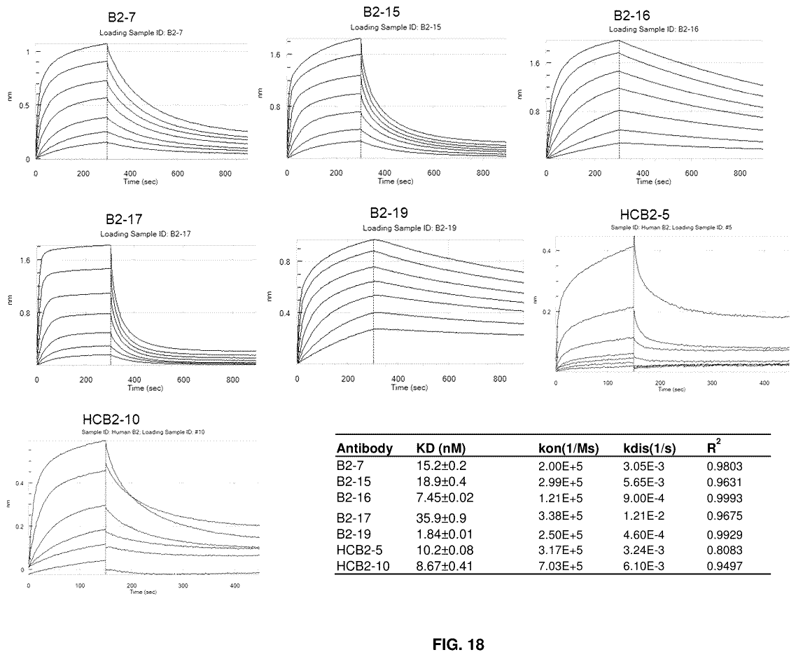

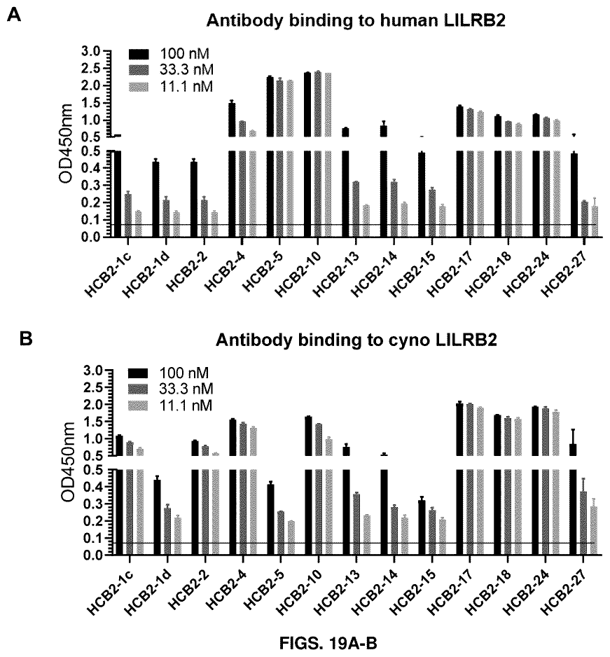

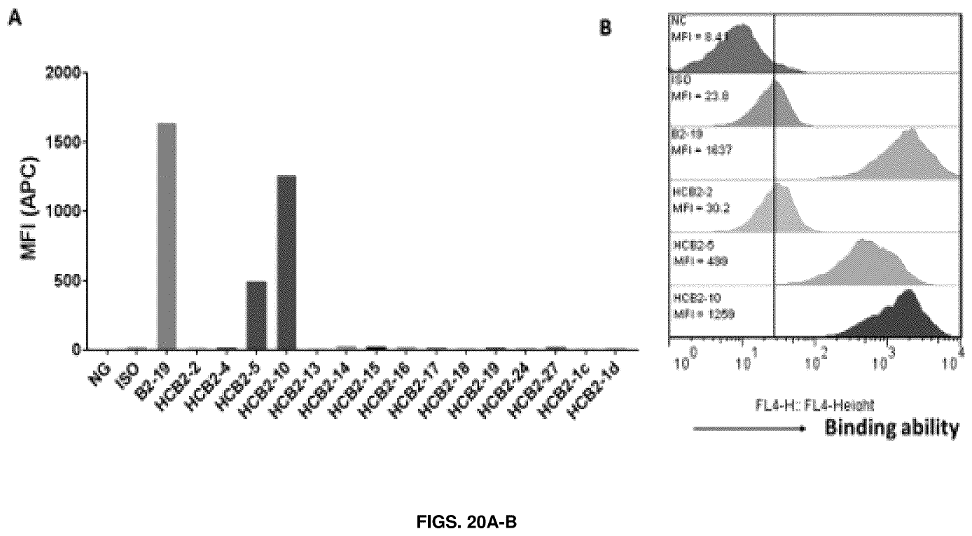

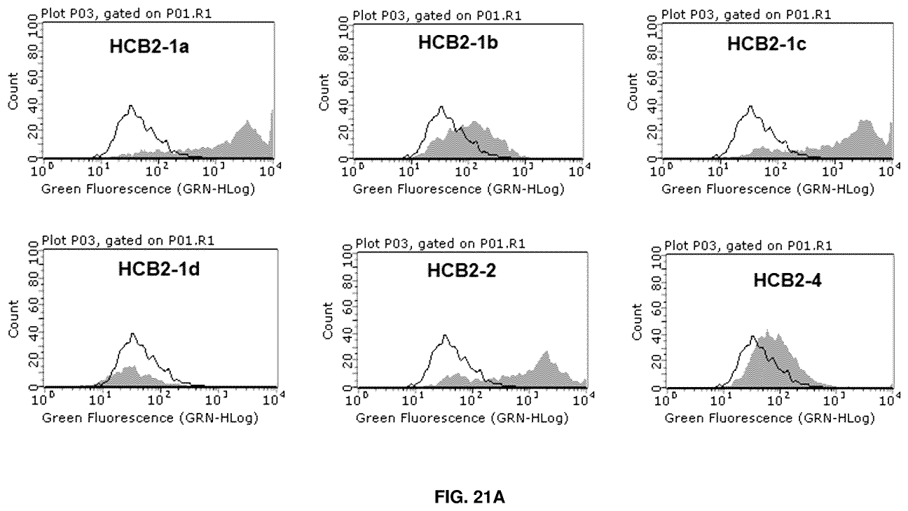

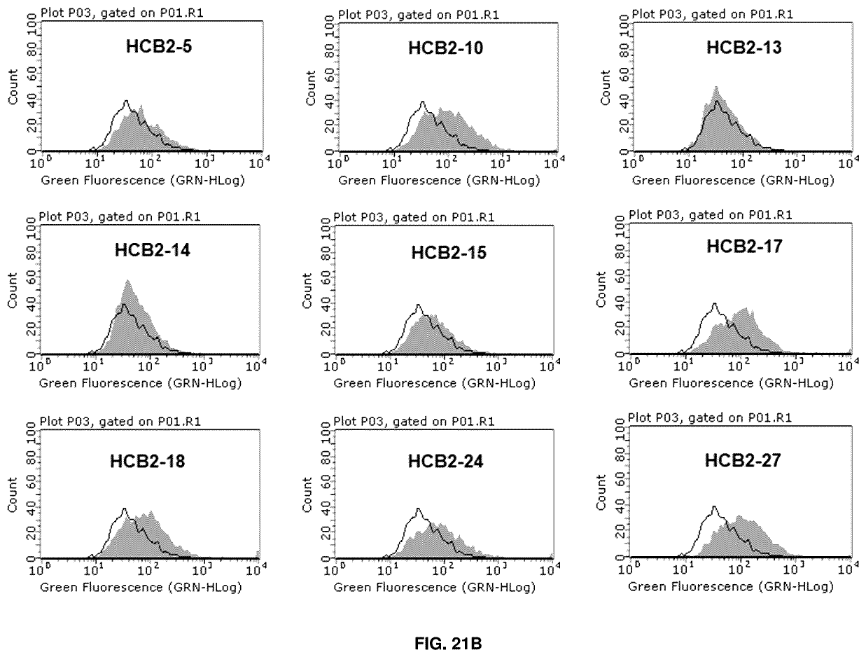

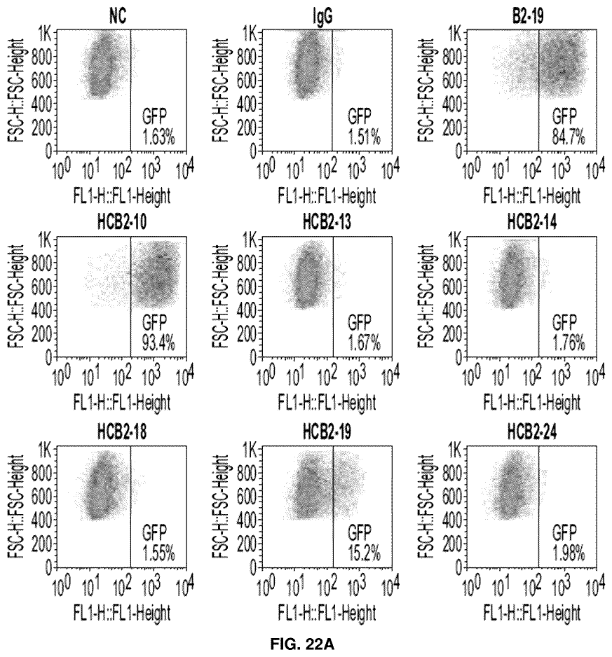

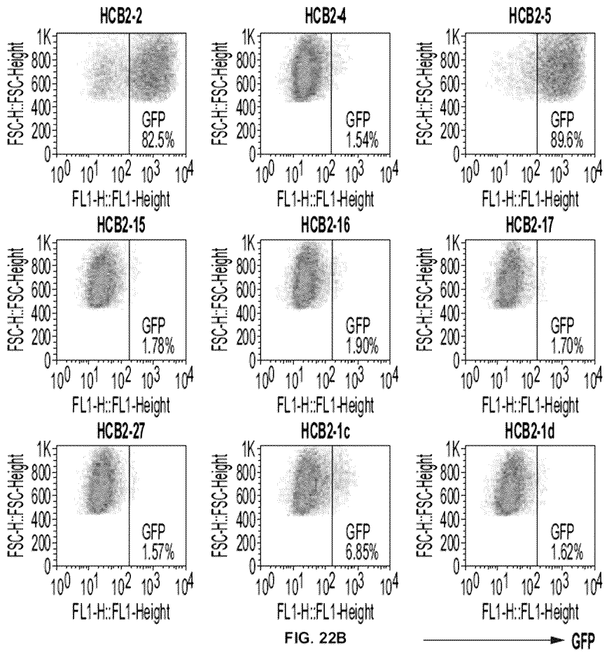

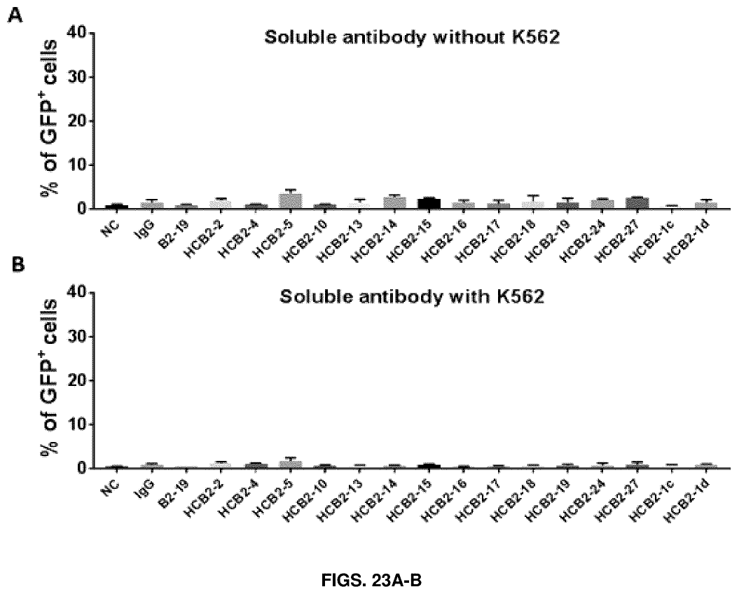

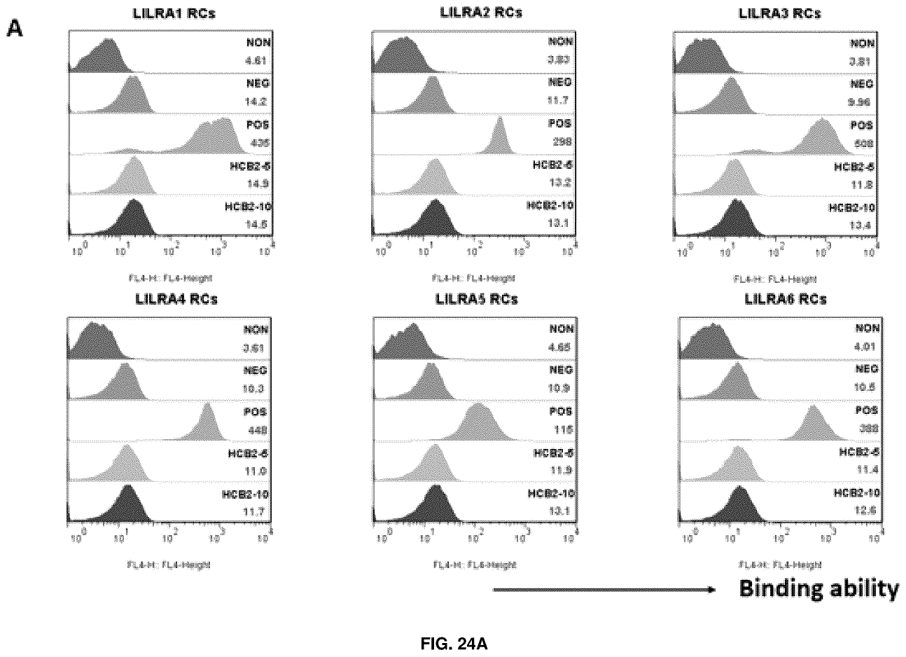

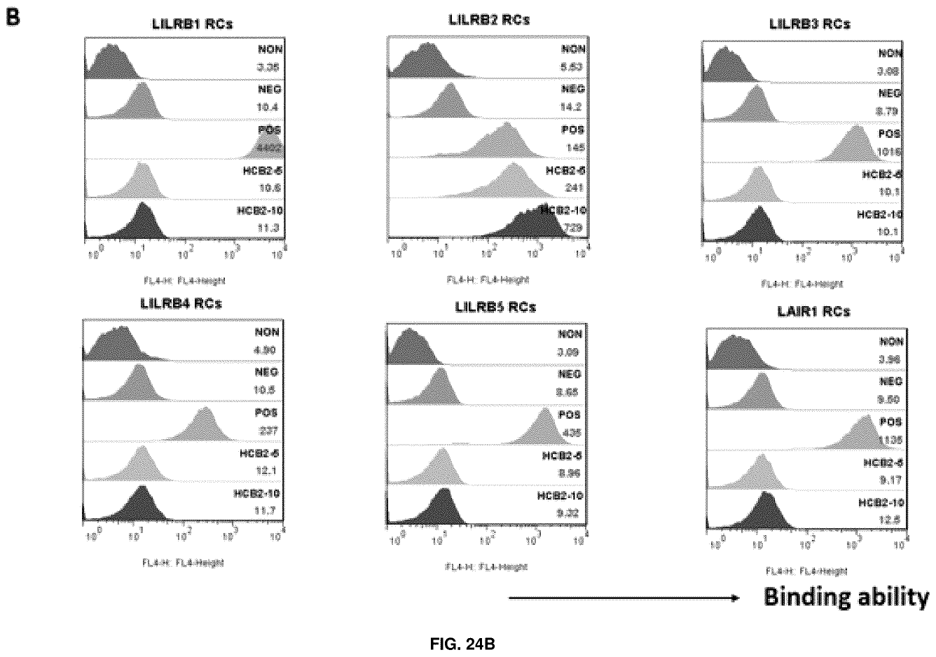

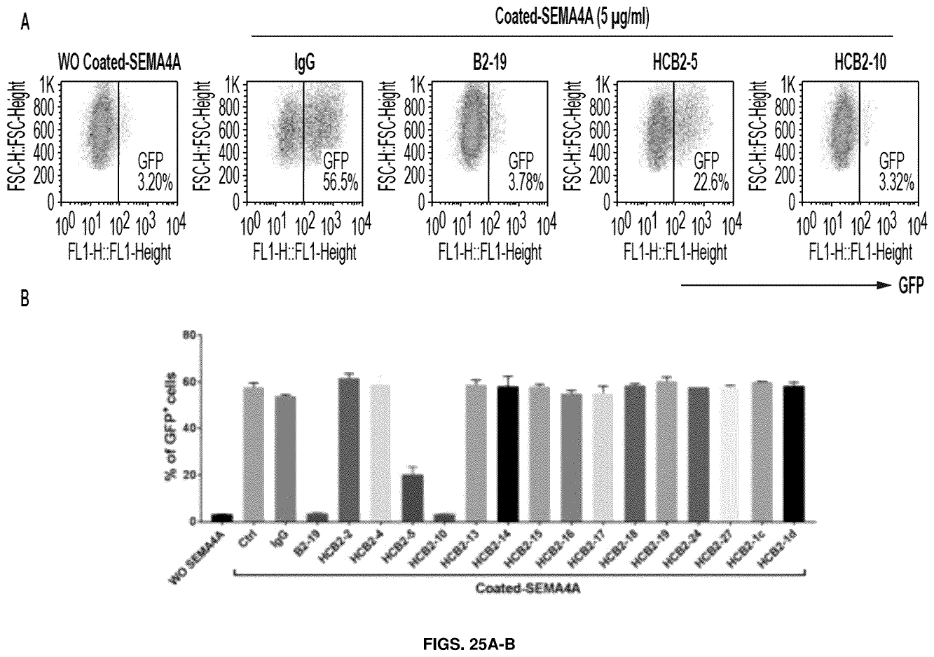

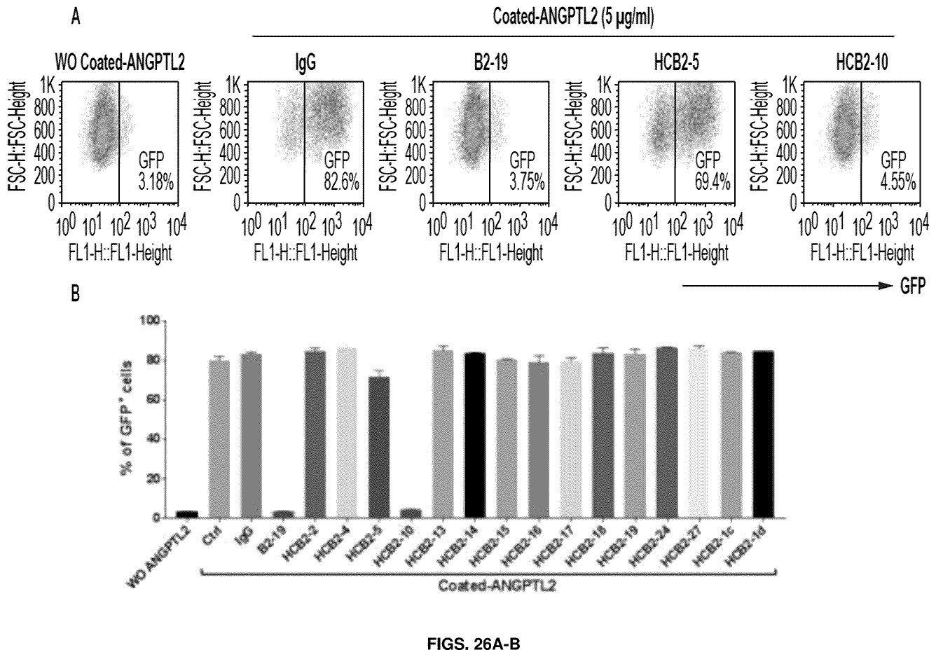

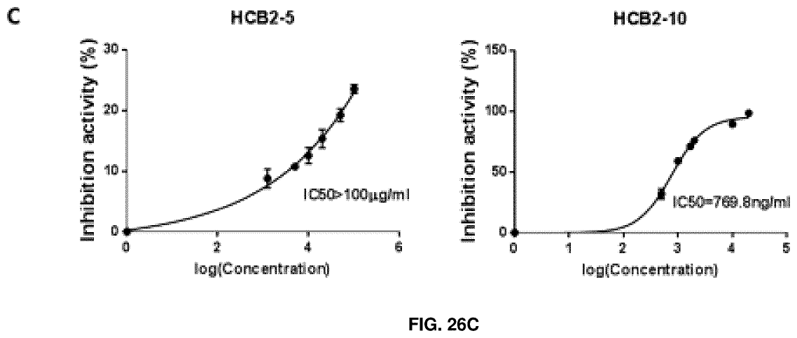

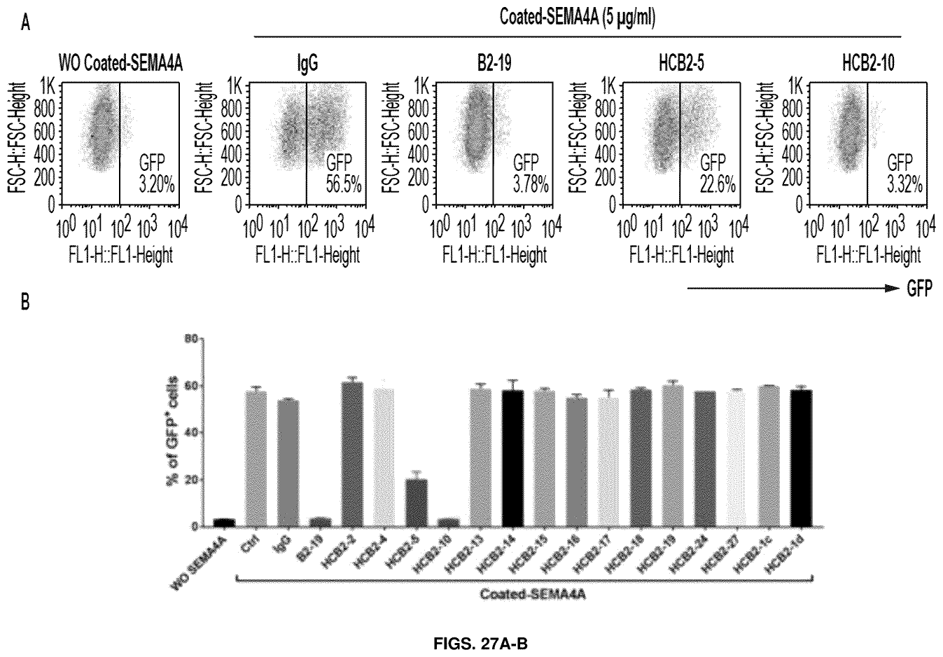

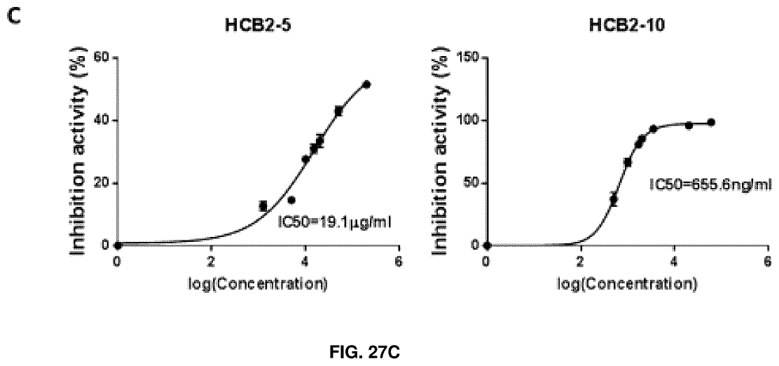

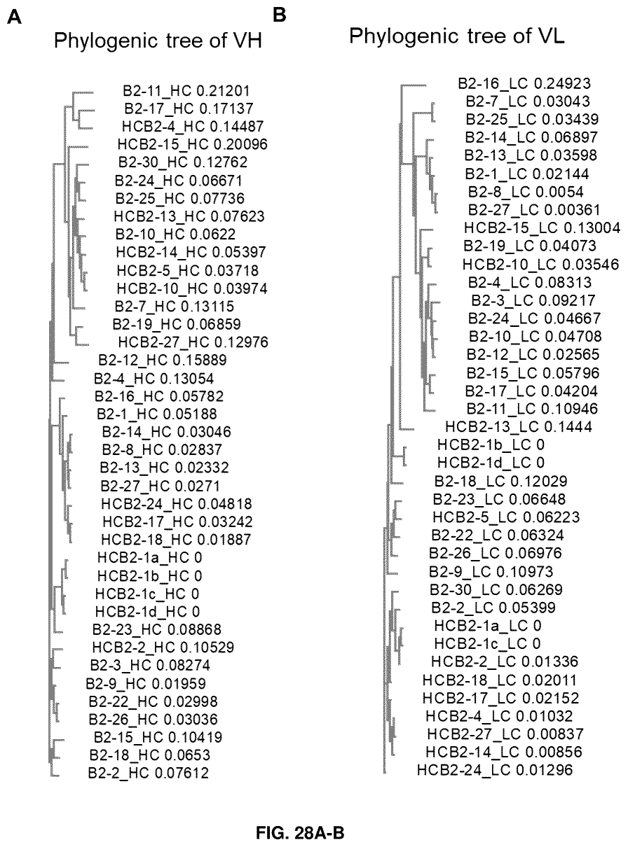

Affinity measurement of blocking antibodies. Affinities of the 5 blocking antibodies were measured on Octet RED 96 system. As shown in FIG. 18 and Table 1, the affinities range from 1.87 to 35.9 nM.

### Example 3

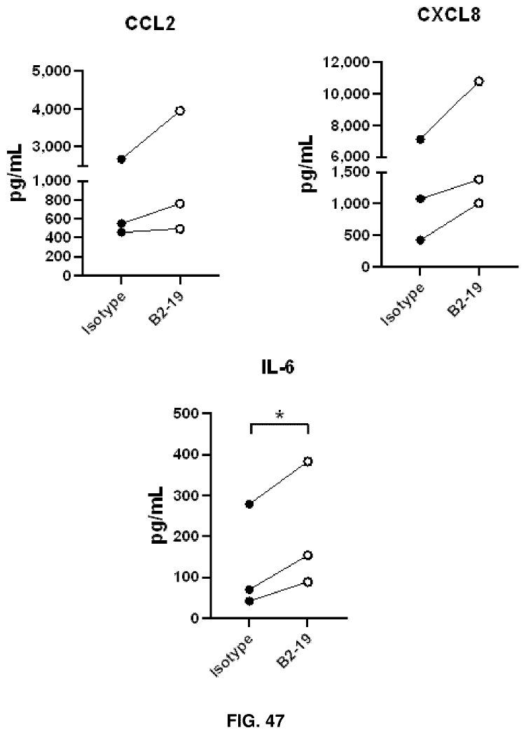

Classical monocytes isolated from PBMC of apparently healthy donors were differentiated into immature DC for 6 days using 50 ng/mL GM-CSF and 35 ng/ml IL-4. On day 6, DC were treated with 40 ng/ml IL-10 (as a tolerogenic stimulus) and with 10 μg/mL B2-19 or its isotype control. After 48 hours, the levels of cytokines and chemokines were measured in the culture media supernatant, using a Luminex assay (R&D Systems). B2-19 triggered enhanced production of the pro-inflammatory cytokine IL-6 and of chemokines known to recruit monocytes (CCL2) and neutrophils (CXCL8) (FIG. 47).

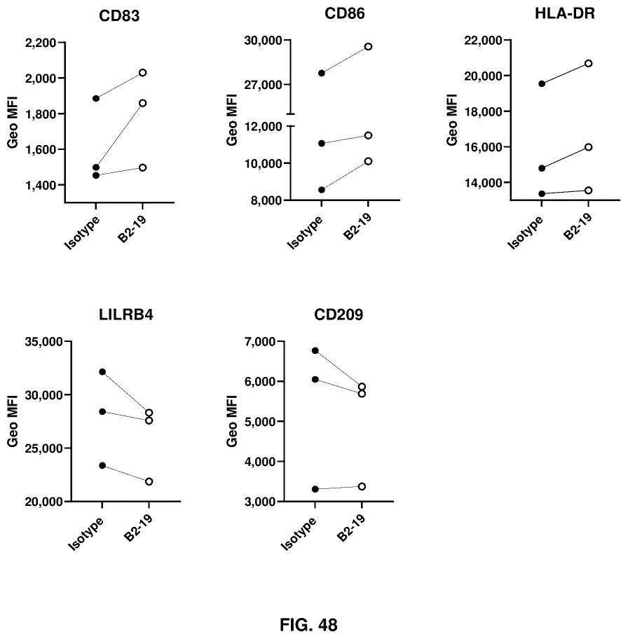

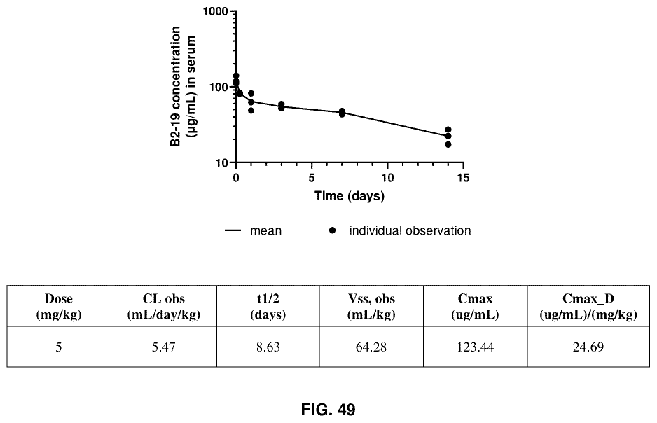

Classical monocytes isolated from PBMC prepared from apparently healthy donors were differentiated into immature DC for 6 days using 50 ng/mL GM-CSF and 35 ng/ml IL-4. On day 6, DC were treated with 100 ng/mL LPS (as a maturation stimulus) and with 10 μg/mL B2-19 or its isotype control. After 48 hours, the expression levels of several cell surface markers were measured by flow cytometry. B2-19 increased the expression of CD83 (a DC maturation marker), CD86 (a co-stimulatory molecule), HLA-DR (antigen presentation). On the other hand, B2-19 decreased the expression levels of the inhibitory receptors LILRB4 and CD209. Altogether, these data indicate that B2-19 further promotes a pro-inflammatory phenotype in LPS-stimulated DC (FIG. 48).

The pharmacokinetics of B2-19 were evaluated in wild-type mice (no LILRB2 expression). Nine C57BL/6J mice received a single 5 mg/kg intravenous dose of B2-19 and were randomized into groups of 3 for blood sampling at alternated time points. Sera were prepared from blood samples and B2-19 concentration determined using ELISA. The antibody in sera was captured by plate-coated antigen (LILRB2-ECD, R&D Systems) and detected using an HRP-conjugated goat-anti-human IgG Fc antibody (Jackson ImmunoResearch) and TMB substrate. The results indicate that B2-19 antibody presents the expected pharmacokinetics profile (CL and half-life) of human IgG4 in C57BL/6J wildtype mice dosed at 5 mg/kg. Since the ELISA format measures active antibody, these results demonstrate that B2-19 in vivo exposure (in mice) does not decrease its binding activity.

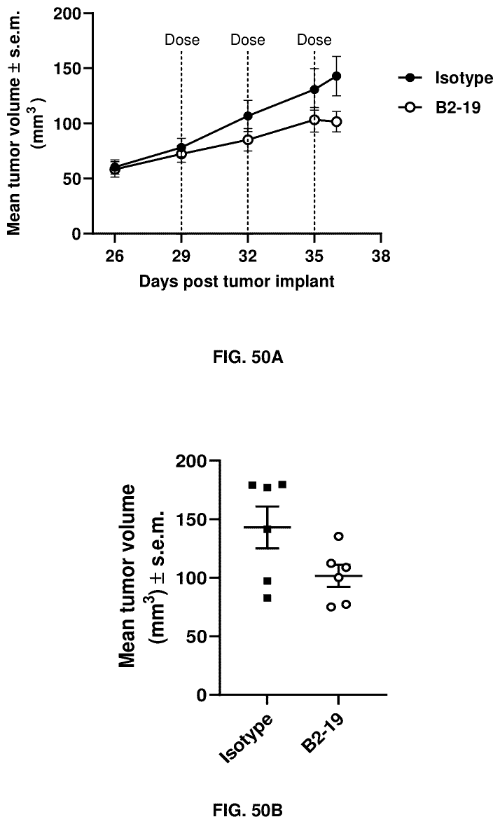

The anti-tumor efficacy of B2-19 antibody was evaluated in humanized NSG-SGM3 mice xenografted with SK-MEL-5 melanoma cell line. NSG-SGM3 mice (The Jackson Laboratory, stock No. 013062) were humanized with 1×10⁵ CD34⁺ hematopoietic cells isolated from umbilical cord blood of individual donors. Six weeks after humanization, mice were implanted subcutaneously with 1×10⁶ SK-MEL-5 cells. When the mean tumor size reached 70-80 mm³ (29 days following tumor implantation), mice were randomized into 2 treatment groups with 6 mice per group, based on the following criteria: % human CD45⁺CD14⁺ cells in blood, tumor volume and donor (2 mice from each CD34⁺ donor per treatment cohort). Mice were treated intravenously with 20 mg/kg B2-19 or its isotype control every 3 days for 3 doses. Compared to its isotype control, B2-19 monotherapy decreased tumor growth rate and caused a 29% reduction in tumor size (FIGS. 50A-B). These data demonstrate that B2-19 antibody monotherapy displays anti-tumor efficacy in a mouse model of solid tumor.

TABLE 1
Affinities of LILRB2 blocking antibodies

| Antibody | KD (nM) | Kon (1/Ms) | Kdis (1/s) | R² |
|---|---|---|---|---|
| B2-7 | 15.2 ± 0.2 | 2.00E+5 | 3.05E−3 | 0.9803 |
| B2-15 | 18.9 ± 0.4 | 2.99E+5 | 5.65E−3 | 0.9631 |
| B2-16 | 7.45 ± 0.02 | 1.21E+5 | 9.00E−4 | 0.9993 |
| B2-17 | 35.9 ± 0.9 | 3.38E+5 | 1.21E−2 | 0.9675 |
| B2-19 | 1.84 ± 0.01 | 2.50E+5 | 4.60E−4 | 0.9929 |
| HCB2-5 | 10.2 ± 0.08 | 3.17E+5 | 3.24E−3 | 0.8083 |
| HCB2-10 | 8.67 ± 0.41 | 7.03E+5 | 6.10E−3 | 0.9497 |

TABLE 2
Amino acid sequences of CDRs for LILRB2 antibodies

| Heavy | | SEQ ID | | SEQ ID | | SEQ ID | Light | | SEQ ID | | SEQ ID | | SEQ ID |
|---|---|---|---|---|---|---|---|---|---|---|---|---|---|
| chain | CDR1 | NO: | CDR2 | NO: | CDR3 | NO: | chain | CDR1 | NO: | CDR2 | NO: | CDR3 | NO: |
| B2-19_HC | GFTFSNAW | 97 | IKSKTDGGTT | 98 | TTDRYSSSW | 99 | B2-19_LC | SSNIGNNV | 100 | YDD | 101 | ATWDDSLNGPV | 102 |
| | | | | | YSPAFDI | | | | | | | | |
| B2-1_HC | GGTFSSYA | 1 | IIPIFGTA | 2 | ARGNQGDTA | 3 | B2-1_LC | SSDVGGYNY | 4 | DVS | 5 | SSYTSSSTPYV | 6 |
| | | | | | FDI | | | | | | | | |
| B2-2_HC | GYTFSDYG | 7 | ISGYNGNT | 8 | ARLLLGYYG | 9 | B2-2_LC | QSIGNY | 10 | AAS | 11 | QQSFSIPPIT | 12 |
| | | | | | MDV | | | | | | | | |
| B2-3_HC | GYTFSSYG | 13 | ISGNTGNT | 14 | ARDRGIAAG | 15 | B2-3_LC | SSNIGTNP | 16 | NNN | 17 | SSWDDSLSAWV | 18 |
| | | | | | RAFDI | | | | | | | | |
| B2-4_HC | GYPFTSNG | 19 | ISTNSGNT | 20 | ARDVQYRMD | 21 | B2-4_LC | RSNIGSNP | 22 | SNN | 23 | AAWDDNVHGV | 24 |
| | | | | | V | | | | | | | | |
| B2-7_HC | GFTISSYG | 25 | ISYDGSNK | 26 | AKDLLLRYF | 27 | B2-7_LC | NIGSKS | 28 | DDS | 29 | QVWDSSSDHPGV | 30 |
| | | | | | DWSPKYYYN | | | | | | | | |
| | | | | | GMDV | | | | | | | | |
| B2-8_HC | GGTFSSYA | 31 | IIPIFGTA | 32 | ARVAVADTF | 33 | B2-8_LC | SSDVGGYNY | 34 | DVS | 35 | SSYTSSSTPYV | 36 |
| | | | | | FDY | | | | | | | | |
| B2-9_HC | GYTFTSYG | 37 | ISAYNGNT | 38 | ARDGYGMDV | 39 | B2-9_LC | HTVNSY | 40 | AAS | 41 | QQSYRTPLT | 42 |
| B2-10_HC | GFTFSSYA | 43 | INDSGGST | 44 | AKDHITIFG | 45 | B2-10_LC | SSNIGGNP | 46 | YND | 47 | ATWDDSLNGWV | 48 |
| | | | | | VAPFDY | | | | | | | | |
| B2-11_HC | GDSVSSNSAA | 49 | TYYRSKWYN | 50 | ARQDESAAD | 51 | B2-11_LC | KSNVGNNF | 52 | SNL | 53 | AAWDDSLPGWV | 54 |
| | | | | | DAFDI | | | | | | | | |
| B2-12_HC | GYTFPDNG | 55 | INVDTGYT | 56 | AREIEGVWT | 57 | B2-12_LC | SSNIGSNP | 58 | NNN | 59 | AAWDDSLNGWV | 60 |
| | | | | | A | | | | | | | | |
| B2-13_HC | GGTFSSYA | 61 | IIPIFGTA | 62 | ARSDYGDSY | 63 | B2-13_LC | SSDVGGFNY | 64 | DVS | 65 | SSYTSSTAPYV | 66 |
| | | | | | FDY | | | | | | | | |
| B2-14_HC | GGTFSSYA | 67 | IIPIFGTA | 68 | ARASMVETY | 69 | B2-14_LC | SSDVGGYNY | 70 | EVS | 71 | SSYAGGYTPYV | 72 |
| | | | | | FDY | | | | | | | | |
| B2-15_HC | GYTFTNYG | 73 | ISGDAGDT | 74 | ARDGEYIPM | 75 | B2-15_LC | NANIGSNP | 76 | SNN | 77 | EGWDDSLNGYV | 78 |
| | | | | | FRGFDNYYG | | | | | | | | |
| | | | | | LDV | | | | | | | | |
| B2-16_HC | GGTFSSYA | 79 | IIPIFGTA | 80 | ASGIIPDNP | 81 | B2-16_LC | QSLVYSDGN | 82 | KVS | 83 | MQGTHWPVT | 84 |
| | | | | | YGMDV | | | TY | | | | | |
| B2-17_HC | GVSTSSTHW | 85 | ILHNGNT | 86 | ASASPNWGA | 87 | B2-17_LC | SSNIGSNP | 88 | SDN | 89 | STWDDSLNGLV | 90 |
| | | | | | SGAFDA | | | | | | | | |
| B2-18_HC | GYTFTRYG | 91 | ISGYNGNT | 92 | ARDRGSGYL | 93 | B2-18_LC | QGIYSS | 94 | GTS | 95 | QQHYNLPIT | 96 |
| | | | | | EY | | | | | | | | |
| B2-22_HC | GYTFTSYG | 103 | ISAYNGNT | 104 | ARVGSYGDY | 105 | B2-22_LC | QGISNY | 106 | AAS | 107 | QKYNSAPLT | 108 |
| B2-23_HC | GYTFTHSY | 109 | INPVGGST | 110 | ARAGGDGYN | 111 | B2-23_LC | QSISSW | 112 | KAS | 113 | QQYNSYLT | 114 |
| | | | | | PYFDY | | | | | | | | |
| B2-24_HC | GFTFSDYY | 115 | ISSSSSYT | 116 | ARVGDLDAF | 117 | B2-24_LC | SSNIGSNY | 118 | RNN | 119 | AAWDDSLSGWV | 120 |
| | | | | | DI | | | | | | | | |
| B2-25_HC | GFTFSDHY | 121 | ITTTGSTM | 122 | ARDSIHVDG | 123 | B2-25_LC | NIGSKS | 124 | DDS | 125 | QVWDSSSDHWV | 126 |
| | | | | | AFDI | | | | | | | | |
| B2-26_HC | GYTFTSYG | 127 | ISAYNGNT | 128 | ARDRGGWFD | 129 | B2-26_LC | QSISNF | 130 | GAS | 131 | QQGYSVPLT | 132 |
| | | | | | P | | | | | | | | |
| B2-27_HC | GGTFSSYA | 133 | IIPIFGTA | 134 | ARESYSSSS | 135 | B2-27_LC | SSDVGGYNY | 136 | DVS | 137 | SSYTSSSTPYV | 138 |
| | | | | | FDY | | | | | | | | |
| B2-30_HC | GFTFSSYD | 139 | IGTAGDT | 140 | ARAKEYCSG | 141 | B2-30_LC | QSIRTY | 142 | AAT | 143 | QQSYSAPVFT | 144 |
| | | | | | GSCQDAFDI | | | | | | | | |
| HCB2-1c_HC | GYTFTDYY | 145 | VNANGGDT | 146 | ARDWYYYSS | 147 | HCB2-lc_LC | QSISSF | 148 | GAS | 149 | QQSYSIPIT | 150 |
| | | | | | GSFPFDY | | | | | | | | |
| HCB2-1d_HC | GYTFTDYY | 151 | VNANGGDT | 152 | ARDWYYYSS | 153 | HCB2-ld_LC | QDVSIA | 154 | SAS | 155 | QQHYITPLT | 156 |
| | | | | | GSFPFDY | | | | | | | | |
| HCB2-2_HC | GYIFNTYG | 157 | TNVYNSNT | 158 | ARDPGSAIF | 159 | HCB2-2_LC | QSISSF | 160 | GAS | 161 | QQSYSIPIT | 162 |
| | | | | | DM | | | | | | | | |
| HCB2-4_HC | GDAIKDKY | 163 | ISNSGST | 164 | ARGSGYTAF | 165 | HCB2-4_LC | QSISSY | 166 | AAS | 167 | QQSYSTPPT | 168 |
| | | | | | DI | | | | | | | | |
| HCB2-5_HC | GFTFSSYA | 169 | ISGSGGST | 170 | AKDVDPYSS | 171 | HCB2-5_LC | QGIGSW | 172 | VAS | 173 | QQANSFPLT | 174 |
| | | | | | GYFDY | | | | | | | | |
| HCB2-10_HC | GFTFSNYA | 175 | ISGSSIST | 176 | ASSGFGSFD | 177 | HCB2-10_LC | SSNIGDNS | 178 | LDD | 179 | ATWDDSLNGVV | 180 |
| | | | | | Y | | | | | | | | |
| HCB2-13_HC | GFTLSSYA | 181 | INSGGST | 182 | AKAKSYSSD | 183 | HCB2-13_LC | QSLLNSGNQ | 184 | GAS | 185 | QNDHSYPFT | 186 |
| | | | | | LDH | | | KTY | | | | | |
| HCB2-14_HC | GFTFSSYA | 187 | ISGSGGST | 188 | AKDLLGDSG | 189 | HCB2-14_LC | QSISSY | 190 | AAS | 191 | QQSYSTPYT | 192 |
| | | | | | SYPAFDI | | | | | | | | |
| HCB2-15_HC | GFPFADYA | 193 | IKSKTRDGTT | 194 | ALGGAAAL | 195 | HCB2-15_LC | SSNIGAGYG | 196 | GDN | 197 | QSYDSSLGVGV | 198 |
| HCB2-17_HC | GGTFSSYA | 199 | IIPIFGTA | 200 | AISSGYYYN | 201 | HCB2-17_LC | QSISSY | 202 | AAS | 203 | QQSYSTPLT | 204 |
| | | | | | PFDY | | | | | | | | |
| HCB2-18_HC | GGTFSSYA | 205 | IIPIFGTA | 206 | ARGGYQPLD | 207 | HCB2-18_LC | QSISSY | 208 | AAS | 209 | QQSYSTPFT | 210 |
| | | | | | Y | | | | | | | | |
| HCB2-24_HC | GGTFSSYA | 211 | IIPIFGTA | 212 | ARAGGIHYY | 213 | HCB2-24_LC | QSISSY | 214 | AAS | 215 | QQSYSTPLT | 216 |
| | | | | | DSLDY | | | | | | | | |
| HCB2-27_HC | GFTFANAW | 217 | IKPSSSGGRT | 218 | CVTGRRVAK | 219 | HCB2-27_LC | QSISSY | 220 | AAS | 221 | QQSYSTPRT | 222 |
| | | | | | TFDF | | | | | | | | |

TABLE 3
DNA sequences of CDRs for LILRB2 antibodies

| | | SEQ | | SEQ | | SEQ | | | SEQ | | SEQ | | SEQ |
|---|---|---|---|---|---|---|---|---|---|---|---|---|---|
| Heavy | | ID | | ID | | ID | Light | | ID | | ID | | ID |
| chain | CDR1 | NO: | CDR2 | NO: | CDR3 | NO: | chain | CDR1 | NO: | CDR2 | NO: | CDR3 | NO: |
| B2-19_HC | ggattcact | 319 | attaaaagcaa | 320 | accacagatcga | 321 | B2-19_LC | agctccaacat | 322 | tatgatgat | 323 | gcaacatggg | 324 |
| | ttcagtaac | | aactgatggtg | | tatagcagcagc | | | cggaaataatg | | | | atgacagcct | |
| | gcctgg | | ggacaaca | | tggtactcccct | | | tt | | | | gaatggccct | |
| | | | | | gcttttgatatc | | | | | | | gta | |
| B2-1_HC | ggaggcacc | 223 | atcatccctat | 224 | gcgagggggaat | 225 | B2-1_LC | agcagtgacgt | 226 | gatgtcagt | 227 | agctcatata | 228 |
| | ttcagcagc | | ctttggtacag | | cagggggatacg | | | tggtggttata | | | | caagcagcag | |
| | tatgct | | ca | | gcttttgatatc | | | actat | | | | cactccttat | |
| | | | | | | | | | | | | gtc | |
| B2-2_HC | ggttacacc | 229 | atcagtggtta | 230 | gcgagattgctg | 231 | B2-2_LC | cagagcattgg | 232 | gctgcatcc | 233 | caacagagtt | 234 |
| | tttagcgac | | caatggtaaca | | ctgggttactac | | | caactat | | | | tcagtatccc | |
| | tatggt | | ca | | ggtatggacgtc | | | | | | | cccgataact | |
| B2-3_HC | ggttacacg | 235 | atcagtggtaa | 236 | gcgagagatcgt | 237 | B2-3_LC | agctccaacat | 238 | aataataat | 239 | tcgtcatggg | 240 |
| | tttagcagc | | cactggtaaca | | ggtatagcggca | | | cggaactaatc | | | | acgacagcct | |
| | tatggt | | ca | | ggccgtgctttt | | | ct | | | | gagtgcgtgg | |
| | | | | | gatatc | | | | | | | gtg | |
| B2-4_HC | ggttacccc | 241 | atcagcactaa | 242 | gcgagagatgtt | 243 | B2-4_LC | aggtccaacat | 244 | agtaataat | 245 | gcagcgtggg | 246 |
| | tttaccagc | | cagcggaaaca | | caatatcgtatg | | | cggaagtaatc | | | | atgacaacgt | |
| | aatggt | | ca | | gacgtc | | | ct | | | | gcacggagtt | |
| B2-7_HC | ggattcacc | 247 | atatcatatga | 248 | gcgaaagatctc | 249 | B2-7_LC | aacattggaag | 250 | gatgatagc | 251 | caggtgtggg | 252 |
| | atcagcagt | | tggaagtaata | | ttattacgatat | | | taaaagt | | | | atagtagtag | |
| | tatggc | | | | tttgactggagc | | | | | | | tgatcatccg | |
| | | | | | cccaaatactac | | | | | | | ggggtc | |
| | | | | | tacaacggtatg | | | | | | | | |
| | | | | | gacgtc | | | | | | | | |
| B2-8_HC | ggaggcacc | 253 | atcatccctat | 254 | gcgagagtggca | 255 | B2-8_LC | agcagtgacgt | 256 | gatgtcagt | 257 | agctcatata | 258 |
| | ttcagcagc | | ctttggtacag | | gtggctgacacc | | | tggtggttata | | | | caagcagcag | |
| | tatgct | | ca | | ttctttgactac | | | actat | | | | caccccttat | |
| | | | | | | | | | | | | gtc | |
| B2-9_HC | ggttacacc | 259 | atcagcgctta | 260 | gcgagagacggc | 261 | B2-9_LC | cacaccgttaa | 262 | gctgcatcc | 263 | caacagagtt | 264 |
| | tttaccagc | | caatggtaaca | | tacggtatggac | | | cagctat | | | | acagaacccc | |
| | tatggt | | ca | | gtc | | | | | | | tctcact | |
| B2-10_HC | ggattcacc | 265 | attaatgatag | 266 | gcgaaagatcat | 267 | B2-10_LC | agctccaacat | 268 | tataatgat | 269 | gcaacatggg | 270 |
| | tttagcagc | | tggtggtagca | | attacgattttt | | | cggaggtaatc | | | | atgacagcct | |
| | tatgcc | | ca | | ggagtggccccg | | | ct | | | | gaatggttgg | |
| | | | | | tttgactac | | | | | | | gtg | |
| B2-11_HC | ggggacagt | 271 | acatactacag | 272 | gcaagacaagat | 273 | B2-11_LC | aagtccaacgt | 274 | agcaatctt | 275 | gcagcatggg | 276 |
| | gtctctagc | | gtccaagtggt | | gaatcggcagcc | | | cggaaataatt | | | | atgacagcct | |
| | aacagtgct | | ataat | | gatgatgctttt | | | tt | | | | gcccggttgg | |
| | gct | | | | gatatc | | | | | | | gtg | |
| B2-12_HC | ggttacacg | 277 | atcaacgttga | 278 | gcgagagaaatc | 279 | B2-12_LC | agctccaacat | 280 | aataataat | 281 | gcggcatggg | 282 |
| | tttcccgac | | cactggataca | | gagggagtgtgg | | | cggaagtaatc | | | | atgacagcct | |
| | aacggt | | cc | | acagct | | | ct | | | | gaatggttgg | |
| | | | | | | | | | | | | gtg | |
| B2-13_HC | ggaggcacc | 283 | atcatccctat | 284 | gcgaggtcggac | 285 | B2-13_LC | agcagtgacgt | 286 | gatgtcagt | 287 | agctcatata | 288 |
| | ttcagcagc | | ctttggtacag | | tacggtgactcc | | | tggtggtttta | | | | caagcagcac | |
| | tatgct | | ca | | tactttgactac | | | actat | | | | cgctccttat | |
| | | | | | | | | | | | | gtc | |
| B2-14_HC | ggaggcacc | 289 | atcatccctat | 290 | gcgagagcctct | 291 | B2-14_LC | agcagtgatgt | 292 | gaggtcagt | 293 | tcctcatatg | 294 |
| | ttcagcagc | | ctttggtacag | | atggtcgagact | | | tggtggttata | | | | caggcgggta | |
| | tatgct | | ca | | tactttgactac | | | actat | | | | caccccctat | |
| | | | | | | | | | | | | gtc | |
| B2-15_HC | ggttacacc | 295 | atcagcggtga | 296 | gcgagagatggg | 297 | B2-15_LC | aacgccaacat | 298 | agtaataat | 299 | gaaggatggg | 300 |
| | tttaccaac | | cgctggtgaca | | gaatatattcct | | | cggaagtaatc | | | | atgacagcct | |
| | tatggt | | ca | | atgtttcgggga | | | ct | | | | gaatggctat | |
| | | | | | tttgacaactac | | | | | | | gtc | |
| | | | | | tacggtctggac | | | | | | | | |
| | | | | | gtc | | | | | | | | |
| B2-16_HC | ggaggcacc | 301 | atcatccctat | 302 | gcgagcgggatt | 303 | B2-16_LC | caaagcctcgt | 304 | aaggtttct | 305 | atgcaaggta | 306 |
| | ttcagcagc | | ctttggtacag | | atacctgataat | | | atacagtgatg | | | | cacactggcc | |
| | tatgct | | ca | | ccctacggtatg | | | gaaacacctac | | | | ggtcacc | |
| | | | | | gacgtc | | | | | | | | |
| B2-17_HC | ggtgtctcc | 307 | atccttcataa | 308 | gcgtcagcgtcg | 309 | B2-17_LC | agctccaacat | 310 | agtgataat | 311 | tcaacatggg | 312 |
| | accagcagt | | tggaaacacc | | cctaactgggga | | | cggaagtaatc | | | | atgacagcct | |
| | actcattgg | | | | gctagcggggct | | | ct | | | | gaatggtctt | |
| | | | | | tttgatgcc | | | | | | | gtc | |
| B2-18_HC | ggttacacc | 313 | atcagcggtta | 314 | gcgagagatcgg | 315 | B2-18_LC | cagggcattta | 316 | ggtacgtcg | 317 | caacaacatt | 318 |
| | tttaccagg | | caatggtaata | | ggtagtggttat | | | cagttct | | | | acaatctacc | |
| | tatggt | | ca | | cttgaatac | | | | | | | catcacc | |
| B2-22_HC | ggttacacc | 325 | atcagcgctta | 326 | gcgagagtgggc | 327 | B2-22_LC | cagggcattag | 328 | gctgcatcc | 329 | caaaagtata | 330 |
| | tttaccagc | | caatggtaaca | | agctatggtgac | | | caattat | | | | acagtgcccc | |
| | tatggt | | ca | | tac | | | | | | | gctcact | |
| B2-23_HC | gggtacacc | 331 | atcaaccctgt | 332 | gctagagcgggg | 333 | B2-23_LC | cagagtattag | 334 | aaggcgtct | 335 | caacagtata | 336 |
| | ttcacccac | | tggtggtagca | | ggagatggctac | | | tagctgg | | | | atagttatct | |
| | tcctat | | ca | | aatccttacttt | | | | | | | cact | |
| | | | | | gactac | | | | | | | | |
| B2-24_HC | ggattcacc | 337 | attagtagtag | 338 | gcgagagtcgga | 339 | B2-24_LC | agctccaacat | 340 | aggaataat | 341 | gcagcatggg | 342 |
| | ttcagtgac | | tagtagttaca | | gatcttgatgct | | | cggaagtaatt | | | | atgacagcct | |
| | tactac | | ca | | tttgatatc | | | at | | | | gagtggttgg | |
| | | | | | | | | | | | | gtg | |
| B2-25_HC | ggattcacc | 343 | attactactac | 344 | gcgagagattcc | 345 | B2-25_LC | aacattggaag | 346 | gatgatagc | 347 | caggtgtggg | 348 |
| | ttcagtgac | | tggtagtacca | | atacatgtggat | | | taaaagt | | | | atagtagtag | |
| | cactac | | tg | | ggtgcttttgat | | | | | | | tgatcattgg | |
| | | | | | att | | | | | | | gtg | |
| B2-26_HC | ggttacacc | 349 | atcagcgctta | 350 | gcgagagatcgg | 351 | B2-26_LC | cagagcataag | 352 | ggtgcatcc | 353 | caacagggtt | 354 |
| | tttaccagc | | caatggtaaca | | ggagggtggttc | | | caacttt | | | | acagtgtccc | |
| | tatggt | | ca | | gacccc | | | | | | | actcact | |
| B2-27_HC | ggaggcacc | 355 | atcatccctat | 356 | gcgagagagtcg | 357 | B2-27_LC | agcagtgacgt | 358 | gatgtcagt | 359 | agctcatata | 360 |
| | ttcagcagc | | ctttggtacag | | tatagcagctcg | | | tggtggttata | | | | caagcagcag | |
| | tatgct | | ca | | tcctttgactac | | | actat | | | | cactccctat | |
| | | | | | | | | | | | | gtc | |
| B2-30_HC | ggattcacc | 361 | attggtactgc | 362 | gcaagggcgaag | 363 | B2-30_LC | cagagcattag | 364 | gctgcaacc | 365 | caacagagtt | 366 |
| | ttcagtagc | | tggtgacaca | | gaatattgtagt | | | gacctac | | | | acagtgcccc | |
| | tacgac | | | | ggtggtagctgc | | | | | | | cgtgttcact | |
| | | | | | caagatgctttt | | | | | | | | |
| | | | | | gatatc | | | | | | | | |
| HCB2- | gggtacac | 367 | gtcaacgcta | 368 | gctagagattg | 369 | HCB2- | cagagcatta | 370 | ggtgcatc | 371 | caacagagt | 372 |
| 1c_HC | cttcaccg | | atggtggtga | | gtattactatt | | 1c_LC | gcagcttt | | c | | tacagtatc | |
| | actattat | | caca | | cttcggggagt | | | | | | | ccgatcacc | |
| | | | | | ttcccttttga | | | | | | | | |
| | | | | | ctac | | | | | | | | |
| HCB2- | gggtacac | 373 | gtcaacgcta | 374 | gctagagattg | 375 | HCB2- | caagatgtgt | 376 | tccgccag | 377 | cagcagcac | 378 |
| 1d_HC | cttcaccg | | atggtggtga | | gtattactatt | | 1d_LC | ccatcgcc | | c | | tacatcacc | |
| | actattat | | caca | | cttcggggagt | | | | | | | cctttaaca | |
| | | | | | ttcccttttga | | | | | | | | |
| | | | | | ctac | | | | | | | | |
| HCB2- | ggttacat | 379 | accaacgttt | 380 | gcgagagaccc | 381 | HCB2- | cagagcatta | 382 | ggtgcatc | 383 | caacagagt | 384 |
| 2_HC | cttcaaca | | acaatagtaa | | gggctcggcta | | 2_LC | gcagcttt | | c | | tacagtatc | |
| | cctatggt | | caca | | tttttgatatg | | | | | | | ccgatcacc | |
| HCB2- | ggtgacgc | 385 | atctctaaca | 386 | gcgagaggcag | 387 | HCB2- | cagagcatta | 388 | gctgcatc | 389 | caacagagt | 390 |
| 4_HC | catcaaag | | gtggaagcac | | cggttatactg | | 4_LC | gcagctat | | c | | tacagtacc | |
| | ataagtac | | c | | cttttgatatc | | | | | | | ccgcccact | |
| HCB2- | ggattcac | 391 | attagtggta | 392 | gcgaaagatgt | 393 | HCB2- | cagggtattg | 394 | gttgcatc | 395 | caacaggct | 396 |
| 5_HC | ctttagca | | gtggtggtag | | cgacccctata | | 5_LC | gcagctgg | | c | | aacagtttc | |
| | gctatgcc | | caca | | gtagtggttat | | | | | | | ccgctcact | |
| | | | | | tttgactac | | | | | | | | |
| HCB2- | ggattcac | 397 | attagtggta | 398 | gcgtcctcggg | 399 | HCB2- | agctccaata | 400 | cttgatga | 401 | gcaacatgg | 402 |
| 10_HC | ctttagca | | gtagtattag | | tttcgggtcct | | 10_LC | tcggagataa | | t | | gatgacagc | |
| | actatgcc | | caca | | ttgactac | | | ttct | | | | ctgaacggt | |
| | | | | | | | | | | | | gtggtt | |
| HCB2- | ggattcac | 403 | attaatagtg | 404 | gcgaaagcgaa | 405 | HCB2- | cagagtctgt | 406 | ggggcatc | 407 | cagaatgat | 408 |
| 13_HC | cttaagta | | gtggtagcac | | gagttatagca | | 13_LC | taaacagtgg | | c | | catagttat | |
| | gctatgcc | | a | | gtgaccttgac | | | aaatcaaaag | | | | ccattcacg | |
| | | | | | cac | | | acctac | | | | | |
| HCB2- | ggattcac | 409 | attagtggta | 410 | gcgaaagatct | 411 | HCB2- | cagagcatta | 412 | gctgcatc | 413 | caacagagt | 414 |
| 14_HC | ctttagca | | gtggtggtag | | tctcggggata | | 14_LC | gcagctat | | c | | tacagtacc | |
| | gctatgcc | | caca | | gtgggagctac | | | | | | | ccgtacact | |
| | | | | | cctgcttttga | | | | | | | | |
| | | | | | tatc | | | | | | | | |
| HCB2- | ggattccc | 415 | attaaaagca | 416 | gccctcggggg | 417 | HCB2- | agctccaaca | 418 | ggtgacaa | 419 | cagtcctat | 420 |
| 15_HC | ctttgcgg | | agactagaga | | agcagcagctc | | 15_LC | tcggggcagg | | c | | gacagcagc | |
| | actatgct | | tgggacaaca | | ta | | | ttatggt | | | | ctgggggtt | |
| | | | | | | | | | | | | ggtgtg | |
| HCB2- | ggaggcac | 421 | atcatcccta | 422 | gcgattagtag | 423 | HCB2- | cagagcatta | 424 | gctgcatc | 425 | caacagagt | 426 |
| 17_HC | cttcagca | | tctttggtac | | tggttattatt | | 17_LC | gcagctat | | c | | tacagtacc | |
| | gctatgct | | agca | | ataacccattt | | | | | | | cccctcact | |
| | | | | | gactac | | | | | | | | |
| HCB2- | ggaggcac | 427 | atcatcccta | 428 | gcgagaggggg | 429 | HCB2- | cagagcatta | 430 | gctgcatc | 431 | caacagagt | 432 |
| 18_HC | cttcagca | | tctttggtac | | ataccagccac | | 18_LC | gcagctat | | c | | tacagtacc | |
| | gctatgct | | agca | | tggattac | | | | | | | ccattcact | |
| HCB2- | ggaggcac | 433 | atcatcccta | 434 | gcgagagctgg | 435 | HCB2- | cagagcatta | 436 | gctgcatc | 437 | caacagagt | 438 |
| 24_HC | cttcagca | | tctttggtac | | gggaatccatt | | 24_LC | gcagctat | | c | | tacagtacc | |
| | gctatgct | | agca | | actatgatagt | | | | | | | cctctgacg | |
| | | | | | ttagactac | | | | | | | | |
| HCB2- | ggattcac | 439 | attaagccca | 440 | tgtgtcacagg | 441 | HCB2- | cagagcatta | 442 | gctgcatc | 443 | caacagagt | 444 |
| 27_HC | cttcgcga | | gttctagcgg | | aagaagggtcg | | 27_LC | gcagctat | | c | | tacagtacc | |
| | atgcctgg | | cgggaggaca | | cgaagactttt | | | | | | | cctcggacg | |
| | | | | | gatttc | | | | | | | | |

TABLE 4
DNA sequences of antibody heavy chain variable regions (VH)

>B2-19_VH (SEQ ID NO: 461)
CAGGTGCAGCTGGTGCAGTCTGGGGGAGGCTTGGTAAAGCCTGGGGGGTCCCTTAGACTCTCCTGTGCAGCCTCT
GGATTCACTTTCAGTAACGCCTGGATGAGCTGGGTCCGCCAGGCTCCAGGGAAGGGGCTGGAGTGGGTTGGCCGT
ATTAAAAGCAAAACTGATGGTGGGACAACAGACTACGCTGCACCCGTGAAAGGCAGATTCACCATCTCAAGAGAT
GATTCAAAAAACACGCTGTATCTGCAAATGAACAGCCTGAAAACCGAGGACACAGCCGTGTATTACTGTACCACA
GATCGATATAGCAGCAGCTGGTACTCCCCTGCTTTTGATATCTGGGGCCAAGGGACAACGGTCAGCGTCTCCTCA
>B2-1_VH (SEQ ID NO: 445)
GAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACAAATCC
ACGAGCACAGCCTACATGGAGCTGAGCAGCCTGAGATCTGAGGACACGGCCGTGTATTACTGTGCGAGGGGGAAT
CAGGGGGATACGGCTTTTGATATCTGGGGCCAAGGGACCACGGTCATCGTCTCCTCA
>B2-2_VH (SEQ ID NO: 446)
CAGGTGCAGCTGGTGCAGTCTGGAGCTGAGGTGAAGAAGCCTGGGGCCTCAGTGAAGGTCTCCTGCAAGGCTTCT
GGTTACACCTTTAGCGACTATGGTTTCAGCTGGGTGCGACAGGCCCCTGGACAAGGGCTTGAGTGGATGGGATGG
ATCAGTGGTTACAATGGTAACACAAACTATGCACAGAAGTTCCAGGGCAGAGTCACCATGACCATAGACGCATCC
ACGAGCACAGCCTACATGGAGCTGAGGAGCCTGAAATCTGACGACACGGCCGTGTATTACTGTGCGAGATTGCTG
CTGGGTTACTACGGTATGGACGTCTGGGGCCAAGGGACCACGGTCACCGTCTCCTCA
>B2-3_VH (SEQ ID NO: 447)
CAGGTGCAGCTGGTGCAGTCTGGAGCTGAGGTGAAGAAGCCTGGGGCCTCAGTGAAGGTCTCCTGCAGGACTTCT
GGTTACACGTTTAGCAGCTATGGTATCAGCTGGGTGCGACAGGCCCCTGGACAAGGGCTTGAGTGGATGGGATGG
ATCAGTGGTAACACTGGTAACACAAAGTATACACCGAAGCTCCAAGGCAGAGTCACCATGACCACAGACACATCC
ACGAGCACAGCCTACATGGAGCTGAGGAGCCTGAGATCTGACGACACGGCCGTGTATTACTGTGCGAGAGATCGT
GGTATAGCGGCAGGCCGTGCTTTTGATATCTGGGGCCAAGGGACAATGGTCACCGTCTCTTCA
>B2-4_VH (SEQ ID NO: 448)
CAGGTGCAGCTGGTGCAGTCTGGACCCACGGTGAGGAAGCCTGGGGCCTCAGTGAGGGTCTCCTGCAAGGCTTCT
GGTTACCCCTTTACCAGCAATGGTATGAGCTGGGTGCGGCAGGCCCCTGGACAAGGACTTGAGTGGATGGGATGG
ATCAGCACTAACAGCGGAAACACAAACTATGCGCAGAAATTTCAGGGCAGAGTCACCTTGACCACAGACACATCC
TCGACCACTACGTACCTGGATCTGAGGAGCCTGACATCTGACGACACGGCCATATATTACTGTGCGAGAGATGTT
CAATATCGTATGGACGTCTGGGGCCAAGGGACCACGGTCACCGTCTCTTCA
>B2-7_VH (SEQ ID NO: 449)
GAGGTGCAGCTGGTGGAGTCTGGGGGAGGCGTGGTCCAGCCCGGGAGGTCCCTGAGACTCTCCTGTGCAGCCTCT
GGATTCACCATCAGCAGTTATGGCATGCACTGGGTCCGCCAGGCTCCAGGCAAGGGGCTGGAGTGGGTGGCAGTT
ATATCATATGATGGAAGTAATAAATACTATGCAGACTCCGTGAAGGGCCGATTCACCATCTCCAGAGACAATTCC
AAGAACACGCTGTATCTGCAAATGAACAGCCTGAGAGTTGAGGACACGGCTGTGTATTACTGTGCGAAAGATCTC
TTATTACGATATTTTGACTGGAGCCCCAAATACTACTACAACGGTATGGACGTCTGGGGCCAAGGGACAATGGTC
ACCGTCTCCTCA
>B2-8_VH (SEQ ID NO: 450)
CAGGTGCAGCTGGTGCAATCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGAGAGTGGCA
GTGGCTGACACCTTCTTTGACTACTGGGGCCAGGGAACCCTGGTCACCGTCTCCTCA
>B2-9_VH (SEQ ID NO: 451)
CAGGTGCAGCTGGTGCAGTCTGGAGCTGAGGTGAAGAAGCCTGGGGCCTCAGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTCACCATGACCACAGACACATCC
ACGAGCACAGCCTACATGGAGCTGAGGAGCCTGAGATCTGACGACACGGCCGTGTATTACTGTGCGAGAGACGGC
TACGGTATGGACGTCTGGGGCCAAGGGACCACGGTCACCGTCTCCTCA
>B2-10_VH (SEQ ID NO: 452)
CAGGTGCAGCTGGTGCAGTCTGGGGGAGGCTTGGTCCAGCCTGGGGGGTCCCTGAGACTCTCCTGTGCAGCCTCT
GGATTCACCTTTAGCAGCTATGCCATGAGCTGGGTCCGCCAGGCTCCAGGGAAGGGGCTGGAGTGGGTCTCAACT
ATTAATGATAGTGGTGGTAGCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTATATTACTGTGCGAAAGATCAT
ATTACGATTTTTGGAGTGGCCCCGTTTGACTACTGGGGCCTGGGAACCCTGGTCACCGTCTCCTCA
>B2-11_VH (SEQ ID NO: 453)
CAGGTGCAGCTACAGCAGTCAGGTCCAGGACTGGTGAAGCCCTCGCAGACCCTCTCACTCACCTGTGCCATCTCC
GGGGACAGTGTCTCTAGCAACAGTGCTGCTTGGAACTGGATCAGGCAGTCCCCATCGAGAGGCCTTGAGTGGCTG
GGAAGGACATACTACAGGTCCAAGTGGTATAATGATTATGCAGTATCTGTGAAAAGTCGAATAACCATCAACCCA
GACACATCCAAGAACCAGTTCTCCCTGCAGCTGAACTCTGTGACTCCCGAGGACACGGCTGTGTATTACTGTGCA
AGACAAGATGAATCGGCAGCCGATGATGCTTTTGATATCTGGGGCCAAGGAACCCTGGTCATCGTCTCCTCA
>B2-12_VH (SEQ ID NO: 454)
CAGGTGCAGCTGGTGCAGTCTGGAGATGAGGTGAAGAAGGCTGGGGCCTCAGTGAGGGTCTCCTGCAAGGCTTCT
GGTTACACGTTTCCCGACAACGGTATCAGTTGGGTGCGACAGGCCCCTGGACACGGGCTTGAGTGGATGGGTTGG
ATCAACGTTGACACTGGATACACCCACTATGCACAGAGGGTCCAGGACAGAGTCGTCATGACCACAGACACGTCC
ACGAACACACTCCACATGACATTGAGGAGCCTGACAACGGACGACACGGCCGTTTATTATTGTGCGAGAGAAATC
GAGGGAGTGTGGACAGCTTGGGGCCAGGGAACCCTGGTCATCGTCTCCTCA
>B2-13_VH (SEQ ID NO: 455)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGAGGTCGGAC
TACGGTGACTCCTACTTTGACTACTGGGGCCAGGGAACCCTGGTCAGCGTCTCCTCA
>B2-14_VH (SEQ ID NO: 456)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGAGAGCCTCT
ATGGTCGAGACTTACTTTGACTACTGGGGCCAGGGAACCATGGTCACCGTCTCTTCA
>B2-15_VH (SEQ ID NO: 457)
GAGGTGCAGCTGGTGCAGTCTGGAGCTGAGGTGAAGAAGTCTGGGGCCTCAGTGAAGGTCTCCTGCAAGTCTTCT
GGTTACACCTTTACCAACTATGGTATCAGTTGGGTGCGACAAGCCCCGGGACAAGGGCTTGAGTGGATGGGCTGG
ATCAGCGGTGACGCTGGTGACACAAAATTTGCACAGAAGTTCCAGGGCAGAGTCACCATGACGACAGACACATCC
ACGACTACAACGTACATGGAGCTGAGGAGCCTGAGATCTGACGACACGGCCGTGTATTACTGTGCGAGAGATGGG
GAATATATTCCTATGTTTCGGGGATTTGACAACTACTACGGTCTGGACGTCTGGGGCCAAGGGACCCTGGTCAGC
GTCTCCTCA
>B2-16_VH (SEQ ID NO: 458)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGAGCGGGATT
ATACCTGATAATCCCTACGGTATGGACGTCTGGGGCCAAGGGACAATGGTCAGCGTCTCCTCA
>B2-17_VH (SEQ ID NO: 459)
GAGGTGCAGCTGTTGGACTCGGGCCCACGACTGTTGAAGCCTTCGGAGACCCTGTCCCTCACCTGCGGTGTGTCA
GGTGTCTCCACCAGCAGTACTCATTGGTGGAGTTGGGTCCGCCAGACCCCAGGGAAGGGGCTGGAGTGGATTGGT
GAAATCCTTCATAATGGAAACACCAACTCCAATCCGTCCCTCAAGAGTCGAGTCTCCATGTCGATTGACAAGTCG
AGGAACCAATTCTCCCTACAACTGAAGTCTATGACCGCCGCGGACACGGCCGTCTACTACTGTGCGTCAGCGTCG
CCTAACTGGGGAGCTAGCGGGGCTTTTGATGCCTGGGCCCAAGGGACAATGGTCACCGTCTCCTCA
>B2-18_VH (SEQ ID NO: 460)
CAGGTGCAGCTGGTGCAGTCTGGAGCTGAGGTGAAGAAGCCTGGGGCCTCAGTGAAGGTCTCCTGCAAGGCTTCT
GGTTACACCTTTACCAGGTATGGTTTCAGCTGGGTGCGACAGGCCCCTGGACAAGGGCTTGAGTGGATGGGATGG
ATCAGCGGTTACAATGGTAATACAAAGTATGCACAGAAGTTCCAGGGCAGAGTCACCATGACTACAGACACATCT
ACGAGCACAGCCTACATGGAGCTGAGGAGCCTAACATCTAACGACACGGCCGTTTATTACTGTGCGAGAGATCGG
GGTAGTGGTTATCTTGAATACTGGGGCCCGGGAACCCTGGTCACCGTCTCTTCA
>B2-22_VH (SEQ ID NO: 462)
CAGGTGCAGCTGGTGCAGTCTGGAGCTGAGGTGAAGAAGCCTGGGGCCTCAGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTCACCATGACCACAGACACATCC
ACGAGCACAGCCTACATGGAGCTGAGGAGCCTGAGATCTGACGACACGGCCGTGTATTACTGTGCGAGAGTGGGC
AGCTATGGTGACTACTGGGGCCAGGGAACCCTGGTCACCGTCTCCTCA
>B2-23_VH (SEQ ID NO: 463)
CAGGTGCAGCTGGTGCAGTCTGGGGCTGAGGTGAAGAAGCCTGGGGCCTCAGTGAAGGTTTCCTGCAAGGCATCT
GGGTACACCTTCACCCACTCCTATATACACTGGGTGCGCCAGGCCCCTGGACAAGGGCTTGAGTGGATGGGAGTA
ATCAACCCTGTTGGTGGTAGCACAACCTACGCACAGAGGTTCCAGGGCAGAGTCACCATGACCAGGGACACGTCC
ACGAGCACAGTCTACATGGAGCTGAGCAGCCTGAGATCTGAGGACACGGCCGTGTATTACTGTGCTAGAGCGGGG
GGAGATGGCTACAATCCTTACTTTGACTACTGGGGCCAGGGAACCCTGGTCACCGTCTCCTCA
>B2-24_VH (SEQ ID NO: 464)
CAGGTGCAGCTGGTGGAGTCTGGGGGAGGCTTGGTCAAGCCTGGAGGGTCCCTGAGACTCTCCTGTGCAGCCTCT
GGATTCACCTTCAGTGACTACTACATGAGCTGGATCCGCCAGGCTCCAGGGAAGGGGCTGGAGTGGGTTTCATAC
ATTAGTAGTAGTAGTAGTTACACAAACTACGCAGACTCTGTGAAGGGCCGATTCACCATCTCCAGAGACAACGCC
AAGAACTCACTGTATCTGCAAATGAACAGCCTGAGAGCCGAGGACACGGCCGTGTATTACTGTGCGAGAGTCGGA
GATCTTGATGCTTTTGATATCTGGGGCCAAGGGACAATGGTCACCGTCTCTTCA
>B2-25_VH (SEQ ID NO: 465)
CAGGTGCAGCTGGTGGAGTCTGGGGGAGGCTTGGTCAAGCCTGGAGGGTCCCTGAGACTCTCCTGTGCAGCCTCT
GGATTCACCTTCAGTGACCACTACATGAGCTGGATCCGCCAGGCTCCAGGAAAGGGGCTGGAGTGGGTTTCATAC
ATTACTACTACTGGTAGTACCATGTCCTACGCAGACTCTGTGAAGGGCCGCTTCACCATTTCCAGGGACAACTCC
AAGAACTCACTGCATCTGCAATTGAGCAGCCTGAGAGCCGAGGACACGGCCGTATATTACTGTGCGAGAGATTCC
ATACATGTGGATGGTGCTTTTGATATTTGGGGCCAAGGGACAATGGTCACCGTCTCCTCA
>B2-26_VH (SEQ ID NO: 466)
CAGGTGCAGCTGGTGCAGTCTGGAGCTGAGGTGAAGAAGCCTGGGGCCTCAGTGAAGGTCTCCTGCAAGGCTTCT
GGTTACACCTTTACCAGCTATGGTATCAGCTGGGTGCGACAGGCCCCTGGACAAGGGCTTGAGTGGATGGGATGG
ATCAGCGCTTACAATGGTAACACAAACTATGCACAGAAGCTCCAGGGCAGAGTCACCATGACCACAGACACATCC
ACGAGCACAGCCTACATGGAGCTGAGGAGCCTGAGATCTGACGACACGGCCGTGTATTACTGTGCGAGAGATCGG
GGAGGGTGGTTCGACCCCTGGGGCCAGGGAACCCTGGTCATCGTCTCCTCA
>B2-27_VH (SEQ ID NO: 467)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGAGAGAGTCG
TATAGCAGCTCGTCCTTTGACTACTGGGGCCAGGGAACCACGGTCAGCGTCTCCTCA
>B2-30_VH (SEQ ID NO: 468)
CAGGTGCAGCTGGTGGAGTCTGGGGGAGGCTTGGTACAGCCTGGGGGGTCCCTGAGACTCTCCTGTGCAGCCTCT
GGATTCACCTTCAGTAGCTACGACATGCACTGGGTCCGCCAAGCTACAGGAAAAGGTCTGGAGTGGGTCTCAGCT
ATTGGTACTGCTGGTGACACATACTATCCAGGCTCCGTGAAGGGCCGATTCACCATCTCCAGAGAAAATGCCAAG
AACTCCTTGTATCTTCAAATGAACAGCCTGAGAGCCGGGGACACGGCTGTGTATTACTGTGCAAGGGCGAAGGAA
TATTGTAGTGGTGGTAGCTGCCAAGATGCTTTTGATATCTGGGGCCAAGGGACAATGGTCAGCGTCTCCTCA
>HCB2-1c_VH (SEQ ID NO: 469)
GAAGTGCAGCTGGTGCAGTCTGGGGCTGAGGTGAAGAAGCCTGGGGCCTCAGTGAAGGTTGCCTGCAAGGCATCT
GGGTACACCTTCACCGACTATTATATACACTGGGTGCGACAGGCCCCTGGACAAGGGCTTGAGTGGATGGGAATA
GTCAACGCTAATGGTGGTGACACAAACTACGCACAACAGTTCCAGGGCAGAGTCACCATGACCAGGGACACGTCC
ACGAGCACAGTCTATTTGGAGTTGACCAGCCTGAGATCTGACGACACGGCCGTATATTACTGTGCTAGAGATTGG
TATTACTATTCTTCGGGGAGTTTCCCTTTTGACTACTGGGGCCAGGGAACCCTGGTCAGCGTCTCCTCA
>HCB2-1d_VH (SEQ ID NO: 470)
GAAGTGCAGCTGGTGCAGTCTGGGGCTGAGGTGAAGAAGCCTGGGGCCTCAGTGAAGGTTGCCTGCAAGGCATCT
GGGTACACCTTCACCGACTATTATATACACTGGGTGCGACAGGCCCCTGGACAAGGGCTTGAGTGGATGGGAATA
GTCAACGCTAATGGTGGTGACACAAACTACGCACAACAGTTCCAGGGCAGAGTCACCATGACCAGGGACACGTCC
ACGAGCACAGTCTATTTGGAGTTGACCAGCCTGAGATCTGACGACACGGCCGTATATTACTGTGCTAGAGATTGG
TATTACTATTCTTCGGGGAGTTTCCCTTTTGACTACTGGGGCCAGGGAACCCTGGTCAGCGTCTCCTCA
>HCB2-2_VH (SEQ ID NO: 471)
CAGGTGCAGCTGGTGCAGTCTGGAGCTGAGGTGAAGAAGCCTGGGGCCTCAGTGAAGGTCTCCTGCAAGGCTTCT
GGTTACATCTTCAACACCTATGGTATCAGTTGGGTGCGACAGGCCCCTGGACAAGGACTTGAGTGGATGGGATGG
ACCAACGTTTACAATAGTAACACAGACAGTGGACACAAGTTCCAGGGCAGAGTCACCATGACCACAGACACATCC
ACGGACACAGCCTACATGGAACTGAGGAGCCTGAGATCTGACGACACGGCCGTGTATTACTGTGCGAGAGACCCG
GGCTCGGCTATTTTTGATATGTGGGGCCAAGGGACAATGGTCACCGTCTCCTCA
>HCB2-4_VH (SEQ ID NO: 472)
CAGGTGGAGCTGCAGGAGTCGGGCCCAGGACTGGTGAAGCCTTCGGAGACCCTGTCCCTCACTTGCACTGTCTCT
GGTGACGCCATCAAAGATAAGTACTGGAGTTGGGTCCGGCAGCCCCCAGGGAAGGGACTGGAGTGGATTGGCTAC
ATCTCTAACAGTGGAAGCACCAACTACAACCCCTCCCTCAAGAGTCGAGTCAGTCTATCAGTAGACACGTCCAAG
AATCAGTTCTCCCTGAAGCAGACCTCTGTGACCGCTGCGGACACGGCCACATATTACTGTGCGAGAGGCAGCGGT
TATACTGCTTTTGATATCTGGGGCCAAGGGACAATGGTCACCGTCTCCTCA
>HCB2-5_VH (SEQ ID NO: 473)
CAGGTGCAGCTGGTGGAGTCTGGGGGAGGCTTGGTACAGCCTGGGGGGTCCCTGAGACTCTCCTGTGCAGCCTCT
GGATTCACCTTTAGCAGCTATGCCATGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTATATTACTGTGCGAAAGATGTC
GACCCCTATAGTAGTGGTTATTTTGACTACTGGGGCCAGGGAACCCTGGTCATCGTCTCCTCA
>HCB2-10_VH (SEQ ID NO: 474)
CAGGTGCAGCTGGTGGAGTCTGGGGGAGGCTTGGTACAGCCTGGGGGGTCCCTGAGACTCTCCTGTGCAGCCTCT
GGATTCACCTTTAGCAACTATGCCATGAGCTGGGTCCGCCAGGCTCCAGGGAAGGGGCTGGAGTGGGTCTCAGCC
ATTAGTGGTAGTAGTATTAGCACATACTACGCAGACTCCGTGAAGGGCCGGTTCACCATCTCCAGAGACAATTCC
AAGAACACGCTTTATCTGCAAATGAACAGCCTGAGAGCCGAGGACACGGCCGTATATTACTGTGCGTCCTCGGGT
TTCGGGTCCTTTGACTACTGGGGCCAGGGCACCCTGGTCACCGTCTCTTCA
>HCB2-13_VH (SEQ ID NO: 475)
CAGGTGCAGCTGGTGGAGTCTGGGGGAGGCTTGGTACAGCCTGGGGGGTCCCTGAGACTCTCCTGTGCAGCCTCT
GGATTCACCTTAAGTAGCTATGCCATGAGCTGGGTCCGCCAGGCTCCAGGGGAGGGGCTGGAGTGGGTCTCAAGT
ATTAATAGTGGTGGTAGCACATACTACGCAGGCTCCGTGAGGGGCCGGTTCACCATCTCCAGAGACAATTCCAAG
AACACGTTGTATCTGCAAATGAACAGCCTGAGAGCCGAGGACACGGCCGTATATTACTGTGCGAAAGCGAAGAGT
TATAGCAGTGACCTTGACCACTGGGGCCAGGGAACCACGGTCACCGTCTCTTCA
>HCB2-14_VH (SEQ ID NO: 476)
CAGGTGCAGATGGTGGAGTCTGGGGGAGGCTTGGTACAGCCTGGGGGGTCCCTGAGACTCTCCTGTGCAGCCTCT
GGATTCACCTTTAGCAGCTATGCCATGAGCTGGGTCCGCCAGGCTCCAGGGAAGGGGCTGGAGTGGGTCTCAGCT
ATTAGTGGTAGTGGTGGTAGCACATACTACGCAGACTCCGTGAAGGGCCGGTTCACCATCTCCAGAGACAATTCC
AAGAACACGCTGTATCTGCAAATGAACAGCCTGAGAGCCGAGGACACGGCCGTATATTACTGTGCGAAAGATCTT
CTCGGGGATAGTGGGAGCTACCCTGCTTTTGATATCTGGGGCCAAGGGACAATGGTCACCGTCTCTTCA
>HCB2-15_VH (SEQ ID NO: 477)
CAGGTGCAGATGGTGGAGTCTGGGGGAGACTTGGTACAGCCAGGGCAATCCCTGAGACTCTCCTGTGTAACTTCT
GGATTCCCCTTTGCGGACTATGCTATGAGCTGGTTCCGCCAGGCTCCAGGGCAGCGGCCGGAGTGGATAGGTTAC
ATTAAAAGCAAGACTAGAGATGGGACAACAAAATACGCCGAGTCTCTGCGAGGCAGATTCACCATCTCAAGAGAT
GATTCCAAAAGCATCGCCTATCTACAAATGAACAACTTAAAAAGGGAAGACACAGCCGTCTATTACTGTGCCCTC
GGGGGAGCAGCAGCTCTATGGGGCCCGGGAACCCTGGTCAGCGTCTCCTCA
>HCB2-17_VH (SEQ ID NO: 478)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGATTAGTAGT
GGTTATTATTATAACCCATTTGACTACTGGGGCCAGGGAACCCTGGTCACCGTCTCCTCA
>HCB2-18_VH (SEQ ID NO: 479)
CAGGTGCAGCTGGTGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACGAATCC
ACGAGCACAGCCTACATGGAGCTGAGCAGCCTGAGATCTGAGGACACGGCCGTGTATTACTGTGCGAGAGGGGGA
TACCAGCCACTGGATTACTGGGGCCAGGGAACCCTGGTCATCGTCTCCTCA
>HCB2-24_VH (SEQ ID NO: 480)
CAAATGCAGCTGGAGCAGTCTGGGGCTGAGGTGAAGAAGCCTGGGTCCTCGGTGAAGGTCTCCTGCAAGGCTTCT
GGAGGCACCTTCAGCAGCTATGCTATCAGCTGGGTGCGACAGGCCCCTGGACAAGGGCTTGAGTGGATGGGAGGG
ATCATCCCTATCTTTGGTACAGCAAACTACGCACAGAAGTTCCAGGGCAGAGTCACGATTACCGCGGACAAATCC
ACGAGCACAGCCTACATGGAGCTGAGCAGCCTGAGATCTGAGGACACGGCCGTGTATTACTGTGCGAGAGCTGGG
GGAATCCATTACTATGATAGTTTAGACTACTGGGGCCAGGGAACCCTGGTCACCGTCTCCTCA
>HCB2-27_VH (SEQ ID NO: 481)
CAGGTGCAGCTGGTGGAGTCTGGGGGAGGCTTGGTAAAGCCGGGGGGGTCTCTAAGACTCTCCTGTGCAGCCTCT
GGATTCACCTTCGCGAATGCCTGGATGGGCTGGGTTCGCCAGGCTCCAGGGAAGGGGCTGCAGTGGGTATCCCAT
ATTAAGCCCAGTTCTAGCGGCGGGAGGACAACAGACTACGATGCACCCGTGAAAGGCCGATTCACCATCTCAAGA
GATGATTCAAACGACACGGTGTATCTGCAAATGAACAGCCTCAAGACCGAAGACACAGGCGTCTATTACTGTGTC
ACAGGAAGAAGGGTCGCGAAGACTTTTGATTTCTGGGGCCAGGGGACAACGGTCAGCGTCTCCTCA

TABLE 5
Amino acid sequences of antibody heavy chain variable regions (VH)

>B2-19_VH (SEQ ID NO: 498)
QVQLVQSGGGLVKPGGSLRLSCAASGFTFSNAWMSWVRQAPGKGLEWVGRIKSKTDGGTTDYAAPVKGRFTISRD
DSKNTLYLQMNSLKTEDTAVYYCTTDRYSSSWYSPAFDIWGQGTTVSVSS
>B2-1_VH (SEQ ID NO: 482)
EVQLVQSGAEVKKPGSSVKVSCKASGGTFSSYAISWVRQAPGQGLEWMGGIIPIFGTANYAQKFQGRVTITADKS
TSTAYMELSSLRSEDTAVYYCARGNQGDTAFDIWGQGTTVIVSS
>B2-2_VH (SEQ ID NO: 483)
QVQLVQSGAEVKKPGASVKVSCKASGYTFSDYGFSWVRQAPGQGLEWMGWISGYNGNTNYAQKFQGRVTMTIDAS
TSTAYMELRSLKSDDTAVYYCARLLLGYYGMDVWGQGTTVTVSS
>B2-3_VH (SEQ ID NO: 484)
QVQLVQSGAEVKKPGASVKVSCRTSGYTFSSYGISWVRQAPGQGLEWMGWISGNTGNTKYTPKLQGRVTMTTDTS
TSTAYMELRSLRSDDTAVYYCARDRGIAAGRAFDIWGQGTMVTVSS
>B2-4_VH (SEQ ID NO: 485)
QVQLVQSGPTVRKPGASVRVSCRASGYPFTSNGMSWVRQAPGQGLEWMGWTSTNSGNTNY
AQKFQGRVTLTTDTSSTTTYLDLRSLTSDDTAIYYCARDVQYRMDVWGQGTTVTVSS
>B2-7_VH (SEQ ID NO: 486)
EVQLVESGGGVVQPGRSLRLSCAASGFTISSYGMHWVRQAPGKGLEWVAVISYDGSNKYYADSVKGRFTISRDNS
KNTLYLQMNSLRVEDTAVYYCAKDLLLRYFDWSPKYYYNGMDVWGQGTMVTVSS
>B2-8_VH (SEQ ID NO: 487)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCARVAVADTFFDYWGQGTLVTVSS
>B2-9_VH (SEQ ID NO: 488)
QVQLVQSGAEVKKPGASVAVSCKASGYTFTSYGISWVRQAPGQGLEWMGWISAYNGNTNYAQKLQGRVTMTTDTS
TSTAYMELRSLRSDDTAVYYCARDGYGMDVWGQGTTVTVSS
>B2-10_VH (SEQ ID NO: 489)
QVQLVQSGGGLVQPGGSLRLSCAASGFTFSSYAMSWVRQAPGKGLEWVSTINDSGGSTYYADSVKGRFTISRDNS
KNTLYLQMNSLRAEDTAVYYCAKDHITIFGVAPFDYWGLGTLVTVSS
>B2-11_VH (SEQ ID NO: 490)
QVQLQQSGPGLVKPSQTLSLTCAISGDSVSSNSAAWNWIRQSPSRGLEWLGRTYYRSKWYNDYAVSVKSRITINP
DTSKNQFSLQLNSVTPEDTAVYYCARQDESAADDAFDIWGQGTLVIVSS
>B2-12_VH (SEQ ID NO: 491)
QVQLVQSGDEVKKAGASVRVSCKASGYTFPDNGISWVRQAPGHGLEWMGWTNVDTGYTHYAQRVQDRVVMTTDTS
TNTLHMTLRSLTTDDTAVYYCAREIEGVWTAWGQGTLVIVSS
>B2-13_VH (SEQ ID NO: 492)
QVQLVQSGAEVKKPGSSVKVSCKASGGTFSSYAISWVRQAPGQGLEWMGGIIPIFGTANYAQRFQGRVTITADES
TSTAYMELSSLRSEDTAVYYCARSDYGDSYFDYWGQGTLVSVSS
>B2-14_VH (SEQ ID NO: 493)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCARASMVETYFDYWGQGTMVTVSS
>B2-15_VH (SEQ ID NO: 494)
EVQLVQSGAEVKKSGASVKVSCKSSGYTFTNYGISWVRQAPGQGLEWMGWISGDAGDTKFAQKFQGRVIMTTDTS
TTTTYMELRSLRSDDTAVYYCARDGEYIPMFRGFDNYYGLDVWGQGTLVSVSS
>B2-16_VH (SEQ ID NO: 495)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCASGIIPDNPYGMDVWGQGTMVSVSS
>B2-17_VH (SEQ ID NO: 496)
EVQLLDSGPRLLKPSETLSLTCGVSGVSTSSTHWWSWVRQTPGKGLEWIGEILHNGNTNSNPSLKSRVSMSIDKS
RNQFSLQLKSMTAADTAVYYCASASPNWGASGAFDAWAQGTMVTVSS
>B2-18_VH (SEQ ID NO: 497)
QVQLVQSGAEVKKPGASVKVSCKASGYTFTRYGFSWVRQAPGQGLEWMGWISGYNGNTKYAQKFQGRVTMTTDTS
TSTAYMELRSLTSNDTAVYYCARDRGSGYLEYWGPGTLVTVSS
>B2-22_VH (SEQ ID NO: 499)
QVQLVQSGAEVKKPGASVKVSCKASGYTFTSYGISWVRQAPGQGLEWMGWISAYNGNTNYAQKLQGRVTMTTDTS
TSTAYMELRSLRSDDTAVYYCARVGSYGDYWGQGTLVTVSS
>B2-23_VH (SEQ ID NO: 500)
QVQLVQSGAEVKKPGASVKVSCRASGYTFTHSYIHWVRQAPGQGLEWMGVINPVGGSTTYAQRFQGRVTMTRDTS
TSTVYMELSSLRSEDTAVYYCARAGGDGYNPYFDYWGQGTLVTVSS
>B2-24_VH (SEQ ID NO: 501)
QVQLVESGGGLVKPGGSLRLSCAASGFTFSDYYMSWIRQAPGKGLEWVSYISSSSSYTNYADSVKGRFTISRDNA
KNSLYLQMNSLRAEDTAVYYCARVGDLDAFDIWGQGTMVTVSS
>B2-25_VH (SEQ ID NO: 502)
QVQLVESGGGLVKPGGSLRLSCAASGFTFSDHYMSWIRQAPGKGLEWVSYITTTGSTMSYADSVKGRFTISRDNS
KNSLHLQLSSLRAEDTAVYYCARDSIHVDGAFDIWGQGTMVTVSS
>B2-26_VH (SEQ ID NO: 503)
QVQLVQSGAEVKKPGASVKVSCKASGYTFTSYGISWVRQAPGQGLEWMGWISAYNGNTNYAQKLQGRVTMTTDTS
TSTAYMELRSLRSDDTAVYYCARDRGGWFDPWGQGTLVIVSS
>B2-27_VH (SEQ ID NO: 504)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCARESYSSSSFDYWGQGTTVSVSS
>B2-30_VH (SEQ ID NO: 505)
QVQLVESGGGLVQPGGSLRLSCAASGFTFSSYDMHWVRQALGKGLEWVSALGIAGDIYYPGSVKGRFTISRENAK
NSLYLQMNSLRAGDTAVYYCARAKEYCSGGSCQDAFDIWGQGTMVSVSS
>HCB2-1c_VH (SEQ ID NO: 506)
EVQLVQSGAEVKKPGASVKVACKASGYTFTDYYIHWVRQAPGQGLEWMGIVNANGGDTNYAQQFQGRVTMTRDTS
TSTVYLELTSLRSDDTAVYYCARDWYYYSSGSFPFDYWGQGTLVSVSS
>HCB2-1d_VH (SEQ ID NO: 507)
EVQLVQSGAEVKKPGASVKVACKASGYTFTDYYIHWVRQAPGQGLEWMGIVNANGGDTNYAQQFQGRVTMTRDTS
TSTVYLELTSLRSDDTAVYYCARDWYYYSSGSFPFDYWGQGTLVSVSS
>HCB2-2_VH (SEQ ID NO: 508)
QVQLVQSGAEVKKPGASVKVSCKASGYIFNTYGISWVRQAPGQGLEWMGWTNVYNSNTDSGHKFQGRVTMTTDTS
TDTAYMELRSLRSDDTAVYYCARDPGSAIFDMWGQGTMVTVSS
>HCB2-4_VH (SEQ ID NO: 509)
QVELQESGPGLVKPSETLSLTCTVSGDAIKDKYWSWVRQPPGKGLEWIGYISNSGSTNYNPSLKSRVSLSVDTSK
NQFSLKQTSVTAADTATYYCARGSGYTAFDIWGQGTMVTVSS
>HCB2-5_VH (SEQ ID NO: 510)
QVQLVESGGGLVQPGGSLRLSCAASGFTFSSYAMSWVRQAPGKGLEWVSAISGSGGSTYYADSVKGRFTISRDNS
KNTLYLQMNSLRAEDTAVYYCAKDVDPYSSGYFDYWGQGFLVIVSS
>HCB2-10_VH (SEQ ID NO: 511)
QVQLVESGGGLVQPGGSLRLSCAASGFFFSNYAMSWVRQAPGKGLEWVSAISGSSISFYYADSVKGRFTISRDNS
KNTLYLQMNSLRAEDTAVYYCASSGFGSFDYWGQGTLVTVSS
>HCB2-13_VH (SEQ ID NO: 512)
QVQLVESGGGLVQPGGSLRLSCAASGFTLSSYAMSWVRQAPGEGLEWVSSINSGGSTYYAGSVRGRFTISRDNSK
NTLYLQMNSLRAEDTAVYYCAKAKSYSSDLDHWGQGTTVTVSS
>HCB2-14_VH (SEQ ID NO: 513)
QVQMVESGGGLVQPGGSLRLSCAASGFTFSSYAMSWVRQAPGKGLEWVSAISGSGGSTYYADSVKGRFTISRDNS
KNTLYLQMNSLRAEDTAVYYCAKDLLGDSGSYPAFDIWGQGTMVTVSS
>HCB2-15_VH (SEQ ID NO: 514)
QVQMVESGGDLVQPGQSLRLSCVTSGFPFADYAMSWFRQAPGQRPEWIGYIKSKTRDGTTKYAESLRGRFTISRD
DSKSIAYLQMNNLKREDTAVYYCALGGAAALWGPGTLVSVSS
>HCB2-17_VH (SEQ ID NO: 515)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCAISSGYYYNPFDYWGQGTLVTVSS
>HCB2-18_VH (SEQ ID NO: 516)
QVQLVQSGAEVKKPGSSVKVSCKASGGTFSSYAISWVRQAPGQGLEWMGGIIPIFGTANYAQKFQGRVTITADES
TSTAYMELSSLRSEDTAVYYCARGGYQPLDYWGQGTLVIVSS
>HCB2-24_VH (SEQ ID NO: 517)
QMQLEQSGAEVKKPGSSVKVSCKASGGFFSSYAISWVRQAPGQGLEWMGGIIPIFGFANYAQKFQGRVTITADKS
TSTAYMELSSLRSEDTAVYYCARAGGIHYYDSLDYWGQGTLVTVSS
>HCB2-27_VH (SEQ ID NO: 518)
QVQLVESGGGLVKPGGSLRLSCAASGFFFANAWMGWVRQAPGKGLQWVSHIKPSSSGGRFFDYDAPVKGRFTISR
DDSNDTVYLQMNSLKTEDTGVYYCVTGRRVAKTFDFWGQGTTVSVSS

TABLE 6
DNA sequences of LILRB2 antibody light chain variable regions (VL)

>B2-19_VL (SEQ ID NO: 535)
CAGCCTGTGCTGACTCAGCCACCCTCAGTGTCTGAAGCCCCCAGGCAGAGGGTCACCATCTCCTGTTCTGGAAGC
AGCTCCAACATCGGAAATAATGTTGTAAACTGGTACCAGCAGCTCCCAGGAAAGGCTCCCAAACTCCTCATCTAT
TATGATGATCTGCTGCCCTCAGGGGTCTCTGACCGATTCTCTGGCTCCAAGTCTGGCACCTCAGCCTCCCTGGCC
ATCAGTGGGCTCCAGTCTGAGGATGAGGCTGATTATTACTGTGCAACATGGGATGACAGCCTGAATGGCCCTGTA
TTCGGCGGAGGGACCAAGCTGACCGTCCTA
>B2-1_VL (SEQ ID NO: 519)
CAGTCTGCCCTGAATCAGCCTGCCTCCGTGTCTGGGTCTCCTGGACAGTCGATCACCATCTCCTGCACTGGAACC
AGCAGTGACGTTGGTGGTTATAACTATGTCTCCTGGTACCAACAACACCCAGGCAAAGCCCCCAAACTCATGATT
TATGATGTCAGTAATCGGCCCTCAGGGGTTTCTAATCGCTTCTCTGGCTCCAAGTCTGGCAACACGGCCTCCCTG
ACCATCTCTGGGCTCCAGGCTGAGGACGAGGCTGATTATTACTGCAGCTCATATACAAGCAGCAGCACTCCTTAT
GTCTTCGGAACTGGGACCAAGGTCACCGTCCTA
>B2-2_VL (SEQ ID NO: 520)
GACATCGTGATGACCCAGTCTCCATCCTCCCTGTCTGCATCTGTAGGAGACAGAGTCACCCTCACTTGCCGGGCA
AGTCAGAGCATTGGCAACTATTTAAATTGGTATCAACAGAAACCAGGGAAAGCCCCTAACCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAGCCTGAAGATTTTGCAACTTACTACTGTCAACAGAGTTTCAGTATCCCCCCGATAACTTTCGGC
CAAGGGACACGACTGGAGATTAAA
>B2-3_VL (SEQ ID NO: 521)
CAGCCTGTGCTGACTCAGCCACCCTCAGCGTCTGGGACCCCCGGCCAGAGGGTCACCATCTCTTGTTCTGGAAGC
AGCTCCAACATCGGAACTAATCCTGTAAACTGGTACCAGCAAGTCCCAGGAACGGGCCCCAAACTCCTCATCTAT
AATAATAATCAGTGGCCCTCAGGGGTCCCTGACCGATTCTCTGGCTCCAAGTCTGCCACCTCAGCCTCCCTGGCC
ATCTATGGGCTCCAGTCTGGGGATGAGGCTCATTATTACTGTTCGTCATGGGACGACAGCCTGAGTGCGTGGGTG
TTCGGCGGAGGGACCAAGGTCACCGTCCTA
>B2-4_VL (SEQ ID NO: 522)
CAGTCTGTGCTGACTCAGCCACCCTCAGCGTCTGGGACCCCCGGGCAGAGGGTCACCATCTCTTGTTCTGGAAGC
AGGTCCAACATCGGAAGTAATCCTGTAAACTGGTATCAGCACCTCCCAGGAACGGCCCCCAAACTCCTCGTCTAT
AGTAATAATCGGCGGCCCTCAGGGGTCCCTGACCGATTCTCTGGCTCCAAGTCTGGCACCTCAGCCTCCCTGTCC
ATCAGTGGGCTCCAGTCTGAGGATGAAGGTGTTTATTACTGTGCAGCGTGGGATGACAACGTGCACGGAGTTTTC
GGCGGAGGGACCAAGGTCACCGTCCTA
>B2-7_VL (SEQ ID NO: 523)
CAGTCTGTGCTGACTCAGCCACCCTCGGTGTCAGTGGCCCCAGGAAAGACGGCCAGGATTACCTGTGGGGGAAAC
AACATTGGAAGTAAAAGTGTGCACTGGTACCAGCAGAAGCCAGGCCAGGCCCCTGTGCTGGTCGTCTATGATGAT
AGCGACCGGCCCTCAGGGATCCCTGAGCGATTCTCTGGCTCCAACTCTGGGAACACGGCCACCCTGACCATCAGC
AGGGTCGAAGCCGGGGATGAGGCCGACTATTACTGTCAGGTGTGGGATAGTAGTAGTGATCATCCGGGGGTCTTC
GGAACTGGGACCAAGGTCACCGTCCTA
>B2-8_VL (SEQ ID NO: 524)
CAGCCTGTGCTGACTCAGCCTGCCTCCGTGTCTGGGTCTCCTGGACAGTCGATCACCATCTCCTGCACTGGAACC
AGCAGTGACGTTGGTGGTTATAACTATGTCTCCTGGTACCAACAACACCCAGGCAAAGCCCCCAAACTCATGATT
TATGATGTCAGTAATCGGCCCTCAGGGGTTTCTAATCGCTTCTCTGGCTCCAAGTCTGGCAACACGGCCTCCCTG
ACCATCTCTGGGCTCCAGGCTGAGGACGAGGCTGATTATTACTGCAGCTCATATACAAGCAGCAGCACCCCTTAT
GTCTTCGGAACTGGGACCAAGGTCACCGTCCTA
>B2-9_VL (SEQ ID NO: 525)
CAGTCTCCATCCTCCCTGTCTGCATCTGTAGGCGACAGAGTCACCATCACTTGCCGGACAAGTCACACCGTTAAC
AGCTATCTAAATTGGTATCAACAAGAACCAGGGAAAGCCCCTAAACTCCTGATCTATGCTGCATCCAATTTGCAA
AGTGGGGTCCCGTCAAGGTTCAGTGGCAGTGGATCTGGGACAGATTTCTCTCTCACCATCCACAATCTGCAACCT
GAAGATTTTGCAACTTACTACTGTCAACAGAGTTACAGAACCCCTCTCACTTTTGGCCAGGGGACCAAAGTGGAT
ATCAAA
>B2-10_VL (SEQ ID NO: 526)
CAGTCTGTGCTGACTCAGCCACCCTCAGCGTCTGGAACCCCCGGGCAGAGGGTCACCATCTCTTGTTCTGGAAGC
AGCTCCAACATCGGAGGTAATCCTGTAAACTGGTACCAGCACCTCCCAGGAACGGCCCCCAAGCTCCTCATCTAT
TATAATGATCAGCGGCCTTCAGGGGTCCCTGACCGATTCTCTGGCTCCAAGTCTGGCACCTCAGCCTCCCTGGCC
ATCAGTGGACTCCAGTCTGAGGATGAGACTGATTATTACTGTGCAACATGGGATGACAGCCTGAATGGTTGGGTG
TTTGGCGGAGGGACCCAGCTGACCGTCCTA
>B2-11_VL (SEQ ID NO: 527)
CAGTCTGTGCTGACTCAGCCACCCTCAGCGTCTGGCACCCCCGGGCAGAGGGTCACCATCTCTTGTTCTGGAAGC
AAGTCCAACGTCGGAAATAATTTTGTACATTGGTACCAGCAACTCCCAGGGACGGCCCCCAAACTCCTCCTCTAT
AGCAATCTTGAGCGGTCCTCAGGGGTCCCTGAGCGATTCTCTGGCTCCAAGTCTGGCACCTCAGCCTACCTGGCC
ATCAGTGGGCTCCGGTCCGACGATGAGGCTGATTATTACTGTGCAGCATGGGATGACAGCCTGCCCGGTTGGGTG
TTCGGCGGAGGGACCAAGCTGACCGTCCTA
>B2-12_VL (SEQ ID NO: 528)
CAGCCTGTGCTGACTCAGCCACCCTCAGCGTCTGGGACCCCCGGGCAGAGGGTCACCATCTCTTGTTCTGGAAGC
AGCTCCAACATCGGAAGTAATCCTCTAAACTGGTACCAGCAACTCCCAGGAACGGCCCCCAAACTCCTCATCTAT
AATAATAATCAGCGGCCCTCAGGGGTCCCTGACCGATTCTCTGGCTCCAAGTCTGGCACCTCAGCCTCCCTGGCC
ATCAGTGGACTCCAGTCTGAGGATGAGGCTGATTATTACTGTGCGGCATGGGATGACAGCCTGAATGGTTGGGTG
TTCGGCGGAGGGACCCAGCTGACCGTCCTA
>B2-13_VL (SEQ ID NO: 529)
CAGGCTGTGGTGACTCAGCCTGCCTCCGTGTCTGGGTCTCCTGGACAGTCGGTCAGCATCTCCTGCACTGGAACC
AGCAGTGACGTTGGTGGTTTTAACTATGTCTCCTGGTATCAACAACACCCAGGCAAAGCCCCCAAACTCATGATT
TATGATGTCAGTAATCGGCCCTCAGGGGTTTCTAATCGCTTCTCTGGCTCCAAGTCTGGCAACACGGCCTCCCTG
ACCATCTCTGGGCTCCAGGCTGAGGACGAGGCTGATTATTACTGCAGCTCATATACAAGCAGCACCGCTCCTTAT
GTCTTCGGAACTGGGACCAAGGTCACCGTCCTA
>B2-14_VL (SEQ ID NO: 530)
CAGTCTGTGCTGACTCAGCCTCGCTCAGTGTCCGGGTCTCCTGGACAGTCAGTCACCATCTCCTGCACTGGAACC
AGCAGTGATGTTGGTGGTTATAACTATGTCTCCTGGTACCAACAACACCCAGGCAAAGCCCCCAAACTCATGATT
TCTGAGGTCAGTAAGCGGCCCTCAGGGGTCCCTGATCGCTTCTCTGGCTCCAAGTCTGGCAACACGGCCTCCCTG
ACCGTCTCTGGGCTCCAGGCTGAGGATGAGGCTGATTATTACTGCTCCTCATATGCAGGCGGGTACACCCCCTAT
GTCTTCGGAACTGGGACCAAGGTCACCGTCCTA
>B2-15_VL (SEQ ID NO: 531)
CAGTCTGTGCTGACTCAGCCACCCTCAGCGTCTGCGACCCCCGGGCAGAGGGTCACCATCTCTTGTTCTGGAAGA
AACGCCAACATCGGAAGTAATCCTGTAAACTGGTACCAGCAGCTCCCAGGGACGGCCCCCAGACTCGTCATGTAT
AGTAATAATCAGCGGCCCTCAGGGGTCCCTGACCGATTCTCTGGCTCCAAGTCTGGCACCTCAGCCTCCCTGGCC
ATCAGTGGGCTCCAGTCTGAGGACGAGGCTGATTATTACTGTGAAGGATGGGATGACAGCCTGAATGGCTATGTC
TTCGGAACTGGGACCAAGGTCACCGTCCTA
>B2-16_VL (SEQ ID NO: 532)
GATATTGTGATGACTCAGTCTCCACTCTCCCTGCCCGTCACCCTTGGACAGCCGGCCTCCATCTCCTGCACGTCT
AGTCAAAGCCTCGTATACAGTGATGGAAACACCTACTTGAATTGGTTTCACCAGAGGCCAGGCCAGTCTCCAAGG
CGCCTATTTTATAAGGTTTCTAACCGGGCCTCTGGGGTCCCAGACAGATTCAGCGGCAGTGGGTCAGGCACTGAT
TTCACACTGAAAATCAGCAGGGTGGAGGCTGAGGATCTTGGGGTTTATTACTGCATGCAAGGTACACACTGGCCG
GTCACCTTCGGCCAAGGGACACGACTGGAGATTAAA
>B2-17_VL (SEQ ID NO: 533)
CAGTCTGTGCTGACTCAGCCACCCTCAGCGTCTGCGACCCCCGGGCAGAGGGTCACCATCTCTTGTTCTGGAAGC
AGCTCCAACATCGGAAGTAATCCTGTAAACTGGTATCAGCAGGTCCCAGGAATGGCCCCCGAACTCGTCATGTAT
AGTGATAATCAGCGGCCCTCAGGGGTCCCTGACCGATTCTCTGGCTCCAACTCTGGCACCTCAGCCTCCCTGGCC
ATCAGTGGCCTCCAGTCTGAGGATGAGGCTGATTATTACTGTTCAACATGGGATGACAGCCTGAATGGTCTTGTC
TTCGGAACTGGGACCAAGGTCACCGTCCTA
>B2-18_VL (SEQ ID NO: 534)
GACGTCCAGATGACCCAGTCTCCATCCTCCCTGTCTGCATCTGTAGGAGAGAGAGTCACCCTCACTTGCCGGGCG
AGTCAGGGCATTTACAGTTCTTTAGCCTGGTATCAGCAAAAACCAGGTAAAGCCCCTAAACTCCTACTATATGGT
ACGTCGCAATTGGAACGTGGGGTCCCATCCAGATTCAGTGGCAGTGGATCTGGGACGGATTACACTCTCACCATC
AGCAGCCTGCAGCCTGAAGATTCTGCAACTTATTATTGTCAACAACATTACAATCTACCCATCACCTTCGGCCAA
GGGACACGACTGGAGATTAAA
>B2-22_VL (SEQ ID NO: 536)
CAGTCTCCATCCTCCCTGTCTGCATCTGTAGGAGACAGAGTCACCATCACTTGCCGGGCGAGTCAGGGCATTAGC
AATTATTTAGCCTGGTATCAGCAGAAACCAGGGAAAGTTCCTAAGCTCCTGATCTATGCTGCATCCACTTTGCAA
TCAGGGGTCCCATCTCGGTTCAGTGGCAGTGGATCTGGGACAGATTTCACTCTCACCATCAGCAGCCTGCAGCCT
GAAGATGTTGCAACTTATTACTGTCAAAAGTATAACAGTGCCCCGCTCACTTTCGGCGGAGGGACCAAGCTGGAG
ATCAAA
>B2-23_VL (SEQ ID NO: 537)
CAGTCTCCTTCCACCCTGTCTGCATCTGTAGGAGACAGAGTCACCATCACTTGCCGGGCCAGTCAGAGTATTAGT
AGCTGGTTGGCCTGGTATCAGCAGAAACCAGGGAAAGCCCCTAAGCTCCTGATCTATAAGGCGTCTAGTTTAGAA
AGTGGGGTCCCATCAAGGTTCAGCGGCAGTGGATCTGGGACAGAATTCACTCTCACCATCAGCAGCCTGCAGCCT
GATGATTTTGCAACTTATTACTGCCAACAGTATAATAGTTATCTCACTTTCGGCGGAGGGACCAAAGTGGATATC
AAA
>B2-24_VL (SEQ ID NO: 538)
CAGTCTGTGCTGACTCAGCCACCCTCAGCGTCTGGGACCCCCGGGCAGAGGGTCACCATCTCTTGTTCTGGAAGC
AGCTCCAACATCGGAAGTAATTATGTATACTGGTACCAGCAGCTCCCAGGAACGGCCCCCAAACTCCTCATCTAT
AGGAATAATCAGCGGCCCTCAGGGGTCCCTGACCGATTCTCTGGCTCCAAGTCTGGCACCTCAGCCTCCCTGGCC
ATCAGTGGGCTCCGGTCCGAGGATGAGGCTGATTATTACTGTGCAGCATGGGATGACAGCCTGAGTGGTTGGGTG
TTCGGCGGAGGGACCAAGGTCACCGTCCTA
>B2-25_VL (SEQ ID NO: 539)
TCCTATGAGCTGACTCAGCCACCCTCGGTGTCAGTGGCCCCAGGACAGACGGCCAGGATTACCTGTGGGGGAAAC
AACATTGGAAGTAAAAGTGTGCACTGGTACCAGCAGAAGCCAGGCCAGGCCCCTGTGCTGGTCGTCTATGATGAT
AGCGACCGGCCCTCAGGGATCCCTGAGCGATTCTCTGGCTCCAACTCTGGGAACACGGCCACCCTGACCATCAGC
AGGGTCGAAGCCGGGGATGAGGCCGACTATTACTGTCAGGTGTGGGATAGTAGTAGTGATCATTGGGTGTTCGGC
GGAGGGACCAAGCTGACCGTCCTA
>B2-26_VL (SEQ ID NO: 540)
CAGTCTCCATCCTCCCTGTCTGCATCTGTAGGAGACAGAGTCACCATCACTTGCCGGGCAAGTCAGAGCATAAGC
AACTTTGTAAATTGGTATCAGCAGAAACCAGGGGAAGTCCCTAAGCTCCTGATCTACGGTGCATCCAGTTTGCAG
AGTGGGGTCCCATCAAGGTTCAGTGGCAGTGGATCTGGGACGGATTTCACTCTCACCATCAGCAGTCTGCAACCT
GAAGATTTTGCGACTTACTACTGTCAACAGGGTTACAGTGTCCCACTCACTTTCGGCGGAGGGACTACAGTGGAT
ATCAAA
>B2-27_VL (SEQ ID NO: 541)
CAGCCTGTGCTGACTCAGCCTGCCTCCGTGTCTGGGTCTCCTGGACAGTCGATCACCATCTCCTGCACTGGAACC
AGCAGTGACGTTGGTGGTTATAACTATGTCTCCTGGTACCAACAACACCCAGGCAAAGCCCCCAAACTCATGATT
TATGATGTCAGTAATCGGCCCTCGGGGGTTTCTAATCGCTTCTCTGGCTCCAAGTCTGGCAACTCGGCCTCCCTG
ACCATCTCTGGGCTCCAGGCTGAGGACGAGGCTGATTATTACTGCAGCTCATATACAAGCAGCAGCACTCCCTAT
GTCTTCGGAACTGGGACCAAGGTCACCGTCCTA
>B2-30_VL (SEQ ID NO: 542)
CAGTCTCCATCCTCCTTGTCTGCATCTGTAGGAGACAGAGTCACCATCACTTGCCGGGCAAGTCAGAGCATTAGG
ACCTACTTAAATTGGTATCAGCAGAAACCAGGCAAAGACCCTGAACTCCTGATCTATGCTGCAACCAGTTTGCAA
AGTGGGGTCCCATCAAGGTTCACTGGCAGCGGATCTGGGACAGACTTCACTCTCACCATCAGCAGTCTGCAGCCT
GAAGATTTTGCAACTTATTACTGTCAACAGAGTTACAGTGCCCCCGTGTTCACTTTTGGCCAGGGGACCAAGCTG
GAGATCAAA
>HCB2-1c_VL (SEQ ID NO: 543)
GCCATCCAGATGACCCAGTCTCCATCCTCCCTGTCTGCATCTGTAGGAGACAGAGTCTCCATCACTTGCCGGGCA
AGTCAGAGCATTAGCAGCTTTTTAAATTGGTATCAGCAGAGACCAGGGAAAGCCCCTGAGCTCCTGATCTATGGT
GCATCCAGCTTGCAAAGTGGGGTCCCATCAAGGTTCAGTGGCAGTGGATCTGGGACAGATTTCACTCTCACCATC
AGTAGTCTGCAACCTGAAGATTTTGCAACTTACTACTGTCAACAGAGTTACAGTATCCCGATCACCTTCGGCCAA
GGGACACAGCTGGAGATCAAA
>HCB2-1d_VL (SEQ ID NO: 544)
GACGTCCAGATGACCCAGTCCCCTTCCTCTTTATCCGCTAGCGTGGGCGATCGTGTGTCCATCACTTGTAAGGCC
TCCCAAGATGTGTCCATCGCCGTGGCTTGGTACCAGCAGAAGCCCGGCAAGGCCCCCAAGCTGCTGATCTACTCC
GCCAGCTATCGTTACACCGGCGTGCCCGATCGTTTCTCCGGCTCCGGATCCGGCACCGACTTCACTTTAACCATC
TCCTCTTTACAGCCCGAGGACTTCGCCGTCTACTACTGCCAGCAGCACTACATCACCCCTTTAACATTCGGCGCC
GGCACCAAGCTGGAGATCAAA
>HCB2-2_VL (SEQ ID NO: 545)
GACGTCCAGATGACCCAGTCTCCATCCTCCCTGTCTGCATCTGTAGGAGACAGAGTCTCCATCACTTGCCGGGCA
AGTCAGAGCATTAGCAGCTTTTTAAATTGGTATCAGCAGAGACCAGGGAAAGCCCCTGAGCTCCTGATCTATGGT
GCATCCAGCTTGCAAAGTGGGGTCCCATCAAGGTTCAGTGGCAGTGGATCTGGGACAGATTTCACTCTCACCATC
AGTAGTCTGCAACCTGAAGATTTTGCAACTTACTACTGTCAACAGAGTTACAGTATCCCGATCACCTTCGGCCAA
GGGACACGACTGGAGATCAAA
>HCB2-4_VL (SEQ ID NO: 546)
AACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCGCCCACTTTCGGCGGA
GGGACTACAGTGGAGATCAAA
>HCB2-5_VL (SEQ ID NO: 547)
GCCATCCAGATGACCCAGTCTCCATCTTCCGTGTCTGCATCTGTAGGAGACAGAGTCACCATCACTTGTCGGGCG
AGTCAGGGTATTGGCAGCTGGTTAGCCTGGTATCAGCAGAAACCAGGGAAAGCCCCTAAGCTCCTGATCTCTGTT
GCATCCAGTTTGCAAAGTGGGGTCCCATCAAGGTTCAGCGGCAGTGGATCTGGGACAGATTTCACTCTCACCATC
AGCAGCCTGCAGCCTGAAGATTTTGCAACTTACTATTGTCAACAGGCTAACAGTTTCCCGCTCACTTTTGGCGGA
GGGACTACAGTGGAGATCAAA
>HCB2-10_VL (SEQ ID NO: 548)
CAGCCACCCTCGGTGTCTGCAGCCCCCAGGCAGAGGGTCACCATCTCCTGTTCTGGAAGCAGCTCCAATATCGGA
GATAATTCTGTTAACTGGTACCAGCAGCTCCCAGGAAAGGCTCCCAAACTCCTCATTTATCTTGATGATCTCCTG
CCCTCAGGGGTCTCTGGCCGATTCTCTGGCTCCAAGTCTGGCACCTCAGCCTCCCTGGTCATCAGTGGCCTCCAG
TCTGAGGATGAGGCTGATTATTACTGTGCAACATGGGATGACAGCCTGAACGGTGTGGTTTTCGGCGGAGGGACC
AAGGTCACCGTCCTA
>HCB2-13_VL (SEQ ID NO: 549)
GATGTTGTGATGACGCAGTCTCCATCCTCCCTGAGTGTGTCAGCAGGAGAGAAGGTCACTATGAGCTGCAAGTCC
AGTCAGAGTCTGTTAAACAGTGGAAATCAAAAGACCTACTTGGCCTGGTACCAGCAGAAACCAGGGCAGCCTCCT
AAACTGTTGATCTACGGGGCATCCACTAGGGAATCTGGGGTCCCTGATCGCTTCACAGGCAGTGGATCTGGAACC
GATTTCACTCTTACCATCAGCAGTGTGCAGGCTGAAGACCTGGCAGTTTATTACTGTCAGAATGATCATAGTTAT
CCATTCACGTTCGGCTCGGGGACAAAGGTGGAAATCAAA
>HCB2-14_VL (SEQ ID NO: 550)
AACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCGTACACTTTTGGCCAG
GGGACCAAGCTGGAGATCAAA
>HCB2-15_VL (SEQ ID NO: 551)
CAGCCGCCCTCGGTGTCTGGGGCCCCAGGGCAGAGGGTCACCATCTCCTGCGCTGGGAGCAGCTCCAACATCGGG
GCAGGTTATGGTGTGCACTGGTATCAACACCTTCCAGGAACAGCCCCCAAACTCCTCATCTATGGTGACAACAAT
CGGCCCTCAGGGGTCCCTTACCGATTCTCTGGGTCCAAGTCTGGCACCTCAGCCTCCCTGGCCATCACTGGACTC
CAGGCTGAGGATGAGGCTGATTATTTCTGCCAGTCCTATGACAGCAGCCTGGGGGTTGGTGTGTTCGGCGGAGGG
ACCAAGGTCACCGTCCTAC
>HCB2-17_VL (SEQ ID NO: 552)
GCCATCCAGTTGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCCCTCACTTTCGGCGGA
GGGACCAAGCTGGAGATCAAA
>HCB2-18_VL (SEQ ID NO: 553)
GACATCGTGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCATTCACTTTCGGCCCT
GGGACCAAGCTGGAGATCAAA
>HCB2-24_VL (SEQ ID NO: 554)
GACGTCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCTGACGTTCGGCCAA
GGGACCAAGCTGGAGATCAAA
>HCB2-27_VL (SEQ ID NO: 555)
AACATCCAGATGACCCAGTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAAGCTCCTGATCTATGCT
GCATCCAGTTTGCAAAGTGGGGTCCCATCAAGGTTCAGTGGCAGTGGATCTGGGACAGATTTCACTCTCACCATC
AGCAGTCTGCAACCTGAAGATTTTGCAACTTACTACTGTCAACAGAGTTACAGTACCCCTCGGACGTTCGGCCAA
GGGACTACGGTGGAAATCAAA

TABLE 7
Amino acid sequences of LILRB2 antibody light chain variable regions (VL)

>B2-19_VL (SEQ ID NO: 572)
QPVLTOPPSVSEAPRQRVTISCSGSSSNIGNNVVNWYQQLPGKAPRLLIYYDDLLPSGVSDRFSGSKSGTSASLA
ISGLOSEDEADYYCATWDDSLNGPVFGGGTKLTVL
>B2-1_VL (SEQ ID NO: 556)
QSALNQPASVSGSPGQSITISCTGTSSDVGGYNYVSWYQQHPGKAPKLMIYDVSNRPSGVSNRFSGSKSGNTASL
TISGLQAEDEADYYCSSYTSSSTPYVFGTGTKVTVL
>B2-2_VL (SEQ ID NO: 557)
DIVMTQSPSSLSASVGDRVTLTCRASQSIGNYLNWYQQKPGKAPNLLIYAASSLQSGVPSRFSGSGSGTDFTLTI
SSLQPEDFATYYCQQSFSIPPITFGQGTRLEIK
>B2-3_VL (SEQ ID NO: 558)
QPVLTQPPSASGTPGQRVTISCSGSSSNIGTNPVNWYQQVPGTGPKLLIYNNNQWPSGVPDRFSGSKSATSASLA
IYGLQSGDEAHYYCSSWDDSLSAWVFGGGTKVTVL
>B2-4_VL (SEQ ID NO: 559)
QSVLTOPPSASGTPGQRVTTSCSGSRSNIGSNPVNWYQHLPGTAPKLLVYSNNRRPSGVPDRFSGSKSGTSASLS
ISGLQSEDEGVYYCAAWDDNVHGVFGGGTRVTVL
>B2-7_VL (SEQ ID NO: 560)
QSVLTQPPSVSVAPGKTARITCGGNNIGSKSVHWYQQKPGQAPVLVVYDDSDRPSGIPERFSGSNSGNTATLTIS
RVEAGDEADYYCQVWDSSSDHPGVFGTGTKVTVL
>B2-8_VL (SEQ ID NO: 561)
QPVLTQPASVSGSPGQSITISCTGTSSDVGGYNYVSWYQQHPGKAPKLMIYDVSNRPSGVSNRFSGSKSGNTASL
TISGLQAEDEADYYCSSYTSSSTPYVFGTGTKVTVL
>B2-9_VL (SEQ ID NO: 562)
QSPSSLSASVGDRVTITCRTSHTVNSYLNWYQQEPGKAPKLLIYAASNLQSGVPSRFSGSGSGTDFSLTIHNLQP
EDFATYYCQQSYRTPLTFGQGTKVDIK
>B2-10_VL (SEQ ID NO: 563)
QSVLTQPPSASGTPGQRVTISCSGSSSNIGGNPVNWYQHLPGTAPKLLIYYNDQRPSGVPDRFSGSKSGTSASLA
ISGLQSEDETDYYCATWDDSLNGWVFGGGTQLTVL
>B2-11_VL (SEQ ID NO: 564)
QSVLTQPPSASGTPGQRVTISCSGSKSNVGNNFVHWYQQLPGTAPKLLLYSNLERSSGVPERFSGSKSGTSAYLA
ISGLRSDDEADYYCAAWDDSLPGWVFGGGTKLTVL
>B2-12_VL (SEQ ID NO: 565)
QPVLTQPPSASGTPGQRVTISCSGSSSNIGSNPLNWYQQLPGTAPKLLIYNNNQRPSGVPDRFSGSKSGTSASLA
ISGLQSEDEADYYCAAWDDSLNGWVFGGGTQLTVL
>B2-13_VL (SEQ ID NO: 566)
QAVVTQPASVSGSPGQSVSISCTGTSSDVGGFNYVSWYQQHPGKAPKLMIYDVSNRPSGVSNRFSGSKSGNTASL
TISGLQAEDEADYYCSSYTSSTAPYVFGTGTKVTVL
>B2-14_VL (SEQ ID NO: 567)
QSVLTQPRSVSGSPGQSVTISCTGTSSDVGGYNYVSWYQQHPGKAPKLMISEVSKRPSGVPDRFSGSKSGNTASL
TVSGLQAEDEADYYCSSYAGGYTPYVFGTGTKVTVL
>B2-15_VL (SEQ ID NO: 568)
QSVLTQPPSASATPGQRVTISCSGRNANIGSNPVNWYQQLPGTAPRLVMYSNNQRPSGVPDRFSGSKSGTSASLA
ISGLQSEDEADYYCEGWDDSLNGYVFGTGTKVTVL
>B2-16_VL (SEQ ID NO: 569)
DIVMTQSPLSLPVTLGQPASISCTSSQSLVYSDGNTYLNWFHQRPGQSPRRLFYKVSNRASGVPDRFSGSGSGTD
FTLKISRVEAEDLGVYYCMQGTHWPVTFGQGTRLEIK
>B2-17_VL (SEQ ID NO: 570)
QSVLTQPPSASATPGQRVTISCSGSSSNIGSNPVNWYQQVPGMAPELVMYSDNQRPSGVPDRFSGSNSGTSASLA
ISGLQSEDEADYYCSTWDDSLNGLVFGTGTKVTVL
>B2-18_VL (SEQ ID NO: 571)
DVQMTQSPSSLSASVGERVTLTCRASQGIYSSLAWYQQKPGKAPKLLLYGTSQLERGVPSRFSGSGSGTDYTLTI
SSLQPEDSATYYCQQHYNLPITFGQGTRLEIK
>B2-22_VL (SEQ ID NO: 573)
QSPSSLSASVGDRVTITCRASQGISNYLAWYQQKPGKVPKLLIYAASTLQSGVPSRFSGSGSGTDFTLTISSLQP
EDVATYYCQKYNSAPLTFGGGTKLEIK
>B2-23_VL (SEQ ID NO: 574)
QSPSTLSASVGDRVTITCRASQSISSWLAWYQQKPGKAPKLLIYKASSLESGVPSRFSGSGSGTEFTLTISSLQP
DDFATYYCQQYNSYLTFGGGTKVDIK
>B2-24_VL (SEQ ID NO: 575)
QSVLTQPPSASGTPGQRVTISCSGSSSNIGSNYVYWYQQLPGTAPKLLIYRNNQRPSGVPDRFSGSKSGTSASLA
ISGLRSEDEADYYCAAWDDSLSGWVFGGGTKVTVL
>B2-25_VL (SEQ ID NO: 576)
SYELTQPPSVSVAPGQTARITCGGNNIGSKSVHWYQQKPGQAPVLVVYDDSDRPSGIPERFSGSNSGNTATLTIS
RVEAGDEADYYCQVWDSSSDHWVFGGGTKLTVL
>B2-26_VL (SEQ ID NO: 577)
QSPSSLSASVGDRVTITCRASQSISNFVNWYQQKPGEVPKLLIYGASSLQSGVPSRFSGSGSGTDFTLTISSLQP
EDFATYYCQQGYSVPLTFGGGTTVDIK
>B2-27_VL (SEQ ID NO: 578)
QPVLTQPASVSGSPGQSITISCTGTSSDVGGYNYVSWYQQHPGKAPKLMIYDVSNRPSGVSNRFSGSKSGNSASL
TISGLQAEDEADYYCSSYTSSSTPYVFGTGTKVTVL
>B2-30_VL (SEQ ID NO: 579)
QSPSSLSASVGDRVTITCRASQSIRTYLNWYQQKPGKDPELLIYAATSLQSGVPSRFTGSGSGTDFTLTISSLQP
EDFATYYCQQSYSAPVFTFGQGTKLEIK
>HCB2-1c_VL (SEQ ID NO: 580)
AIQMTQSPSSLSASVGDRVSITCRASQSISSFLNWYQQRPGKAPELLIYGASSLQSGVPSRFSGSGSGTDFTLTI
SSLQPEDFATYYCQQSYSIPITFGQGTQLEIK
>HCB2-1d_VL (SEQ ID NO: 581)
DVQMTQSPSSLSASVGDRVSITCKASQDVSIAVAWYQQKPGKAPKLLIYSASYRYTGVPDRFSGSGSGTDFTLTI
SSLQPEDFAVYYCQQHYITPLTFGAGTKLEIK
>HCB2-2_VL (SEQ ID NO: 582)
DVQMTQSPSSLSASVGDRVSITCRASQSISSFLNWYQQRPGKAPELLIYGASSLQSGVPSRFSGSGSGTDFTLTI
SSLQPEDFATYYCQQSYSIPITFGQGTRLEIK
>HCB2-4_VL (SEQ ID NO: 583)
NIQMTQSPSSLSASVGDRVTITCRASQSISSYLNWYQQKPGKAPKLLIYAASSLQSGVPSRFSGSGSGTDFTLTI
SSLQPEDFATYYCQQSYSTPPTFGGGTTVEIK
>HCB2-5_VL (SEQ ID NO: 584)
AIQMTQSPSSVSASVGDRVTITCRASQGIGSWLAWYQQKPGKAPKLLISVASSLQSGVPSRFSGSGSGTDFTLTI
SSLQPEDFATYYCQQANSFPLTFGGGTTVEIK
>HCB2-10_VL (SEQ ID NO: 585)
QPPSVSAAPRQRVTISCSGSSSNIGDNSVNWYQQLPGKAPKLLIYLDDLLPSGVSGRFSGSKSGTSASLVISGLQ
SEDEADYYCATWDDSLNGVVFGGGTKVTVL
>HCB2-13_VL (SEQ ID NO: 586)
DVVMTQSPSSLSVSAGEKVTMSCKSSQSLLNSGNQKTYLAWYQQKPGQPPKLLIYGASTRESGVPDRFTGSGSGT
DFTLTISSVQAEDLAVYYCQNDHSYPFTFGSGTKVEIKRTVAA
>HCB2-14_VL (SEQ ID NO: 587)
NIQMTQSPSSLSASVGDRVTITCRASQSISSYLNWYQQKPGKAPKLLIYAASSLQSGVPSRFSGSGSGTDFTLTI
SSLQPEDFATYYCQQSYSTPYTFGQGTKLEIK
>HCB2-15_VL (SEQ ID NO: 588)
QPPSVSGAPGQRVTISCAGSSSNIGAGYGVHWYQHLPGTAPKLLIYGDNNRPSGVPYRFSGSKSGTSASLAITGL
QAEDEADYFCQSYDSSLGVGVFGGGTKVTVL
>HCB2-17_VL (SEQ ID NO: 589)
AIQLTQSPSSLSASVGDRVTITCRASQSISSYLNWYQQKPGKAPKLLIYAASSLQSGVPSRFSGSGSGTDFTLTI
SSLQPEDFATYYCQQSYSTPLTFGGGTKLEIK
>HCB2-18_VL (SEQ ID NO: 590)
DIVMTQSPSSLSASVGDRVTITCRASQSISSYLNWYQQKPGKAPKLLIYAASSLQSGVPSRFSGSGSGTDFTLTI
SSLQPEDFATYYCQQSYSTPFTFGPGTKLEIK
>HCB2-24_VL (SEQ ID NO: 591)
DVQMTQSPSSLSASVGDRVTITCRASQSISSYLNWYQQKPGKAPKLLIYAASSLQSGVPSRFSGSGSGTDFTLTI
SSLQPEDFATYYCQQSYSTPLTFGQGTKLEIK
>HCB2-27_VL (SEQ ID NO: 592)
NIQMTQSPSSLSASVGDRVTITCRASQSISSYLNWYQQKPGKAPKLLIYAASSLQSGVPSRFSGSGSGTDFTLTI
SSLQPEDFATYYCQQSYSTPRTFGQGTTVEIK

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

REFERENCES

The following references, to the extent that they provide exemplary procedural or other
details supplementary to those set forth herein, are specifically incorporated herein by reference.
Swerdlow, S., Campo, E., Harris, N.L., Jaffe, E.S., Pileri, S.A., Stein, H., Thiele, J., Arber, D.,
Hasserjian, R., and Le Beau, M. (2017). WHO classification of tumors of haematopoietic
and lymphoid tissues (WHO).
Schuler, E., Schroeder, M., Neukirchen, J., Strupp, C., Xicoy, B., Kundgen, A., Hildebrandt, B.,
Haas, R., Gattermann, N., and Germing, U. (2014). Refined medullary blast and white
blood cell count based classification of chronic myelomonocytic leukemias. Leuk Res 38,
1413-1419.

1-59. (canceled)

60. An isolated monoclonal antibody or an antigen-binding fragment thereof that binds to LILRB2 comprising a heavy chain (HC) variable region (VH) and a light chain (LC) variable region (VL) comprising heavy chain complementarity determining regions 1-3 (HCDR1-3) and light chain complementarity determining regions 1-3 (LCDR1-3) as set out below:
HCDR1 comprising SEQ ID NO: 121,
HCDR2 comprising SEQ ID NO: 122,
HCDR3 comprising SEQ ID NO: 123,
LCDR1 comprising SEQ ID NO: 124,
LCDR2 comprising SEQ ID NO: 125, and
LCDR3 comprising SEQ ID NO: 126.

61. The isolated monoclonal antibody or an antigen binding fragment thereof of claim 60, wherein the isolated monoclonal antibody is a murine, a rodent, a rabbit, a chimeric, or a humanized antibody.

62. The isolated monoclonal antibody or an antigen-binding fragment thereof of claim 60, wherein the antigen-binding fragment is a recombinant single chain fragment variable (ScFv) antibody, Fab fragment, F(ab′)2 fragment, or Fv fragment.

63. The isolated monoclonal antibody or an antigen-binding fragment thereof of claim 60, wherein the VH and VL chains have amino acid sequences at least 90% or 95% identical to SEQ ID NO: 502 and SEQ ID NO: 576, respectively.

64. The isolated monoclonal antibody or an antigen-binding fragment thereof of claim 60, wherein the VH and VL chains are encoded by nucleic acid sequences at least 80% or 90% identical to SEQ ID NO: 465 and SEQ ID NO: 539, respectively.

65. The isolated monoclonal antibody or an antigen-binding fragment thereof of claim 64, wherein the VH and VL chains have amino acid sequences identical to SEQ ID NO: 502 and SEQ ID NO: 576, respectively.

66. The isolated monoclonal antibody or an antigen binding fragment thereof of claim 64, wherein the VH and VL chains are encoded by nucleic acid sequences identical to SEQ ID NO: 465 and SEQ ID NO: 539, respectively.

67. A pharmaceutical composition comprising the isolated monoclonal antibody or an antigen-binding fragment thereof according to claim 60, and a pharmaceutically acceptable carrier.

68. An isolated nucleic acid that encodes the isolated monoclonal antibody according to claim 60.

69. A vector comprising the isolated nucleic acid of claim 68.

70. A host cell comprising the vector of claim 69, wherein the host cell is a mammalian cell or a CHO cell.

71. An engineered cell encoding and/or producing the isolated monoclonal antibody according to claim 60.

72. The engineered cell of claim 71, wherein the VH and VL chains have amino acid sequences at least 90% or 95% identical to SEQ ID NO: 502 and SEQ ID NO: 576, respectively.

73. A process of producing an antibody, comprising culturing the host cell of claim 70 under conditions suitable for expressing the antibody, and recovering the antibody.

74. A chimeric antigen receptor (CAR) protein comprising an antigen-binding fragment according to claim 60.

75. An isolated nucleic acid that encodes a CAR protein of claim 74.

76. A vector comprising the isolated nucleic acid of claim 75.

77. An engineered cell comprising the isolated nucleic acid of claim 75, wherein the cell is a T cell, NK cell, or macrophage.

78. A method of detecting an LILRB2-expressing cancer cell or an LILRB2-expressing cancer stem cell in a sample or subject comprising:
(a) contacting a subject or a sample from the subject with the antibody or an antigen-binding fragment thereof according to claim 60; and
(b) detecting binding of said antibody to a cancer cell or cancer stem cell in said subject or sample.

79. A method for treating or ameliorating the effect of an autoimmune disease in a subject, the method comprising administering to the subject the antibody or an antigen-binding fragment thereof according to claim 60.


SEQUENCE LISTING (613 sequences)

SEQ ID NO: 1 - protein, 8 aa, synthetic construct
  REGION 1..8: note = Synthetic antibody
GGTFSSYA

SEQ ID NO: 2 - protein, 8 aa, synthetic construct
  REGION 1..8: note = Synthetic antibody
IIPIFGTA

SEQ ID NO: 3 - protein, 12 aa, synthetic construct
  REGION 1..12: note = Synthetic antibody
ARGNQGDTAFDI

SEQ ID NO: 4 - protein, 9 aa, synthetic construct
  REGION 1..9: note = Synthetic antibody
SSDVGGYNY

SEQ ID NO: 5 - skipped

SEQ ID NO: 6 - protein, 11 aa, synthetic construct
  REGION 1..11: note = Synthetic antibody
SSYTSSSTPYV

SEQ ID NO: 7 - protein, 8 aa, synthetic construct
  REGION 1..8: note = Synthetic antibody
GYTFSDYG

SEQ ID NO: 8 - protein, 8 aa, synthetic construct
  REGION 1..8: note = Synthetic antibody
ISGYNGNT

SEQ ID NO: 9 - protein, 12 aa, synthetic construct
  REGION 1..12: note = Synthetic antibody
ARLLLGYYGMDV

SEQ ID NO: 10 - protein, 6 aa, synthetic construct
  REGION 1..6: note = Synthetic antibody
QSIGNY

SEQ ID NO: 11 - skipped

SEQ ID NO: 12 - protein, 10 aa, synthetic construct
  REGION 1..10: note = Synthetic antibody
QQSFSIPPIT

SEQ ID NO: 13 - protein, 8 aa, synthetic construct
  REGION 1..8: note = Synthetic antibody
GYTFSSYG

SEQ ID NO: 14 - protein, 8 aa, synthetic construct
  REGION 1..8: note = Synthetic antibody
ISGNTGNT

SEQ ID NO: 15 - protein, 14 aa, synthetic construct
  REGION 1..14: note = Synthetic antibody
ARDRGIAAGRAFDI

SEQ ID NO: 16 - protein, 8 aa, synthetic construct
  REGION 1..8: note = Synthetic antibody
SSNIGTNP

SEQ ID NO: 17 - skipped

SEQ ID NO: 18 - protein, 11 aa, synthetic construct
  REGION 1..11: note = Synthetic antibody
SSWDDSLSAWV

SEQ ID NO: 19 - protein, 8 aa, synthetic construct
  REGION 1..8: note = Synthetic antibody
GYPFTSNG

SEQ ID NO: 20 - protein, 8 aa, synthetic construct
  REGION 1..8: note = Synthetic antibody
ISTNSGNT

SEQ ID NO: 21 - protein, 10 aa, synthetic construct
  REGION 1..10: note = Synthetic antibody
ARDVQYRMDV

SEQ ID NO: 22 - protein, 8 aa, synthetic construct
  REGION 1..8: note = Synthetic antibody
RSNIGSNP

SEQ ID NO: 23 - skipped

SEQ ID NO: 24 - protein, 10 aa, synthetic construct
  REGION 1..10: note = Synthetic antibody
AAWDDNVHGV

SEQ ID NO: 25 - protein, 8 aa, synthetic construct
  REGION 1..8: note = Synthetic antibody
GFTISSYG

SEQ ID NO: 26 - protein, 8 aa, synthetic construct
  REGION 1..8: note = Synthetic antibody
ISYDGSNK

SEQ ID NO: 27 - protein, 22 aa, synthetic construct
  REGION 1..22: note = Synthetic antibody
AKDLLLRYFDWSPKYYYNGMDV

SEQ ID NO: 28 - protein, 6 aa, synthetic construct
  REGION 1..6: note = Synthetic antibody
NIGSKS

SEQ ID NO: 29 - skipped

SEQ ID NO: 30 - protein, 12 aa, synthetic construct
  REGION 1..12: note = Synthetic antibody
QVWDSSSDHPGV

SEQ ID NO: 31 - protein, 8 aa, synthetic construct
  REGION 1..8: note = Synthetic antibody
GGTFSSYA

SEQ ID NO: 32 - protein, 8 aa, synthetic construct
  REGION 1..8: note = Synthetic antibody
IIPIFGTA

SEQ ID NO: 33 - protein, 12 aa, synthetic construct
  REGION 1..12: note = Synthetic antibody
ARVAVADTFFDY

SEQ ID NO: 34 - protein, 9 aa, synthetic construct
  REGION 1..9: note = Synthetic antibody
SSDVGGYNY

SEQ ID NO: 35 - skipped

SEQ ID NO: 36 - protein, 11 aa, synthetic construct
  REGION 1..11: note = Synthetic antibody
SSYTSSSTPYV

SEQ ID NO: 37 - protein, 8 aa, synthetic construct
  REGION 1..8: note = Synthetic antibody
GYTFTSYG

SEQ ID NO: 38 - protein, 8 aa, synthetic construct
  REGION 1..8: note = Synthetic antibody
ISAYNGNT

SEQ ID NO: 39 - protein, 9 aa, synthetic construct
  REGION 1..9: note = Synthetic antibody
ARDGYGMDV

SEQ ID NO: 40 - protein, 6 aa, synthetic construct
  REGION 1..6: note = Synthetic antibody
HTVNSY

SEQ ID NO: 41 - skipped

SEQ ID NO: 42 - protein, 9 aa, synthetic construct
  REGION 1..9: note = Synthetic antibody
QQSYRTPLT

SEQ ID NO: 43 - protein, 8 aa, synthetic construct
  REGION 1..8: note = Synthetic antibody
GFTFSSYA

SEQ ID NO: 44 - protein, 8 aa, synthetic construct
  REGION 1..8: note = Synthetic antibody
INDSGGST

SEQ ID NO: 45 - protein, 15 aa, synthetic construct
  REGION 1..15: note = Synthetic antibody
AKDHITIFGVAPFDY

SEQ ID NO: 46 - protein, 8 aa, synthetic construct
  REGION 1..8: note = Synthetic antibody
SSNIGGNP

SEQ ID NO: 47 - skipped

SEQ ID NO: 48 - protein, 11 aa, synthetic construct
  REGION 1..11: note = Synthetic antibody
ATWDDSLNGWV

SEQ ID NO: 49 - protein, 10 aa, synthetic construct
  REGION 1..10: note = Synthetic antibody
GDSVSSNSAA

SEQ ID NO: 50 - protein, 9 aa, synthetic construct
  REGION 1..9: note = Synthetic antibody
TYYRSKWYN

SEQ ID NO: 51 - protein, 14 aa, synthetic construct
  REGION 1..14: note = Synthetic antibody
ARQDESAADDAFDI

SEQ ID NO: 52 - protein, 8 aa, synthetic construct
  REGION 1..8: note = Synthetic antibody
KSNVGNNF

SEQ ID NO: 53 - skipped

SEQ ID NO: 54 - protein, 11 aa, synthetic construct
  REGION 1..11: note = Synthetic antibody
AAWDDSLPGWV

SEQ ID NO: 55 - protein, 8 aa, synthetic construct
  REGION 1..8: note = Synthetic antibody
GYTFPDNG

SEQ ID NO: 56 - protein, 8 aa, synthetic construct
  REGION 1..8: note = Synthetic antibody
INVDTGYT

SEQ ID NO: 57 - protein, 10 aa, synthetic construct
  REGION 1..10: note = Synthetic antibody
AREIEGVWTA

SEQ ID NO: 58 - protein, 8 aa, synthetic construct
  REGION 1..8: note = Synthetic antibody
SSNIGSNP

SEQ ID NO: 59 - skipped

SEQ ID NO: 60 - protein, 11 aa, synthetic construct
  REGION 1..11: note = Synthetic antibody
AAWDDSLNGWV

SEQ ID NO: 61 - protein, 8 aa, synthetic construct
  REGION 1..8: note = Synthetic antibody
GGTFSSYA

SEQ ID NO: 62 - protein, 8 aa, synthetic construct
  REGION 1..8: note = Synthetic antibody
IIPIFGTA

SEQ ID NO: 63 - protein, 12 aa, synthetic construct
  REGION 1..12: note = Synthetic antibody
ARSDYGDSYFDY

SEQ ID NO: 64 - protein, 9 aa, synthetic construct
  REGION 1..9: note = Synthetic antibody
SSDVGGFNY

SEQ ID NO: 65 - skipped

SEQ ID NO: 66 - protein, 11 aa, synthetic construct
  REGION 1..11: note = Synthetic antibody
SSYTSSTAPYV

SEQ ID NO: 67 - protein, 8 aa, synthetic construct
  REGION 1..8: note = Synthetic antibody
GGTFSSYA

SEQ ID NO: 68 - protein, 8 aa, synthetic construct
  REGION 1..8: note = Synthetic antibody
IIPIFGTA

SEQ ID NO: 69 - protein, 12 aa, synthetic construct
  REGION 1..12: note = Synthetic antibody
ARASMVETYFDY

SEQ ID NO: 70 - protein, 9 aa, synthetic construct
  REGION 1..9: note = Synthetic antibody
SSDVGGYNY

SEQ ID NO: 71 - skipped

SEQ ID NO: 72 - protein, 11 aa, synthetic construct
  REGION 1..11: note = Synthetic antibody
SSYAGGYTPYV

SEQ ID NO: 73 - protein, 8 aa, synthetic construct
  REGION 1..8: note = Synthetic antibody
GYTFTNYG

SEQ ID NO: 74 - protein, 8 aa, synthetic construct
  REGION 1..8: note = Synthetic antibody
ISGDAGDT

SEQ ID NO: 75 - protein, 21 aa, synthetic construct
  REGION 1..21: note = Synthetic antibody
ARDGEYIPMFRGFDNYYGLDV

SEQ ID NO: 76 - protein, 8 aa, synthetic construct
  REGION 1..8: note = Synthetic antibody
NANIGSNP

SEQ ID NO: 77 - skipped

SEQ ID NO: 78 - protein, 11 aa, synthetic construct
  REGION 1..11: note = Synthetic antibody
EGWDDSLNGYV

SEQ ID NO: 79 - protein, 8 aa, synthetic construct
  REGION 1..8: note = Synthetic antibody
GGTFSSYA

SEQ ID NO: 80 - protein, 8 aa, synthetic construct
  REGION 1..8: note = Synthetic antibody
IIPIFGTA

SEQ ID NO: 81 - protein, 14 aa, synthetic construct
  REGION 1..14: note = Synthetic antibody
ASGIIPDNPYGMDV

SEQ ID NO: 82 - protein, 11 aa, synthetic construct
  REGION 1..11: note = Synthetic antibody
QSLVYSDGNTY

SEQ ID NO: 83 - skipped

SEQ ID NO: 84 - protein, 9 aa, synthetic construct
  REGION 1..9: note = Synthetic antibody
MQGTHWPVT

SEQ ID NO: 85 - protein, 9 aa, synthetic construct
  REGION 1..9: note = Synthetic antibody
GVSTSSTHW

SEQ ID NO: 86 - protein, 7 aa, synthetic construct
  REGION 1..7: note = Synthetic antibody
ILHNGNT

SEQ ID NO: 87 - protein, 15 aa, synthetic construct
  REGION 1..15: note = Synthetic antibody
ASASPNWGASGAFDA

SEQ ID NO: 88 - protein, 8 aa, synthetic construct
  REGION 1..8: note = Synthetic antibody
SSNIGSNP

SEQ ID NO: 89 - skipped

SEQ ID NO: 90 - protein, 11 aa, synthetic construct
  REGION 1..11: note = Synthetic antibody
STWDDSLNGLV

SEQ ID NO: 91 - protein, 8 aa, synthetic construct
  REGION 1..8: note = Synthetic antibody
GYTFTRYG

SEQ ID NO: 92 - protein, 8 aa, synthetic construct
  REGION 1..8: note = Synthetic antibody
ISGYNGNT

SEQ ID NO: 93 - protein, 11 aa, synthetic construct
  REGION 1..11: note = Synthetic antibody
ARDRGSGYLEY

SEQ ID NO: 94 - protein, 6 aa, synthetic construct
  REGION 1..6: note = Synthetic antibody
QGIYSS

SEQ ID NO: 95 - skipped

SEQ ID NO: 96 - protein, 9 aa, synthetic construct
  REGION 1..9: note = Synthetic antibody
QQHYNLPIT

SEQ ID NO: 97 - protein, 8 aa, synthetic construct
  REGION 1..8: note = Synthetic antibody
GFTFSNAW

SEQ ID NO: 98 - protein, 10 aa, synthetic construct
  REGION 1..10: note = Synthetic antibody
IKSKTDGGTT

SEQ ID NO: 99 - protein, 16 aa, synthetic construct
  REGION 1..16: note = Synthetic antibody
TTDRYSSSWYSPAFDI

SEQ ID NO: 100 - protein, 8 aa, synthetic construct
  REGION 1..8: note = Synthetic antibody
SSNIGNNV

SEQ ID NO: 101 - skipped

SEQ ID NO: 102 - protein, 11 aa, synthetic construct
  REGION 1..11: note = Synthetic antibody
ATWDDSLNGPV

SEQ ID NO: 103 - protein, 8 aa, synthetic construct
  REGION 1..8: note = Synthetic antibody
GYTFTSYG

SEQ ID NO: 104 - protein, 8 aa, synthetic construct
  REGION 1..8: note = Synthetic antibody
ISAYNGNT

SEQ ID NO: 105 - protein, 9 aa, synthetic construct
  REGION 1..9: note = Synthetic antibody
ARVGSYGDY

SEQ ID NO: 106 - protein, 6 aa, synthetic construct
  REGION 1..6: note = Synthetic antibody
QGISNY

SEQ ID NO: 107 - skipped

SEQ ID NO: 108 - protein, 9 aa, synthetic construct
  REGION 1..9: note = Synthetic antibody
QKYNSAPLT

SEQ ID NO: 109 - protein, 8 aa, synthetic construct
  REGION 1..8: note = Synthetic antibody
GYTFTHSY

SEQ ID NO: 110 - protein, 8 aa, synthetic construct
  REGION 1..8: note = Synthetic antibody
INPVGGST

SEQ ID NO: 111 - protein, 14 aa, synthetic construct
  REGION 1..14: note = Synthetic antibody
ARAGGDGYNPYFDY

SEQ ID NO: 112 - protein, 6 aa, synthetic construct
  REGION 1..6: note = Synthetic antibody
QSISSW

SEQ ID NO: 113 - skipped

SEQ ID NO: 114 - protein, 8 aa, synthetic construct
  REGION 1..8: note = Synthetic antibody
QQYNSYLT

SEQ ID NO: 115 - protein, 8 aa, synthetic construct
  REGION 1..8: note = Synthetic antibody
GFTFSDYY

SEQ ID NO: 116 - protein, 8 aa, synthetic construct
  REGION 1..8: note = Synthetic antibody
ISSSSSYT

SEQ ID NO: 117 - protein, 11 aa, synthetic construct
  REGION 1..11: note = Synthetic antibody
ARVGDLDAFDI

SEQ ID NO: 118 - protein, 8 aa, synthetic construct
  REGION 1..8: note = Synthetic antibody
SSNIGSNY

SEQ ID NO: 119 - skipped

SEQ ID NO: 120 - protein, 11 aa, synthetic construct
  REGION 1..11: note = Synthetic antibody
AAWDDSLSGWV

SEQ ID NO: 121 - protein, 8 aa, synthetic construct
  REGION 1..8: note = Synthetic antibody
GFTFSDHY

SEQ ID NO: 122 - protein, 8 aa, synthetic construct
  REGION 1..8: note = Synthetic antibody
ITTTGSTM

SEQ ID NO: 123 - protein, 13 aa, synthetic construct
  REGION 1..13: note = Synthetic antibody
ARDSIHVDGAFDI

SEQ ID NO: 124 - protein, 6 aa, synthetic construct
  REGION 1..6: note = Synthetic antibody
NIGSKS

SEQ ID NO: 125 - skipped

SEQ ID NO: 126 - protein, 11 aa, synthetic construct
  REGION 1..11: note = Synthetic antibody
QVWDSSSDHWV

SEQ ID NO: 127 - protein, 8 aa, synthetic construct
  REGION 1..8: note = Synthetic antibody
GYTFTSYG

SEQ ID NO: 128 - protein, 8 aa, synthetic construct
  REGION 1..8: note = Synthetic antibody
ISAYNGNT

SEQ ID NO: 129 - protein, 10 aa, synthetic construct
  REGION 1..10: note = Synthetic antibody
ARDRGGWFDP

SEQ ID NO: 130 - protein, 6 aa, synthetic construct
  REGION 1..6: note = Synthetic antibody
QSISNF

SEQ ID NO: 131 - skipped

SEQ ID NO: 132 - protein, 9 aa, synthetic construct
  REGION 1..9: note = Synthetic antibody
QQGYSVPLT

SEQ ID NO: 133 - protein, 8 aa, synthetic construct
  REGION 1..8: note = Synthetic antibody
GGTFSSYA

SEQ ID NO: 134 - protein, 8 aa, synthetic construct
  REGION 1..8: note = Synthetic antibody
IIPIFGTA

SEQ ID NO: 135 - protein, 12 aa, synthetic construct
  REGION 1..12: note = Synthetic antibody
ARESYSSSSFDY

SEQ ID NO: 136 - protein, 9 aa, synthetic construct
  REGION 1..9: note = Synthetic antibody
SSDVGGYNY

SEQ ID NO: 137 - skipped

SEQ ID NO: 138 - protein, 11 aa, synthetic construct
  REGION 1..11: note = Synthetic antibody
SSYTSSSTPYV

SEQ ID NO: 139 - protein, 8 aa, synthetic construct
  REGION 1..8: note = Synthetic antibody
GFTFSSYD

SEQ ID NO: 140 - protein, 7 aa, synthetic construct
  REGION 1..7: note = Synthetic antibody
IGTAGDT

SEQ ID NO: 141 - protein, 18 aa, synthetic construct
  REGION 1..18: note = Synthetic antibody
ARAKEYCSGGSCQDAFDI

SEQ ID NO: 142 - protein, 6 aa, synthetic construct
  REGION 1..6: note = Synthetic antibody
QSIRTY

SEQ ID NO: 143 - skipped

SEQ ID NO: 144 - protein, 10 aa, synthetic construct
  REGION 1..10: note = Synthetic antibody
QQSYSAPVFT

SEQ ID NO: 145 - protein, 8 aa, synthetic construct
  REGION 1..8: note = Synthetic antibody
GYTFTDYY

SEQ ID NO: 146 - protein, 8 aa, synthetic construct
  REGION 1..8: note = Synthetic antibody
VNANGGDT

SEQ ID NO: 147 - protein, 16 aa, synthetic construct
  REGION 1..16: note = Synthetic antibody
ARDWYYYSSGSFPFDY

SEQ ID NO: 148 - protein, 6 aa, synthetic construct
  REGION 1..6: note = Synthetic antibody
QSISSF

SEQ ID NO: 149 - skipped

SEQ ID NO: 150 - protein, 9 aa, synthetic construct
  REGION 1..9: note = Synthetic antibody
QQSYSIPIT

SEQ ID NO: 151 - protein, 8 aa, synthetic construct
  REGION 1..8: note = Synthetic antibody
GYTFTDYY

SEQ ID NO: 152 - protein, 8 aa, synthetic construct
  REGION 1..8: note = Synthetic antibody
VNANGGDT

SEQ ID NO: 153 - protein, 16 aa, synthetic construct
  REGION 1..16: note = Synthetic antibody
ARDWYYYSSGSFPFDY

SEQ ID NO: 154 - protein, 6 aa, synthetic construct
  REGION 1..6: note = Synthetic antibody
QDVSIA

SEQ ID NO: 155 - skipped

SEQ ID NO: 156 - protein, 9 aa, synthetic construct
  REGION 1..9: note = Synthetic antibody
QQHYITPLT

SEQ ID NO: 157 - protein, 8 aa, synthetic construct
  REGION 1..8: note = Synthetic antibody
GYIFNTYG

SEQ ID NO: 158 - protein, 8 aa, synthetic construct
  REGION 1..8: note = Synthetic antibody
TNVYNSNT

SEQ ID NO: 159 - protein, 11 aa, synthetic construct
  REGION 1..11: note = Synthetic antibody
ARDPGSAIFDM

SEQ ID NO: 160 - protein, 6 aa, synthetic construct
  REGION 1..6: note = Synthetic antibody
QSISSF

SEQ ID NO: 161 - skipped

SEQ ID NO: 162 - protein, 9 aa, synthetic construct
  REGION 1..9: note = Synthetic antibody
QQSYSIPIT

SEQ ID NO: 163 - protein, 8 aa, synthetic construct
  REGION 1..8: note = Synthetic antibody
GDAIKDKY

SEQ ID NO: 164 - protein, 7 aa, synthetic construct
  REGION 1..7: note = Synthetic antibody
ISNSGST

SEQ ID NO: 165 - protein, 11 aa, synthetic construct
  REGION 1..11: note = Synthetic antibody
ARGSGYTAFDI

SEQ ID NO: 166 - protein, 6 aa, synthetic construct
  REGION 1..6: note = Synthetic antibody
QSISSY

SEQ ID NO: 167 - skipped

SEQ ID NO: 168 - protein, 9 aa, synthetic construct
  REGION 1..9: note = Synthetic antibody
QQSYSTPPT

SEQ ID NO: 169 - protein, 8 aa, synthetic construct
  REGION 1..8: note = Synthetic antibody
GFTFSSYA

SEQ ID NO: 170 - protein, 8 aa, synthetic construct
  REGION 1..8: note = Synthetic antibody
ISGSGGST

SEQ ID NO: 171 - protein, 14 aa, synthetic construct
  REGION 1..14: note = Synthetic antibody
AKDVDPYSSGYFDY

SEQ ID NO: 172 - protein, 6 aa, synthetic construct
  REGION 1..6: note = Synthetic antibody
QGIGSW

SEQ ID NO: 173 - skipped

SEQ ID NO: 174 - protein, 9 aa, synthetic construct
  REGION 1..9: note = Synthetic antibody
QQANSFPLT

SEQ ID NO: 175 - protein, 8 aa, synthetic construct
  REGION 1..8: note = Synthetic antibody
GFTFSNYA

SEQ ID NO: 176 - protein, 8 aa, synthetic construct
  REGION 1..8: note = Synthetic antibody
ISGSSIST

SEQ ID NO: 177 - protein, 10 aa, synthetic construct
  REGION 1..10: note = Synthetic antibody
ASSGFGSFDY

SEQ ID NO: 178 - protein, 8 aa, synthetic construct
  REGION 1..8: note = Synthetic antibody
SSNIGDNS

SEQ ID NO: 179 - skipped

SEQ ID NO: 180 - protein, 11 aa, synthetic construct
  REGION 1..11: note = Synthetic antibody
ATWDDSLNGVV

SEQ ID NO: 181 - protein, 8 aa, synthetic construct
  REGION 1..8: note = Synthetic antibody
GFTLSSYA

SEQ ID NO: 182 - protein, 7 aa, synthetic construct
  REGION 1..7: note = Synthetic antibody
INSGGST

SEQ ID NO: 183 - protein, 12 aa, synthetic construct
  REGION 1..12: note = Synthetic antibody
AKAKSYSSDLDH

SEQ ID NO: 184 - protein, 12 aa, synthetic construct
  REGION 1..12: note = Synthetic antibody
QSLLNSGNQKTY

SEQ ID NO: 185 - skipped

SEQ ID NO: 186 - protein, 9 aa, synthetic construct
  REGION 1..9: note = Synthetic antibody
QNDHSYPFT

SEQ ID NO: 187 - protein, 8 aa, synthetic construct
  REGION 1..8: note = Synthetic antibody
GFTFSSYA

SEQ ID NO: 188 - protein, 8 aa, synthetic construct
  REGION 1..8: note = Synthetic antibody
ISGSGGST

SEQ ID NO: 189 - protein, 16 aa, synthetic construct
  REGION 1..16: note = Synthetic antibody
AKDLLGDSGSYPAFDI

SEQ ID NO: 190 - protein, 6 aa, synthetic construct
  REGION 1..6: note = Synthetic antibody
QSISSY

SEQ ID NO: 191 - skipped

SEQ ID NO: 192 - protein, 9 aa, synthetic construct
  REGION 1..9: note = Synthetic antibody
QQSYSTPYT

SEQ ID NO: 193 - protein, 8 aa, synthetic construct
  REGION 1..8: note = Synthetic antibody
GFPFADYA

SEQ ID NO: 194 - protein, 10 aa, synthetic construct
  REGION 1..10: note = Synthetic antibody
IKSKTRDGTT

SEQ ID NO: 195 - protein, 8 aa, synthetic construct
  REGION 1..8: note = Synthetic antibody
ALGGAAAL

SEQ ID NO: 196 - protein, 9 aa, synthetic construct
  REGION 1..9: note = Synthetic antibody
SSNIGAGYG

SEQ ID NO: 197 - skipped

SEQ ID NO: 198 - protein, 11 aa, synthetic construct
  REGION 1..11: note = Synthetic antibody
QSYDSSLGVGV

SEQ ID NO: 199 - protein, 8 aa, synthetic construct
  REGION 1..8: note = Synthetic antibody
GGTFSSYA

SEQ ID NO: 200 - protein, 8 aa, synthetic construct
  REGION 1..8: note = Synthetic antibody
IIPIFGTA

SEQ ID NO: 201 - protein, 13 aa, synthetic construct
  REGION 1..13: note = Synthetic antibody
AISSGYYYNPFDY

SEQ ID NO: 202 - protein, 6 aa, synthetic construct
  REGION 1..6: note = Synthetic antibody
QSISSY

SEQ ID NO: 203 - skipped

SEQ ID NO: 204 - protein, 9 aa, synthetic construct
  REGION 1..9: note = Synthetic antibody
QQSYSTPLT

SEQ ID NO: 205 - protein, 8 aa, synthetic construct
  REGION 1..8: note = Synthetic antibody
GGTFSSYA

SEQ ID NO: 206 - protein, 8 aa, synthetic construct
  REGION 1..8: note = Synthetic antibody
IIPIFGTA

SEQ ID NO: 207 - protein, 10 aa, synthetic construct
  REGION 1..10: note = Synthetic antibody
ARGGYQPLDY

SEQ ID NO: 208 - protein, 6 aa, synthetic construct
  REGION 1..6: note = Synthetic antibody
QSISSY

SEQ ID NO: 209 - skipped

SEQ ID NO: 210 - protein, 9 aa, synthetic construct
  REGION 1..9: note = Synthetic antibody
QQSYSTPFT

SEQ ID NO: 211 - protein, 8 aa, synthetic construct
  REGION 1..8: note = Synthetic antibody
GGTFSSYA

SEQ ID NO: 212 - protein, 8 aa, synthetic construct
  REGION 1..8: note = Synthetic antibody
IIPIFGTA

SEQ ID NO: 213 - protein, 14 aa, synthetic construct
  REGION 1..14: note = Synthetic antibody
ARAGGIHYYDSLDY

SEQ ID NO: 214 - protein, 6 aa, synthetic construct
  REGION 1..6: note = Synthetic antibody
QSISSY

SEQ ID NO: 215 - skipped

SEQ ID NO: 216 - protein, 9 aa, synthetic construct
  REGION 1..9: note = Synthetic antibody
QQSYSTPLT

SEQ ID NO: 217 - protein, 8 aa, synthetic construct
  REGION 1..8: note = Synthetic antibody
GFTFANAW

SEQ ID NO: 218 - protein, 10 aa, synthetic construct
  REGION 1..10: note = Synthetic antibody
IKPSSSGGRT

SEQ ID NO: 219 - protein, 13 aa, synthetic construct
  REGION 1..13: note = Synthetic antibody
CVTGRRVAKTFDF

SEQ ID NO: 220 - protein, 6 aa, synthetic construct
  REGION 1..6: note = Synthetic antibody
QSISSY

SEQ ID NO: 221 - skipped

SEQ ID NO: 222 - protein, 9 aa, synthetic construct
  REGION 1..9: note = Synthetic antibody
QQSYSTPRT

SEQ ID NO: 223 - DNA, 24 bp, synthetic construct
  misc_feature 1..24: note = Synthetic antibody
  source 1..24: mol_type = other DNA
ggaggcaccttcagcagctatgct

SEQ ID NO: 224 - DNA, 24 bp, synthetic construct
  misc_feature 1..24: note = Synthetic antibody
  source 1..24: mol_type = other DNA
atcatccctatctttggtacagca

SEQ ID NO: 225 - DNA, 36 bp, synthetic construct
  misc_feature 1..36: note = Synthetic antibody
  source 1..36: mol_type = other DNA
gcgagggggaatcagggggatacggcttttgatatc

SEQ ID NO: 226 - DNA, 27 bp, synthetic construct
  misc_feature 1..27: note = Synthetic antibody
  source 1..27: mol_type = other DNA
agcagtgacgttggtggttataactat

SEQ ID NO: 227 - skipped

SEQ ID NO: 228 - DNA, 33 bp, synthetic construct
  misc_feature 1..33: note = Synthetic antibody
  source 1..33: mol_type = other DNA
agctcatatacaagcagcagcactccttatgtc

SEQ ID NO: 229 - DNA, 24 bp, synthetic construct
  misc_feature 1..24: note = Synthetic antibody
  source 1..24: mol_type = other DNA
ggttacacctttagcgactatggt

SEQ ID NO: 230 - DNA, 24 bp, synthetic construct
  misc_feature 1..24: note = Synthetic antibody
  source 1..24: mol_type = other DNA
atcagtggttacaatggtaacaca

SEQ ID NO: 231 - DNA, 36 bp, synthetic construct
  misc_feature 1..36: note = Synthetic antibody
  source 1..36: mol_type = other DNA
gcgagattgctgctgggttactacggtatggacgtc

SEQ ID NO: 232 - DNA, 18 bp, synthetic construct
  misc_feature 1..18: note = Synthetic antibody
  source 1..18: mol_type = other DNA
cagagcattggcaactat

SEQ ID NO: 233 - skipped

SEQ ID NO: 234 - DNA, 30 bp, synthetic construct
  misc_feature 1..30: note = Synthetic antibody
  source 1..30: mol_type = other DNA
caacagagtttcagtatccccccgataact

SEQ ID NO: 235 - DNA, 24 bp, synthetic construct
  misc_feature 1..24: note = Synthetic antibody
  source 1..24: mol_type = other DNA
ggttacacgtttagcagctatggt

SEQ ID NO: 236 - DNA, 24 bp, synthetic construct
  misc_feature 1..24: note = Synthetic antibody
  source 1..24: mol_type = other DNA
atcagtggtaacactggtaacaca

SEQ ID NO: 237 - DNA, 42 bp, synthetic construct
  misc_feature 1..42: note = Synthetic antibody
  source 1..42: mol_type = other DNA
gcgagagatcgtggtatagcggcaggccgtgcttttgatatc

SEQ ID NO: 238 - DNA, 24 bp, synthetic construct
  misc_feature 1..24: note = Synthetic antibody
  source 1..24: mol_type = other DNA
agctccaacatcggaactaatcct

SEQ ID NO: 239 - skipped

SEQ ID NO: 240 - DNA, 33 bp, synthetic construct
  misc_feature 1..33: note = Synthetic antibody
  source 1..33: mol_type = other DNA
tcgtcatgggacgacagcctgagtgcgtgggtg

SEQ ID NO: 241 - DNA, 24 bp, synthetic construct
  misc_feature 1..24: note = Synthetic antibody
  source 1..24: mol_type = other DNA
ggttacccctttaccagcaatggt

SEQ ID NO: 242 - DNA, 24 bp, synthetic construct
  misc_feature 1..24: note = Synthetic antibody
  source 1..24: mol_type = other DNA
atcagcactaacagcggaaacaca

SEQ ID NO: 243 - DNA, 30 bp, synthetic construct
  misc_feature 1..30: note = Synthetic antibody
  source 1..30: mol_type = other DNA
gcgagagatgttcaatatcgtatggacgtc

SEQ ID NO: 244 - DNA, 24 bp, synthetic construct
  misc_feature 1..24: note = Synthetic antibody
  source 1..24: mol_type = other DNA
aggtccaacatcggaagtaatcct

SEQ ID NO: 245 - skipped

SEQ ID NO: 246 - DNA, 30 bp, synthetic construct
  misc_feature 1..30: note = Synthetic antibody
  source 1..30: mol_type = other DNA
gcagcgtgggatgacaacgtgcacggagtt

SEQ ID NO: 247 - DNA, 24 bp, synthetic construct
  misc_feature 1..24: note = Synthetic antibody
  source 1..24: mol_type = other DNA
ggattcaccatcagcagttatggc

SEQ ID NO: 248 - DNA, 24 bp, synthetic construct
  misc_feature 1..24: note = Synthetic antibody
  source 1..24: mol_type = other DNA
atatcatatgatggaagtaataaa

SEQ ID NO: 249 - DNA, 66 bp, synthetic construct
  misc_feature 1..66: note = Synthetic antibody
  source 1..66: mol_type = other DNA
gcgaaagatctcttattacgatattttgactggagccccaaatactactacaacggtatggacgtc

SEQ ID NO: 250 - DNA, 18 bp, synthetic construct
  misc_feature 1..18: note = Synthetic antibody
  source 1..18: mol_type = other DNA
aacattggaagtaaaagt

SEQ ID NO: 251 - skipped

SEQ ID NO: 252 - DNA, 36 bp, synthetic construct
  misc_feature 1..36: note = Synthetic antibody
  source 1..36: mol_type = other DNA
caggtgtgggatagtagtagtgatcatccgggggtc

SEQ ID NO: 253 - DNA, 24 bp, synthetic construct
  misc_feature 1..24: note = Synthetic antibody
  source 1..24: mol_type = other DNA
ggaggcaccttcagcagctatgct

SEQ ID NO: 254 - DNA, 24 bp, synthetic construct
  misc_feature 1..24: note = Synthetic antibody
  source 1..24: mol_type = other DNA
atcatccctatctttggtacagca

SEQ ID NO: 255 - DNA, 36 bp, synthetic construct
  misc_feature 1..36: note = Synthetic antibody
  source 1..36: mol_type = other DNA
gcgagagtggcagtggctgacaccttctttgactac

SEQ ID NO: 256 - DNA, 27 bp, synthetic construct
  misc_feature 1..27: note = Synthetic antibody
  source 1..27: mol_type = other DNA
agcagtgacgttggtggttataactat

SEQ ID NO: 257 - skipped

SEQ ID NO: 258 - DNA, 33 bp, synthetic construct
  misc_feature 1..33: note = Synthetic antibody
  source 1..33: mol_type = other DNA
agctcatatacaagcagcagcaccccttatgtc

SEQ ID NO: 259 - DNA, 24 bp, synthetic construct
  misc_feature 1..24: note = Synthetic antibody
  source 1..24: mol_type = other DNA
ggttacacctttaccagctatggt

SEQ ID NO: 260 - DNA, 24 bp, synthetic construct
  misc_feature 1..24: note = Synthetic antibody
  source 1..24: mol_type = other DNA
atcagcgcttacaatggtaacaca

SEQ ID NO: 261 - DNA, 27 bp, synthetic construct
  misc_feature 1..27: note = Synthetic antibody
  source 1..27: mol_type = other DNA
gcgagagacggctacggtatggacgtc

SEQ ID NO: 262 - DNA, 18 bp, synthetic construct
  misc_feature 1..18: note = Synthetic antibody
  source 1..18: mol_type = other DNA
cacaccgttaacagctat

SEQ ID NO: 263 - skipped

SEQ ID NO: 264 - DNA, 27 bp, synthetic construct
  misc_feature 1..27: note = Synthetic antibody
  source 1..27: mol_type = other DNA
caacagagttacagaacccctctcact

SEQ ID NO: 265 - DNA, 24 bp, synthetic construct
  misc_feature 1..24: note = Synthetic antibody
  source 1..24: mol_type = other DNA
ggattcacctttagcagctatgcc

SEQ ID NO: 266 - DNA, 24 bp, synthetic construct
  misc_feature 1..24: note = Synthetic antibody
  source 1..24: mol_type = other DNA
attaatgatagtggtggtagcaca

SEQ ID NO: 267 - DNA, 45 bp, synthetic construct
  misc_feature 1..45: note = Synthetic antibody
  source 1..45: mol_type = other DNA
gcgaaagatcatattacgatttttggagtggccccgtttgactac

SEQ ID NO: 268 - DNA, 24 bp, synthetic construct
  misc_feature 1..24: note = Synthetic antibody
  source 1..24: mol_type = other DNA
agctccaacatcggaggtaatcct

SEQ ID NO: 269 - skipped

SEQ ID NO: 270 - DNA, 33 bp, synthetic construct
  misc_feature 1..33: note = Synthetic antibody
  source 1..33: mol_type = other DNA
gcaacatgggatgacagcctgaatggttgggtg

SEQ ID NO: 271 - DNA, 30 bp, synthetic construct
  misc_feature 1..30: note = Synthetic antibody
  source 1..30: mol_type = other DNA
ggggacagtgtctctagcaacagtgctgct

SEQ ID NO: 272 - DNA, 27 bp, synthetic construct
  misc_feature 1..27: note = Synthetic antibody
  source 1..27: mol_type = other DNA
acatactacaggtccaagtggtataat

SEQ ID NO: 273 - DNA, 42 bp, synthetic construct
  misc_feature 1..42: note = Synthetic antibody
  source 1..42: mol_type = other DNA
gcaagacaagatgaatcggcagccgatgatgcttttgatatc

SEQ ID NO: 274 - DNA, 24 bp, synthetic construct
  misc_feature 1..24: note = Synthetic antibody
  source 1..24: mol_type = other DNA
aagtccaacgtcggaaataatttt

SEQ ID NO: 275 - skipped

SEQ ID NO: 276 - DNA, 33 bp, synthetic construct
  misc_feature 1..33: note = Synthetic antibody
  source 1..33: mol_type = other DNA
gcagcatgggatgacagcctgcccggttgggtg

SEQ ID NO: 277 - DNA, 24 bp, synthetic construct
  misc_feature 1..24: note = Synthetic antibody
  source 1..24: mol_type = other DNA
ggttacacgtttcccgacaacggt

SEQ ID NO: 278 - DNA, 24 bp, synthetic construct
  misc_feature 1..24: note = Synthetic antibody
  source 1..24: mol_type = other DNA
atcaacgttgacactggatacacc

SEQ ID NO: 279 - DNA, 30 bp, synthetic construct
  misc_feature 1..30: note = Synthetic antibody
  source 1..30: mol_type = other DNA
gcgagagaaatcgagggagtgtggacagct

SEQ ID NO: 280 - DNA, 24 bp, synthetic construct
  misc_feature 1..24: note = Synthetic antibody
  source 1..24: mol_type = other DNA
agctccaacatcggaagtaatcct

SEQ ID NO: 281 - skipped

SEQ ID NO: 282 - DNA, 33 bp, synthetic construct
  misc_feature 1..33: note = Synthetic antibody
  source 1..33: mol_type = other DNA
gcggcatgggatgacagcctgaatggttgggtg

SEQ ID NO: 283 - DNA, 24 bp, synthetic construct
  misc_feature 1..24: note = Synthetic antibody
  source 1..24: mol_type = other DNA
ggaggcaccttcagcagctatgct

SEQ ID NO: 284 - DNA, 24 bp, synthetic construct
  misc_feature 1..24: note = Synthetic antibody
  source 1..24: mol_type = other DNA
atcatccctatctttggtacagca

SEQ ID NO: 285 - DNA, 36 bp, synthetic construct
  misc_feature 1..36: note = Synthetic antibody
  source 1..36: mol_type = other DNA
gcgaggtcggactacggtgactcctactttgactac

SEQ ID NO: 286 - DNA, 27 bp, synthetic construct
  misc_feature 1..27: note = Synthetic antibody
  source 1..27: mol_type = other DNA
agcagtgacgttggtggttttaactat

SEQ ID NO: 287 - skipped

SEQ ID NO: 288 - DNA, 33 bp, synthetic construct
  misc_feature 1..33: note = Synthetic antibody
  source 1..33: mol_type = other DNA
agctcatatacaagcagcaccgctccttatgtc

SEQ ID NO: 289 - DNA, 24 bp, synthetic construct
  misc_feature 1..24: note = Synthetic antibody
  source 1..24: mol_type = other DNA
ggaggcaccttcagcagctatgct

SEQ ID NO: 290 - DNA, 24 bp, synthetic construct
  misc_feature 1..24: note = Synthetic antibody
  source 1..24: mol_type = other DNA
atcatccctatctttggtacagca

SEQ ID NO: 291 - DNA, 36 bp, synthetic construct
  misc_feature 1..36: note = Synthetic antibody
  source 1..36: mol_type = other DNA
gcgagagcctctatggtcgagacttactttgactac

SEQ ID NO: 292 - DNA, 27 bp, synthetic construct
  misc_feature 1..27: note = Synthetic antibody
  source 1..27: mol_type = other DNA
agcagtgatgttggtggttataactat

SEQ ID NO: 293 - skipped

SEQ ID NO: 294 - DNA, 33 bp, synthetic construct
  misc_feature 1..33: note = Synthetic antibody
  source 1..33: mol_type = other DNA
tcctcatatgcaggcgggtacaccccctatgtc

SEQ ID NO: 295 - DNA, 24 bp, synthetic construct
  misc_feature 1..24: note = Synthetic antibody
  source 1..24: mol_type = other DNA
ggttacacctttaccaactatggt

SEQ ID NO: 296 - DNA, 24 bp, synthetic construct
  misc_feature 1..24: note = Synthetic antibody
  source 1..24: mol_type = other DNA
atcagcggtgacgctggtgacaca

SEQ ID NO: 297 - DNA, 63 bp, synthetic construct
  misc_feature 1..63: note = Synthetic antibody
  source 1..63: mol_type = other DNA
gcgagagatggggaatatattcctatgtttcggggatttgacaactactacggtctggacgtc

SEQ ID NO: 298 - DNA, 24 bp, synthetic construct
  misc_feature 1..24: note = Synthetic antibody
  source 1..24: mol_type = other DNA
aacgccaacatcggaagtaatcct

SEQ ID NO: 299 - skipped

SEQ ID NO: 300 - DNA, 33 bp, synthetic construct
  misc_feature 1..33: note = Synthetic antibody
  source 1..33: mol_type = other DNA
gaaggatgggatgacagcctgaatggctatgtc

SEQ ID NO: 301 - DNA, 24 bp, synthetic construct
  misc_feature 1..24: note = Synthetic antibody
  source 1..24: mol_type = other DNA
ggaggcaccttcagcagctatgct

SEQ ID NO: 302 - DNA, 24 bp, synthetic construct
  misc_feature 1..24: note = Synthetic antibody
  source 1..24: mol_type = other DNA
atcatccctatctttggtacagca

SEQ ID NO: 303 - DNA, 42 bp, synthetic construct
  misc_feature 1..42: note = Synthetic antibody
  source 1..42: mol_type = other DNA
gcgagcgggattatacctgataatccctacggtatggacgtc

SEQ ID NO: 304 - DNA, 33 bp, synthetic construct
  misc_feature 1..33: note = Synthetic antibody
  source 1..33: mol_type = other DNA
caaagcctcgtatacagtgatggaaacacctac

SEQ ID NO: 305 - skipped

SEQ ID NO: 306 - DNA, 27 bp, synthetic construct
  misc_feature 1..27: note = Synthetic antibody
  source 1..27: mol_type = other DNA
atgcaaggtacacactggccggtcacc

SEQ ID NO: 307 - DNA, 27 bp, synthetic construct
  misc_feature 1..27: note = Synthetic antibody
  source 1..27: mol_type = other DNA
ggtgtctccaccagcagtactcattgg

SEQ ID NO: 308 - DNA, 21 bp, synthetic construct
  misc_feature 1..21: note = Synthetic antibody
  source 1..21: mol_type = other DNA
atccttcataatggaaacacc

SEQ ID NO: 309 - DNA, 45 bp, synthetic construct
  misc_feature 1..45: note = Synthetic antibody
  source 1..45: mol_type = other DNA
gcgtcagcgtcgcctaactggggagctagcggggcttttgatgcc

SEQ ID NO: 310 - DNA, 24 bp, synthetic construct
  misc_feature 1..24: note = Synthetic antibody
  source 1..24: mol_type = other DNA
agctccaacatcggaagtaatcct

SEQ ID NO: 311 - skipped

SEQ ID NO: 312 - DNA, 33 bp, synthetic construct
  misc_feature 1..33: note = Synthetic antibody
  source 1..33: mol_type = other DNA
tcaacatgggatgacagcctgaatggtcttgtc

SEQ ID NO: 313 - DNA, 24 bp, synthetic construct
  misc_feature 1..24: note = Synthetic antibody
  source 1..24: mol_type = other DNA
ggttacacctttaccaggtatggt

SEQ ID NO: 314 - DNA, 24 bp, synthetic construct
  misc_feature 1..24: note = Synthetic antibody
  source 1..24: mol_type = other DNA
atcagcggttacaatggtaataca

SEQ ID NO: 315 - DNA, 33 bp, synthetic construct
  misc_feature 1..33: note = Synthetic antibody
  source 1..33: mol_type = other DNA
gcgagagatcggggtagtggttatcttgaatac

SEQ ID NO: 316 - DNA, 18 bp, synthetic construct
  misc_feature 1..18: note = Synthetic antibody
  source 1..18: mol_type = other DNA
cagggcatttacagttct

SEQ ID NO: 317 - skipped

SEQ ID NO: 318 - DNA, 27 bp, synthetic construct
  misc_feature 1..27: note = Synthetic antibody
  source 1..27: mol_type = other DNA
caacaacattacaatctacccatcacc

SEQ ID NO: 319 - DNA, 24 bp, synthetic construct
  misc_feature 1..24: note = Synthetic antibody
  source 1..24: mol_type = other DNA
ggattcactttcagtaacgcctgg

SEQ ID NO: 320 - DNA, 30 bp, synthetic construct
  misc_feature 1..30: note = Synthetic antibody
  source 1..30: mol_type = other DNA
attaaaagcaaaactgatggtgggacaaca

SEQ ID NO: 321 - DNA, 48 bp, synthetic construct
  misc_feature 1..48: note = Synthetic antibody
  source 1..48: mol_type = other DNA
accacagatcgatatagcagcagctggtactcccctgcttttgatatc

SEQ ID NO: 322 - DNA, 24 bp, synthetic construct
  misc_feature 1..24: note = Synthetic antibody
  source 1..24: mol_type = other DNA
agctccaacatcggaaataatgtt

SEQ ID NO: 323 - skipped

SEQ ID NO: 324 - DNA, 33 bp, synthetic construct
  misc_feature 1..33: note = Synthetic antibody
  source 1..33: mol_type = other DNA
gcaacatgggatgacagcctgaatggccctgta

SEQ ID NO: 325 - DNA, 24 bp, synthetic construct
  misc_feature 1..24: note = Synthetic antibody
  source 1..24: mol_type = other DNA
ggttacacctttaccagctatggt

SEQ ID NO: 326 - DNA, 24 bp, synthetic construct
  misc_feature 1..24: note = Synthetic antibody
  source 1..24: mol_type = other DNA
atcagcgcttacaatggtaacaca

SEQ ID NO: 327 - DNA, 27 bp, synthetic construct
  misc_feature 1..27: note = Synthetic antibody
  source 1..27: mol_type = other DNA
gcgagagtgggcagctatggtgactac

SEQ ID NO: 328 - DNA, 18 bp, synthetic construct
  misc_feature 1..18: note = Synthetic antibody
  source 1..18: mol_type = other DNA
cagggcattagcaattat

SEQ ID NO: 329 - skipped

SEQ ID NO: 330 - DNA, 27 bp, synthetic construct
  misc_feature 1..27: note = Synthetic antibody
  source 1..27: mol_type = other DNA
caaaagtataacagtgccccgctcact

SEQ ID NO: 331 - DNA, 24 bp, synthetic construct
  misc_feature 1..24: note = Synthetic antibody
  source 1..24: mol_type = other DNA
gggtacaccttcacccactcctat

SEQ ID NO: 332 - DNA, 24 bp, synthetic construct
  misc_feature 1..24: note = Synthetic antibody
  source 1..24: mol_type = other DNA
atcaaccctgttggtggtagcaca

SEQ ID NO: 333 - DNA, 42 bp, synthetic construct
  misc_feature 1..42: note = Synthetic antibody
  source 1..42: mol_type = other DNA
gctagagcggggggagatggctacaatccttactttgactac

SEQ ID NO: 334 - DNA, 18 bp, synthetic construct
  misc_feature 1..18: note = Synthetic antibody
  source 1..18: mol_type = other DNA
cagagtattagtagctgg

SEQ ID NO: 335 - skipped

SEQ ID NO: 336 - DNA, 24 bp, synthetic construct
  misc_feature 1..24: note = Synthetic antibody
  source 1..24: mol_type = other DNA
caacagtataatagttatctcact

SEQ ID NO: 337 - DNA, 24 bp, synthetic construct
  misc_feature 1..24: note = Synthetic antibody
  source 1..24: mol_type = other DNA
ggattcaccttcagtgactactac

SEQ ID NO: 338 - DNA, 24 bp, synthetic construct
  misc_feature 1..24: note = Synthetic antibody
  source 1..24: mol_type = other DNA
attagtagtagtagtagttacaca

SEQ ID NO: 339 - DNA, 33 bp, synthetic construct
  misc_feature 1..33: note = Synthetic antibody
  source 1..33: mol_type = other DNA
gcgagagtcggagatcttgatgcttttgatatc

SEQ ID NO: 340 - DNA, 24 bp, synthetic construct
  misc_feature 1..24: note = Synthetic antibody
  source 1..24: mol_type = other DNA
agctccaacatcggaagtaattat

SEQ ID NO: 341 - skipped

SEQ ID NO: 342 - DNA, 33 bp, synthetic construct
  misc_feature 1..33: note = Synthetic antibody
  source 1..33: mol_type = other DNA
gcagcatgggatgacagcctgagtggttgggtg

SEQ ID NO: 343 - DNA, 24 bp, synthetic construct
  misc_feature 1..24: note = Synthetic antibody
  source 1..24: mol_type = other DNA
ggattcaccttcagtgaccactac

SEQ ID NO: 344 - DNA, 24 bp, synthetic construct
  misc_feature 1..24: note = Synthetic antibody
  source 1..24: mol_type = other DNA
attactactactggtagtaccatg

SEQ ID NO: 345 - DNA, 39 bp, synthetic construct
  misc_feature 1..39: note = Synthetic antibody
  source 1..39: mol_type = other DNA
gcgagagattccatacatgtggatggtgcttttgatatt

SEQ ID NO: 346 - DNA, 18 bp, synthetic construct
  misc_feature 1..18: note = Synthetic antibody
  source 1..18: mol_type = other DNA
aacattggaagtaaaagt

SEQ ID NO: 347 - skipped

SEQ ID NO: 348 - DNA, 33 bp, synthetic construct
  misc_feature 1..33: note = Synthetic antibody
  source 1..33: mol_type = other DNA
caggtgtgggatagtagtagtgatcattgggtg

SEQ ID NO: 349 - DNA, 24 bp, synthetic construct
  misc_feature 1..24: note = Synthetic antibody
  source 1..24: mol_type = other DNA
ggttacacctttaccagctatggt

SEQ ID NO: 350 - DNA, 24 bp, synthetic construct
  misc_feature 1..24: note = Synthetic antibody
  source 1..24: mol_type = other DNA
atcagcgcttacaatggtaacaca

SEQ ID NO: 351 - DNA, 30 bp, synthetic construct
  misc_feature 1..30: note = Synthetic antibody
  source 1..30: mol_type = other DNA
gcgagagatcggggagggtggttcgacccc

SEQ ID NO: 352 - DNA, 18 bp, synthetic construct
  misc_feature 1..18: note = Synthetic antibody
  source 1..18: mol_type = other DNA
cagagcataagcaacttt

SEQ ID NO: 353 - skipped

SEQ ID NO: 354 - DNA, 27 bp, synthetic construct
  misc_feature 1..27: note = Synthetic antibody
  source 1..27: mol_type = other DNA
caacagggttacagtgtcccactcact

SEQ ID NO: 355 - DNA, 24 bp, synthetic construct
  misc_feature 1..24: note = Synthetic antibody
  source 1..24: mol_type = other DNA
ggaggcaccttcagcagctatgct

SEQ ID NO: 356 - DNA, 24 bp, synthetic construct
  misc_feature 1..24: note = Synthetic antibody
  source 1..24: mol_type = other DNA
atcatccctatctttggtacagca

SEQ ID NO: 357 - DNA, 36 bp, synthetic construct
  misc_feature 1..36: note = Synthetic antibody
  source 1..36: mol_type = other DNA
gcgagagagtcgtatagcagctcgtcctttgactac

SEQ ID NO: 358 - DNA, 27 bp, synthetic construct
  misc_feature 1..27: note = Synthetic antibody
  source 1..27: mol_type = other DNA
agcagtgacgttggtggttataactat

SEQ ID NO: 359 - skipped

SEQ ID NO: 360 - DNA, 33 bp, synthetic construct
  misc_feature 1..33: note = Synthetic antibody
  source 1..33: mol_type = other DNA
agctcatatacaagcagcagcactccctatgtc

SEQ ID NO: 361 - DNA, 24 bp, synthetic construct
  misc_feature 1..24: note = Synthetic antibody
  source 1..24: mol_type = other DNA
ggattcaccttcagtagctacgac

SEQ ID NO: 362 - DNA, 21 bp, synthetic construct
  misc_feature 1..21: note = Synthetic antibody
  source 1..21: mol_type = other DNA
attggtactgctggtgacaca

SEQ ID NO: 363 - DNA, 54 bp, synthetic construct
  misc_feature 1..54: note = Synthetic antibody
  source 1..54: mol_type = other DNA
gcaagggcgaaggaatattgtagtggtggtagctgccaagatgcttttgatatc

SEQ ID NO: 364 - DNA, 18 bp, synthetic construct
  misc_feature 1..18: note = Synthetic antibody
  source 1..18: mol_type = other DNA
cagagcattaggacctac

SEQ ID NO: 365 - skipped

SEQ ID NO: 366 - DNA, 30 bp, synthetic construct
  misc_feature 1..30: note = Synthetic antibody
  source 1..30: mol_type = other DNA
caacagagttacagtgcccccgtgttcact

SEQ ID NO: 367 - DNA, 24 bp, synthetic construct
  misc_feature 1..24: note = Synthetic antibody
  source 1..24: mol_type = other DNA
gggtacaccttcaccgactattat

SEQ ID NO: 368 - DNA, 24 bp, synthetic construct
  misc_feature 1..24: note = Synthetic antibody
  source 1..24: mol_type = other DNA
gtcaacgctaatggtggtgacaca

SEQ ID NO: 369 - DNA, 48 bp, synthetic construct
  misc_feature 1..48: note = Synthetic antibody
  source 1..48: mol_type = other DNA
gctagagattggtattactattcttcggggagtttcccttttgactac

SEQ ID NO: 370 - DNA, 18 bp, synthetic construct
  misc_feature 1..18: note = Synthetic antibody
  source 1..18: mol_type = other DNA
cagagcattagcagcttt

SEQ ID NO: 371 - skipped

SEQ ID NO: 372 - DNA, 27 bp, synthetic construct
  misc_feature 1..27: note = Synthetic antibody
  source 1..27: mol_type = other DNA
caacagagttacagtatcccgatcacc

SEQ ID NO: 373 - DNA, 24 bp, synthetic construct
  misc_feature 1..24: note = Synthetic antibody
  source 1..24: mol_type = other DNA
gggtacaccttcaccgactattat

SEQ ID NO: 374 - DNA, 24 bp, synthetic construct
  misc_feature 1..24: note = Synthetic antibody
  source 1..24: mol_type = other DNA
gtcaacgctaatggtggtgacaca

SEQ ID NO: 375 - DNA, 48 bp, synthetic construct; identical to SEQ ID NO: 369
  misc_feature 1..48: note = Synthetic antibody
  source 1..48: mol_type = other DNA

SEQ ID NO: 376 - DNA, 18 bp, synthetic construct
  misc_feature 1..18: note = Synthetic antibody
  source 1..18: mol_type = other DNA
caagatgtgtccatcgcc

SEQ ID NO: 377 - skipped

SEQ ID NO: 378 - DNA, 27 bp, synthetic construct
  misc_feature 1..27: note = Synthetic antibody
  source 1..27: mol_type = other DNA
cagcagcactacatcacccctttaaca

SEQ ID NO: 379 - DNA, 24 bp, synthetic construct
  misc_feature 1..24: note = Synthetic antibody
  source 1..24: mol_type = other DNA
ggttacatcttcaacacctatggt

SEQ ID NO: 380 - DNA, 24 bp, synthetic construct
  misc_feature 1..24: note = Synthetic antibody
  source 1..24: mol_type = other DNA
accaacgtttacaatagtaacaca

SEQ ID NO: 381 - DNA, 33 bp, synthetic construct
  misc_feature 1..33: note = Synthetic antibody
  source 1..33: mol_type = other DNA
gcgagagacccgggctcggctatttttgatatg

SEQ ID NO: 382 - DNA, 18 bp, synthetic construct
  misc_feature 1..18: note = Synthetic antibody
  source 1..18: mol_type = other DNA
cagagcattagcagcttt

SEQ ID NO: 383 - skipped

SEQ ID NO: 384 - DNA, 27 bp, synthetic construct
  misc_feature 1..27: note = Synthetic antibody
  source 1..27: mol_type = other DNA
caacagagttacagtatcccgatcacc

SEQ ID NO: 385 - DNA, 24 bp, synthetic construct
  misc_feature 1..24: note = Synthetic antibody
  source 1..24: mol_type = other DNA
ggtgacgccatcaaagataagtac

SEQ ID NO: 386 - DNA, 21 bp, synthetic construct
  misc_feature 1..21: note = Synthetic antibody
  source 1..21: mol_type = other DNA
atctctaacagtggaagcacc

SEQ ID NO: 387 - DNA, 33 bp, synthetic construct
  misc_feature 1..33: note = Synthetic antibody
  source 1..33: mol_type = other DNA
gcgagaggcagcggttatactgcttttgatatc

SEQ ID NO: 388 - DNA, 18 bp, synthetic construct
  misc_feature 1..18: note = Synthetic antibody
  source 1..18: mol_type = other DNA
cagagcattagcagctat

SEQ ID NO: 389 - skipped

SEQ ID NO: 390 - DNA, 27 bp, synthetic construct
  misc_feature 1..27: note = Synthetic antibody
  source 1..27: mol_type = other DNA
caacagagttacagtaccccgcccact

SEQ ID NO: 391 - DNA, 24 bp, synthetic construct
  misc_feature 1..24: note = Synthetic antibody
  source 1..24: mol_type = other DNA
ggattcacctttagcagctatgcc

SEQ ID NO: 392 - DNA, 24 bp, synthetic construct
  misc_feature 1..24: note = Synthetic antibody
  source 1..24: mol_type = other DNA
attagtggtagtggtggtagcaca

SEQ ID NO: 393 - DNA, 42 bp, synthetic construct
  misc_feature 1..42: note = Synthetic antibody
  source 1..42: mol_type = other DNA
gcgaaagatgtcgacccctatagtagtggttattttgactac

SEQ ID NO: 394 - DNA, 18 bp, synthetic construct
  misc_feature 1..18: note = Synthetic antibody
  source 1..18: mol_type = other DNA
cagggtattggcagctgg

SEQ ID NO: 395 - skipped

SEQ ID NO: 396 - DNA, 27 bp, synthetic construct
  misc_feature 1..27: note = Synthetic antibody
  source 1..27: mol_type = other DNA
caacaggctaacagtttcccgctcact

SEQ ID NO: 397 - DNA, 24 bp, synthetic construct
  misc_feature 1..24: note = Synthetic antibody
  source 1..24: mol_type = other DNA
ggattcacctttagcaactatgcc

SEQ ID NO: 398 - DNA, 24 bp, synthetic construct
  misc_feature 1..24: note = Synthetic antibody
  source 1..24: mol_type = other DNA
attagtggtagtagtattagcaca

SEQ ID NO: 399 - DNA, 30 bp, synthetic construct
  misc_feature 1..30: note = Synthetic antibody
  source 1..30: mol_type = other DNA
gcgtcctcgggtttcgggtcctttgactac

SEQ ID NO: 400 - DNA, 24 bp, synthetic construct
  misc_feature 1..24: note = Synthetic antibody
  source 1..24: mol_type = other DNA
agctccaatatcggagataattct

SEQ ID NO: 401 - skipped

SEQ ID NO: 402 - DNA, 33 bp, synthetic construct
  misc_feature 1..33: note = Synthetic antibody
  source 1..33: mol_type = other DNA
gcaacatgggatgacagcctgaacggtgtggtt

SEQ ID NO: 403 - DNA, 24 bp, synthetic construct
  misc_feature 1..24: note = Synthetic antibody
  source 1..24: mol_type = other DNA
ggattcaccttaagtagctatgcc

SEQ ID NO: 404 - DNA, 21 bp, synthetic construct
  misc_feature 1..21: note = Synthetic antibody
  source 1..21: mol_type = other DNA
attaatagtggtggtagcaca

SEQ ID NO: 405 - DNA, 36 bp, synthetic construct
  misc_feature 1..36: note = Synthetic antibody
  source 1..36: mol_type = other DNA
gcgaaagcgaagagttatagcagtgaccttgaccac

SEQ ID NO: 406 - DNA, 36 bp, synthetic construct
  misc_feature 1..36: note = Synthetic antibody
  source 1..36: mol_type = other DNA
cagagtctgttaaacagtggaaatcaaaagacctac

SEQ ID NO: 407 - skipped

SEQ ID NO: 408 - DNA, 27 bp, synthetic construct
  misc_feature 1..27: note = Synthetic antibody
  source 1..27: mol_type = other DNA
cagaatgatcatagttatccattcacg

SEQ ID NO: 409 - DNA, 24 bp, synthetic construct
  misc_feature 1..24: note = Synthetic antibody
  source 1..24: mol_type = other DNA
ggattcacctttagcagctatgcc

SEQ ID NO: 410 - DNA, 24 bp, synthetic construct
  misc_feature 1..24: note = Synthetic antibody
  source 1..24: mol_type = other DNA
attagtggtagtggtggtagcaca

SEQ ID NO: 411 - DNA, 48 bp, synthetic construct
  misc_feature 1..48: note = Synthetic antibody
  source 1..48: mol_type = other DNA
gcgaaagatcttctcggggatagtgggagctaccctgcttttgatatc

SEQ ID NO: 412 - DNA, 18 bp, synthetic construct
  misc_feature 1..18: note = Synthetic antibody
  source 1..18: mol_type = other DNA
cagagcattagcagctat

SEQ ID NO: 413 - skipped

SEQ ID NO: 414 - DNA, 27 bp, synthetic construct
  misc_feature 1..27: note = Synthetic antibody
  source 1..27: mol_type = other DNA
caacagagttacagtaccccgtacact

SEQ ID NO: 415 - DNA, 24 bp, synthetic construct
  misc_feature 1..24: note = Synthetic antibody
  source 1..24: mol_type = other DNA
ggattcccctttgcggactatgct

SEQ ID NO: 416 - DNA, 30 bp, synthetic construct
  misc_feature 1..30: note = Synthetic antibody
  source 1..30: mol_type = other DNA
attaaaagcaagactagagatgggacaaca

SEQ ID NO: 417 - DNA, 24 bp, synthetic construct
  misc_feature 1..24: note = Synthetic antibody
  source 1..24: mol_type = other DNA
gccctcgggggagcagcagctcta

SEQ ID NO: 418 - DNA, 27 bp, synthetic construct
  misc_feature 1..27: note = Synthetic antibody
  source 1..27: mol_type = other DNA
agctccaacatcggggcaggttatggt

SEQ ID NO: 419 - skipped

SEQ ID NO: 420 - DNA, 33 bp, synthetic construct
  misc_feature 1..33: note = Synthetic antibody
  source 1..33: mol_type = other DNA
cagtcctatgacagcagcctgggggttggtgtg

SEQ ID NO: 421 - DNA, 24 bp, synthetic construct
  misc_feature 1..24: note = Synthetic antibody
  source 1..24: mol_type = other DNA
ggaggcaccttcagcagctatgct

SEQ ID NO: 422 - DNA, 24 bp, synthetic construct
  misc_feature 1..24: note = Synthetic antibody
  source 1..24: mol_type = other DNA
atcatccctatctttggtacagca

SEQ ID NO: 423 - DNA, 39 bp, synthetic construct
  misc_feature 1..39: note = Synthetic antibody
  source 1..39: mol_type = other DNA
gcgattagtagtggttattattataacccatttgactac

SEQ ID NO: 424 - DNA, 18 bp, synthetic construct
  misc_feature 1..18: note = Synthetic antibody
  source 1..18: mol_type = other DNA
cagagcattagcagctat

SEQ ID NO: 425 - skipped

SEQ ID NO: 426 - DNA, 27 bp, synthetic construct
  misc_feature 1..27: note = Synthetic antibody
  source 1..27: mol_type = other DNA
caacagagttacagtacccccctcact

SEQ ID NO: 427 - DNA, 24 bp, synthetic construct
  misc_feature 1..24: note = Synthetic antibody
  source 1..24: mol_type = other DNA
ggaggcaccttcagcagctatgct

SEQ ID NO: 428 - DNA, 24 bp, synthetic construct
  misc_feature 1..24: note = Synthetic antibody
  source 1..24: mol_type = other DNA
atcatccctatctttggtacagca

SEQ ID NO: 429 - DNA, 30 bp, synthetic construct
  misc_feature 1..30: note = Synthetic antibody
  source 1..30: mol_type = other DNA
gcgagagggggataccagccactggattac

SEQ ID NO: 430 - DNA, 18 bp, synthetic construct
  misc_feature 1..18: note = Synthetic antibody
  source 1..18: mol_type = other DNA
cagagcattagcagctat

SEQ ID NO: 431 - skipped

SEQ ID NO: 432 - DNA, 27 bp, synthetic construct
  misc_feature 1..27: note = Synthetic antibody
  source 1..27: mol_type = other DNA
caacagagttacagtaccccattcact

SEQ ID NO: 433 - DNA, 24 bp, synthetic construct
  misc_feature 1..24: note = Synthetic antibody
  source 1..24: mol_type = other DNA
ggaggcaccttcagcagctatgct

SEQ ID NO: 434 - DNA, 24 bp, synthetic construct
  misc_feature 1..24: note = Synthetic antibody
  source 1..24: mol_type = other DNA
atcatccctatctttggtacagca

SEQ ID NO: 435 - DNA, 42 bp, synthetic construct
  misc_feature 1..42: note = Synthetic antibody
  source 1..42: mol_type = other DNA
gcgagagctgggggaatccattactatgatagtttagactac

SEQ ID NO: 436 - DNA, 18 bp, synthetic construct
  misc_feature 1..18: note = Synthetic antibody
  source 1..18: mol_type = other DNA
cagagcattagcagctat

SEQ ID NO: 437 - skipped

SEQ ID NO: 438 - DNA, 27 bp, synthetic construct
  misc_feature 1..27: note = Synthetic antibody
  source 1..27: mol_type = other DNA
caacagagttacagtacccctctgacg

SEQ ID NO: 439 - DNA, 24 bp, synthetic construct
  misc_feature 1..24: note = Synthetic antibody
  source 1..24: mol_type = other DNA
ggattcaccttcgcgaatgcctgg

SEQ ID NO: 440 - DNA, 30 bp, synthetic construct
  misc_feature 1..30: note = Synthetic antibody
  source 1..30: mol_type = other DNA
attaagcccagttctagcggcgggaggaca

SEQ ID NO: 441 - DNA, 39 bp, synthetic construct
  misc_feature 1..39: note = Synthetic antibody
  source 1..39: mol_type = other DNA
tgtgtcacaggaagaagggtcgcgaagacttttgatttc

SEQ ID NO: 442 - DNA, 18 bp, synthetic construct
  misc_feature 1..18: note = Synthetic antibody
  source 1..18: mol_type = other DNA
cagagcattagcagctat

SEQ ID NO: 443 - skipped

SEQ ID NO: 444 - DNA, 27 bp, synthetic construct
  misc_feature 1..27: note = Synthetic antibody
  source 1..27: mol_type = other DNA
caacagagttacagtacccctcggacg

SEQ ID NO: 445 - DNA, 357 bp, synthetic construct
  misc_feature 1..357: note = Synthetic antibody
  source 1..357: mol_type = other DNA
gaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagggggaatcagggggatacggcttttgatatctggggccaagggaccacggtcatcgtctcctca

SEQ ID NO: 446 - DNA, 357 bp, synthetic construct
  misc_feature 1..357: note = Synthetic antibody
  source 1..357: mol_type = other DNA
caggtgcagctggtgcagtctggagctgaggtgaagaagcctggggcctcagtgaaggtctcctgcaaggcttctggttacacctttagcgactatggtttcagctgggtgcgacaggcccctggacaagggcttgagtggatgggatggatcagtggttacaatggtaacacaaactatgcacagaagttccagggcagagtcaccatgaccatagacgcatccacgagcacagcctacatggagctgaggagcctgaaatctgacgacacggccgtgtattactgtgcgagattgctgctgggttactacggtatggacgtctggggccaagggaccacggtcaccgtctcctca

SEQ ID NO: 447 - DNA, 363 bp, synthetic construct
  misc_feature 1..363: note = Synthetic antibody
  source 1..363: mol_type = other DNA
caggtgcagctggtgcagtctggagctgaggtgaagaagcctggggcctcagtgaaggtctcctgcaggacttctggttacacgtttagcagctatggtatcagctgggtgcgacaggcccctggacaagggcttgagtggatgggatggatcagtggtaacactggtaacacaaagtatacaccgaagctccaaggcagagtcaccatgaccacagacacatccacgagcacagcctacatggagctgaggagcctgagatctgacgacacggccgtgtattactgtgcgagagatcgtggtatagcggcaggccgtgcttttgatatctggggccaagggacaatggtcaccgtctcttca

SEQ ID NO: 448 - DNA, 351 bp, synthetic construct
  misc_feature 1..351: note = Synthetic antibody
  source 1..351: mol_type = other DNA
caggtgcagctggtgcagtctggacccacggtgaggaagcctggggcctcagtgagggtctcctgcaaggcttctggttacccctttaccagcaatggtatgagctgggtgcggcaggcccctggacaaggacttgagtggatgggatggatcagcactaacagcggaaacacaaactatgcgcagaaatttcagggcagagtcaccttgaccacagacacatcctcgaccactacgtacctggatctgaggagcctgacatctgacgacacggccatatattactgtgcgagagatgttcaatatcgtatggacgtctggggccaagggaccacggtcaccgtctcttca

SEQ ID NO: 449 - DNA, 387 bp, synthetic construct
  misc_feature 1..387: note = Synthetic antibody
  source 1..387: mol_type = other DNA
gaggtgcagctggtggagtctgggggaggcgtggtccagcccgggaggtccctgagactctcctgtgcagcctctggattcaccatcagcagttatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagttgaggacacggctgtgtattactgtgcgaaagatctcttattacgatattttgactggagccccaaatactactacaacggtatggacgtctggggccaagggacaatggtcaccgtctcctca

SEQ ID NO: 450 - DNA, 357 bp, synthetic construct
  misc_feature 1..357: note = Synthetic antibody
  source 1..357: mol_type = other DNA
caggtgcagctggtgcaatctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagtggcagtggctgacaccttctttgactactggggccagggaaccctggtcaccgtctcctca

SEQ ID NO: 451 - DNA, 348 bp, synthetic construct
  misc_feature 1..348: note = Synthetic antibody
  source 1..348: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagacggctacggtatggacgtctggggccaagggaccacggtcaccgtctcctca

SEQ ID NO: 452 - DNA, 366 bp, synthetic construct
  misc_feature 1..366: note = Synthetic antibody
  source 1..366: mol_type = other DNA
caggtgcagctggtgcagtctgggggaggcttggtccagcctggggggtccctgagactctcctgtgcagcctctggattcacctttagcagctatgccatgagctgggtccgccaggctccagggaaggggctggagtgggtctcaactattaatgatagtggtggtagcacatactacgcagactccgtgaagggccggttcaccatctccagagacaattccaagaacacgctgtatctgcaaatgaacagcctgagagccgaggacacggccgtatattactgtgcgaaagatcatattacgatttttggagtggccccgtttgactactggggcctgggaaccctggtcaccgtctcctca

SEQ ID NO: 453 - DNA, 372 bp, synthetic construct
  misc_feature 1..372: note = Synthetic antibody
  source 1..372: mol_type = other DNA
caggtgcagctacagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagacaagatgaatcggcagccgatgatgcttttgatatctggggccaaggaaccctggtcatcgtctcctca

SEQ ID NO: 454 - DNA, 351 bp, synthetic construct
  misc_feature 1..351: note = Synthetic antibody
  source 1..351: mol_type = other DNA
caggtgcagctggtgcagtctggagatgaggtgaagaaggctggggcctcagtgagggtctcctgcaaggcttctggttacacgtttcccgacaacggtatcagttgggtgcgacaggcccctggacacgggcttgagtggatgggttggatcaacgttgacactggatacacccactatgcacagagggtccaggacagagtcgtcatgaccacagacacgtccacgaacacactccacatgacattgaggagcctgacaacggacgacacggccgtttattattgtgcgagagaaatcgagggagtgtggacagcttggggccagggaaccctggtcatcgtctcctca

SEQ ID NO: 455 - DNA, 357 bp, synthetic construct
  misc_feature 1..357: note = Synthetic antibody
  source 1..357: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgaggtcggactacggtgactcctactttgactactggggccagggaaccctggtcagcgtctcctca

SEQ ID NO: 456 - DNA, 357 bp, synthetic construct
  misc_feature 1..357: note = Synthetic antibody
  source 1..357: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagcctctatggtcgagacttactttgactactggggccagggaaccatggtcaccgtctcttca

SEQ ID NO: 457 - DNA, 384 bp, synthetic construct
  misc_feature 1..384: note = Synthetic antibody
  source 1..384: mol_type = other DNA
gaggtgcagctggtgcagtctggagctgaggtgaagaagtctggggcctcagtgaaggtctcctgcaagtcttctggttacacctttaccaactatggtatcagttgggtgcgacaagccccgggacaagggcttgagtggatgggctggatcagcggtgacgctggtgacacaaaatttgcacagaagttccagggcagagtcaccatgacgacagacacatccacgactacaacgtacatggagctgaggagcctgagatctgacgacacggccgtgtattactgtgcgagagatggggaatatattcctatgtttcggggatttgacaactactacggtctggacgtctggggccaagggaccctggtcagcgtctcctca

SEQ ID NO: 458 - DNA, 363 bp, synthetic construct
  misc_feature 1..363: note = Synthetic antibody
  source 1..363: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagcgggattatacctgataatccctacggtatggacgtctggggccaagggacaatggtcagcgtctcctca

SEQ ID NO: 459 - DNA, 366 bp, synthetic construct
  misc_feature 1..366: note = Synthetic antibody
  source 1..366: mol_type = other DNA
gaggtgcagctgttggactcgggcccacgactgttgaagccttcggagaccctgtccctcacctgcggtgtgtcaggtgtctccaccagcagtactcattggtggagttgggtccgccagaccccagggaaggggctggagtggattggtgaaatccttcataatggaaacaccaactccaatccgtccctcaagagtcgagtctccatgtcgattgacaagtcgaggaaccaattctccctacaactgaagtctatgaccgccgcggacacggccgtctactactgtgcgtcagcgtcgcctaactggggagctagcggggcttttgatgcctgggcccaagggacaatggtcaccgtctcctca

SEQ ID NO: 460 - DNA, 354 bp, synthetic construct
  misc_feature 1..354: note = Synthetic antibody
  source 1..354: mol_type = other DNA
caggtgcagctggtgcagtctggagctgaggtgaagaagcctggggcctcagtgaaggtctcctgcaaggcttctggttacacctttaccaggtatggtttcagctgggtgcgacaggcccctggacaagggcttgagtggatgggatggatcagcggttacaatggtaatacaaagtatgcacagaagttccagggcagagtcaccatgactacagacacatctacgagcacagcctacatggagctgaggagcctaacatctaacgacacggccgtttattactgtgcgagagatcggggtagtggttatcttgaatactggggcccgggaaccctggtcaccgtctcttca

SEQ ID NO: 461 - DNA, 375 bp, synthetic construct
  misc_feature 1..375: note = Synthetic antibody
  source 1..375: mol_type = other DNA
caggtgcagctggtgcagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatcgatatagcagcagctggtactcccctgcttttgatatctggggccaagggacaacggtcagcgtctcctca

SEQ ID NO: 462 - DNA, 348 bp, synthetic construct
  misc_feature 1..348: note = Synthetic antibody
  source 1..348: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagtgggcagctatggtgactactggggccagggaaccctggtcaccgtctcctca

SEQ ID NO: 463 - DNA, 363 bp, synthetic construct
  misc_feature 1..363: note = Synthetic antibody
  source 1..363: mol_type = other DNA
caggtgcagctggtgcagtctggggctgaggtgaagaagcctggggcctcagtgaaggtttcctgcaaggcatctgggtacaccttcacccactcctatatacactgggtgcgccaggcccctggacaagggcttgagtggatgggagtaatcaaccctgttggtggtagcacaacctacgcacagaggttccagggcagagtcaccatgaccagggacacgtccacgagcacagtctacatggagctgagcagcctgagatctgaggacacggccgtgtattactgtgctagagcggggggagatggctacaatccttactttgactactggggccagggaaccctggtcaccgtctcctca

SEQ ID NO: 464 - DNA, 354 bp, synthetic construct
  misc_feature 1..354: note = Synthetic antibody
  source 1..354: mol_type = other DNA
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggccgtgtattactgtgcgagagtcggagatcttgatgcttttgatatctggggccaagggacaatggtcaccgtctcttca

SEQ ID NO: 465 - DNA, 360 bp, synthetic construct
  misc_feature 1..360: note = Synthetic antibody
  source 1..360: mol_type = other DNA
caggtgcagctggtggagtctgggggaggcttggtcaagcctggagggtccctgagactctcctgtgcagcctctggattcaccttcagtgaccactacatgagctggatccgccaggctccaggaaaggggctggagtgggtttcatacattactactactggtagtaccatgtcctacgcagactctgtgaagggccgcttcaccatttccagggacaactccaagaactcactgcatctgcaattgagcagcctgagagccgaggacacggccgtatattactgtgcgagagattccatacatgtggatggtgcttttgatatttggggccaagggacaatggtcaccgtctcctca

SEQ ID NO: 466 - DNA, 351 bp, synthetic construct
  misc_feature 1..351: note = Synthetic antibody
  source 1..351: mol_type = other DNA
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatcggggagggtggttcgacccctggggccagggaaccctggtcatcgtctcctca

SEQ ID NO: 467 - DNA, 357 bp, synthetic construct
  misc_feature 1..357: note = Synthetic antibody
  source 1..357: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagagtcgtatagcagctcgtcctttgactactggggccagggaaccacggtcagcgtctcctca

SEQ ID NO: 468 - DNA, 372 bp, synthetic construct
  misc_feature 1..372: note = Synthetic antibody
  source 1..372: mol_type = other DNA
caggtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagggcgaaggaatattgtagtggtggtagctgccaagatgcttttgatatctggggccaagggacaatggtcagcgtctcctca

SEQ ID NO: 469 - DNA, 369 bp, synthetic construct
  misc_feature 1..369: note = Synthetic antibody
  source 1..369: mol_type = other DNA
gaagtgcagctggtgcagtctggggctgaggtgaagaagcctggggcctcagtgaaggttgcctgcaaggcatctgggtacaccttcaccgactattatatacactgggtgcgacaggcccctggacaagggcttgagtggatgggaatagtcaacgctaatggtggtgacacaaactacgcacaacagttccagggcagagtcaccatgaccagggacacgtccacgagcacagtctatttggagttgaccagcctgagatctgacgacacggccgtatattactgtgctagagattggtattactattcttcggggagtttcccttttgactactggggccagggaaccctggtcagcgtctcctca

SEQ ID NO: 470 - DNA, 369 bp, synthetic construct; identical to SEQ ID NO: 469
  misc_feature 1..369: note = Synthetic antibody
  source 1..369: mol_type = other DNA

SEQ ID NO: 471 - DNA, 354 bp, synthetic construct
  misc_feature 1..354: note = Synthetic antibody
  source 1..354: mol_type = other DNA
caggtgcagctggtgcagtctggagctgaggtgaagaagcctggggcctcagtgaaggtctcctgcaaggcttctggttacatcttcaacacctatggtatcagttgggtgcgacaggcccctggacaaggacttgagtggatgggatggaccaacgtttacaatagtaacacagacagtggacacaagttccagggcagagtcaccatgaccacagacacatccacggacacagcctacatggaactgaggagcctgagatctgacgacacggccgtgtattactgtgcgagagacccgggctcggctatttttgatatgtggggccaagggacaatggtcaccgtctcctca

SEQ ID NO: 472 - DNA, 351 bp, synthetic construct
  misc_feature 1..351: note = Synthetic antibody
  source 1..351: mol_type = other DNA
caggtggagctgcaggagtcgggcccaggactggtgaagccttcggagaccctgtccctcacttgcactgtctctggtgacgccatcaaagataagtactggagttgggtccggcagcccccagggaagggactggagtggattggctacatctctaacagtggaagcaccaactacaacccctccctcaagagtcgagtcagtctatcagtagacacgtccaagaatcagttctccctgaagcagacctctgtgaccgctgcggacacggccacatattactgtgcgagaggcagcggttatactgcttttgatatctggggccaagggacaatggtcaccgtctcctca

SEQ ID NO: 473 - DNA, 363 bp, synthetic construct
  misc_feature 1..363: note = Synthetic antibody
  source 1..363: mol_type = other DNA
caggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgtcgacccctatagtagtggttattttgactactggggccagggaaccctggtcatcgtctcctca

SEQ ID NO: 474 - DNA, 351 bp, synthetic construct
  misc_feature 1..351: note = Synthetic antibody
  source 1..351: mol_type = other DNA
caggtgcagctggtggagtctgggggaggcttggtacagcctggggggtccctgagactctcctgtgcagcctctggattcacctttagcaactatgccatgagctgggtccgccaggctccagggaaggggctggagtgggtctcagccattagtggtagtagtattagcacatactacgcagactccgtgaagggccggttcaccatctccagagacaattccaagaacacgctttatctgcaaatgaacagcctgagagccgaggacacggccgtatattactgtgcgtcctcgggtttcgggtcctttgactactggggccagggcaccctggtcaccgtctcttca

SEQ ID NO: 475 - DNA, 354 bp, synthetic construct
  misc_feature 1..354: note = Synthetic antibody
  source 1..354: mol_type = other DNA
caggtgcagctggtggagtctgggggaggcttggtacagcctggggggtccctgagactctcctgtgcagcctctggattcaccttaagtagctatgccatgagctgggtccgccaggctccaggggaggggctggagtgggtctcaagtattaatagtggtggtagcacatactacgcaggctccgtgaggggccggttcaccatctccagagacaattccaagaacacgttgtatctgcaaatgaacagcctgagagccgaggacacggccgtatattactgtgcgaaagcgaagagttatagcagtgaccttgaccactggggccagggaaccacggtcaccgtctcttca

SEQ ID NO: 476 - DNA, 369 bp, synthetic construct
  misc_feature 1..369: note = Synthetic antibody
  source 1..369: mol_type = other DNA
caggtgcagatggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatcttctcggggatagtgggagctaccctgcttttgatatctggggccaagggacaatggtcaccgtctcttca

SEQ ID NO: 477 - DNA, 351 bp, synthetic construct
  misc_feature 1..351: note = Synthetic antibody
  source 1..351: mol_type = other DNA
caggtgcagatggtggagtctgggggagacttggtacagccagggcaatccctgagactctcctgtgtaacttctggattcccctttgcggactatgctatgagctggttccgccaggctccagggcagcggccggagtggataggttacattaaaagcaagactagagatgggacaacaaaatacgccgagtctctgcgaggcagattcaccatctcaagagatgattccaaaagcatcgcctatctacaaatgaacaacttaaaaagggaagacacagccgtctattactgtgccctcgggggagcagcagctctatggggcccgggaaccctggtcagcgtctcctca

SEQ ID NO: 478 - DNA, 360 bp, synthetic construct
  misc_feature 1..360: note = Synthetic antibody
  source 1..360: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgattagtagtggttattattataacccatttgactactggggccagggaaccctggtcaccgtctcctca

SEQ ID NO: 479 - DNA, 351 bp, synthetic construct
  misc_feature 1..351: note = Synthetic antibody
  source 1..351: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagggggataccagccactggattactggggccagggaaccctggtcatcgtctcctca

SEQ ID NO: 480 - DNA, 363 bp, synthetic construct
  misc_feature 1..363: note = Synthetic antibody
  source 1..363: mol_type = other DNA
caaatgcagctggagcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagctgggggaatccattactatgatagtttagactactggggccagggaaccctggtcaccgtctcctca

SEQ ID NO: 481 - DNA, 366 bp, synthetic construct
  misc_feature 1..366: note = Synthetic antibody
  source 1..366: mol_type = other DNA
caggtgcagctggtggagtctgggggaggcttggtaaagccgggggggtctctaagactctcctgtgcagcctctggattcaccttcgcgaatgcctggatgggctgggttcgccaggctccagggaaggggctgcagtgggtatcccatattaagcccagttctagcggcgggaggacaacagactacgatgcacccgtgaaaggccgattcaccatctcaagagatgattcaaacgacacggtgtatctgcaaatgaacagcctcaagaccgaagacacaggcgtctattactgtgtcacaggaagaagggtcgcgaagacttttgatttctggggccaggggacaacggtcagcgtctcctca

SEQ ID NO: 482 - protein, 119 aa, synthetic construct
  REGION 1..119: note = Synthetic antibody
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARGNQGDTAFDIWGQGTTVIVSS

SEQ ID NO: 483 - protein, 119 aa, synthetic construct
  REGION 1..119: note = Synthetic antibody
QVQLVQSGAEVKKPGASVKVSCKASGYTFSDYGFSWVRQAPGQGLEWMGWISGYNGNTNYAQKFQGRVTMTIDASTSTAYMELRSLKSDDTAVYYCARLLLGYYGMDVWGQGTTVTVSS

SEQ ID NO: 484 - protein, 121 aa, synthetic construct
  REGION 1..121: note = Synthetic antibody
QVQLVQSGAEVKKPGASVKVSCRTSGYTFSSYGISWVRQAPGQGLEWMGWISGNTGNTKYTPKLQGRVTMTTDTSTSTAYMELRSLRSDDTAVYYCARDRGIAAGRAFDIWGQGTMVTVSS

SEQ ID NO: 485 - protein, 117 aa, synthetic construct
  REGION 1..117: note = Synthetic antibody
QVQLVQSGPTVRKPGASVRVSCKASGYPFTSNGMSWVRQAPGQGLEWMGWISTNSGNTNYAQKFQGRVTLTTDTSSTTTYLDLRSLTSDDTAIYYCARDVQYRMDVWGQGTTVTVSS

SEQ ID NO: 486 - protein, 129 aa, synthetic construct
  REGION 1..129: note = Synthetic antibody
EVQLVESGGGVVQPGRSLRLSCAASGFTISSYGMHWVRQAPGKGLEWVAVISYDGSNKYYADSVKGRFTISRDNSKNTLYLQMNSLRVEDTAVYYCAKDLLLRYFDWSPKYYYNGMDVWGQGTMVTVSS

SEQ ID NO: 487 - protein, 119 aa, synthetic construct
  REGION 1..119: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVAVADTFFDYWGQGTLVTVSS

SEQ ID NO: 488 - protein, 116 aa, synthetic construct
  REGION 1..116: note = Synthetic antibody
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDGYGMDVWGQGTTVTVSS

SEQ ID NO: 489 - protein, 122 aa, synthetic construct
  REGION 1..122: note = Synthetic antibody
QVQLVQSGGGLVQPGGSLRLSCAASGFTFSSYAMSWVRQAPGKGLEWVSTINDSGGSTYYADSVKGRFTISRDNSKNTLYLQMNSLRAEDTAVYYCAKDHITIFGVAPFDYWGLGTLVTVSS

SEQ ID NO: 490 - protein, 124 aa, synthetic construct
  REGION 1..124: note = Synthetic antibody
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARQDESAADDAFDIWGQGTLVIVSS

SEQ ID NO: 491 - protein, 117 aa, synthetic construct
  REGION 1..117: note = Synthetic antibody
QVQLVQSGDEVKKAGASVRVSCKASGYTFPDNGISWVRQAPGHGLEWMGWINVDTGYTHYAQRVQDRVVMTTDTSTNTLHMTLRSLTTDDTAVYYCAREIEGVWTAWGQGTLVIVSS

SEQ ID NO: 492 - protein, 119 aa, synthetic construct
  REGION 1..119: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSDYGDSYFDYWGQGTLVSVSS

SEQ ID NO: 493 - protein, 119 aa, synthetic construct
  REGION 1..119: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARASMVETYFDYWGQGTMVTVSS

SEQ ID NO: 494 - protein, 128 aa, synthetic construct
  REGION 1..128: note = Synthetic antibody
EVQLVQSGAEVKKSGASVKVSCKSSGYTFTNYGISWVRQAPGQGLEWMGWISGDAGDTKFAQKFQGRVTMTTDTSTTTTYMELRSLRSDDTAVYYCARDGEYIPMFRGFDNYYGLDVWGQGTLVSVSS

SEQ ID NO: 495 - protein, 121 aa, synthetic construct
  REGION 1..121: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASGIIPDNPYGMDVWGQGTMVSVSS

SEQ ID NO: 496 - protein, 122 aa, synthetic construct
  REGION 1..122: note = Synthetic antibody
EVQLLDSGPRLLKPSETLSLTCGVSGVSTSSTHWWSWVRQTPGKGLEWIGEILHNGNTNSNPSLKSRVSMSIDKSRNQFSLQLKSMTAADTAVYYCASASPNWGASGAFDAWAQGTMVTVSS

SEQ ID NO: 497 - protein, 118 aa, synthetic construct
  REGION 1..118: note = Synthetic antibody
QVQLVQSGAEVKKPGASVKVSCKASGYTFTRYGFSWVRQAPGQGLEWMGWISGYNGNTKYAQKFQGRVTMTTDTSTSTAYMELRSLTSNDTAVYYCARDRGSGYLEYWGPGTLVTVSS

SEQ ID NO: 498 - protein, 125 aa, synthetic construct
  REGION 1..125: note = Synthetic antibody
QVQLVQSGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTDRYSSSWYSPAFDIWGQGTTVSVSS

SEQ ID NO: 499 - protein, 116 aa, synthetic construct
  REGION 1..116: note = Synthetic antibody
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVGSYGDYWGQGTLVTVSS

SEQ ID NO: 500 - protein, 121 aa, synthetic construct
  REGION 1..121: note = Synthetic antibody
QVQLVQSGAEVKKPGASVKVSCKASGYTFTHSYIHWVRQAPGQGLEWMGVINPVGGSTTYAQRFQGRVTMTRDTSTSTVYMELSSLRSEDTAVYYCARAGGDGYNPYFDYWGQGTLVTVSS

SEQ ID NO: 501 - protein, 118 aa, synthetic construct
  REGION 1..118: note = Synthetic antibody
QVQLVESGGGLVKPGGSLRLSCAASGFTFSDYYMSWIRQAPGKGLEWVSYISSSSSYTNYADSVKGRFTISRDNAKNSLYLQMNSLRAEDTAVYYCARVGDLDAFDIWGQGTMVTVSS

SEQ ID NO: 502 - protein, 120 aa, synthetic construct
  REGION 1..120: note = Synthetic antibody
QVQLVESGGGLVKPGGSLRLSCAASGFTFSDHYMSWIRQAPGKGLEWVSYITTTGSTMSYADSVKGRFTISRDNSKNSLHLQLSSLRAEDTAVYYCARDSIHVDGAFDIWGQGTMVTVSS

SEQ ID NO: 503 - protein, 117 aa, synthetic construct
  REGION 1..117: note = Synthetic antibody
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDRGGWFDPWGQGTLVIVSS

SEQ ID NO: 504 - protein, 119 aa, synthetic construct
  REGION 1..119: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARESYSSSSFDYWGQGTTVSVSS

SEQ ID NO: 505 - protein, 124 aa, synthetic construct
  REGION 1..124: note = Synthetic antibody
QVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARAKEYCSGGSCQDAFDIWGQGTMVSVSS

SEQ ID NO: 506 - protein, 123 aa, synthetic construct
  REGION 1..123: note = Synthetic antibody
EVQLVQSGAEVKKPGASVKVACKASGYTFTDYYIHWVRQAPGQGLEWMGIVNANGGDTNYAQQFQGRVTMTRDTSTSTVYLELTSLRSDDTAVYYCARDWYYYSSGSFPFDYWGQGTLVSVSS

SEQ ID NO: 507 - protein, 123 aa, synthetic construct; identical to SEQ ID NO: 506
  REGION 1..123: note = Synthetic antibody

SEQ ID NO: 508 - protein, 118 aa, synthetic construct
  REGION 1..118: note = Synthetic antibody
QVQLVQSGAEVKKPGASVKVSCKASGYIFNTYGISWVRQAPGQGLEWMGWTNVYNSNTDSGHKFQGRVTMTTDTSTDTAYMELRSLRSDDTAVYYCARDPGSAIFDMWGQGTMVTVSS

SEQ ID NO: 509 - protein, 117 aa, synthetic construct
  REGION 1..117: note = Synthetic antibody
QVELQESGPGLVKPSETLSLTCTVSGDAIKDKYWSWVRQPPGKGLEWIGYISNSGSTNYNPSLKSRVSLSVDTSKNQFSLKQTSVTAADTATYYCARGSGYTAFDIWGQGTMVTVSS

SEQ ID NO: 510 - protein, 121 aa, synthetic construct
  REGION 1..121: note = Synthetic antibody
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDVDPYSSGYFDYWGQGTLVIVSS

SEQ ID NO: 511 - protein, 117 aa, synthetic construct
  REGION 1..117: note = Synthetic antibody
QVQLVESGGGLVQPGGSLRLSCAASGFTFSNYAMSWVRQAPGKGLEWVSAISGSSISTYYADSVKGRFTISRDNSKNTLYLQMNSLRAEDTAVYYCASSGFGSFDYWGQGTLVTVSS

SEQ ID NO: 512 - protein, 118 aa, synthetic construct
  REGION 1..118: note = Synthetic antibody
QVQLVESGGGLVQPGGSLRLSCAASGFTLSSYAMSWVRQAPGEGLEWVSSINSGGSTYYAGSVRGRFTISRDNSKNTLYLQMNSLRAEDTAVYYCAKAKSYSSDLDHWGQGTTVTVSS

SEQ ID NO: 513 - protein, 123 aa, synthetic construct
  REGION 1..123: note = Synthetic antibody
QVQMVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLLGDSGSYPAFDIWGQGTMVTVSS

SEQ ID NO: 514 - protein, 117 aa, synthetic construct
  REGION 1..117: note = Synthetic antibody
QVQMVESGGDLVQPGQSLRLSCVTSGFPFADYAMSWFRQAPGQRPEWIGYIKSKTRDGTTKYAESLRGRFTISRDDSKSIAYLQMNNLKREDTAVYYCALGGAAALWGPGTLVSVSS

SEQ ID NO: 515 - protein, 120 aa, synthetic construct
  REGION 1..120: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAISSGYYYNPFDYWGQGTLVTVSS

SEQ ID NO: 516 - protein, 117 aa, synthetic construct
  REGION 1..117: note = Synthetic antibody
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGYQPLDYWGQGTLVIVSS

SEQ ID NO: 517 - protein, 121 aa, synthetic construct
  REGION 1..121: note = Synthetic antibody
QMQLEQSGAEVKKPGSSVKVSCKASGGTFSSYAISWVRQAPGQGLEWMGGIIPIFGTANYAQKFQGRVTITADKSTSTAYMELSSLRSEDTAVYYCARAGGIHYYDSLDYWGQGTLVTVSS

SEQ ID NO: 518 - protein, 122 aa, synthetic construct
  REGION 1..122: note = Synthetic antibody
QVQLVESGGGLVKPGGSLRLSCAASGFTFANAWMGWVRQAPGKGLQWVSHIKPSSSGGRTTDYDAPVKGRFTISRDDSNDTVYLQMNSLKTEDTGVYYCVTGRRVAKTFDFWGQGTTVSVSS

SEQ ID NO: 519 - DNA, 333 bp, synthetic construct
  misc_feature 1..333: note = Synthetic antibody
  source 1..333: mol_type = other DNA
cagtctgccctgaatcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcactccttatgtcttcggaactgggaccaaggtcaccgtccta

SEQ ID NO: 520 - DNA, 324 bp, synthetic construct
  misc_feature 1..324: note = Synthetic antibody
  source 1..324: mol_type = other DNA
gacatcgtgatgacccagtctccatcctccctgtctgcatctgtaggagacagagtcaccctcacttgccgggcaagtcagagcattggcaactatttaaattggtatcaacagaaaccagggaaagcccctaacctcctgatctatgctgcatccagtttgcaaagtggggtcccatcaaggttcagtggcagtggatctgggacagatttcactctcaccatcagcagtctgcagcctgaagattttgcaacttactactgtcaacagagtttcagtatccccccgataactttcggccaagggacacgactggagattaaa

SEQ ID NO: 521 - DNA, 330 bp, synthetic construct
  misc_feature 1..330: note = Synthetic antibody
  source 1..330: mol_type = other DNA
cagcctgtgctgactcagccaccctcagcgtctgggacccccggccagagggtcaccatctcttgttctggaagcagctccaacatcggaactaatcctgtaaactggtaccagcaagtcccaggaacgggccccaaactcctcatctataataataatcagtggccctcaggggtccctgaccgattctctggctccaagtctgccacctcagcctccctggccatctatgggctccagtctggggatgaggctcattattactgttcgtcatgggacgacagcctgagtgcgtgggtgttcggcggagggaccaaggtcaccgtccta

SEQ ID NO: 522 - DNA, 327 bp, synthetic construct
  misc_feature 1..327: note = Synthetic antibody
  source 1..327: mol_type = other DNA
cagtctgtgctgactcagccaccctcagcgtctgggacccccgggcagagggtcaccatctcttgttctggaagcaggtccaacatcggaagtaatcctgtaaactggtatcagcacctcccaggaacggcccccaaactcctcgtctatagtaataatcggcggccctcaggggtccctgaccgattctctggctccaagtctggcacctcagcctccctgtccatcagtgggctccagtctgaggatgaaggtgtttattactgtgcagcgtgggatgacaacgtgcacggagttttcggcggagggaccaaggtcaccgtccta

SEQ ID NO: 523 - DNA, 327 bp, synthetic construct
  misc_feature 1..327: note = Synthetic antibody
  source 1..327: mol_type = other DNA
cagtctgtgctgactcagccaccctcggtgtcagtggccccaggaaagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatccgggggtcttcggaactgggaccaaggtcaccgtccta

SEQ ID NO: 524 - DNA, 333 bp, synthetic construct
  misc_feature 1..333: note = Synthetic antibody
  source 1..333: mol_type = other DNA
cagcctgtgctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcagcaccccttatgtcttcggaactgggaccaaggtcaccgtccta

SEQ ID NO: 525 - DNA, 306 bp, synthetic construct
  misc_feature 1..306: note = Synthetic antibody
  source 1..306: mol_type = other DNA
cagtctccatcctccctgtctgcatctgtaggcgacagagtcaccatcacttgccggacaagtcacaccgttaacagctatctaaattggtatcaacaagaaccagggaaagcccctaaactcctgatctatgctgcatccaatttgcaaagtggggtcccgtcaaggttcagtggcagtggatctgggacagatttctctctcaccatccacaatctgcaacctgaagattttgcaacttactactgtcaacagagttacagaacccctctcacttttggccaggggaccaaagtggatatcaaa

SEQ ID NO: 526 - DNA, 330 bp, synthetic construct
  misc_feature 1..330: note = Synthetic antibody
  source 1..330: mol_type = other DNA
cagtctgtgctgactcagccaccctcagcgtctggaacccccgggcagagggtcaccatctcttgttctggaagcagctccaacatcggaggtaatcctgtaaactggtaccagcacctcccaggaacggcccccaagctcctcatctattataatgatcagcggccttcaggggtccctgaccgattctctggctccaagtctggcacctcagcctccctggccatcagtggactccagtctgaggatgagactgattattactgtgcaacatgggatgacagcctgaatggttgggtgtttggcggagggacccagctgaccgtccta

SEQ ID NO: 527 - DNA, 330 bp, synthetic construct
  misc_feature 1..330: note = Synthetic antibody
  source 1..330: mol_type = other DNA
cagtctgtgctgactcagccaccctcagcgtctggcacccccgggcagagggtcaccatctcttgttctggaagcaagtccaacgtcggaaataattttgtacattggtaccagcaactcccagggacggcccccaaactcctcctctatagcaatcttgagcggtcctcaggggtccctgagcgattctctggctccaagtctggcacctcagcctacctggccatcagtgggctccggtccgacgatgaggctgattattactgtgcagcatgggatgacagcctgcccggttgggtgttcggcggagggaccaagctgaccgtccta

SEQ ID NO: 528 - DNA, 330 bp, synthetic construct
  misc_feature 1..330: note = Synthetic antibody
  source 1..330: mol_type = other DNA
cagcctgtgctgactcagccaccctcagcgtctgggacccccgggcagagggtcaccatctcttgttctggaagcagctccaacatcggaagtaatcctctaaactggtaccagcaactcccaggaacggcccccaaactcctcatctataataataatcagcggccctcaggggtccctgaccgattctctggctccaagtctggcacctcagcctccctggccatcagtggactccagtctgaggatgaggctgattattactgtgcggcatgggatgacagcctgaatggttgggtgttcggcggagggacccagctgaccgtccta

SEQ ID NO: 529 - DNA, 333 bp, synthetic construct
  misc_feature 1..333: note = Synthetic antibody
  source 1..333: mol_type = other DNA
caggctgtggtgactcagcctgcctccgtgtctgggtctcctggacagtcggtcagcatctcctgcactggaaccagcagtgacgttggtggttttaactatgtctcctggtatcaacaacacccaggcaaagcccccaaactcatgatttatgatgtcagtaatcggccctcaggggtttctaatcgcttctctggctccaagtctggcaacacggcctccctgaccatctctgggctccaggctgaggacgaggctgattattactgcagctcatatacaagcagcaccgctccttatgtcttcggaactgggaccaaggtcaccgtccta

SEQ ID NO: 530 - DNA, 333 bp, synthetic construct
  misc_feature 1..333: note = Synthetic antibody
  source 1..333: mol_type = other DNA
cagtctgtgctgactcagcctcgctcagtgtccgggtctcctggacagtcagtcaccatctcctgcactggaaccagcagtgatgttggtggttataactatgtctcctggtaccaacaacacccaggcaaagcccccaaactcatgatttctgaggtcagtaagcggccctcaggggtccctgatcgcttctctggctccaagtctggcaacacggcctccctgaccgtctctgggctccaggctgaggatgaggctgattattactgctcctcatatgcaggcgggtacaccccctatgtcttcggaactgggaccaaggtcaccgtccta

SEQ ID NO: 531 - DNA, 330 bp, synthetic construct
  misc_feature 1..330: note = Synthetic antibody
  source 1..330: mol_type = other DNA
cagtctgtgctgactcagccaccctcagcgtctgcgacccccgggcagagggtcaccatctcttgttctggaagaaacgccaacatcggaagtaatcctgtaaactggtaccagcagctcccagggacggcccccagactcgtcatgtatagtaataatcagcggccctcaggggtccctgaccgattctctggctccaagtctggcacctcagcctccctggccatcagtgggctccagtctgaggacgaggctgattattactgtgaaggatgggatgacagcctgaatggctatgtcttcggaactgggaccaaggtcaccgtccta

SEQ ID NO: 532 - DNA, 336 bp, synthetic construct
  misc_feature 1..336: note = Synthetic antibody
  source 1..336: mol_type = other DNA
gatattgtgatgactcagtctccactctccctgcccgtcacccttggacagccggcctccatctcctgcacgtctagtcaaagcctcgtatacagtgatggaaacacctacttgaattggtttcaccagaggccaggccagtctccaaggcgcctattttataaggtttctaaccgggcctctggggtcccagacagattcagcggcagtgggtcaggcactgatttcacactgaaaatcagcagggtggaggctgaggatcttggggtttattactgcatgcaaggtacacactggccggtcaccttcggccaagggacacgactggagattaaa

SEQ ID NO: 533 - DNA, 330 bp, synthetic construct
  misc_feature 1..330: note = Synthetic antibody
  source 1..330: mol_type = other DNA
cagtctgtgctgactcagccaccctcagcgtctgcgacccccgggcagagggtcaccatctcttgttctggaagcagctccaacatcggaagtaatcctgtaaactggtatcagcaggtcccaggaatggcccccgaactcgtcatgtatagtgataatcagcggccctcaggggtccctgaccgattctctggctccaactctggcacctcagcctccctggccatcagtggcctccagtctgaggatgaggctgattattactgttcaacatgggatgacagcctgaatggtcttgtcttcggaactgggaccaaggtcaccgtccta

SEQ ID NO: 534 - DNA, 321 bp, synthetic construct
  misc_feature 1..321: note = Synthetic antibody
  source 1..321: mol_type = other DNA
gacgtccagatgacccagtctccatcctccctgtctgcatctgtaggagagagagtcaccctcacttgccgggcgagtcagggcatttacagttctttagcctggtatcagcaaaaaccaggtaaagcccctaaactcctactatatggtacgtcgcaattggaacgtggggtcccatccagattcagtggcagtggatctgggacggattacactctcaccatcagcagcctgcagcctgaagattctgcaacttattattgtcaacaacattacaatctacccatcaccttcggccaagggacacgactggagattaaa

SEQ ID NO: 535 - DNA, 330 bp, synthetic construct
  misc_feature 1..330: note = Synthetic antibody
  source 1..330: mol_type = other DNA
cagcctgtgctgactcagccaccctcagtgtctgaagcccccaggcagagggtcaccatctcctgttctggaagcagctccaacatcggaaataatgttgtaaactggtaccagcagctcccaggaaaggctcccaaactcctcatctattatgatgatctgctgccctcaggggtctctgaccgattctctggctccaagtctggcacctcagcctccctggccatcagtgggctccagtctgaggatgaggctgattattactgtgcaacatgggatgacagcctgaatggccctgtattcggcggagggaccaagctgaccgtccta

SEQ ID NO: 536 - DNA, 306 bp, synthetic construct
  misc_feature 1..306: note = Synthetic antibody
  source 1..306: mol_type = other DNA
cagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgctcactttcggcggagggaccaagctggagatcaaa

SEQ ID NO: 537 - DNA, 303 bp, synthetic construct
  misc_feature 1..303: note = Synthetic antibody
  source 1..303: mol_type = other DNA
cagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttatctcactttcggcggagggaccaaagtggatatcaaa

SEQ ID NO: 538 - DNA, 330 bp, synthetic construct
  misc_feature 1..330: note = Synthetic antibody
  source 1..330: mol_type = other DNA
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaaggtcaccgtccta

SEQ ID NO: 539 - DNA, 324 bp, synthetic construct
  misc_feature 1..324: note = Synthetic antibody
  source 1..324: mol_type = other DNA
tcctatgagctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcattgggtgttcggcggagggaccaagctgaccgtccta

SEQ ID NO: 540 - DNA, 306 bp, synthetic construct
  misc_feature 1..306: note = Synthetic antibody
  source 1..306: mol_type = other DNA
cagtctccatcctccctgtctgcatctgtaggagacagagtcaccatcacttgccgggcaagtcagagcataagcaactttgtaaattggtatcagcagaaaccaggggaagtccctaagctcctgatctacggtgcatccagtttgcagagtggggtcccatcaaggttcagtggcagtggatctgggacggatttcactctcaccatcagcagtctgcaacctgaagattttgcgacttactactgtcaacagggttacagtgtcccactcactttcggcggagggactacagtggatatcaaa

SEQ ID NO: 541 - DNA, 333 bp, synthetic construct
  misc_feature 1..333: note = Synthetic antibody
  source 1..333: mol_type = other DNA
cagcctgtgctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaatcggccctcgggggtttctaatcgcttctctggctccaagtctggcaactcggcctccctgaccatctctgggctccaggctgaggacgaggctgattattactgcagctcatatacaagcagcagcactccctatgtcttcggaactgggaccaaggtcaccgtccta

SEQ ID NO: 542 - DNA, 309 bp, synthetic construct
  misc_feature 1..309: note = Synthetic antibody
  source 1..309: mol_type = other DNA
cagtctccatcctccttgtctgcatctgtaggagacagagtcaccatcacttgccgggcaagtcagagcattaggacctacttaaattggtatcagcagaaaccaggcaaagaccctgaactcctgatctatgctgcaaccagtttgcaaagtggggtcccatcaaggttcactggcagcggatctgggacagacttcactctcaccatcagcagtctgcagcctgaagattttgcaacttattactgtcaacagagttacagtgcccccgtgttcacttttggccaggggaccaagctggagatcaaa

SEQ ID NO: 543 - DNA, 321 bp, synthetic construct
  misc_feature 1..321: note = Synthetic antibody
  source 1..321: mol_type = other DNA
gccatccagatgacccagtctccatcctccctgtctgcatctgtaggagacagagtctccatcacttgccgggcaagtcagagcattagcagctttttaaattggtatcagcagagaccagggaaagcccctgagctcctgatctatggtgcatccagcttgcaaagtggggtcccatcaaggttcagtggcagtggatctgggacagatttcactctcaccatcagtagtctgcaacctgaagattttgcaacttactactgtcaacagagttacagtatcccgatcaccttcggccaagggacacagctggagatcaaa

SEQ ID NO: 544 - DNA, 321 bp, synthetic construct
  misc_feature 1..321: note = Synthetic antibody
  source 1..321: mol_type = other DNA
gacgtccagatgacccagtccccttcctctttatccgctagcgtgggcgatcgtgtgtccatcacttgtaaggcctcccaagatgtgtccatcgccgtggcttggtaccagcagaagcccggcaaggcccccaagctgctgatctactccgccagctatcgttacaccggcgtgcccgatcgtttctccggctccggatccggcaccgacttcactttaaccatctcctctttacagcccgaggacttcgccgtctactactgccagcagcactacatcacccctttaacattcggcgccggcaccaagctggagatcaaa

SEQ ID NO: 545 - DNA, 321 bp, synthetic construct
  misc_feature 1..321: note = Synthetic antibody
  source 1..321: mol_type = other DNA
gacgtccagatgacccagtctccatcctccctgtctgcatctgtaggagacagagtctccatcacttgccgggcaagtcagagcattagcagctttttaaattggtatcagcagagaccagggaaagcccctgagctcctgatctatggtgcatccagcttgcaaagtggggtcccatcaaggttcagtggcagtggatctgggacagatttcactctcaccatcagtagtctgcaacctgaagattttgcaacttactactgtcaacagagttacagtatcccgatcaccttcggccaagggacacgactggagatcaaa

SEQ ID NO: 546 - DNA, 321 bp, synthetic construct
  misc_feature 1..321: note = Synthetic antibody
  source 1..321: mol_type = other DNA
aacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgcccactttcggcggagggactacagtggagatcaaa

SEQ ID NO: 547 - DNA, 321 bp, synthetic construct
  misc_feature 1..321: note = Synthetic antibody
  source 1..321: mol_type = other DNA
gccatccagatgacccagtctccatcttccgtgtctgcatctgtaggagacagagtcaccatcacttgtcgggcgagtcagggtattggcagctggttagcctggtatcagcagaaaccagggaaagcccctaagctcctgatctctgttgcatccagtttgcaaagtggggtcccatcaaggttcagcggcagtggatctgggacagatttcactctcaccatcagcagcctgcagcctgaagattttgcaacttactattgtcaacaggctaacagtttcccgctcacttttggcggagggactacagtggagatcaaa

SEQ ID NO: 548 - DNA, 315 bp, synthetic construct
  misc_feature 1..315: note = Synthetic antibody
  source 1..315: mol_type = other DNA
cagccaccctcggtgtctgcagcccccaggcagagggtcaccatctcctgttctggaagcagctccaatatcggagataattctgttaactggtaccagcagctcccaggaaaggctcccaaactcctcatttatcttgatgatctcctgccctcaggggtctctggccgattctctggctccaagtctggcacctcagcctccctggtcatcagtggcctccagtctgaggatgaggctgattattactgtgcaacatgggatgacagcctgaacggtgtggttttcggcggagggaccaaggtcaccgtccta

SEQ ID NO: 549 - DNA, 339 bp, synthetic construct
  misc_feature 1..339: note = Synthetic antibody
  source 1..339: mol_type = other DNA
gatgttgtgatgacgcagtctccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaaggtggaaatcaaa

SEQ ID NO: 550 - DNA, 321 bp, synthetic construct
  misc_feature 1..321: note = Synthetic antibody
  source 1..321: mol_type = other DNA
aacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtacacttttggccaggggaccaagctggagatcaaa

SEQ ID NO: 551 - DNA, 319 bp, synthetic construct
  misc_feature 1..319: note = Synthetic antibody
  source 1..319: mol_type = other DNA
cagccgccctcggtgtctggggccccagggcagagggtcaccatctcctgcgctgggagcagctccaacatcggggcaggttatggtgtgcactggtatcaacaccttccaggaacagcccccaaactcctcatctatggtgacaacaatcggccctcaggggtcccttaccgattctctgggtccaagtctggcacctcagcctccctggccatcactggactccaggctgaggatgaggctgattatttctgccagtcctatgacagcagcctgggggttggtgtgttcggcggagggaccaaggtcaccgtcctac

SEQ ID NO: 552 - DNA, 321 bp, synthetic construct
  misc_feature 1..321: note = Synthetic antibody
  source 1..321: mol_type = other DNA
gccatccagttgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccccctcactttcggcggagggaccaagctggagatcaaa

SEQ ID NO: 553 - DNA, 321 bp, synthetic construct
  misc_feature 1..321: note = Synthetic antibody
  source 1..321: mol_type = other DNA
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccattcactttcggccctgggaccaagctggagatcaaa

SEQ ID NO: 554 - DNA, 321 bp, synthetic construct
  misc_feature 1..321: note = Synthetic antibody
  source 1..321: mol_type = other DNA
gacgtccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctctgacgttcggccaagggaccaagctggagatcaaa

SEQ ID NO: 555 - DNA, 321 bp, synthetic construct
  misc_feature 1..321: note = Synthetic antibody
  source 1..321: mol_type = other DNA
aacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcggacgttcggccaagggactacggtggaaatcaaa

SEQ ID NO: 556 - protein, 111 aa, synthetic construct
  REGION 1..111: note = Synthetic antibody
QSALNQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVTVL

SEQ ID NO: 557 - protein, 108 aa, synthetic construct
  REGION 1..108: note = Synthetic antibody
DIVMTQSPSSLSASVGDRVTLTCRASQSIGNYLNWYQQKPGKAPNLLIYAASSLQSGVPSRFSGSGSGTDFTLTISSLQPEDFATYYCQQSFSIPPITFGQGTRLEIK

SEQ ID NO: 558 - protein, 110 aa, synthetic construct
  REGION 1..110: note = Synthetic antibody
QPVLTQPPSASGTPGQRVTISCSGSSSNIGTNPVNWYQQVPGTGPKLLIYNNNQWPSGVPDRFSGSKSATSASLAIYGLQSGDEAHYYCSSWDDSLSAWVFGGGTKVTVL

SEQ ID NO: 559 - protein, 109 aa, synthetic construct
  REGION 1..109: note = Synthetic antibody
QSVLTQPPSASGTPGQRVTISCSGSRSNIGSNPVNWYQHLPGTAPKLLVYSNNRRPSGVPDRFSGSKSGTSASLSISGLQSEDEGVYYCAAWDDNVHGVFGGGTKVTVL

SEQ ID NO: 560 - protein, 109 aa, synthetic construct
  REGION 1..109: note = Synthetic antibody
QSVLTQPPSVSVAPGKTARITCGGNNIGSKSVHWYQQKPGQAPVLVVYDDSDRPSGIPERFSGSNSGNTATLTISRVEAGDEADYYCQVWDSSSDHPGVFGTGTKVTVL

SEQ ID NO: 561 - protein, 111 aa, synthetic construct
  REGION 1..111: note = Synthetic antibody
QPVLTQPASVSGSPGQSITISCTGTSSDVGGYNYVSWYQQHPGKAPKLMIYDVSNRPSGVSNRFSGSKSGNTASLTISGLQAEDEADYYCSSYTSSSTPYVFGTGTKVTVL

SEQ ID NO: 562 - protein, 102 aa, synthetic construct
  REGION 1..102: note = Synthetic antibody
QSPSSLSASVGDRVTITCRTSHTVNSYLNWYQQEPGKAPKLLIYAASNLQSGVPSRFSGSGSGTDFSLTIHNLQPEDFATYYCQQSYRTPLTFGQGTKVDIK

SEQ ID NO: 563 - protein, 110 aa, synthetic construct
  REGION 1..110: note = Synthetic antibody
QSVLTQPPSASGTPGQRVTISCSGSSSNIGGNPVNWYQHLPGTAPKLLIYYNDQRPSGVPDRFSGSKSGTSASLAISGLQSEDETDYYCATWDDSLNGWVFGGGTQLTVL

SEQ ID NO: 564 - protein, 110 aa, synthetic construct
  REGION 1..110: note = Synthetic antibody
QSVLTQPPSASGTPGQRVTISCSGSKSNVGNNFVHWYQQLPGTAPKLLLYSNLERSSGVPERFSGSKSGTSAYLAISGLRSDDEADYYCAAWDDSLPGWVFGGGTKLTVL

SEQ ID NO: 565 - protein, 110 aa, synthetic construct
  REGION 1..110: note = Synthetic antibody
QPVLTQPPSASGTPGQRVTISCSGSSSNIGSNPLNWYQQLPGTAPKLLIYNNNQRPSGVPDRFSGSKSGTSASLAISGLQSEDEADYYCAAWDDSLNGWVFGGGTQLTVL

SEQ ID NO: 566 - protein, 111 aa, synthetic construct
  REGION 1..111: note = Synthetic antibody
QAVVTQPASVSGSPGQSVSISCTGTSSDVGGFNYVSWYQQHPGKAPKLMIYDVSNRPSGVSNRFSGSKSGNTASLTISGLQAEDEADYYCSSYTSSTAPYVFGTGTKVTVL

SEQ ID NO: 567 - protein, 111 aa, synthetic construct
  REGION 1..111: note = Synthetic antibody
QSVLTQPRSVSGSPGQSVTISCTGTSSDVGGYNYVSWYQQHPGKAPKLMISEVSKRPSGVPDRFSGSKSGNTASLTVSGLQAEDEADYYCSSYAGGYTPYVFGTGTKVTVL

SEQ ID NO: 568 - protein, 110 aa, synthetic construct
  REGION 1..110: note = Synthetic antibody
QSVLTQPPSASATPGQRVTISCSGRNANIGSNPVNWYQQLPGTAPRLVMYSNNQRPSGVPDRFSGSKSGTSASLAISGLQSEDEADYYCEGWDDSLNGYVFGTGTKVTVL

SEQ ID NO: 569 - protein, 112 aa, synthetic construct
  REGION 1..112: note = Synthetic antibody
DIVMTQSPLSLPVTLGQPASISCTSSQSLVYSDGNTYLNWFHQRPGQSPRRLFYKVSNRASGVPDRFSGSGSGTDFTLKISRVEAEDLGVYYCMQGTHWPVTFGQGTRLEIK

SEQ ID NO: 570 - protein, 110 aa, synthetic construct
  REGION 1..110: note = Synthetic antibody
QSVLTQPPSASATPGQRVTISCSGSSSNIGSNPVNWYQQVPGMAPELVMYSDNQRPSGVPDRFSGSNSGTSASLAISGLQSEDEADYYCSTWDDSLNGLVFGTGTKVTVL

SEQ ID NO: 571 - protein, 107 aa, synthetic construct
  REGION 1..107: note = Synthetic antibody
DVQMTQSPSSLSASVGERVTLTCRASQGIYSSLAWYQQKPGKAPKLLLYGTSQLERGVPSRFSGSGSGTDYTLTISSLQPEDSATYYCQQHYNLPITFGQGTRLEIK

SEQ ID NO: 572 - protein, 110 aa, synthetic construct
  REGION 1..110: note = Synthetic antibody
QPVLTQPPSVSEAPRQRVTISCSGSSSNIGNNVVNWYQQLPGKAPKLLIYYDDLLPSGVSDRFSGSKSGTSASLAISGLQSEDEADYYCATWDDSLNGPVFGGGTKLTVL

SEQ ID NO: 573 - protein, 102 aa, synthetic construct
  REGION 1..102: note = Synthetic antibody
QSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKLEIK

SEQ ID NO: 574 - protein, 101 aa, synthetic construct
  REGION 1..101: note = Synthetic antibody
QSPSTLSASVGDRVTITCRASQSISSWLAWYQQKPGKAPKLLIYKASSLESGVPSRFSGSGSGTEFTLTISSLQPDDFATYYCQQYNSYLTFGGGTKVDIK

SEQ ID NO: 575 - protein, 110 aa, synthetic construct
  REGION 1..110: note = Synthetic antibody
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKVTVL

SEQ ID NO: 576 - protein, 108 aa, synthetic construct
  REGION 1..108: note = Synthetic antibody
SYELTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVL

SEQ ID NO: 577 - protein, 102 aa, synthetic construct
  REGION 1..102: note = Synthetic antibody
QSPSSLSASVGDRVTITCRASQSISNFVNWYQQKPGEVPKLLIYGASSLQSGVPSRFSGSGSGTDFTLTISSLQPEDFATYYCQQGYSVPLTFGGGTTVDIK

SEQ ID NO: 578 - protein, 111 aa, synthetic construct
  REGION 1..111: note = Synthetic antibody
QPVLTQPASVSGSPGQSITISCTGTSSDVGGYNYVSWYQQHPGKAPKLMIYDVSNRPSGVSNRFSGSKSGNSASLTISGLQAEDEADYYCSSYTSSSTPYVFGTGTKVTVL

SEQ ID NO: 579 - protein, 103 aa, synthetic construct
  REGION 1..103: note = Synthetic antibody
QSPSSLSASVGDRVTITCRASQSIRTYLNWYQQKPGKDPELLIYAATSLQSGVPSRFTGSGSGTDFTLTISSLQPEDFATYYCQQSYSAPVFTFGQGTKLEIK

SEQ ID NO: 580 - protein, 107 aa, synthetic construct
  REGION 1..107: note = Synthetic antibody
AIQMTQSPSSLSASVGDRVSITCRASQSISSFLNWYQQRPGKAPELLIYGASSLQSGVPSRFSGSGSGTDFTLTISSLQPEDFATYYCQQSYSIPITFGQGTQLEIK

SEQ ID NO: 581 - protein, 107 aa, synthetic construct
  REGION 1..107: note = Synthetic antibody
DVQMTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKLEIK

SEQ ID NO: 582 - protein, 107 aa, synthetic construct
  REGION 1..107: note = Synthetic antibody
DVQMTQSPSSLSASVGDRVSITCRASQSISSFLNWYQQRPGKAPELLIYGASSLQSGVPSRFSGSGSGTDFTLTISSLQPEDFATYYCQQSYSIPITFGQGTRLEIK

SEQ ID NO: 583 - protein, 107 aa, synthetic construct
  REGION 1..107: note = Synthetic antibody
NIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGGGTTVEIK

SEQ ID NO: 584 - protein, 107 aa, synthetic construct
  REGION 1..107: note = Synthetic antibody
AIQMTQSPSSVSASVGDRVTITCRASQGIGSWLAWYQQKPGKAPKLLISVASSLQSGVPSRFSGSGSGTDFTLTISSLQPEDFATYYCQQANSFPLTFGGGTTVEIK

SEQ ID NO: 585 - protein, 105 aa, synthetic construct
  REGION 1..105: note = Synthetic antibody
QPPSVSAAPRQRVTISCSGSSSNIGDNSVNWYQQLPGKAPKLLIYLDDLLPSGVSGRFSGSKSGTSASLVISGLQSEDEADYYCATWDDSLNGVVFGGGTKVTVL

SEQ ID NO: 586 - protein, 118 aa, synthetic construct
  REGION 1..118: note = Synthetic antibody
DVVMTQSPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKVEIKRTVAA

SEQ ID NO: 587 - protein, 107 aa, synthetic construct
  REGION 1..107: note = Synthetic antibody
NIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 588 - protein, 106 aa, synthetic construct
  REGION 1..106: note = Synthetic antibody
QPPSVSGAPGQRVTISCAGSSSNIGAGYGVHWYQHLPGTAPKLLIYGDNNRPSGVPYRFSGSKSGTSASLAITGLQAEDEADYFCQSYDSSLGVGVFGGGTKVTVL

SEQ ID NO: 589 - protein, 107 aa, synthetic construct
  REGION 1..107: note = Synthetic antibody
AIQLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKLEIK

SEQ ID NO: 590 - protein, 107 aa, synthetic construct
  REGION 1..107: note = Synthetic antibody
DIVMTQSPSSLSASVGDRVTITCRASQSISSYLNWYQQKPGKAPKLLIYAASSLQSGVPSRFSGSGSGTDFTLTISSLQPEDFATYYCQQSYSTPFTFGPGTKLEIK

SEQ ID NO: 591 - protein, 107 aa, synthetic construct
  REGION 1..107: note = Synthetic antibody
DVQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKLEIK

SEQ ID NO: 592 - protein, 107 aa, synthetic construct
  REGION 1..107: note = Synthetic antibody
NIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTTVEIK

SEQ ID NO: 593 - protein, 89 aa, synthetic construct
  REGION 1..89: note = Synthetic antibody
PKPTLWAEPGSVITQGSPVTLRCQGGQETQEYRLYREKKTALWITRIPQELVKKGQFPIPSITWEHAGRYRCYYGSKTAGRSESSDPLE

SEQ ID NO: 594 - protein, 84 aa, synthetic construct
  REGION 1..84: note = Synthetic antibody
PKPTLWAEPDSVITQGSPVTLSCQGSLEAQEYRLYREKKSASWITRIRPELVKNGQFHIPSITWEHTGRYGCQYYSRARWSELS

SEQ ID NO: 595 - protein, 63 aa, synthetic construct
  REGION 1..63: note = Synthetic antibody
PKPTLWAEPSVITQGSPVTLCQGEQEYRLYREKKSAWITRIELVKGQFIPSITWEHGRYCYSS

SEQ ID NO: 596 - protein, 155 aa, synthetic construct
  REGION 1..155: note = Synthetic antibody
WSAPSDPLDILIAGQFYDRVSLSVQPGPTCASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGV

SEQ ID NO: 597 - protein, 157 aa, synthetic construct
  REGION 1..157: note = Synthetic antibody
CSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSTHPSEPLELVVSGPSMGSSPPPTGPISTPAGPEDQPLTPTGSDPQSGLGRHLGV

SEQ ID NO: 598 - protein, 4 aa, synthetic construct
  REGION 1..4: note = Synthetic antibody
SLEA

SEQ ID NO: 599 - protein, 4 aa, synthetic construct
  REGION 1..4: note = Synthetic antibody
GQET

SEQ ID NO: 600 - protein, 4 aa, synthetic construct
  REGION 1..4: note = Synthetic antibody
KSAS

SEQ ID NO: 601 - protein, 4 aa, synthetic construct
  REGION 1..4: note = Synthetic antibody
KTAL

SEQ ID NO: 602 - protein, 4 aa, synthetic construct
  REGION 1..4: note = Synthetic antibody
IRPE

SEQ ID NO: 603 - protein, 4 aa, synthetic construct
  REGION 1..4: note = Synthetic antibody
IPQE

SEQ ID NO: 604 - protein, 7 aa, synthetic construct
  REGION 1..7: note = Synthetic antibody
RARWSEL

SEQ ID NO: 605 - protein, 8 aa, synthetic construct
  REGION 1..8: note = Synthetic antibody
DTAGRSES

SEQ ID NO: 606-607 - skipped (2 entries)

SEQ ID NO: 608 - protein, 5 aa, synthetic construct
  REGION 1..5: note = Synthetic antibody
IHEYP

SEQ ID NO: 609 - protein, 5 aa, synthetic construct
  REGION 1..5: note = Synthetic antibody
TYQSQ

SEQ ID NO: 610 - protein, 4 aa, synthetic construct
  REGION 1..4: note = Synthetic antibody
LNSD

SEQ ID NO: 611 - protein, 4 aa, synthetic construct
  REGION 1..4: note = Synthetic antibody
QSSK

SEQ ID NO: 612-613 - skipped (2 entries)